(19) United States
(12) Patent Application Publication
(10) Pub. No.: US 2011/0178092 A1
(43) Pub. Date: Jul. 21, 2011

(54) HIV-1 Protease Inhibitors

(76) Inventors: Akbar Ali, Worcester, MA (US); Michael D. Altman, Cambridge, MA (US); Saima G. Anjum, Shrewsbury, MA (US); Hong Cao, Westborough, MA (US); Sripriya Chellappan, (US); Miguel X. Fernandes, (US); Michael K. Gilson, North Potomac, MD (US); Visvaldas Kairys, (US); Nancy King, Oxford, MA (US); Ellen Nalivaika, Worcester, MA (US); Moses Prabu, Worcester, MA (US); Tariq M. Rana, Shrewsbury, MA (US); Kiran Kumar Sai, Worcester, MA (US); Celia A. Schiffer, Shrewsbury, MA (US); Bruce Tidor, Lexington, MA (US)

(21) Appl. No.: 11/960,120

(22) Filed: Dec. 19, 2007

Related U.S. Application Data
(63) Continuation-in-part of application No. PCT/US06/24109, filed on Jun. 21, 2006.
Application No. PCT/US06/24109 is a continuation-in-part of application No. PCT/US06/24108, filed on Jun. 21, 2006.
(60) Provisional application No. 60/693,134, filed on Jun. 22, 2005.
(60) Provisional application No. 60/749,902, filed on Dec. 12, 2005.
(60) Provisional application No. 60/810,234, filed on Jun. 2, 2006.
(60) Provisional application No. 60/919,896, filed on Mar. 23, 2007.
(60) Provisional application No. 60/919,819, filed on Mar. 23, 2007.
(60) Provisional application No. 60/941,786, filed on Jun. 4, 2007.
(60) Provisional application No. 60/941,829, filed on Jun. 4, 2007.

(51) Int. Cl. A61K 31/501; C07D 403/12; A61K 31/4178; C07D 401/14; A61K 31/4439; C07D 413/12; A61K 31/422; C07C 311/29; A61K 31/18; C07D 277/36; A61K 31/426; C07D 277/64; A61K 31/428; C07C 233/81; A61K 31/166; C07D 215/48; A61K 31/47; A61P 31/18

(52) U.S. Cl. 514/252.01; 548/315.7; 514/397; 546/274.4; 514/341; 544/238; 548/243; 514/380; 564/89; 514/600; 548/187; 514/369; 548/178; 514/367; 564/158; 514/616; 546/169; 514/311

(57) ABSTRACT

Described are novel protease inhibitors and methods for using said protease inhibitors in the treatment of human immunodeficiency virus (HIV) infection.

### RELATED APPLICATIONS

The present application is a continuation-in-part of International Application No. PCT/US06/024109, filed Jun. 21, 2006, which claims priority to U.S. Provisional Patent Application Ser. No. 60/693,134, filed on Jun. 22, 2005, U.S. Provisional Patent Application Ser. No. 60/749,902, filed on Dec. 12, 2005, and U.S. Provisional Patent Application Ser. No. 60/810,234 filed on Jun. 2, 2006; a continuation-in-part of International Application No. PCT/US06/024108, filed Jun. 21, 2006; which claims priority to U.S. Provisional Patent Application Ser. No. 60/693,134, filed on Jun. 22, 2005; and claims priority to U.S. Provisional Application Ser. No. 60/919,896, filed Mar. 23, 2007, U.S. Provisional Application Ser. No. 60/919,819, filed Mar. 23, 2007, U.S. Provisional Application Ser. No. 60/941,786, filed Jun. 4, 2007, and U.S. Provisional Application Ser. No. 60/941,829, filed Jun. 4, 2007, all of which are hereby incorporated by reference in their entirety.

### GOVERNMENT SUPPORT

This invention was made with government support under Grant No. P01 GM 066524 awarded by the National Institutes of Health/National Institute of Allergy and Infectious Diseases. The government has certain rights in the invention.

### BACKGROUND OF THE INVENTION

Human immunodeficiency virus type 1 (HIV-1) protease plays a critical role in the virus life cycle by processing the viral Gag and Gag-Pol polyproteins into structural and functional proteins essential for viral maturation. Inhibition of HIV-1 protease leads to the production of noninfectious virus particles and hence is a promising therapeutic target for antiviral therapy in AIDS patients.

Protease inhibitors (PIs) are potent antiretroviral drugs for the treatment of patients infected with Human Immunodeficiency Virus (HIV). Several known PIs are recommended as part of the “preferred regimen” for patients in the guidelines of International AIDS Society-USA (IAS-USA) and the U.S. Department of Health and Human Services (DHHS). However, use of these drugs has sometimes been associated with the development of irreversible HIV resistance, due to mutation of the virus.

In fact, HIV-1 protease inhibitors represent the most potent anti-AIDS drugs reported to date and are essential components of highly active antiretroviral therapy (HAART). In the last decade, structure based drug design has led to the discovery of eight FDA approved drugs and several others in advanced clinical trials. Currently marketed HIV-1 protease inhibitors, saquinavir, indinavir, ritonavir, nelfinavir, amprenavir, lopinavir, atazanavir, and tipranavir, are all competitive inhibitors that bind in the active site of the enzyme. Except the newly approved drug tipranavir, all approved inhibitors have been developed based on transition state mimetic concept and contain various noncleavable dipeptide isosteres as core scaffolds to mimic the transition state of HIV-1 protease substrates. The development and clinical introduction of anti-AIDS HIV-1 protease inhibitors is regarded as major success of structure based drug design.

Anti-AIDS chemotherapy based on HIV-1 protease and reverse-transcriptase inhibitors has been remarkably successful in decreasing the mortality rate in HIV-1 infected patients. However, the emergence of HIV-1 mutants that are resistant to current drug regimens is a critical factor in the clinical failure of antiviral therapy. In general, drug resistance occurs when mutations in a target protein allow that protein to retain function while in the presence of a drug. In the case of HIV-1 protease, drug resistance typically occurs when, even in the presence of protease inhibitors, the enzyme is able to cleave the Gal and Pol polypeptides in at least nine different locations, allowing viral mutation. Viral resistance is regarded as a critical factor in clinical failure of antiviral therapy. The relatively rapid appearance of resistant viral mutants among treated HIV patients is attributable to the virus' high rate of replication, coupled with a high intrinsic rate of mutation due to the infidelity of the HIV reverse transcriptase. Further, current HIV-1 protease inhibitors were designed to inhibit a single variant of HIV-1 protease.

For most of the currently approved protease inhibitors, the emergence of multi drug resistant (MDR) mutants poses a great challenge to the efficacy of these drugs. (Condra, J. H. et al. Nature 1995, 374, 569-571; and Clavel, F. et al. N. Engl. J. Med. 2004, 350, 1023-1035.) Development of next generation HIV-1 protease inhibitors active against MDR virus has been the focus of intense research efforts in recent years. (Koh, Y. et al. Antimicrob. Agents Chemother. 2003, 47, 3123-3129; Surleraux, D. L. N. G. et al. J. Med. Chem. 2005, 48, 1813-1822; and Surleraux, D. L. N. G. et al. J. Med. Chem. 2005, 48, 1965-1973.)

Developing different classes of therapeutic agents is not likely to be an adequate solution to the problem of resistance to protease inhibitors, primarily because the same basic mechanisms readily generate viral strains resistant to other agents. Thus, resistance is a major clinical problem for the other major class of HIV drugs, the reverse transcriptase inhibitors, and resistance to newer, preclinical agents, such as the fusion inhibitors is readily elicited in culture.

The challenge for the research community is therefore to develop drugs, e.g., HIV-1 protease inhibitors, that are less vulnerable to drug resistance and/or more active against current protease resistant HIV-1 isolates. The present inventions address this challenge by integrating clinical data, in vitro virology, protein crystallography, computational modeling and chemical design, and high-throughput chemistry and compound screening. HIV protease is a particularly appealing target, as inhibition of its activity is clinical effective; however, it can evolve to tolerate extensive mutation that confers drug resistance while retaining enzymatic function. As the design of the initial protease inhibitors was structure based, a huge knowledge reservoir exists for this protein.

### SUMMARY OF THE INVENTION

The present invention is based, at least in part, on the discovery of new small molecule protease inhibitors (PIs). These inhibitors, and methods of making and using them, are described herein. Because these inhibitors do not protrude beyond the substrate binding envelope on the protease, it is expected that these inhibitors will be less likely to induce the development of resistant strains.

In one aspect, the invention features PIs described herein, or an enantiomer, diastereomer or a pharmaceutically acceptable salt thereof, and pharmaceutical compositions for inhibiting HIV protease that include a pharmaceutical carrier and a therapeutically effective amount of a PI described herein.

In another aspect, the invention features methods for treating HIV in a subject, by administering a therapeutically effective amount of a compound or pharmaceutical composition described herein. In some embodiments, the methods further include administering a second therapeutic agent, e.g., a non-nucleoside reverse transcriptase inhibitor (NNRTI) such as efavirenz (Sustiva™), nevirapine (Viramune™) and delavirdine (Rescriptor™); an nucleoside reverse transcriptase inhibitor (NRTI) such as AZT (zidovudine, Retrovir™)/3TC (lamivudine, Epivir™) and d4T (stavudine, Zerit™)/3TC, and d-drugs (ddI [didanosine, Videx™/VidexEC™], ddC [zalcitabine, Hivid™], d4T); a nucleotide reverse transcriptase inhibitor, such as tenofovir (Viread™); and a fusion inhibitor, such as enfuvirtide (Fuzeon™). In some embodiments, the compound or pharmaceutical composition is administered as part of a highly active antiretroviral therapy (HAART) regimen.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1a-b depict possible synthetic routes to selected inventive compounds.

FIGS. 2a-f depict selected compounds of formula I and associated Kᵢ values.

FIGS. 3a-d depict selected compounds of formula III.

FIGS. 4a-c depicts additional selected compounds of formula I.

FIG. 5 depicts additional selected compounds of formula I.

FIGS. 6a-k depict anti-HIV drugs by class.

FIG. 7 depicts the synthesis of protease inhibitors containing [A] a hydroxyethylamine (HEA) core or [B] a hydroxyethylene (HE) core. Key: (a) EtOH, 70° C.; (b) aq. Na₂CO₃, CH₂Cl₂, r. t; (c) TFA, CH₂Cl₂; (d) Et₃N, THF; (e) R₄X₂CO₂H, EDCI, HOBt, DIPEA, 0° C. to r. t.; (f) H₂, Pd/C, MeOH, r. t; and (g) aq. NaHCO₃, EtOAc, 0° C. to r. t.

FIG. 8 depicts the synthesis of protease inhibitors containing an aza-hydroxyethylamine (Aza-HEA) core. Key: (a) (CH₃)₂CHOH, 80° C. (b) H₂, Pd/C, MeOH, r. t; (c) R₄X₂CO₂H, EDCI, HOBt, DIPEA, 0° C. to r. t.; (d) TFA, CH₂Cl₂; and (e) R₃X₁CO₂H, EDCI, HOBt, DIPEA, 0° C. to r. t.; aq. NaHCO₃, EtOAc.

FIG. 9 depicts a comparison of isobutyl and (S)-2-methylbutyl moieties as P1′ ligands: Inhibitory activities against wild-type and MDR mutant variants of HIV-1 protease.

FIG. 10 depicts selected compounds of formula I and associated Kᵢ values.

FIG. 11a-b depicts selected compounds of formula I.

FIG. 12 depicts the chemical structures of amprenavir (APV) 1, TMC 114 2 and selected compounds of the invention 3.

FIG. 13 depicts a scheme showing the synthesis of intermediates N-Phenyloxazolidine-5-carboxylic acids 9 and 10. Key: (a) n-BuLi, THF, −78° C. to r. t. overnight; (b) RuCl₃.H₂O, CH₃CN—CCl₄-H₂O (2:2:3), 0° C. to r. t. 4-10 h.

FIG. 14 depicts a scheme showing the synthesis of inventive compounds 20-29. Key: (a) EtOH, 80° C., 3-4 h; (b) aq. Na₂CO₃, CH₂Cl₂, 0° C. to r. t., 4-8 h; (c) TFA, CH₂Cl₂, 1 h; (d) (OCOCl)₂, r. t., overnight; (e) Et₃N, THF, 0° C. to r. t., 4-8 h; (f) SnCl₂.2H₂O, EtOAc, 70° C., 2 h.

FIG. 15 depicts a scheme showing the synthesis of compounds 36-39. Key: (a) iPrOH or EtOH, 80° C., 3-4 h; (b) aq. Na₂CO₃, CH₂Cl₂, 0° C. to r. t., 4-8 h; (c) TFA, CH₂Cl₂, 1 h; (d) (OCOCl)₂, r. t., overnight; (e) Et₃N, THF, 0° C. to r. t., 4-8 h.

FIG. 16 depicts a table showing the inhibitory activities of selected inventive compounds, amprenavir (APV) and lopinavir (LPV) against wild type HIV-1 Protease.

FIG. 17 depicts selected structure and inhibitory activities of novel protease inhibitors of the invention.

FIG. 18 depicts a table showing inhibitory activities of selected compounds of the invention against wild type HIV-1 protease.

FIG. 19 depicts a table of inhibitory activities of selected compounds of the invention, and known inhibitors, against MDR Mutant Proteases (Wt: Q7K; M1: L10I, G48V, I54V, L63P, V82A; M2: D30N, L63P, N88D; M3: L10I, L63P, A71V, G73S, I84V, L90M; APV: amprenavir; LPV: lopinavir.)

FIG. 20 depicts a table showing the purity of selected target compounds as determined by HPLC using two different systems. First system: column, Waters Nova-Pak RP-C18 (4 μm, 3.9 mm×150 mm); mobile phase A, 10 mM ammonium acetate in water; mobile phase B, acetonitrile. Using a flow rate of 0.8 mL/min, gradient elution was performed from 50% B to 100% B over 10 min. Second system: column, Agilent Zorbax 300SB-C8 (5 μm, 4.6 mm×250 mm); mobile phase A, 0.1% trifluoroacetic acid in water; mobile phase B, 0.1% trifluoroacetic acid in acetonitrile. Gradient elution was performed from 50% B to 100% B over 10 min at a flow rate of 1 mL/min.

FIG. 21 depicts graphically HIV protease kinetics in the absence and presence of an inhibitor (Amprenavir).

FIG. 22 depicts a graph used for the calculation of initial velocities.

FIG. 23 depicts a graph and equation used to determine binding dissociation constants.

FIG. 24 depicts graphically data enabling the calculation of the Kᵢ for an HIV-1 protease inhibitor (KB-19=21f) of the present invention.

FIG. 25 depicts various classes of HIV-1 protease inhibitors of the present invention.

FIG. 26 depicts various embodiments of the cyclic carbamate portion of the HIV-1 protease inhibitors of the present invention, e.g., those represented by any of Formulas XI to XIX. The present invention expressly encompasses the combination of any of the embodiments depicted in the Figure with any of the other structural features described herein.

FIG. 27 depicts selected compounds of formula XVA/XVB and associated Kᵢ values.

FIG. 28 depicts selected compounds of formula XIIIA/XIIIB and associated Kᵢ values.

FIG. 29 depicts selected compounds of formula XVIIA/XVIIB and associated Kᵢ values.

FIG. 30a-b depict possible synthetic routes to selected inventive compounds.

FIG. 31 depicts inhibitory activities of selected compounds against wild-type and MDR mutant variants of HIV-1 protease.

FIG. 32a-c depicts selected compounds.

FIG. 33a-e depicts selected compounds and associated Kᵢ values.

FIG. 34a-f depicts selected compounds.

### DETAILED DESCRIPTION

One aspect of the present invention addresses the challenge of developing HIV-1 protease inhibitors that are less vulnerable to drug resistance and/or more active against current protease-resistant HIV-1 isolates than other HIV drugs. The present invention addresses this challenge by integrating clinical data, in vitro virology, and high-throughput chemistry and compound screening. HIV protease is a particularly appealing target because inhibiton of its activity is clinically effective; however, it can evolve to tolerate extensive mutations conferring drug resistance while retaining enzymatic function.

Some of the protease inhibitors described herein were designed rationally using an ensemble of HIV-1 protease variant sequences (available online at hivdb.stanford.edu) and three-dimensional structures that the homodimeric HIV protease can tolerate, to maximize the likelihood that these HIV-1 protease inhibitors that will evade mutational resistance. Recently, a structure-based strategy was proposed to reduce the probability of drug resistance by designing inhibitors that interact only with the same residues that are necessary to recognize substrate. (King, N. M. et al. Chem. Biol. 2004, 11, 1333-1338; and Prabu-Jeyabalan, M. et al. J. Virol. 2003, 77, 1306-1315.) Analysis of the crystal structures of HIV-1 protease in complex with substrate peptides suggested that substrate specificity for HIV-1 protease is based not on a particular amino acid sequence, but on a conserved shape (“substrate envelope”). (Prabu-Jeyabalan, M. et al. Structure 2002, 10, 369-381.) Comparison of the substrate structures with protease inhibitor structures reveal critical differences between inhibitor and substrate binding to the enzyme. In case of substrates, most of the conserved hydrogen bonds occur primarily between the backbone of the protease and the backbone of the substrate. Thus, it was determined that it is important that the inhibitors are designed to form hydrogen bonds with relatively conserved residues and preferably with the backbone atoms of the protease rather than the side chain atoms.

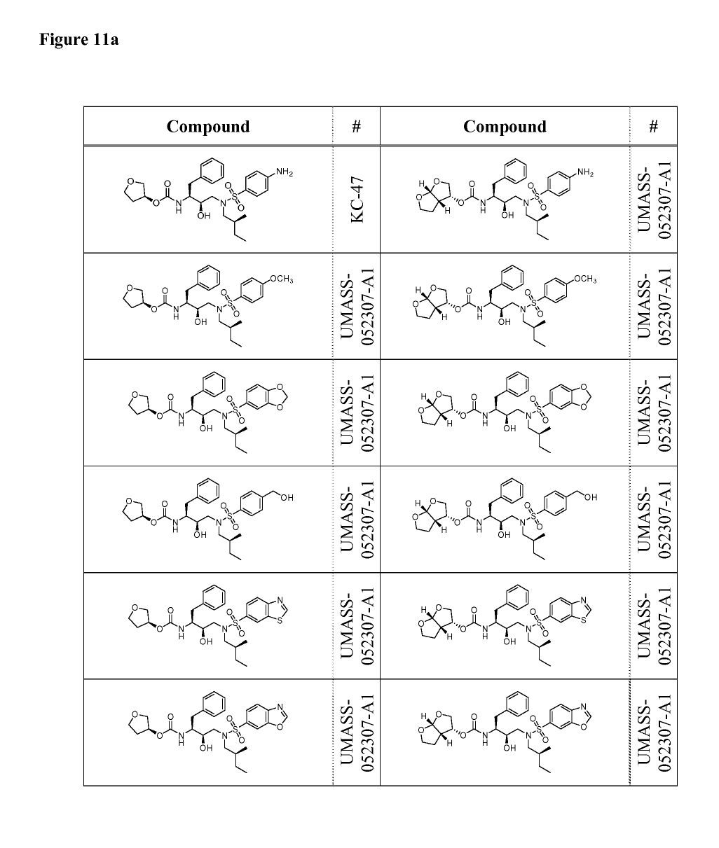

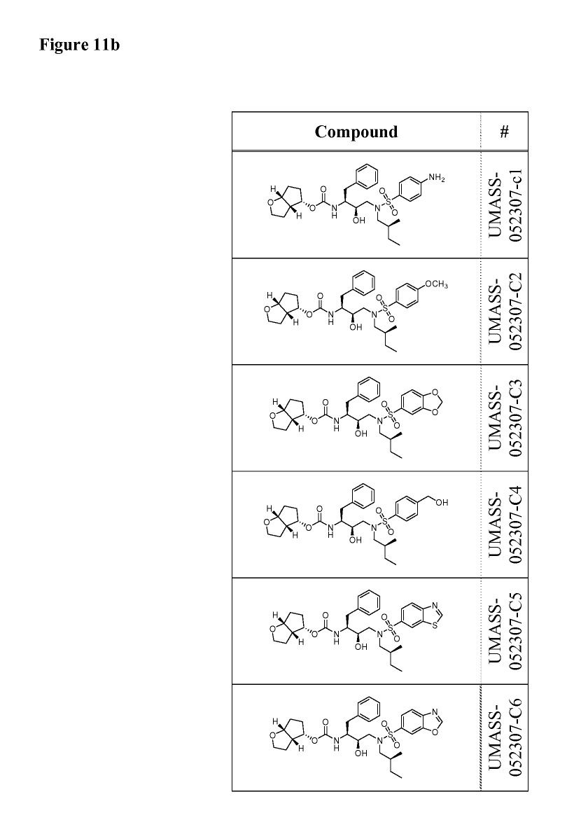

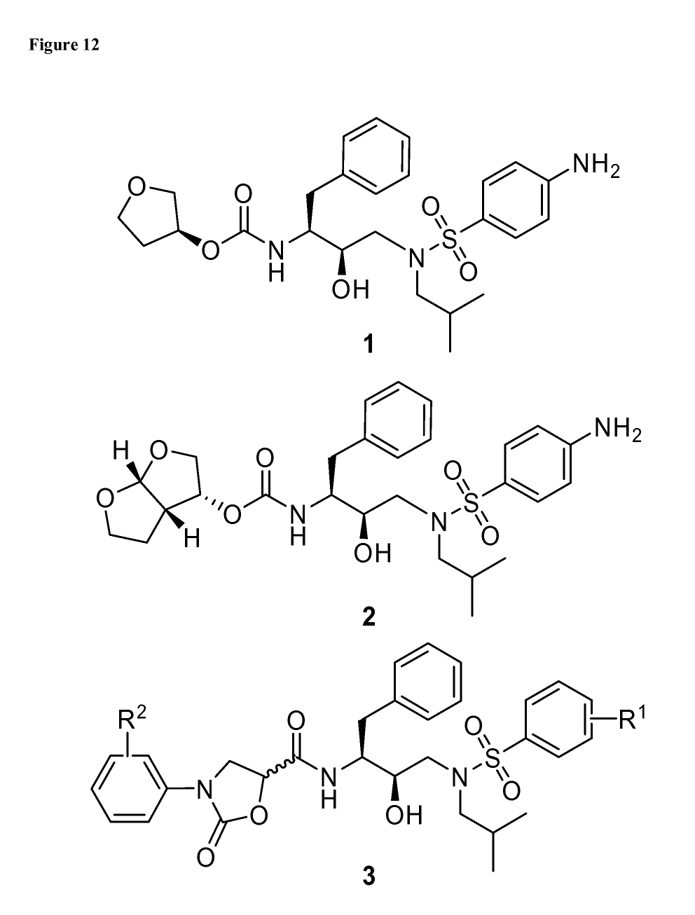

In certain embodiments, the invention relates to designed inhibitors based on the (R)-(hydroxyethylamino)sulfonamide isostere present in 1 and 2 (FIG. 12). Among the approved protease inhibitors, amprenavir (APV) 1 fits reasonably well within the substrate envelope (FIG. 12). It has been suggested that the inhibitors designed on the APV template may be less susceptible to drug resistance. (King, N. M. et al. J. Virol. 2004, 78, 12012-12021.) Protease inhibitor TMC114 2, structurally similar to 1, has been recently shown to possess very potent in vitro and in vivo antiviral activity against wild type as well as MDR mutants (FIG. 12). (De Meyer, S. et al. Antimicrob. Agents & Chemother. 2005, 49, 2314-2321.) Compared to the THF moiety in 1, the additional interactions of bis-THF moiety in 2 with the backbone atoms of fairly conserved Asp29 and Asp30 residues in protease explains its potent and broad spectrum activity against MDR mutants. (Koh, Y. et al. Antimicrob. Agents Chemother. 2003, 47, 3123-3129; and Surleraux, D. L. N. G. et al. J. Med. Chem. 2005, 48, 1813-1822.)

Therefore, it was envisioned that a small heterocyclic moiety with multiple polar atoms located at the P2 position would mimic the critical interactions of the THF/bis-THF moieties present in APV/TMC114. Since the oxygen atom of the carbamate linking THF/bis-THF moieties to the hydroxyethylamine core in 1 and 2 does not make any hydrogen bond contacts with the protease, it was decided to attach the heterocyclic moiety to the core isostere via amide linkage. Previous reports of inhibitors with heteroaryl and other polar heterocyclic groups as P2 ligands directly linked to the (hydroxyethylamino)sulfonamide fragment did not show promising activities. (Ghosh, A. K. et al. Bioorg. Med. Chem. Lett. 1998, 8, 979-982; and Ghosh, A. K. et al. Farmaco 2001, 56, 29-32.) However, inhibitors incorporating substituted hydroxybenzamides as P2 ligands have shown potent inhibitory activities against HIV-1 protease. (Freskos, J. N. et al. Bioorg. Med. Chem. Lett. 1996, 6, 445-450; Cheng, T.-J. et al. Antimicrob. Agents Chemother. 2004, 48, 2437-2447; Nagarajan, S. R. et al. Bioorg. Med. Chem. 2003, 11, 4769-4777; and Vazquez, M. L. U.S. Pat. No. 6,046,190, hereby incorporated by reference.) Recently, protease inhibitors incorporating 2,3-dihydroxybenzoic acid derived macrocyclic P1-P2 ligands have been reported to exhibit low nanomolar protease inhibitory activities. (Ghosh, A. K. et al J. Med. Chem. 2005, 48, 3576-3585.)

In one embodiment of the invention, N-phenyloxazolidine-S-carboxamides were selected to be utilized as P2 ligands in HIV-1 protease inhibitors. The oxazolidines represent a class of synthetic antimicrobial agents that are highly stable and exhibit exceptional bioavailability profiles. (Barbachyn, M. R. et al. Angew. Chem., Int. Ed. 2003, 42, 2010-2023.) For example, Linezolid is an FDA approved antibacterial drug that contains N-phenyloxazolidine nucleus. (Brickner, S. J. et al. J. Med. Chem. 1996, 39, 673-679.) In addition, N-phenyloxazolidine-5-carboxamides were recently reported to possess better antibacterial activities compared to linezolid with enhanced solubility and bioavailability properties. (Hester, J. B. WO 2003/006440, hereby incorporated by reference; and Thomas, R. C. WO 2003/072553, hereby incorporated by reference). It was reasoned that the carbonyl group of the oxazolidine ring would mimic critical hydrogen bond interactions of THF/bis-THF moieties of APV and TMC114 in the S2 binding pocket of the protease active site. The phenyl group at the ring nitrogen can be utilized to introduce functional groups to make additional contacts with the protease. In addition, the selected heterocyclic moiety can be linked to the (hydroxyethylamino)sulfonamide isostere in stereochemically defined manner using either (R)- or (S)-enantiomer of N-phenyloxazolidine-5-carboxylic acids.

Remarkably, the N-phenyloxazolidine based cyclic carbamate ligands have not been utilized previously in protease inhibitors. However, there are reports of inhibitors containing other cyclic carbamate ligands, but with poor protease inhibitory activities. (Salituro, F. G. et al. Bioorg. Med. Chem. Lett. 1998, 8, 3637-3642.) On the other hand, cyclic urea based ligands have been widely used in protease inhibitors; approved drug lopinavir contains cyclic urea at P3 position. (Sham, H. L. et al. Antimicrob. Agents Chemother. 1998, 42, 3218-3224.) In addition, substituted imidazolidines have been incorporated as cyclic P1-P2 scaffolds in protease inhibitors based on hydroxyethylene and hydroxyethylamine isosteres. These conformationally restricted molecules displayed potent inhibitory activities against HIV-1 protease with Ki values in nanomolar range. (Kazmierski, W. M. et al. Bioorg. Med. Chem. Lett. 2004, 14, 5685-5687.) Recently, potent oximinoarylsulfonamide based HIV-1 protease inhibitors that contain N-substituted five membered cyclic urea moiety linked to the hydroxyethylamine core analogous to lopinavir P3 side chain have been reported. (Yeung, C. M. et al. Bioorg. Med. Chem. Lett. 2005, 15, 2275-2278; and Randolph, J. T. et al. Bioorg. Med. Chem. 2006, 14, 4035-4046.) In certain embodiments the compounds of the invention are a novel series of HIV-1 protease inhibitors incorporating N-phenyloxazolidine-S-carboxamides as P2 ligands.

Certain compounds of the invention are competitive inhibitors that appear to bind in the center of the “substrate envelope” (i.e., at the active site of the protease). Importantly, the compounds of the invention are designed such that when bound they do not significantly protrude beyond the substrate envelope; therefore, they are less likely to induce escape mutations. The protease inhibitors of the invention are useful in the treatment of HIV in susceptible mammals, e.g., humans and certain other primates. In addition the compounds have shown activity against a panel of multi-drug resistant (MDR) mutant variants of HIV-1 protease. Moreover, as mentioned above, the inhibitors of the invention can be administered as a monotherapy, or in combination with other therapeutic agents, e.g., as part of a highly active antiretroviral therapy (HAART) regime.

Selected Protease Inhibitors of the Invention. One aspect of the present invention relates to a compound, or a pharmaceutically acceptable salt thereof, of formula I:

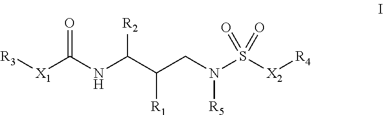

wherein, independently for each occurrence,

X₁ is absent, —O—, —S—, —NR— or

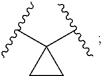

X₂ is absent, —O—, —S—, —NR— or

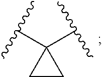

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, amino, (amino)alkyl, amido, amido, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, (cycloalkyl)alkyl, amino, (amino)alkyl, amido, (amido)alkyl, (keto)alkyl, cycloalkyl, heterocyclyl, aryl, heteroaryl, (heterocyclyl)alkyl, aralkyl or heteroaralkyl; and

the stereochemical configuration at any stereocenter is R, S, or a mixture of these configurations;

provided that when X₁ is absent; R₃ is not

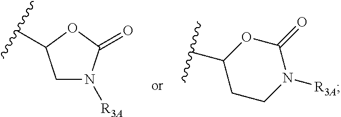

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein X₁ is

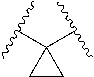

In certain embodiments, the present invention relates to the aforementioned compound and any of the attendant definitions, wherein X₂ is absent.

In certain embodiments, the present invention relates to the aforementioned compound and any of the attendant definitions, wherein X₁ is absent; and X₂ is absent.

In certain embodiments, the present invention relates to the aforementioned compound and any of the attendant definitions, wherein R₁ is OH.

In certain embodiments, the present invention relates to the aforementioned compound and any of the attendant definitions, wherein R₂ is aralkyl or heteroaralkyl.

In certain embodiments, the present invention relates to the aforementioned compound and any of the attendant definitions, wherein R₂ is aralkyl.

In certain embodiments, the present invention relates to the aforementioned compound and any of the attendant definitions, wherein R₂ is

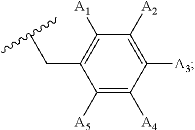

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to the aforementioned compound and any of the attendant definitions, wherein R₂ is

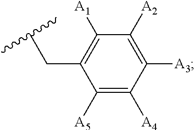

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₂ is

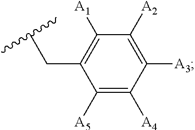

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₃ is alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₃ is aryl or heteroaryl.

In certain embodiments, the present invention relates to the aforementioned compound and any of the attendant definitions, wherein R₄ is alkyl, aryl or heteroaryl.

In certain embodiments, the present invention relates to the aforementioned compound and any of the attendant definitions, wherein R₅ is alkyl, (cycloalkyl)alkyl, amino, (amino)alkyl, amido, (amido)alkyl, (heterocyclyl)alkyl, aralkyl or heteroaralkyl.

In certain embodiments, the present invention relates to the aforementioned compound and any of the attendant definitions, wherein R₅ is alkyl, (cycloalkyl)alkyl or (heterocyclyl)alkyl.

In certain embodiments, the present invention relates to the aforementioned compound and any of the attendant definitions, wherein X₁ is absent; X₂ is absent; R₁ is OH; R₂ is aralkyl; R₃ is aryl or heteroaryl; R₄ is alkyl, aryl or heteroaryl; and R₅ is alkyl, (cycloalkyl)alkyl or (heterocyclyl)alkyl.

Another aspect of the present invention relates to a compound, or a pharmaceutically acceptable salt thereof, of formula II:

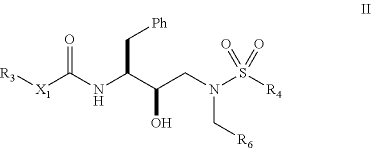

wherein, independently for each occurrence,

X₁ is absent, —O— or

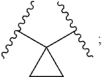

R₃ is alkyl, alkenyl, amino, (amino)alkyl, amido, (amido)alkyl, cycloalkyl, heterocyclyl, aryl, heteroaryl, (heterocyclyl)alkyl, aralkyl or heteroaralkyl;

R₄ is aryl, heteroaryl, aralkyl or heteroaralkyl; and

R₆ is alkyl, cycloalkyl, heterocyclyl, aryl, heteroaryl, aralkyl or heteroaralkyl;

provided that when X₁ is absent; R₃ is not

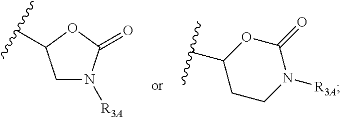

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein X₁ is

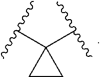

In certain embodiments, the present invention relates to the aforementioned compound and any of the attendant definitions, wherein R₃ is alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₃ is aryl or heteroaryl.

In certain embodiments, the present invention relates to the aforementioned compound and any of the attendant definitions, wherein R₄ is alkyl, aryl or heteroaryl.

In certain embodiments, the present invention relates to the aforementioned compound and any of the attendant definitions, wherein R₆ is alkyl, (cycloalkyl)alkyl, amino, (amino)alkyl, amido, (amido)alkyl, (heterocyclyl)alkyl, aralkyl or heteroaralkyl.

In certain embodiments, the present invention relates to the aforementioned compound and any of the attendant definitions, wherein R₆ is alkyl, (cycloalkyl)alkyl or (heterocyclyl)alkyl.

In certain embodiments, the present invention relates to the aforementioned compound and any of the attendant definitions, wherein R₃ is

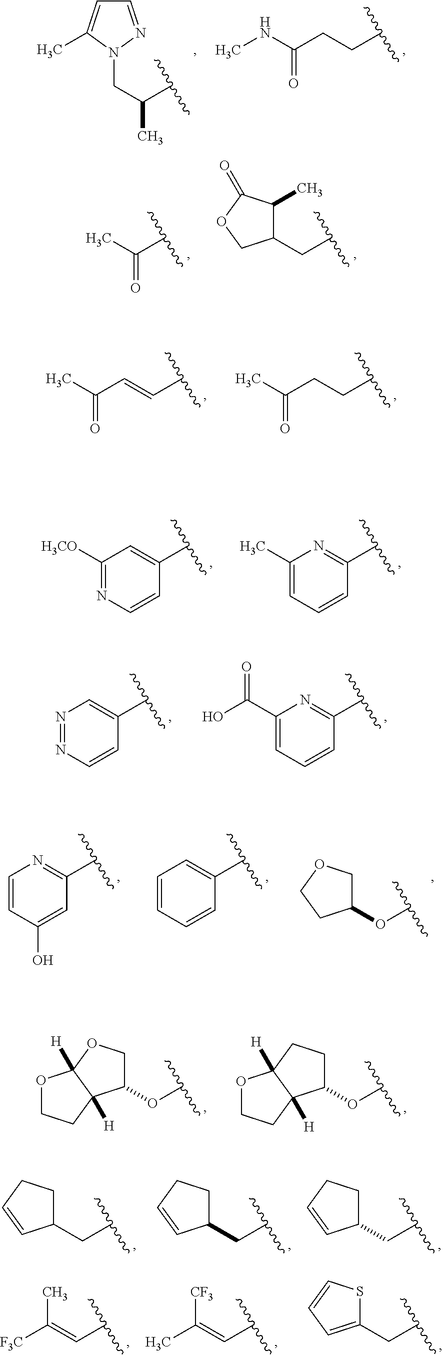

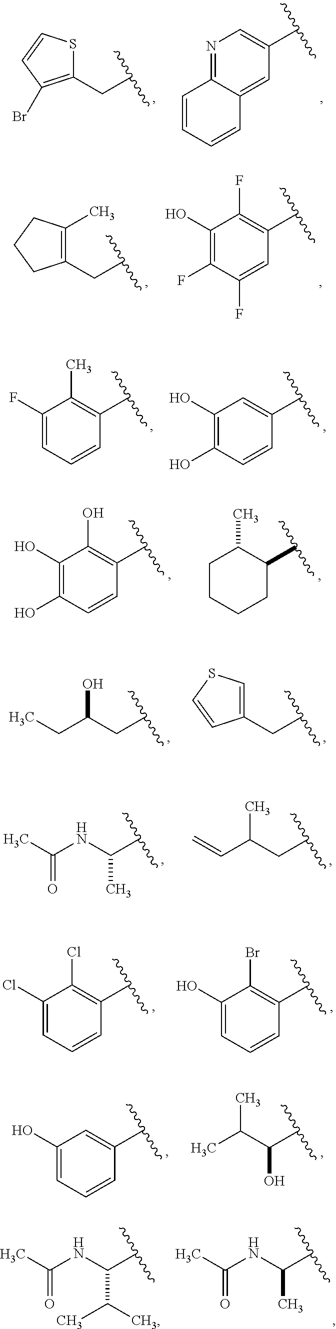

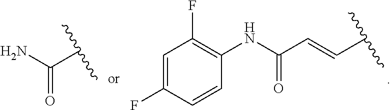

In certain embodiments, the present invention relates to the aforementioned compound and any of the attendant definitions, wherein R₄ is

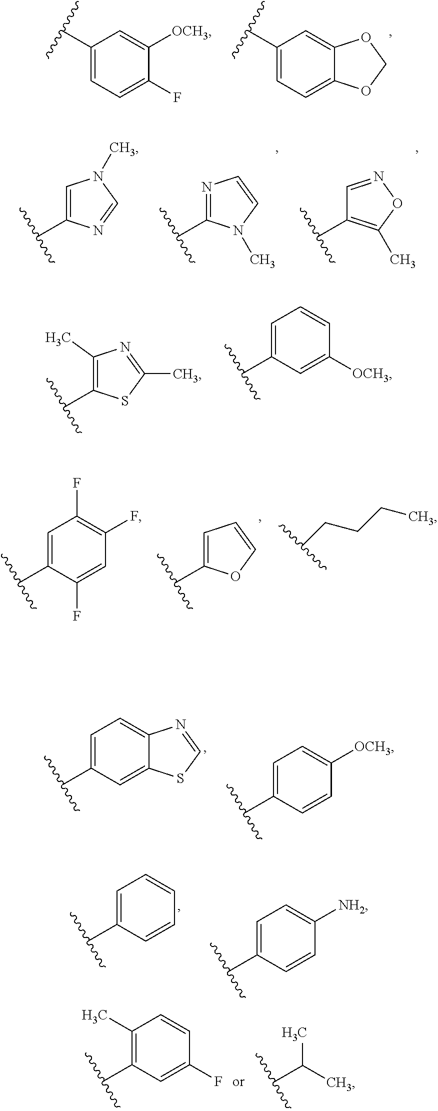

In certain embodiments, the present invention relates to the aforementioned compound and any of the attendant definitions, wherein R₆ is

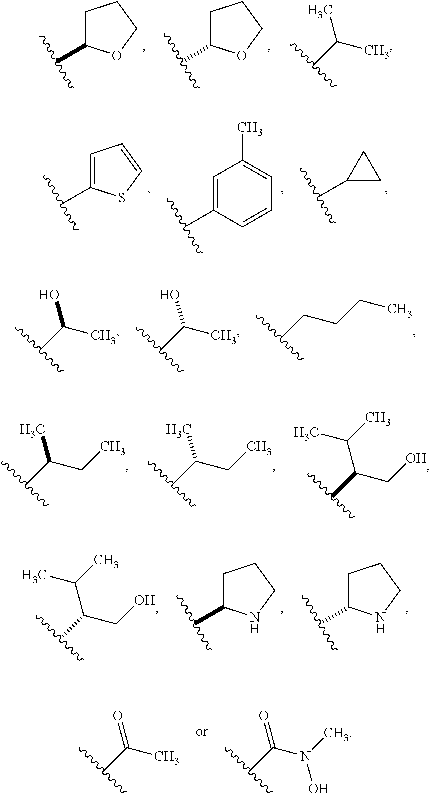

In certain embodiments, the present invention relates to the aforementioned compound and any of the attendant definitions, wherein X₁ is absent; and R₃ is

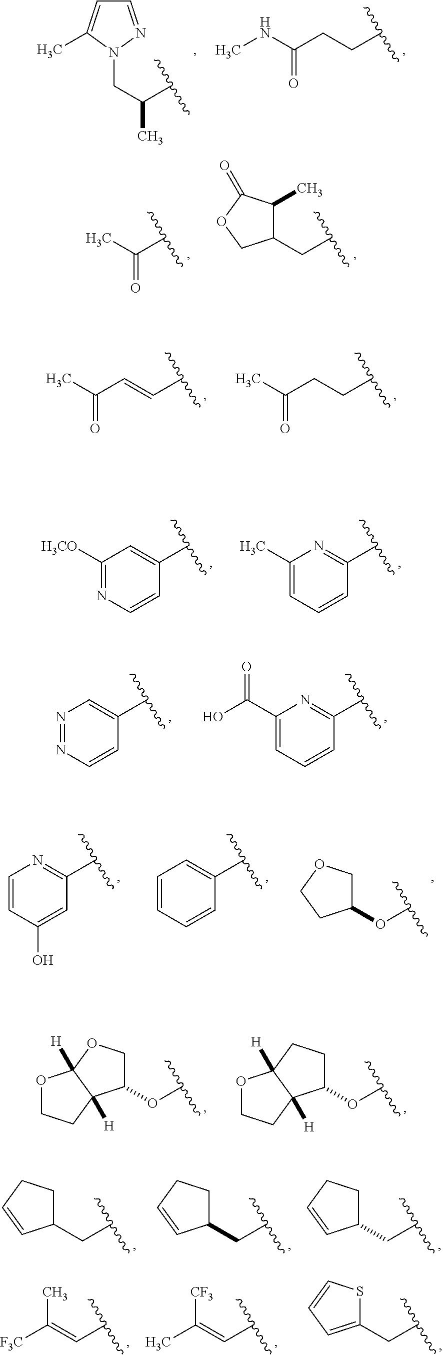

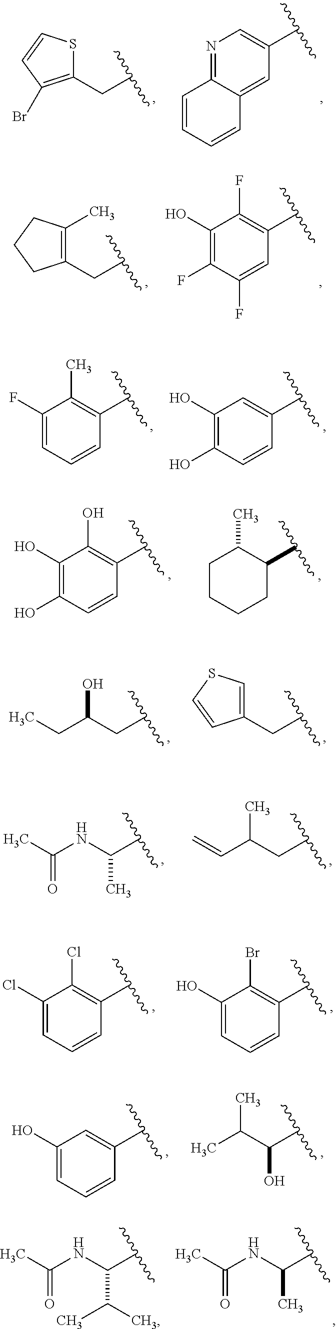

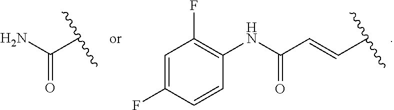

In certain embodiments, the present invention relates to the aforementioned compound and any of the attendant definitions, wherein X₁ is absent; R₃ is

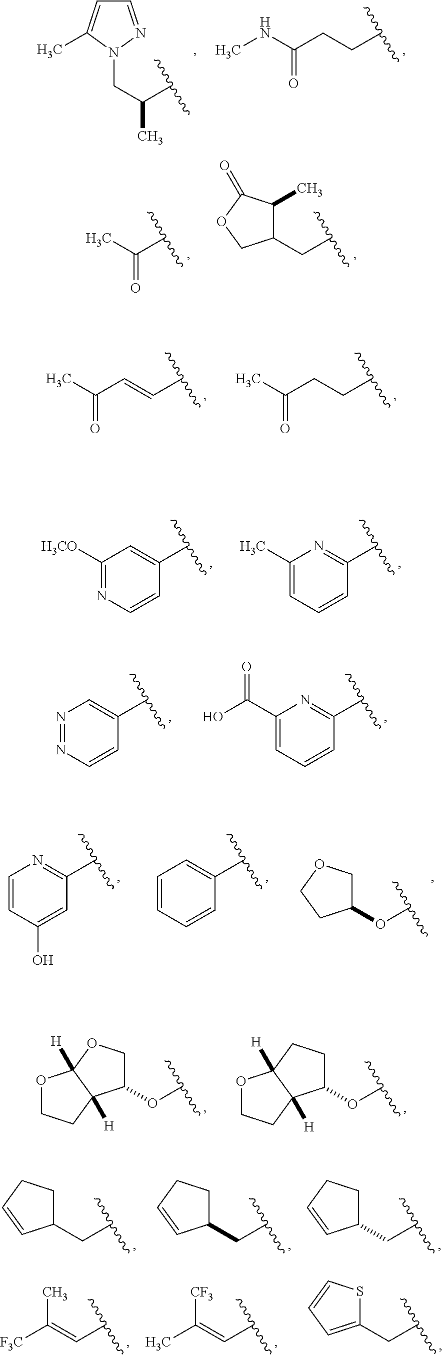

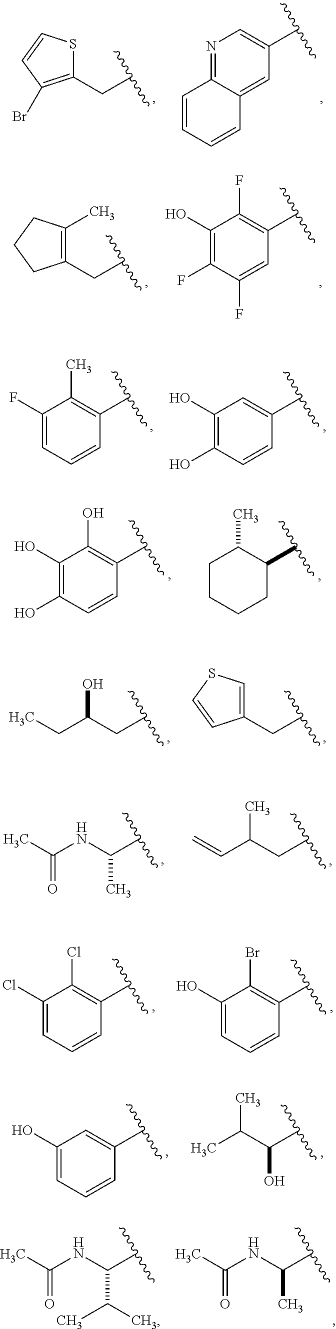

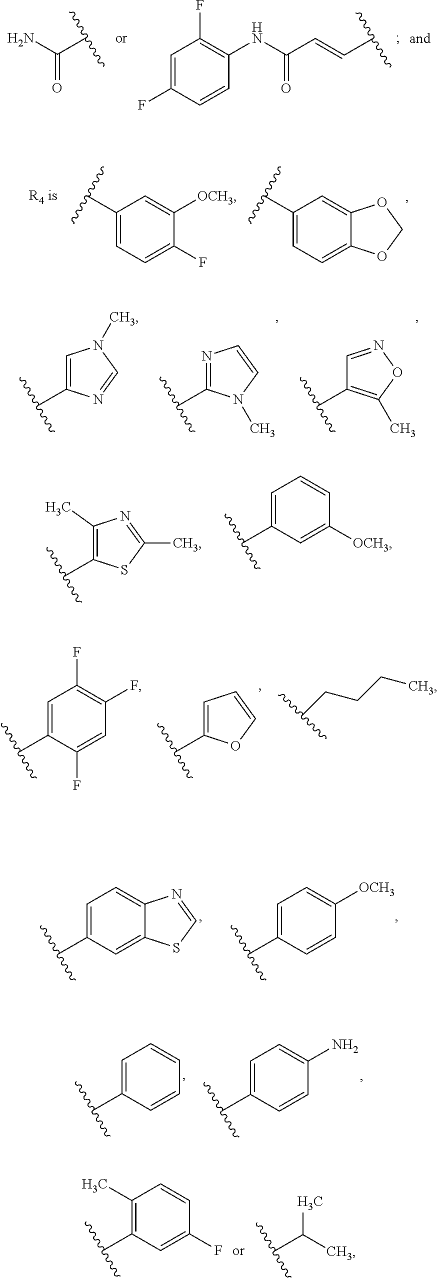

In certain embodiments, the present invention relates to the aforementioned compound and any of the attendant definitions, wherein X₁ is absent; R₃ is

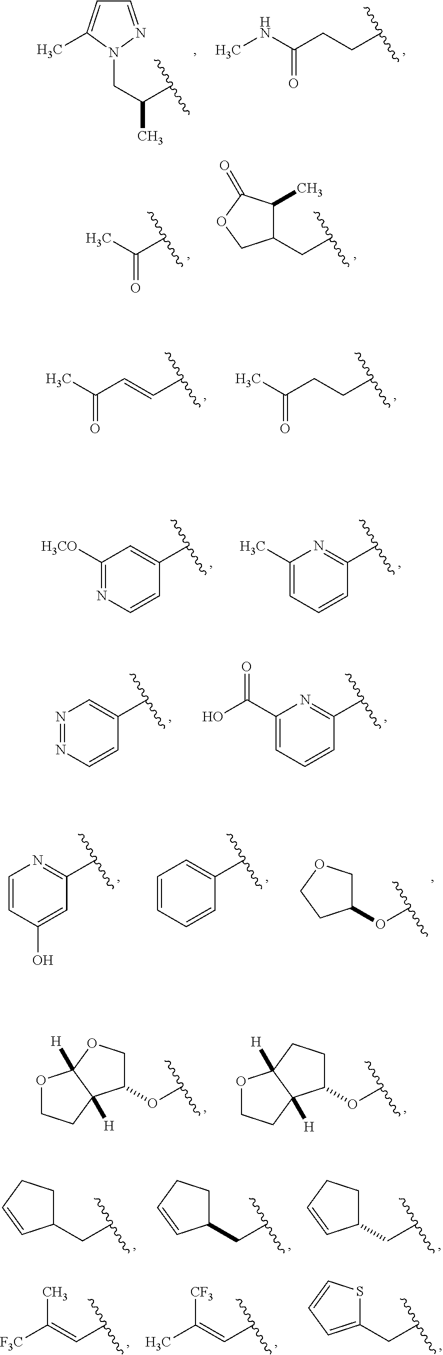

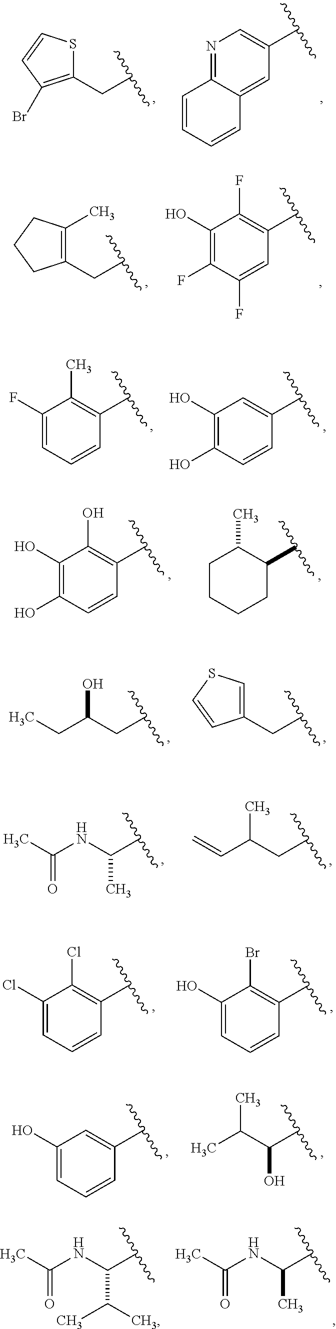

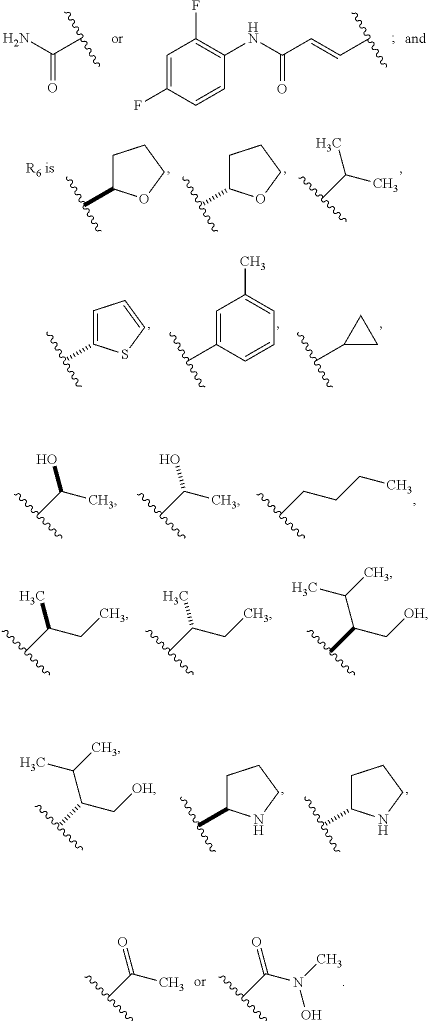

In certain embodiments, the present invention relates to the aforementioned compound and any of the attendant definitions, wherein R₄ is

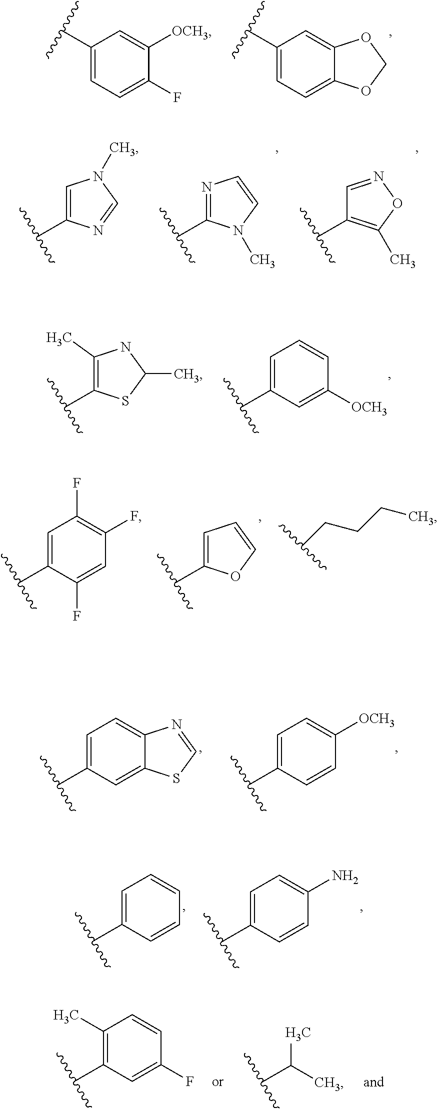

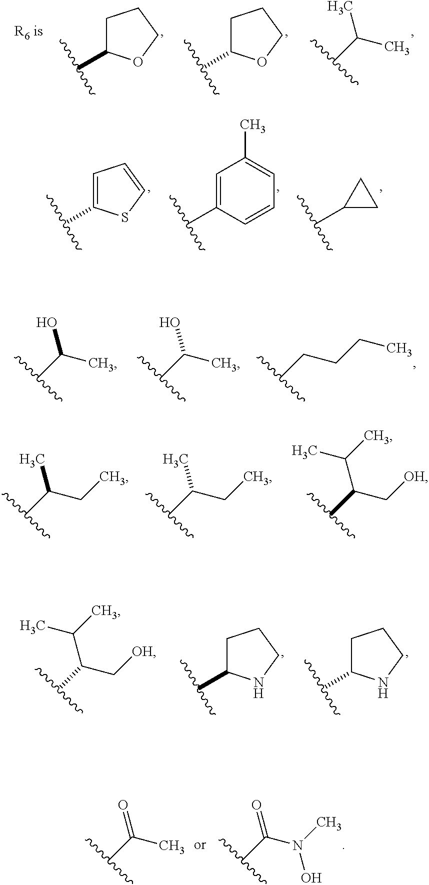

In certain embodiments, the present invention relates to the aforementioned compound and any of the attendant definitions, wherein X₁ is absent; R₃ is

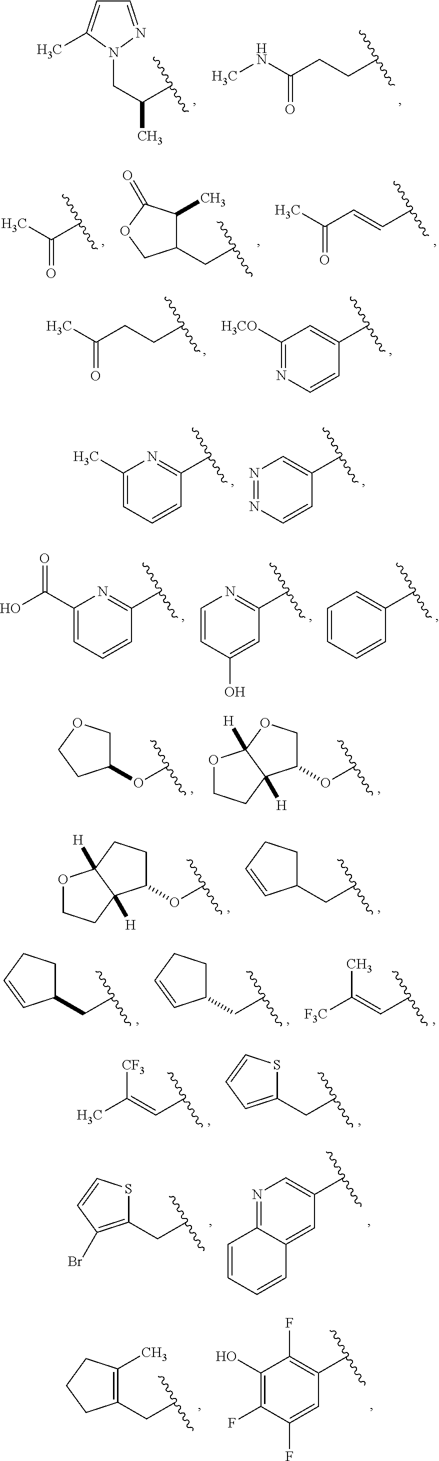

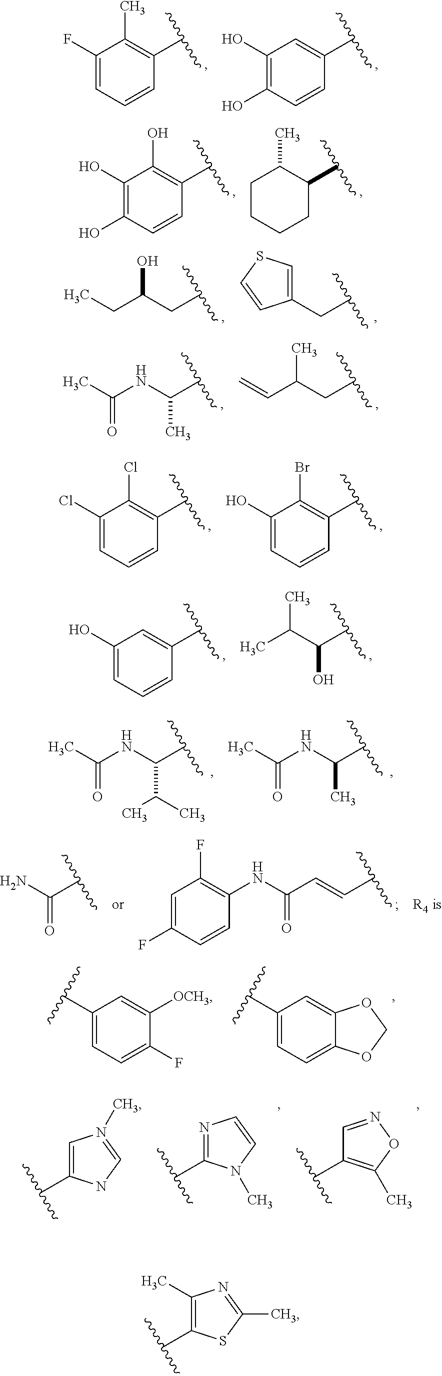

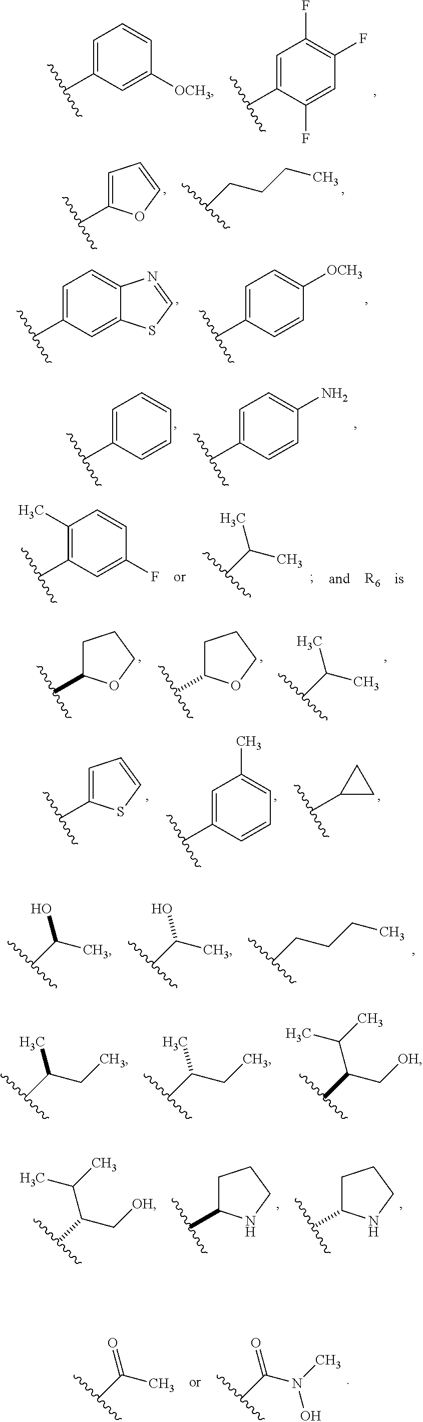

Another aspect of the present invention relates to a compound, or a pharmaceutically acceptable salt thereof, selected from the group consisting of

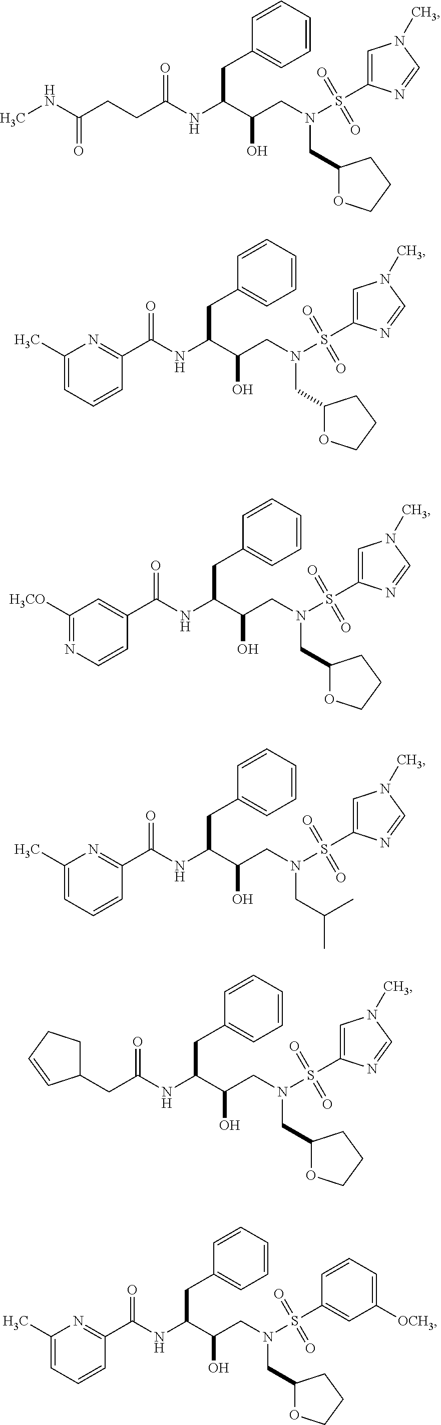

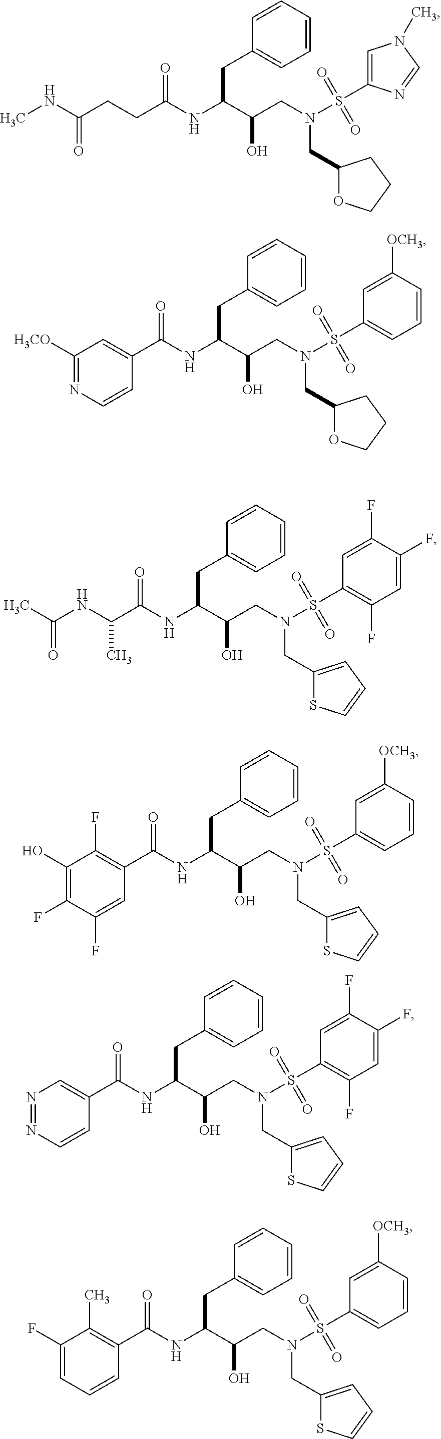

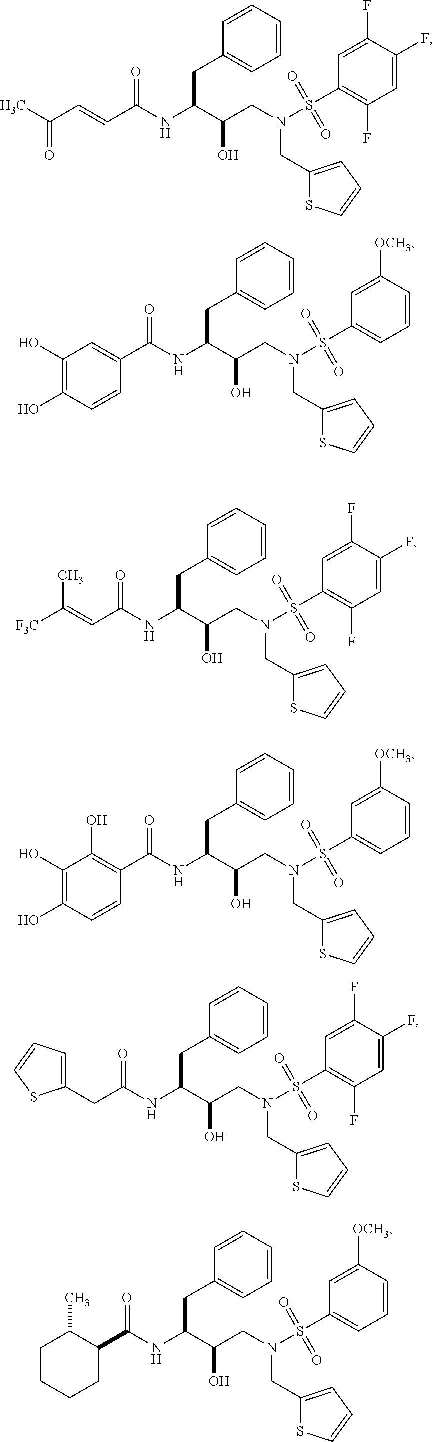

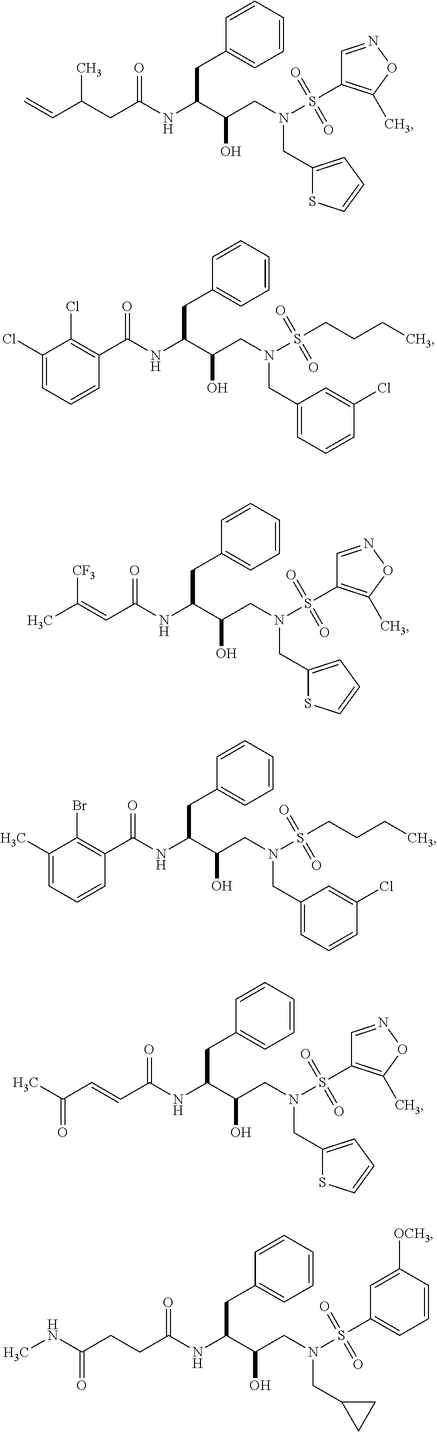

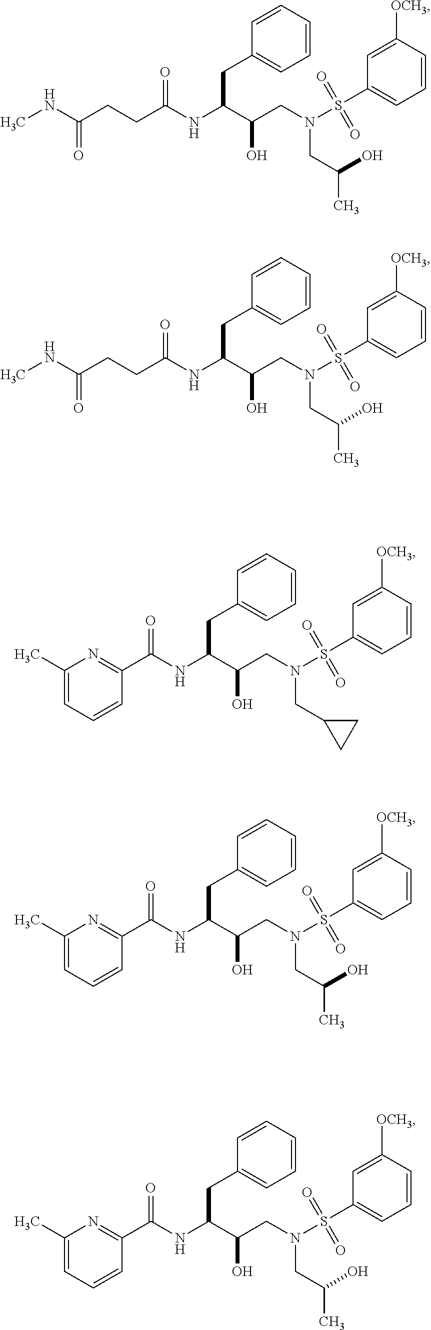

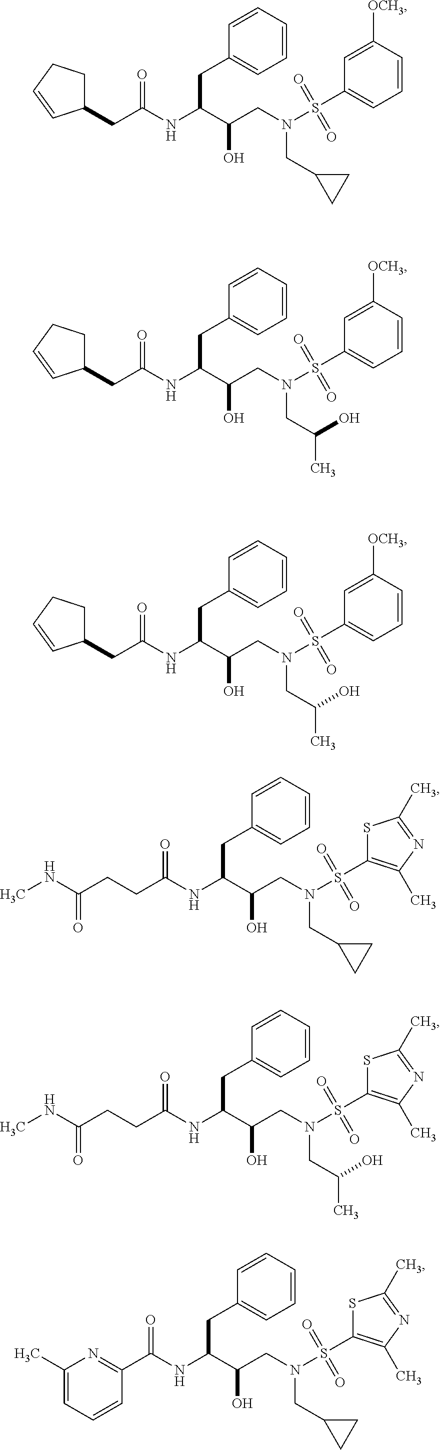

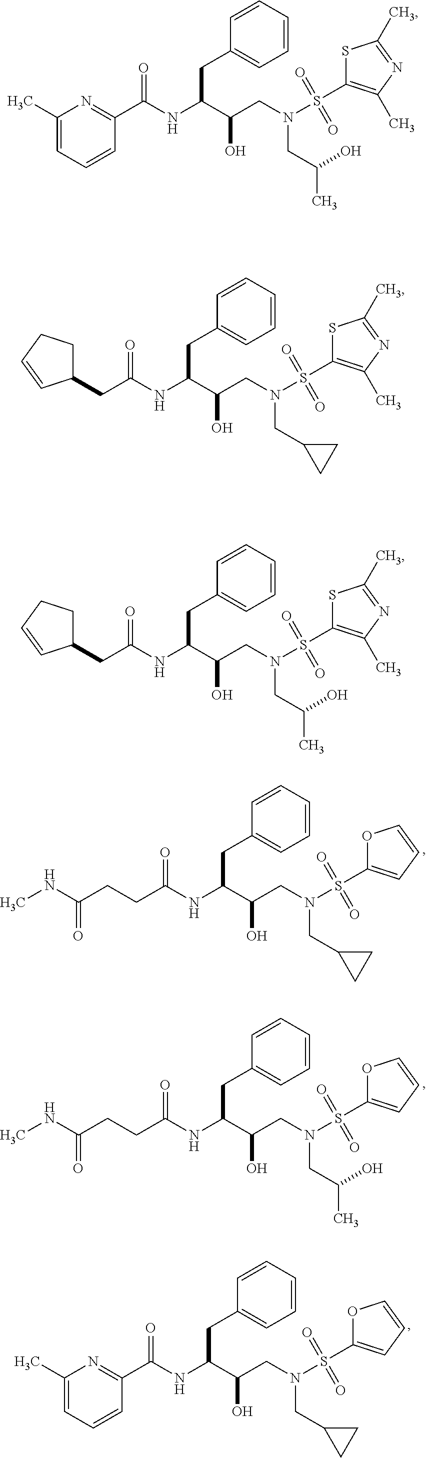

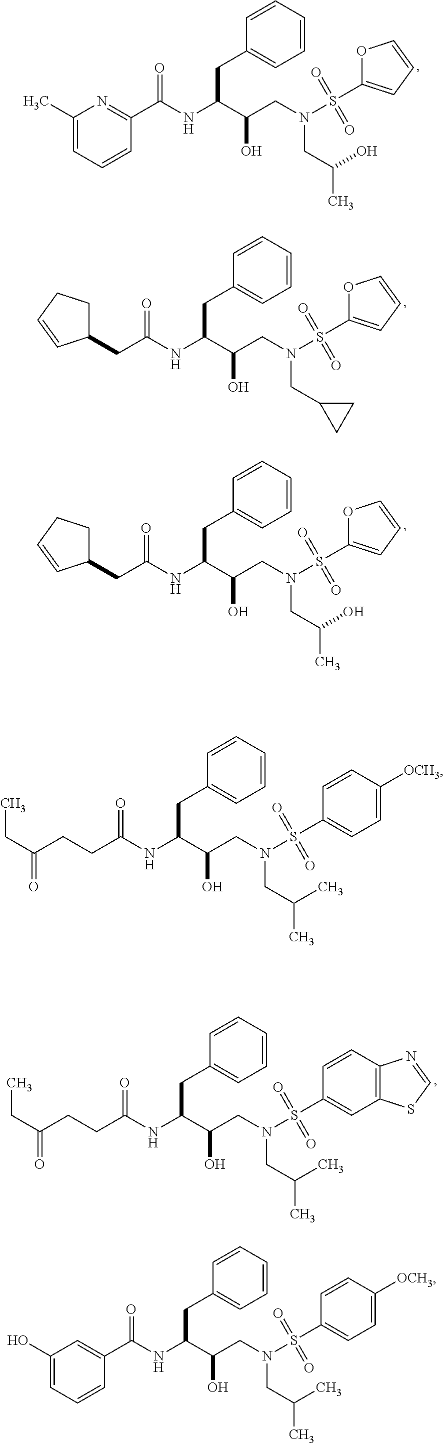

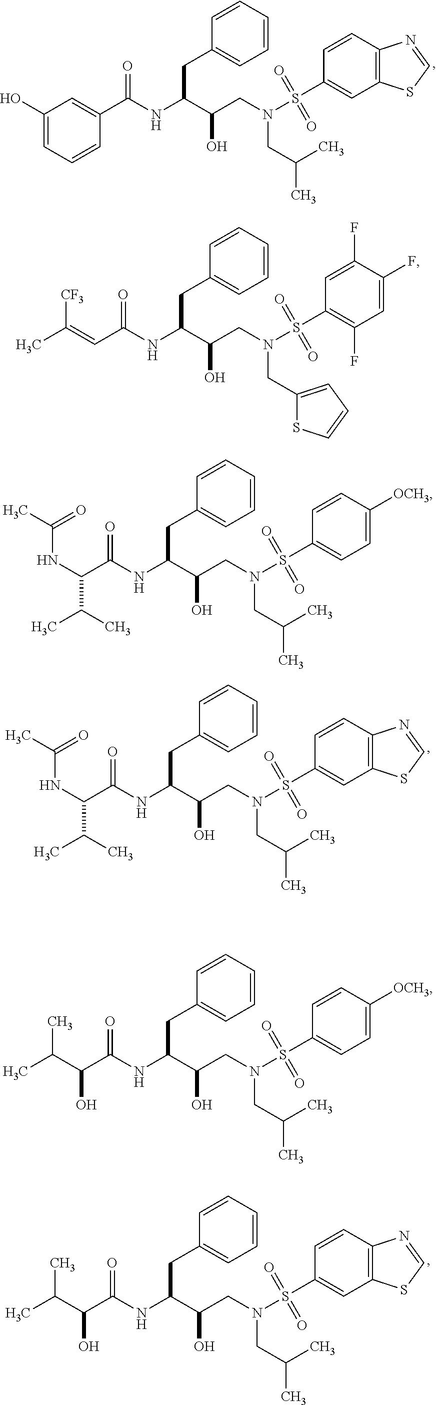

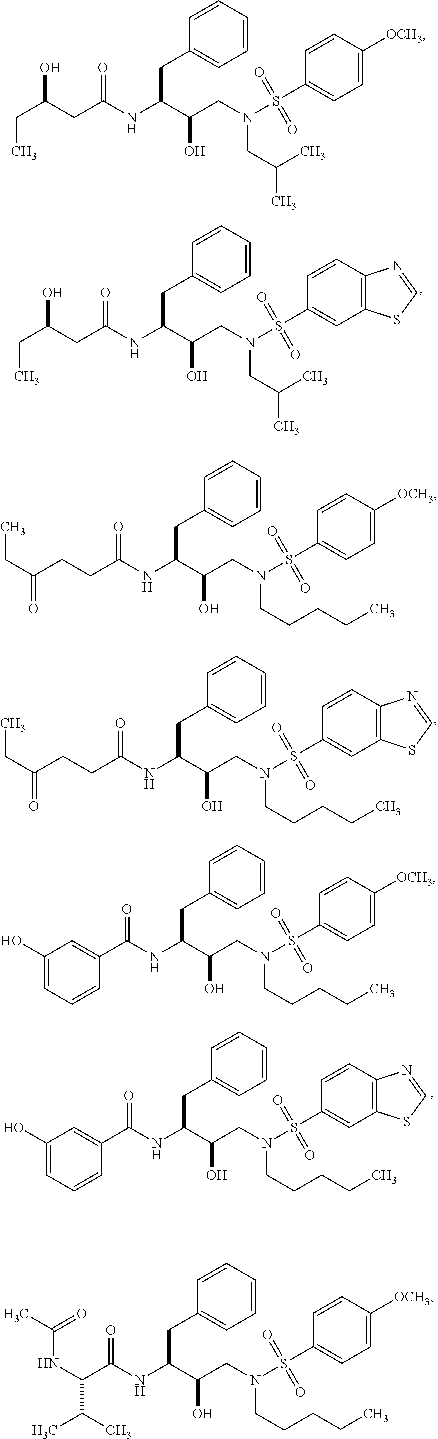

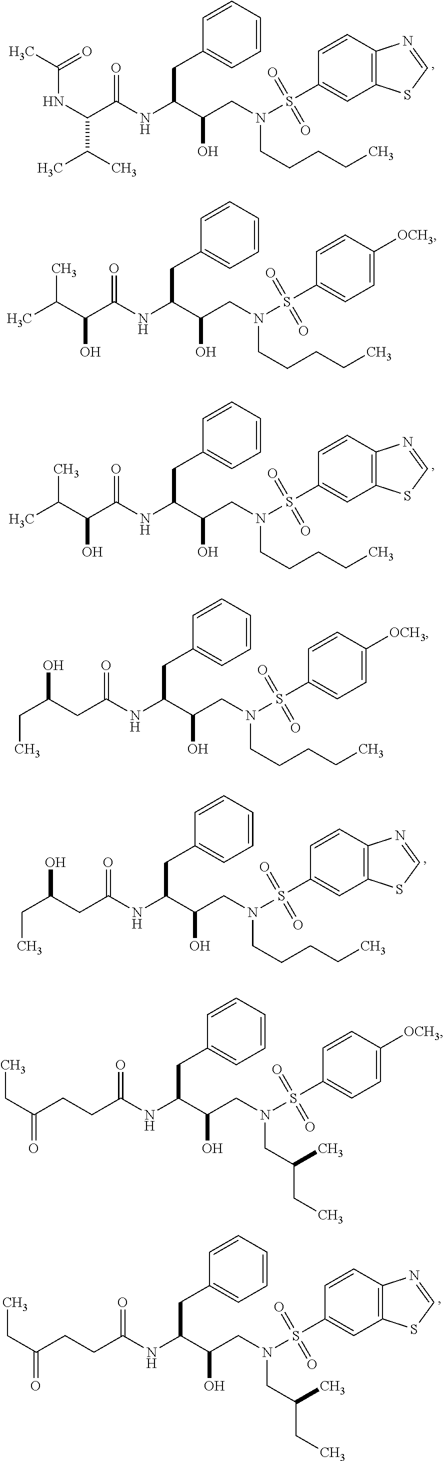

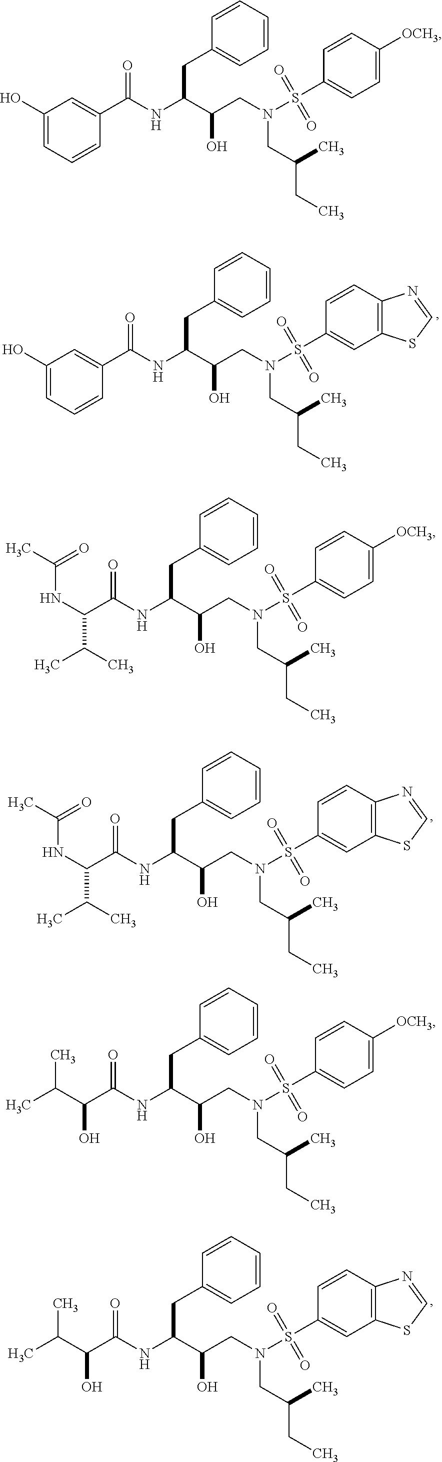

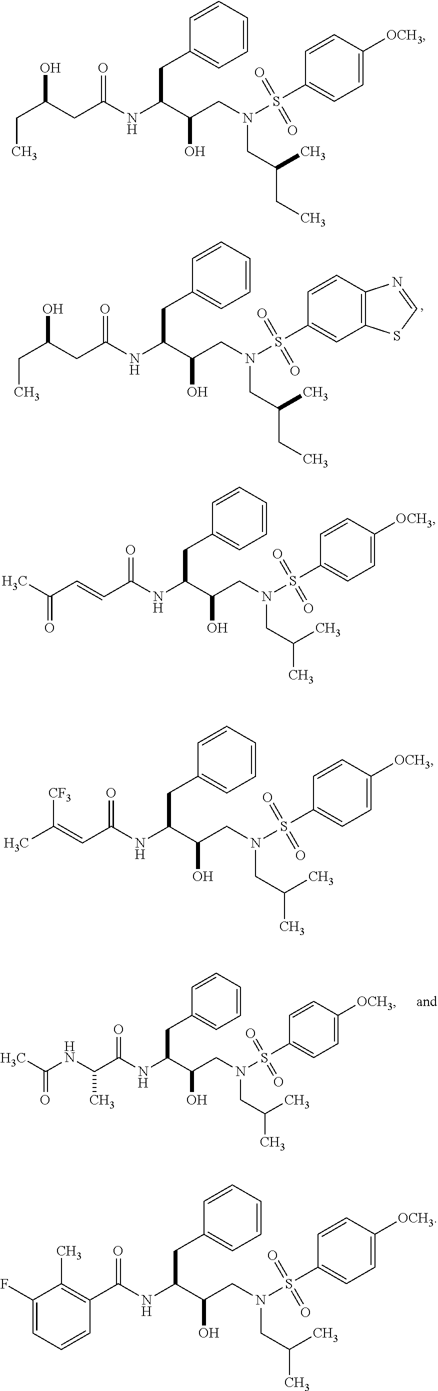

Another aspect of the present invention relates to a compound, or a pharmaceutically acceptable salt thereof, selected from the group consisting of

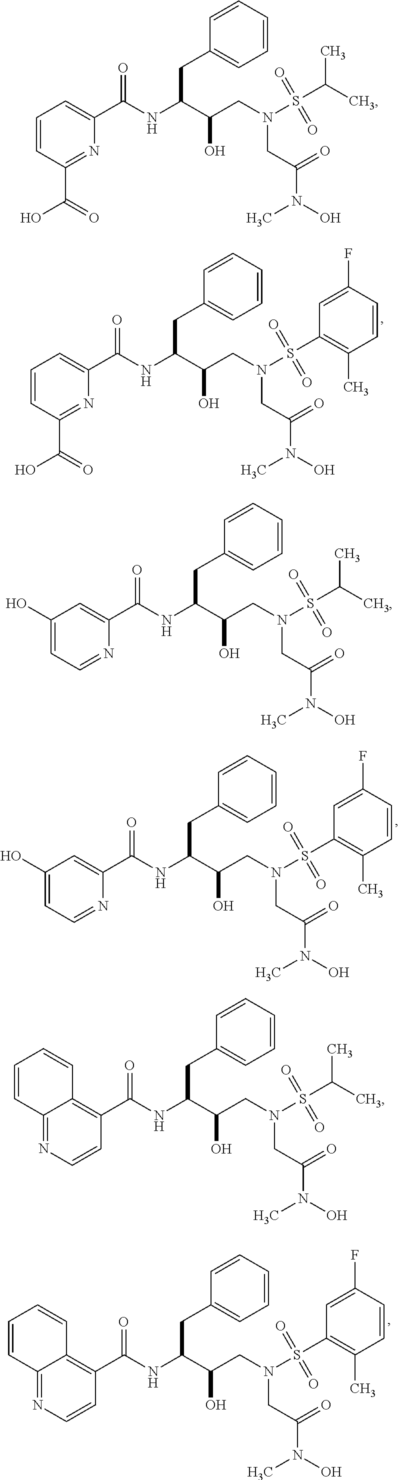

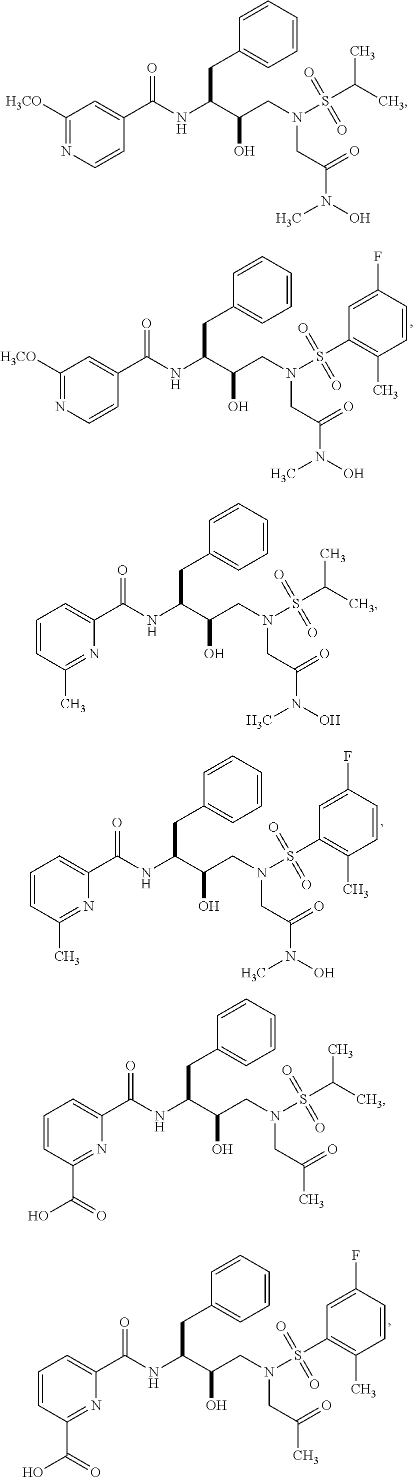

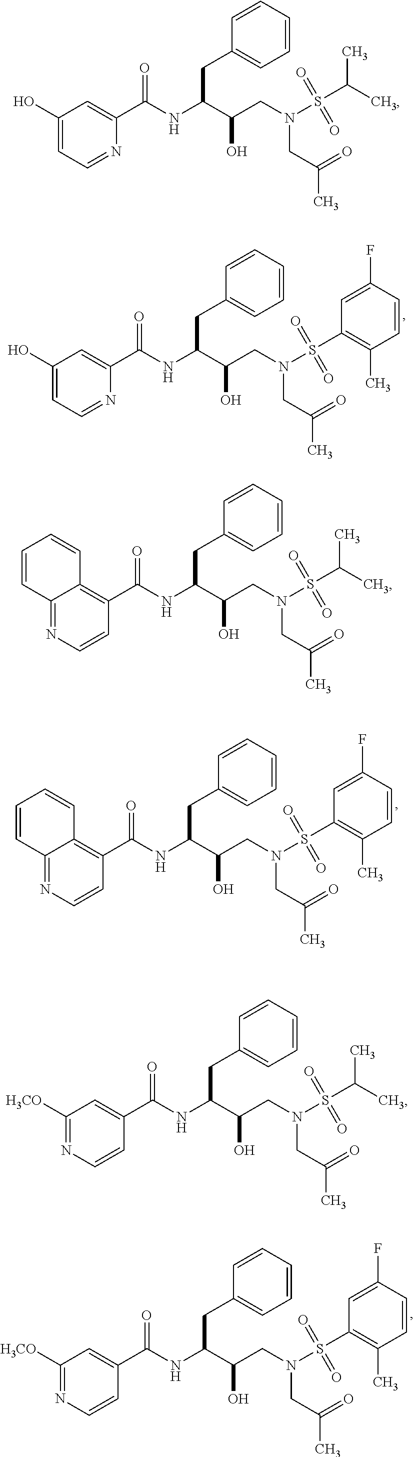

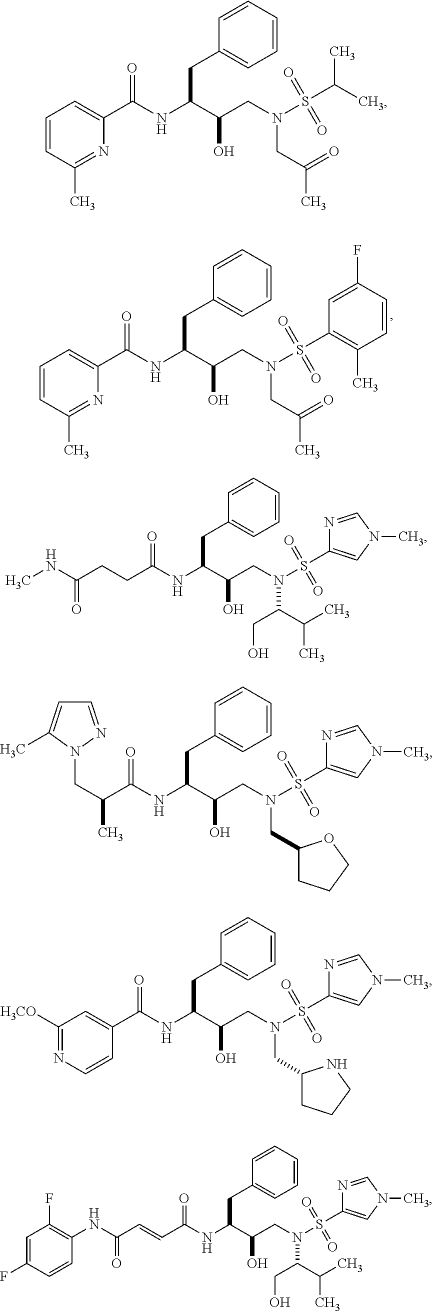

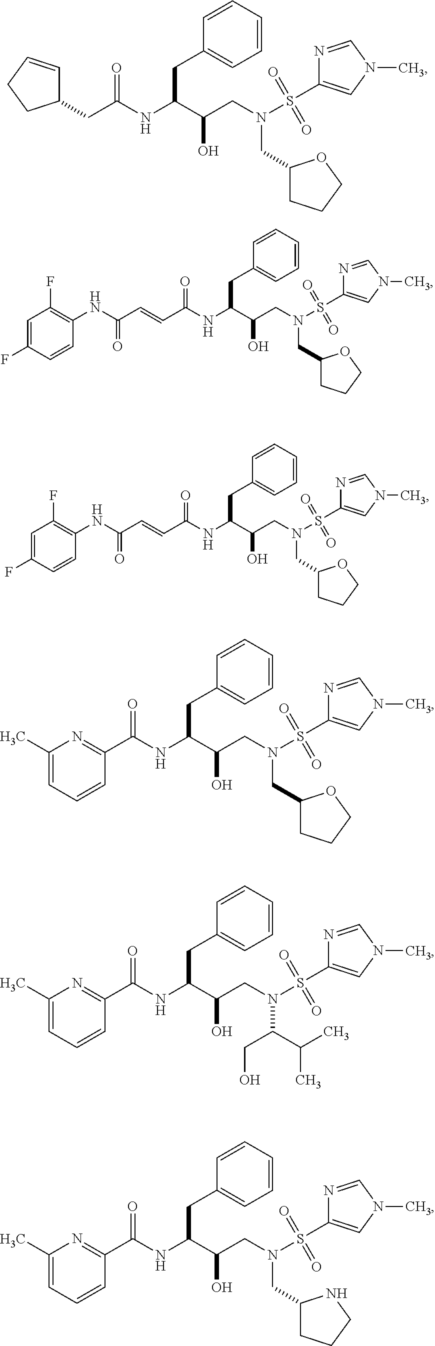

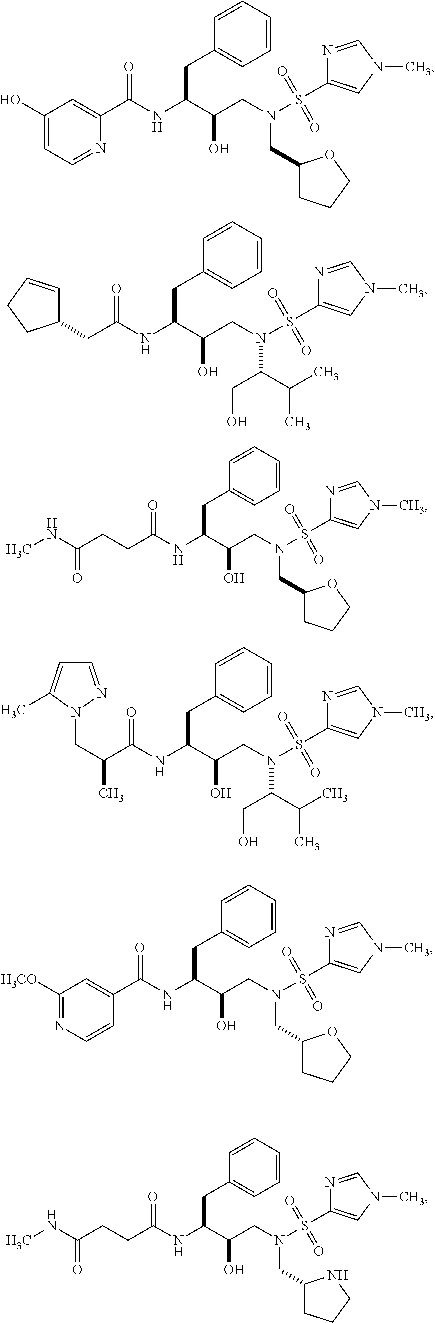

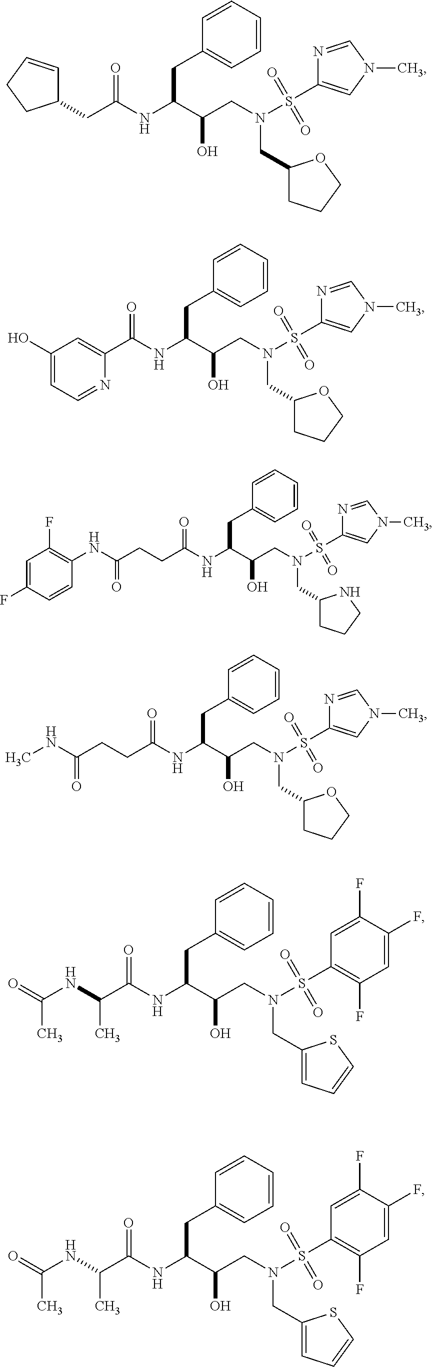

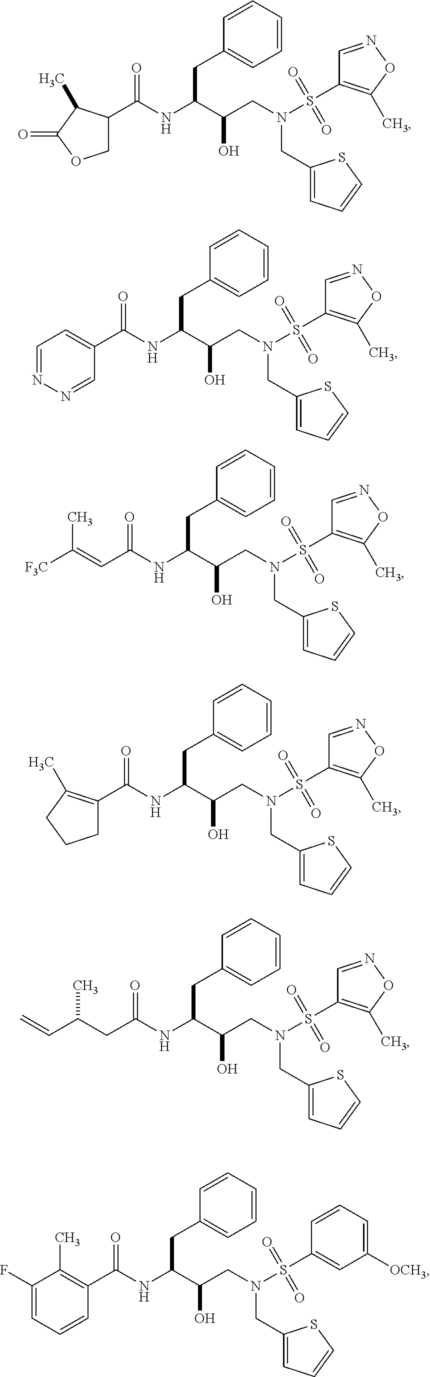

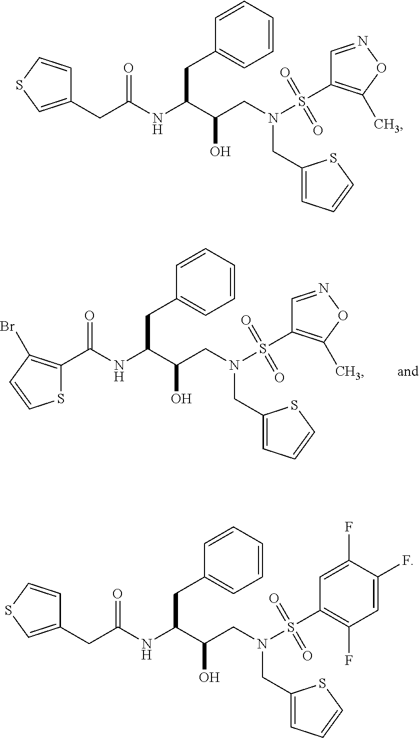

Another aspect of the present invention relates to a compound, or a pharmaceutically acceptable salt thereof, selected from the group consisting of

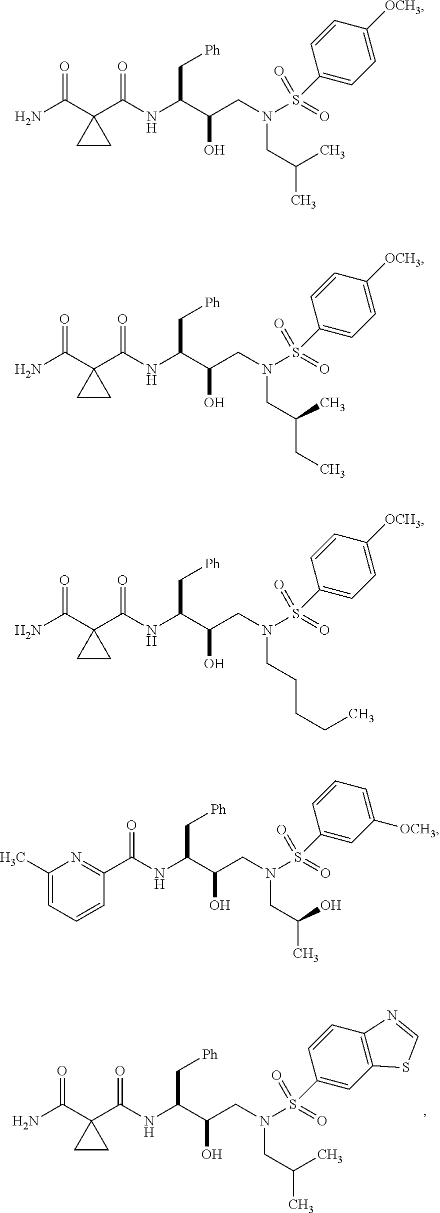

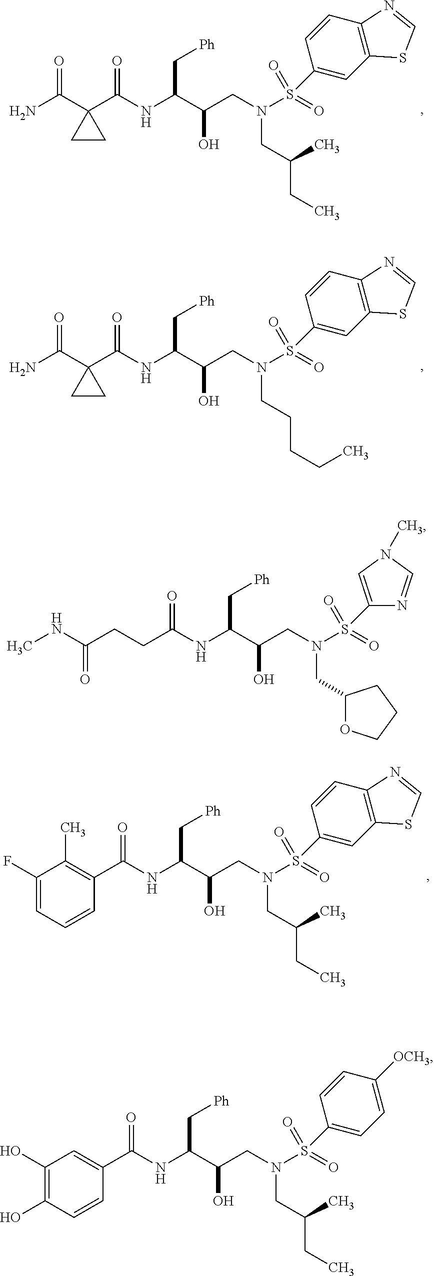

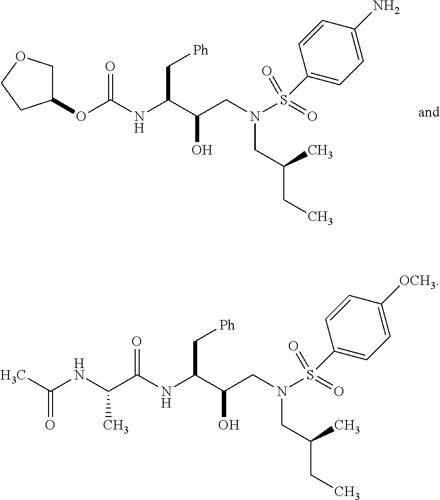

Another aspect of the present invention relates to a compound, or a pharmaceutically acceptable salt thereof, selected from the group consisting of

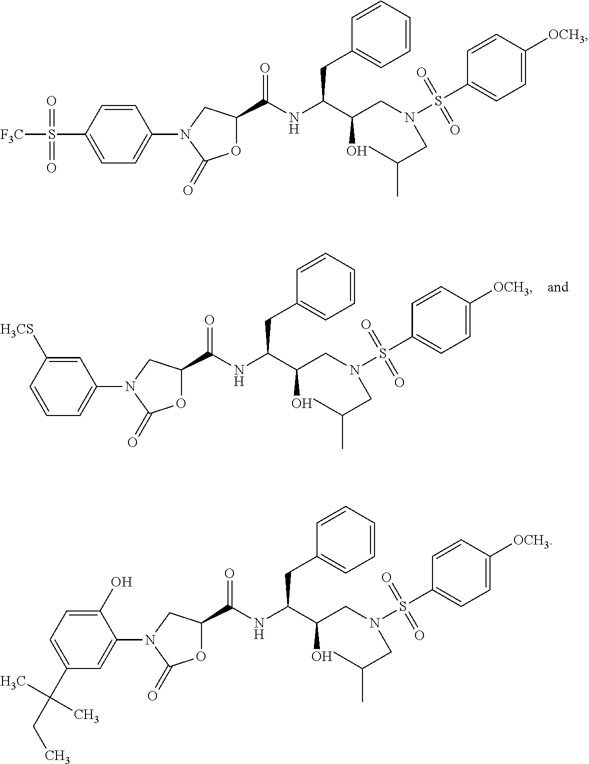

Another aspect of the present invention relates to a compound, or a pharmaceutically acceptable salt thereof, selected from the group consisting of

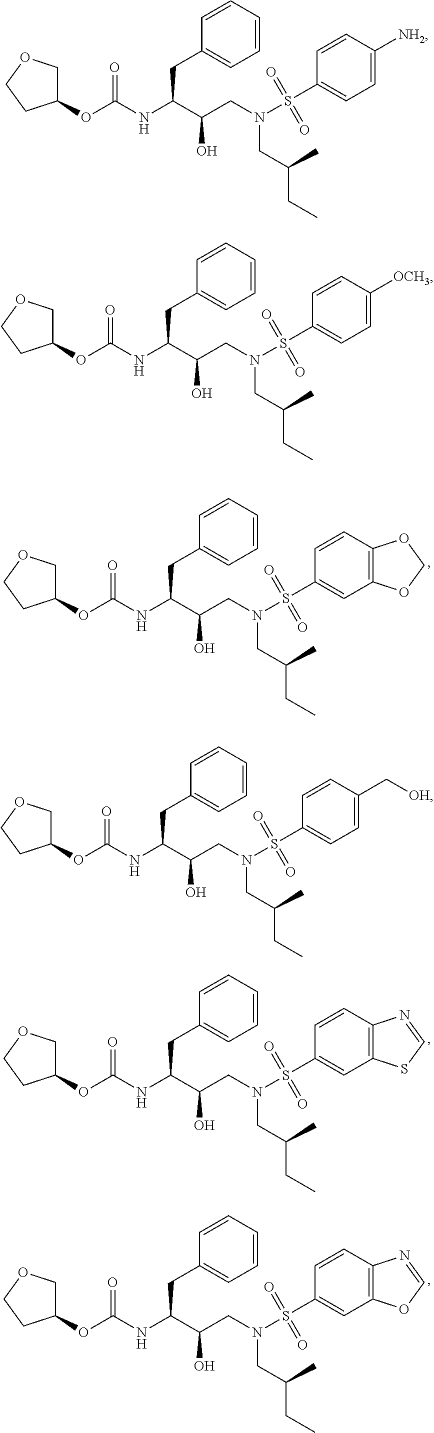

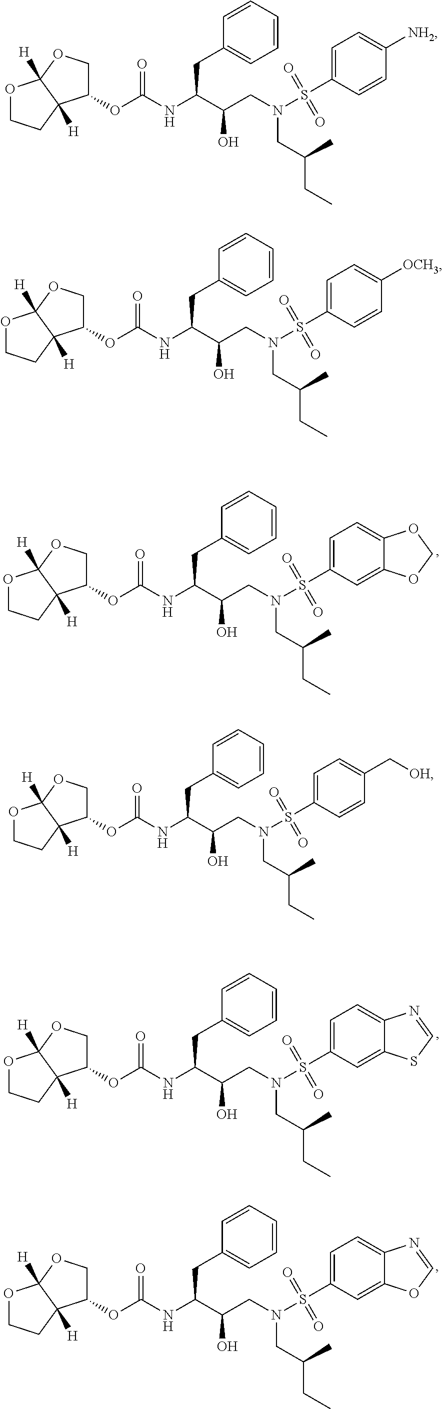

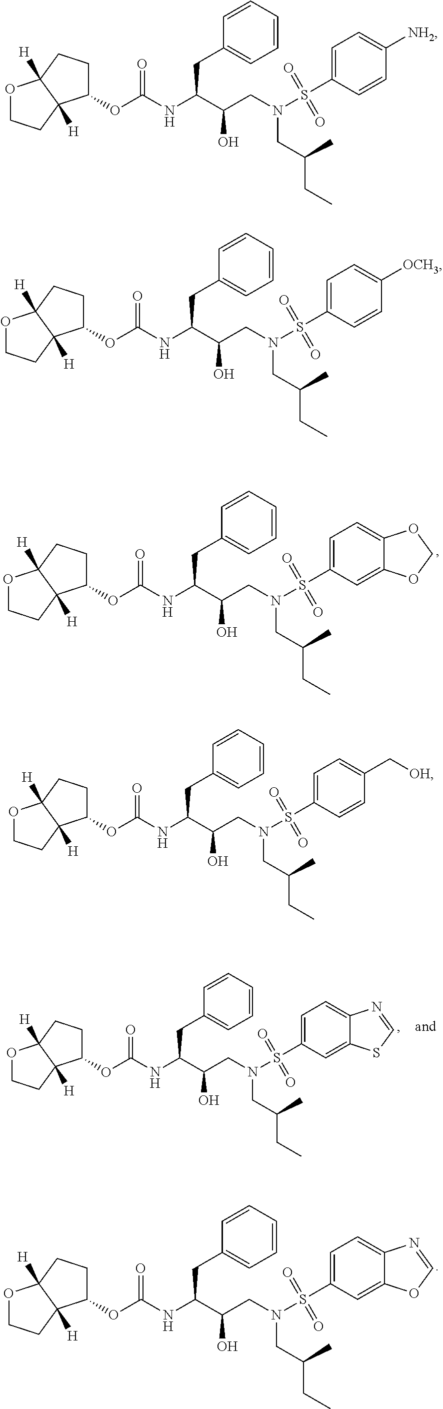

Another aspect of the present invention relates to a compound, or a pharmaceutically acceptable salt thereof, of formula III:

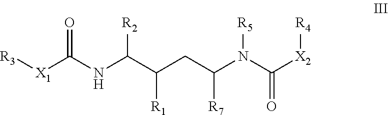

wherein, independently for each occurrence,

X₁ is absent, —O—, —S—, —NR— or

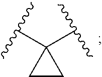

X₂ is absent, —O—, —S—, —NR— or

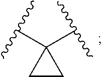

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, aralkyl, heteroaralkyl or acyl;

R₇ is hydrogen, alkyl, (cycloalkyl)alkyl, amino, (amino)alkyl, amido, (amido)alkyl, (keto)alkyl, cycloalkyl, heterocyclyl, aryl, heteroaryl, (heterocyclyl)alkyl, aralkyl or heteroaralkyl; and

the stereochemical configuration at any stereocenter is R, S, or a mixture of these configurations;

provided that when X₁ is absent; R₃ is not

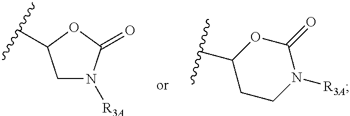

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

and provided that when X₂ is absent; R₄ is not

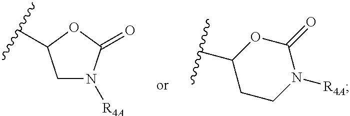

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein X₂ is absent.

In certain embodiments, the present invention relates to the aforementioned compound and any of the attendant definitions, wherein X₁ is absent; and X₂ is absent.

In certain embodiments, the present invention relates to the aforementioned compound and any of the attendant definitions, wherein R₁ is OH.

In certain embodiments, the present invention relates to the aforementioned compound and any of the attendant definitions, wherein R₂ is aralkyl or heteroaralkyl.

In certain embodiments, the present invention relates to the aforementioned compound and any of the attendant definitions, wherein R₂ is aralkyl.

In certain embodiments, the present invention relates to the aforementioned compound and any of the attendant definitions, wherein R₂ is

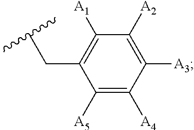

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to the aforementioned compound and any of the attendant definitions, wherein R₂ is

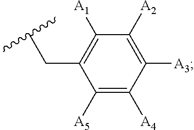

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₂ is

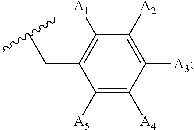

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₃ is alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₃ is aryl or heteroaryl.

In certain embodiments, the present invention relates to the aforementioned compound and any of the attendant definitions, wherein R₄ is alkyl, aryl or heteroaryl.

In certain embodiments, the present invention relates to the aforementioned compound and any of the attendant definitions, wherein R₅ is hydrogen.

In certain embodiments, the present invention relates to the aforementioned compound and any of the attendant definitions, wherein R₇ is

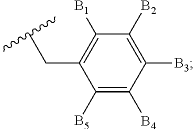

and B₁, B₂, B₃, B₄ and B₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to the aforementioned compound and any of the attendant definitions, wherein R₇ is

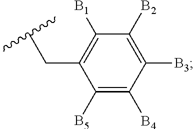

B₁, B₂, B₄ and B₅ are hydrogen; and B₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₇ is

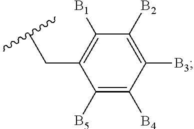

B₁, B₂, B₃ and B₅ are hydrogen; and B₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₇ is alkyl, (cycloalkyl)alkyl, amino, (amino)alkyl, amido, (amido)alkyl, (heterocyclyl)alkyl, aralkyl or heteroaralkyl.

In certain embodiments, the present invention relates to the aforementioned compound and any of the attendant definitions, wherein R₇ is alkyl, (cycloalkyl)alkyl or aralkyl.

In certain embodiments, the present invention relates to the aforementioned compound and any of the attendant definitions, wherein X₁ is absent; X₂ is absent; R₁ is OH; R₂ is aralkyl; R₃ is aryl or heteroaryl; R₄ is alkyl, aryl or heteroaryl; R₅ is hydrogen; and R₇ is alkyl, (cycloalkyl)alkyl or aralkyl.

Another aspect of the present invention relates to a compound, or a pharmaceutically acceptable salt thereof, of formula IV:

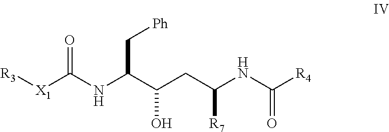

wherein, independently for each occurrence,

X₁ is absent or —O—;

R₃ is alkyl, alkenyl, amino, (amino)alkyl, amido, (amido)alkyl, cycloalkyl, heterocyclyl, aryl, heteroaryl, (heterocyclyl)alkyl, aralkyl or heteroaralkyl;

R₄ is aryl, amino, (amino)alkyl, amido, (amido)alkyl, heterocyclyl, (heterocyclyl)alkyl, heteroaryl, aralkyl or heteroaralkyl; and

R₇ is alkyl, cycloalkyl, (cycloalkyl)alkyl or aralkyl;

provided that when X₁ is absent; R₃ is not

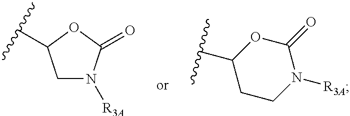

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

and provided that when X₂ is absent; R₄ is not

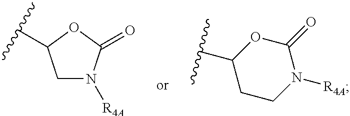

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein R₃ is amino, (amino)alkyl, amido, (amido)alkyl, (keto)alkyl, cycloalkyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₄ is aryl, amino, (amino)alkyl, amido, (amido)alkyl, (keto)alkyl (heterocyclyl)alkyl or heterocyclyl.

In certain embodiments, the present invention relates to the aforementioned compound and any of the attendant definitions, wherein R₇ is alkyl, cycloalkyl or (cycloalkyl)alkyl.

In certain embodiments, the present invention relates to the aforementioned compound and any of the attendant definitions, wherein X₁ is absent; and R₃ is amino, (amino)alkyl, amido, (amido)alkyl, (keto)alkyl, cycloalkyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent; R₃ is amino, (amino)alkyl, amido, (amido)alkyl, (keto)alkyl, cycloalkyl, heterocyclyl, aryl, heteroaryl, (heterocyclyl)alkyl, aralkyl or heteroaralkyl; R₄ is aryl, amino, (amino)alkyl, amido, (amido)alkyl, (keto)alkyl (heterocyclyl)alkyl or heterocyclyl; and R₇ is alkyl, cycloalkyl or (cycloalkyl)alkyl.

In certain embodiments, the present invention relates to the aforementioned compound and any of the attendant definitions, wherein R₃ is

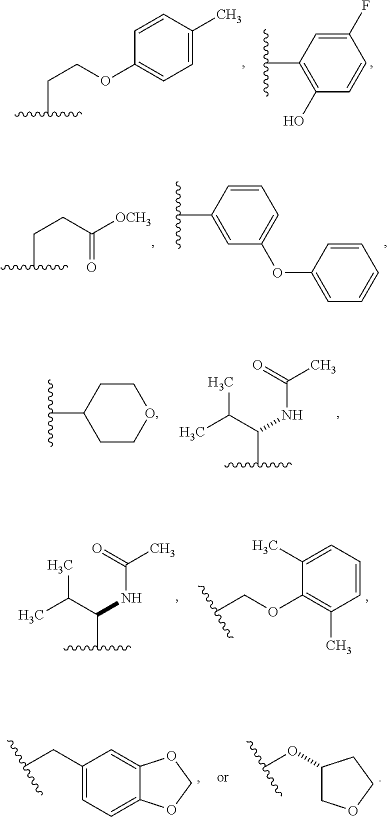

In certain embodiments, the present invention relates to the aforementioned compound and any of the attendant definitions, wherein R₄ is

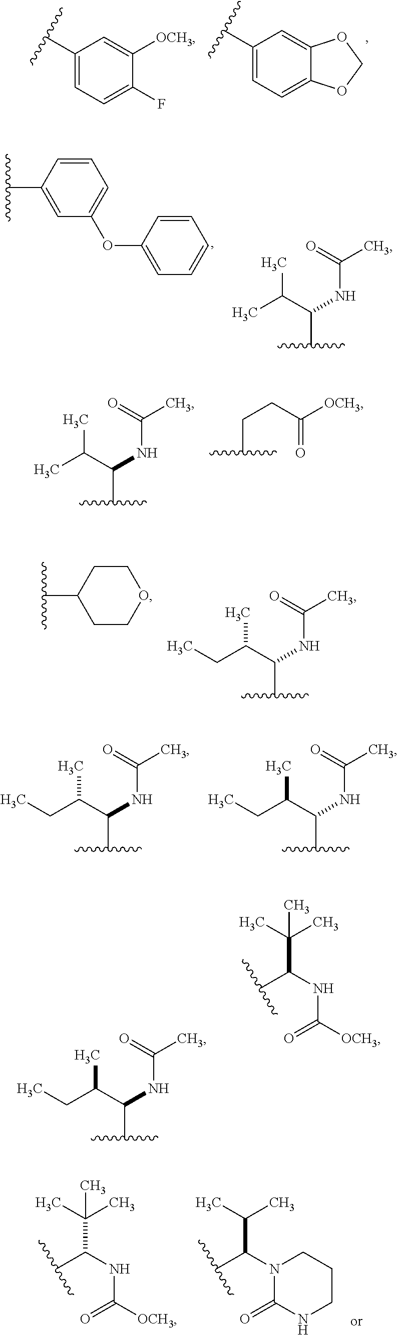

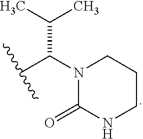

In certain embodiments, the present invention relates to the aforementioned compound and any of the attendant definitions, wherein R₇ is

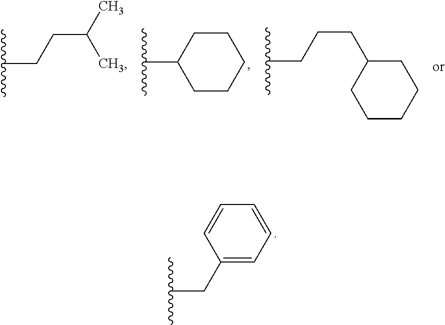

In certain embodiments, the present invention relates to the aforementioned compound and any of the attendant definitions, wherein X₁ is absent; and R₃ is

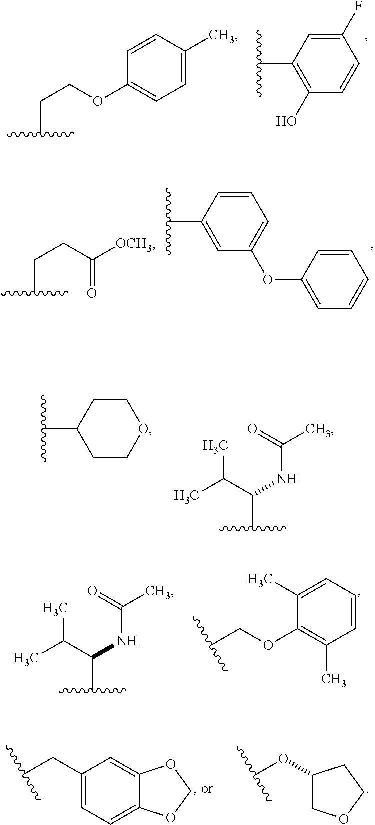

In certain embodiments, the present invention relates to the aforementioned compound and any of the attendant definitions, wherein X₁ is absent; R₃ is

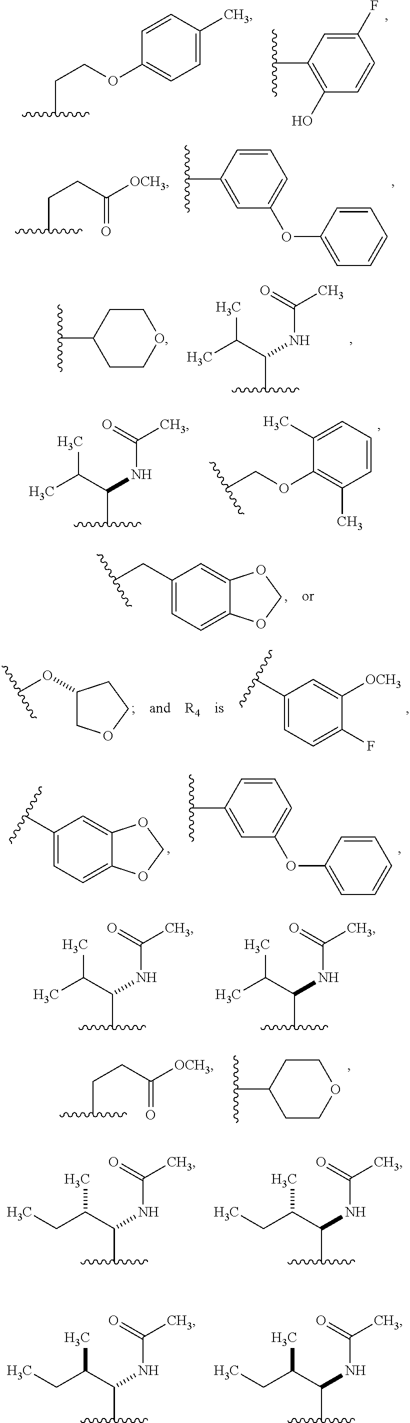

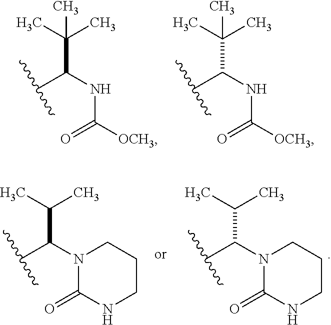

In certain embodiments, the present invention relates to the aforementioned compound and any of the attendant definitions, wherein X₁ is absent; R₃ is

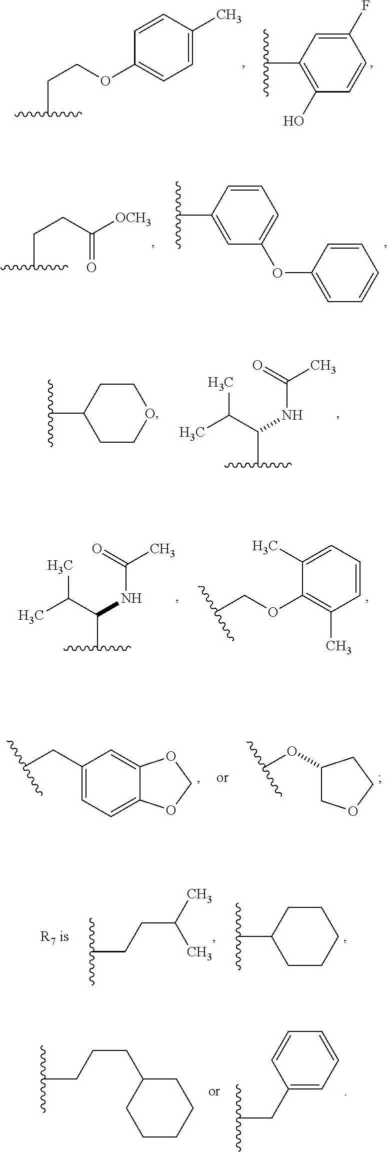

In certain embodiments, the present invention relates to the aforementioned compound and any of the attendant definitions, wherein R₄ is

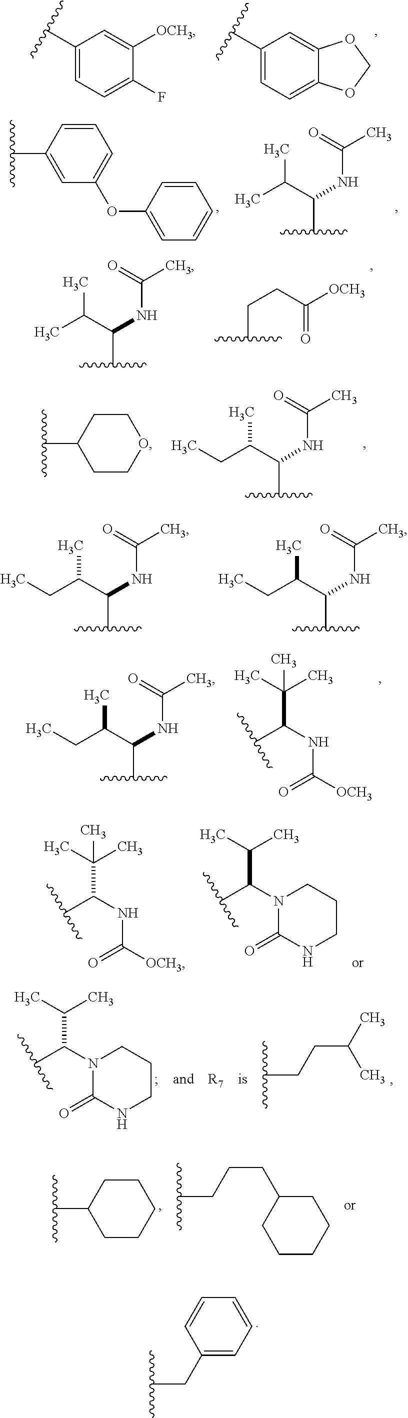

In certain embodiments, the present invention relates to the aforementioned compound and any of the attendant definitions, wherein X₁ is absent; R₃ is

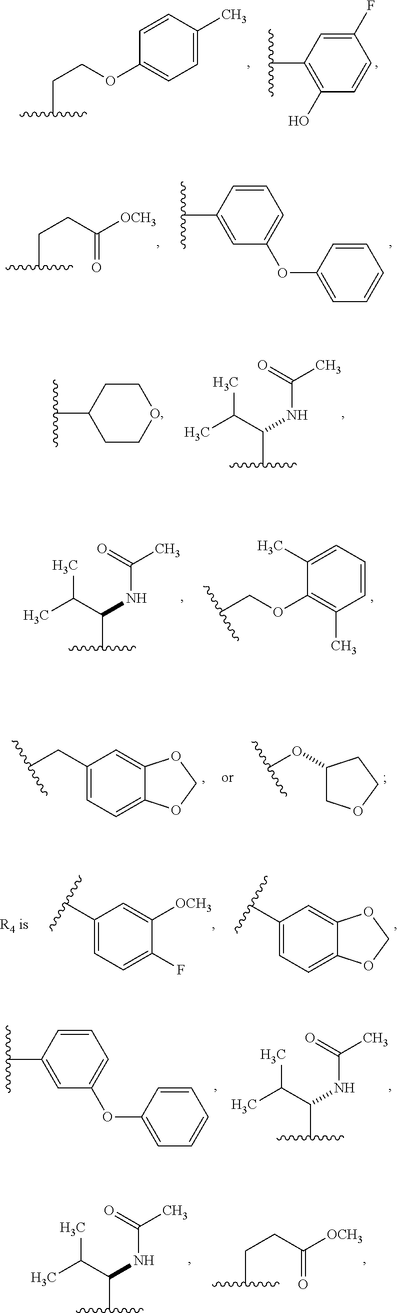

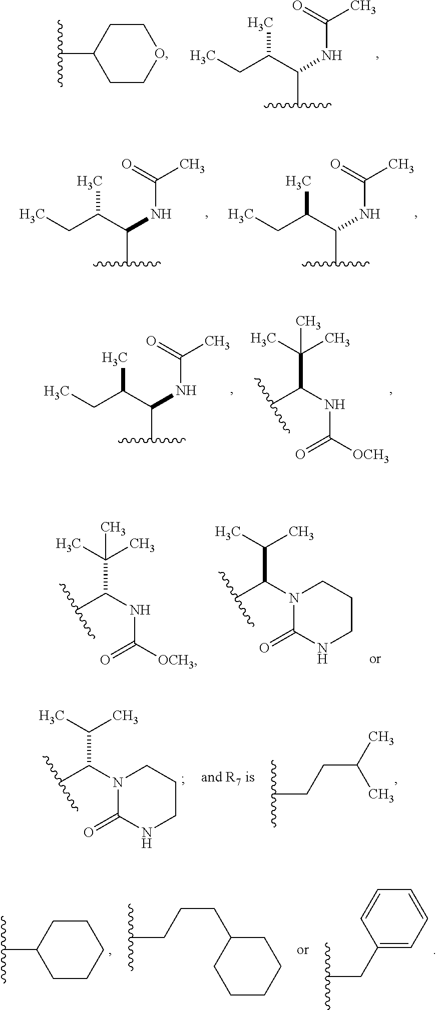

Another aspect of the present invention relates to a compound, or a pharmaceutically acceptable salt thereof, selected from the group consisting of

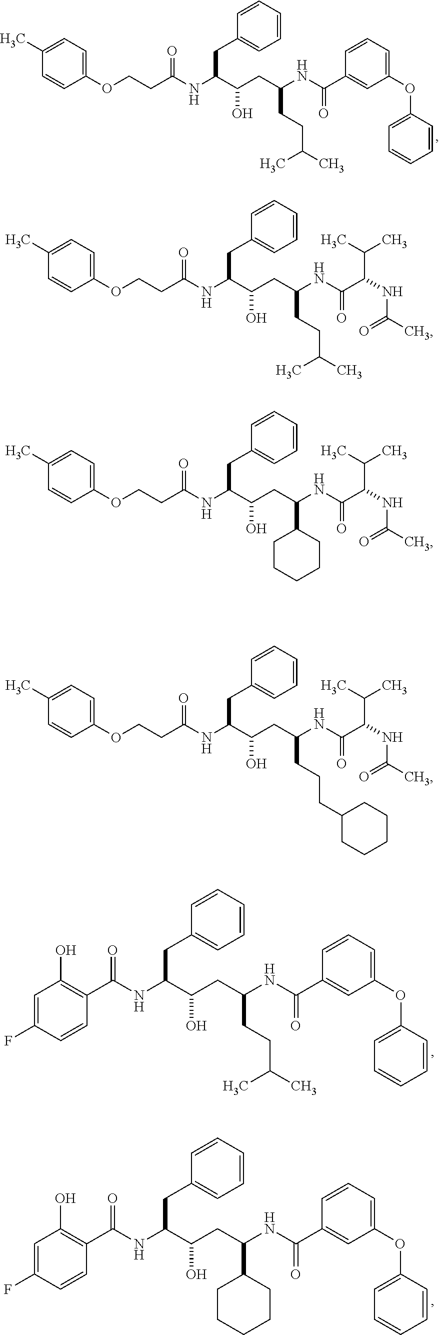

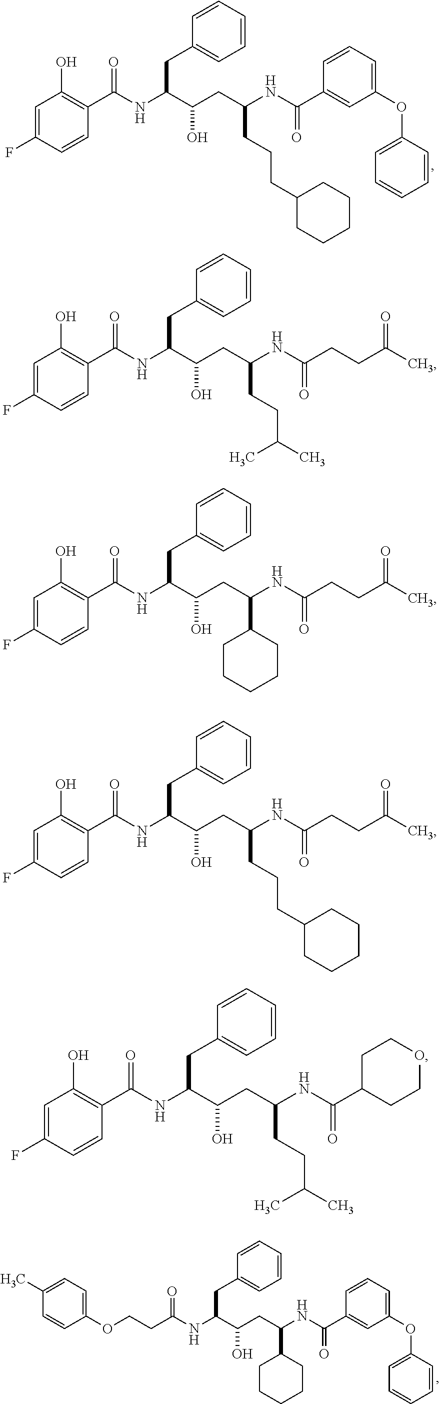

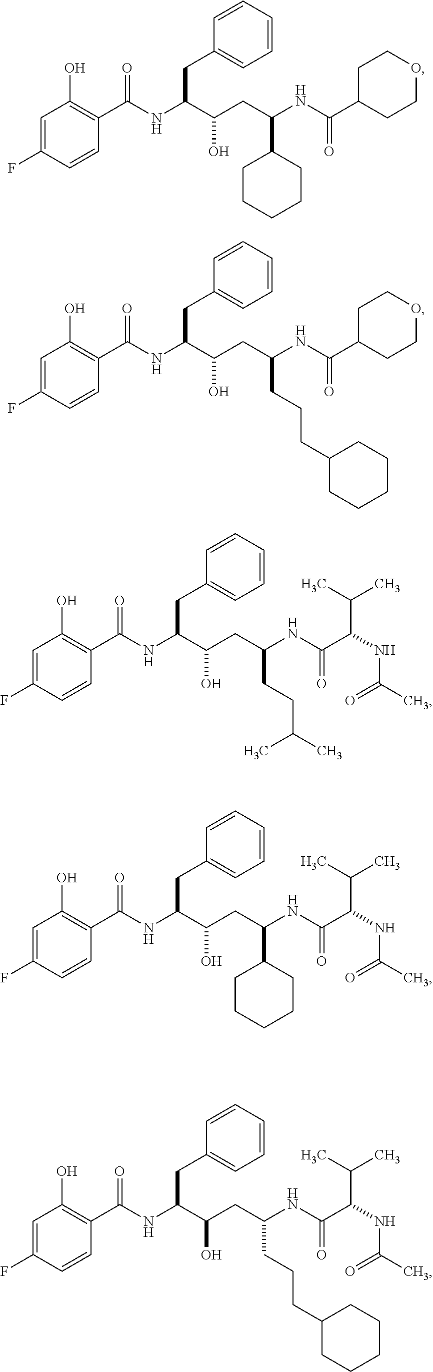

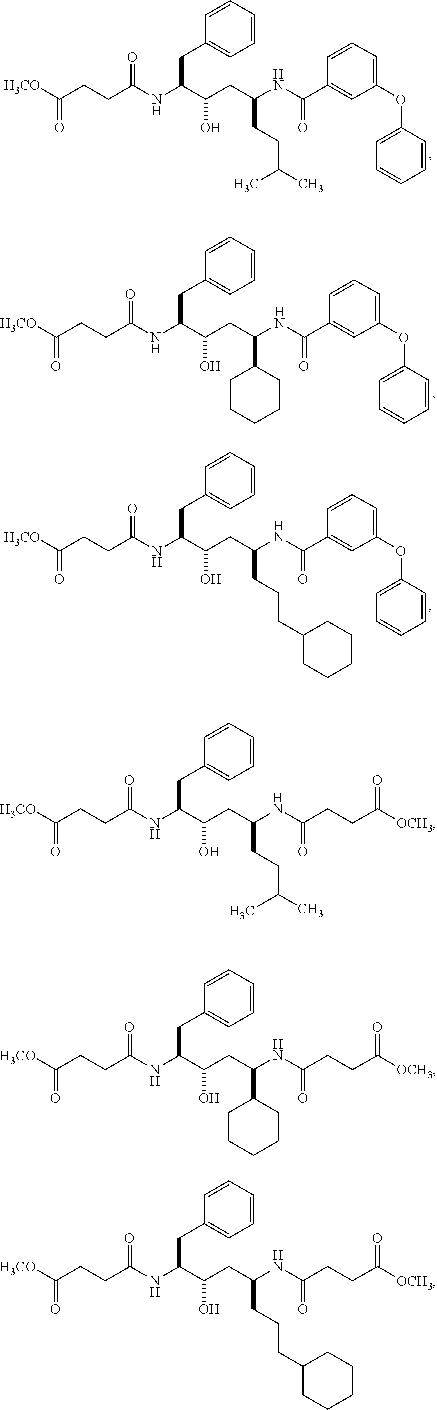

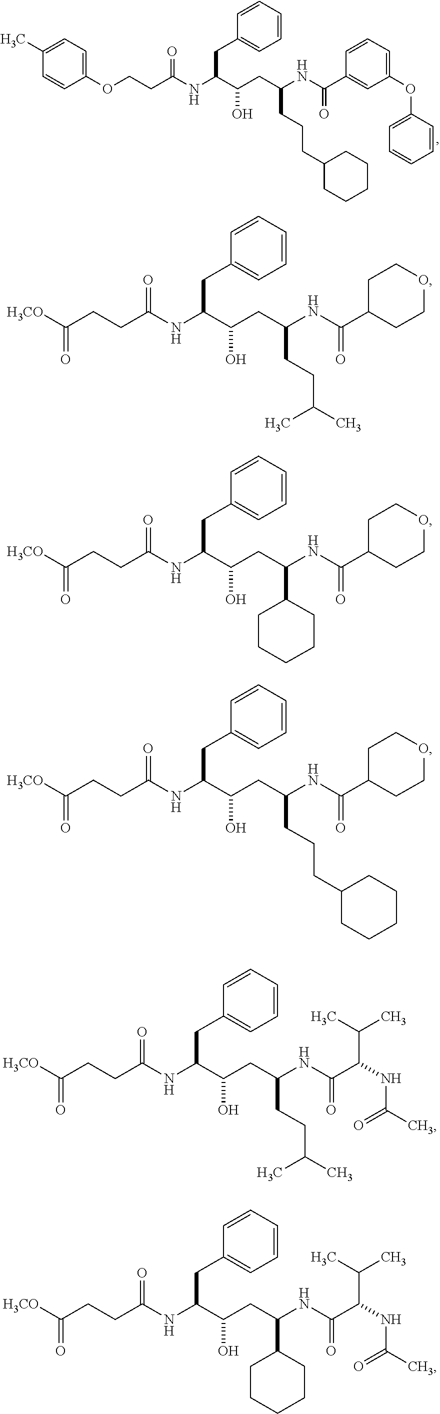

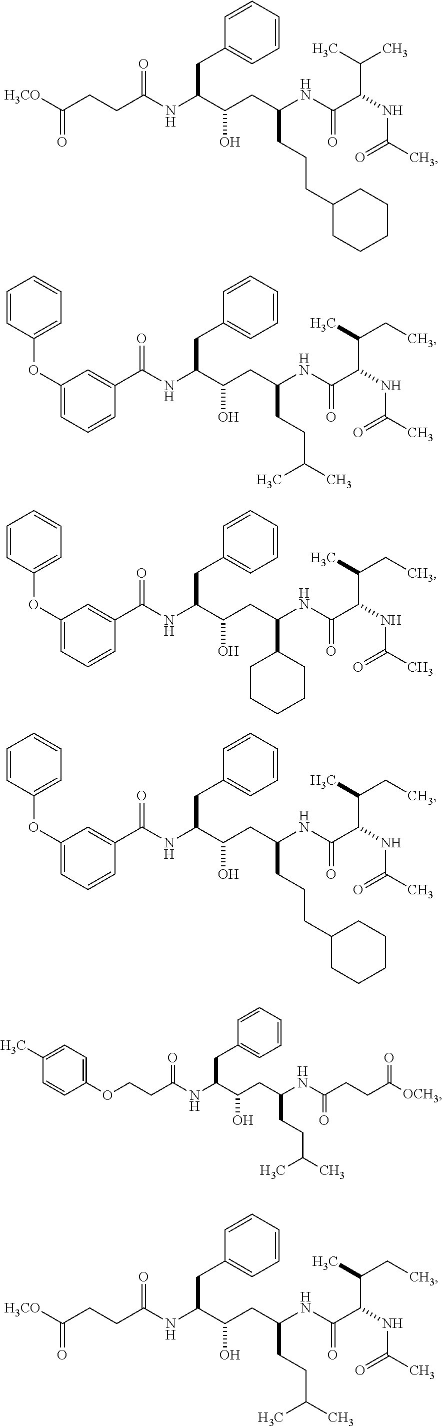

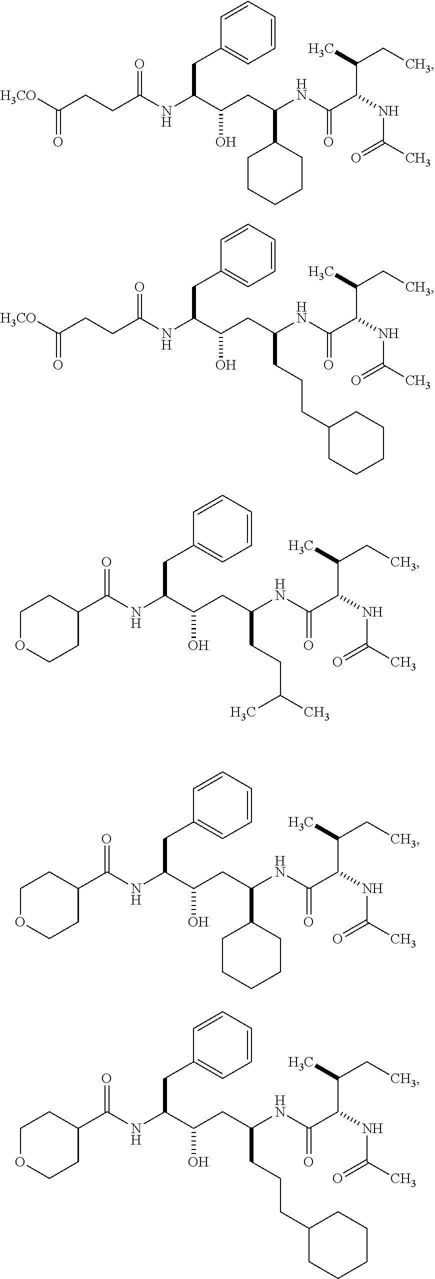

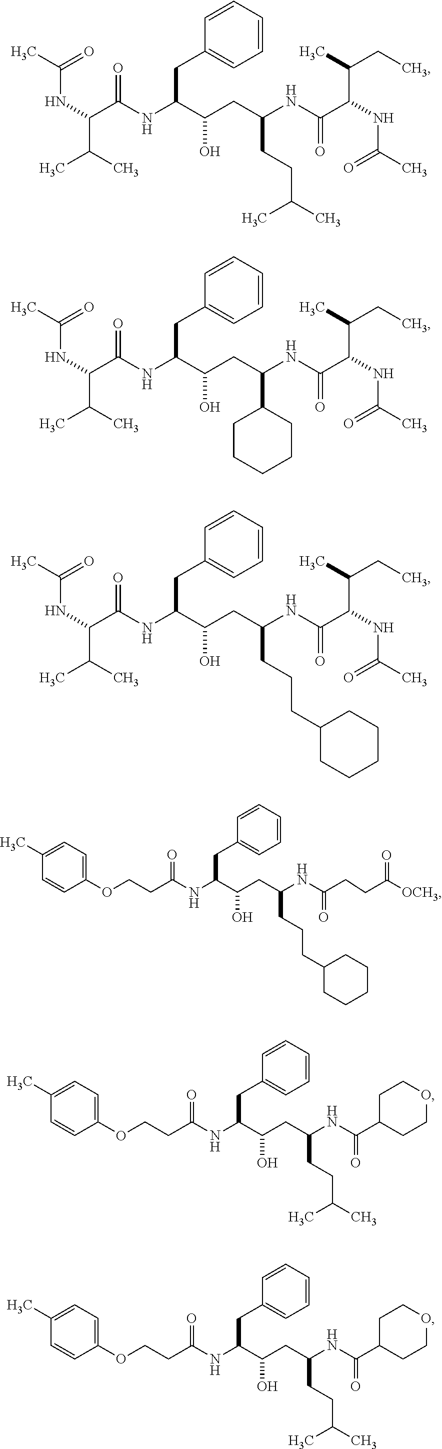

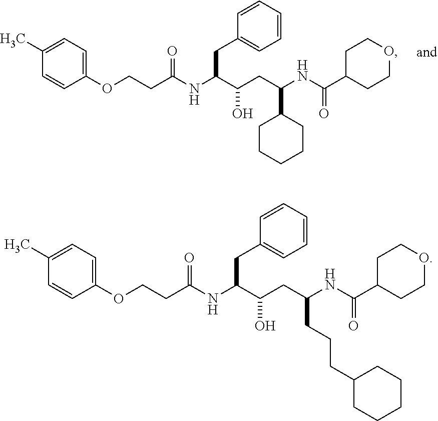

Another aspect of the present invention relates to a compound, or a pharmaceutically acceptable salt thereof, of formula V:

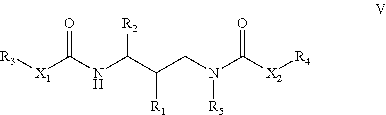

wherein, independently for each occurrence,

X₁ is absent, —O—, —S—, —NR— or

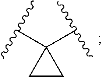

X₂ is absent, —O—, —S—, —NR— or

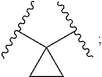

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, (cycloalkyl)alkyl, amino, (amino)alkyl, amido, (amido)alkyl, (keto)alkyl, cycloalkyl, heterocyclyl, aryl, heteroaryl, (heterocyclyl)alkyl, aralkyl or heteroaralkyl; and

the stereochemical configuration at any stereocenter is R, S, or a mixture of these configurations;

provided that when X₁ is absent; R₃ is not

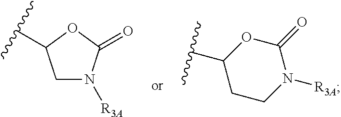

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

and provided that when X₂ is absent; R₄ is not

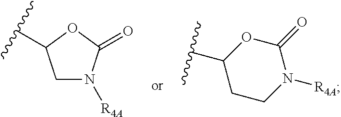

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein X₁ is —O—.

In certain embodiments, the present invention relates to the aforementioned compound and any of the attendant definitions, wherein X₂ is absent.

In certain embodiments, the present invention relates to the aforementioned compound and any of the attendant definitions, wherein X₁ is absent or —O—; and X₂ is absent.

In certain embodiments, the present invention relates to the aforementioned compound and any of the attendant definitions, wherein R₁ is OH.

In certain embodiments, the present invention relates to the aforementioned compound and any of the attendant definitions, wherein R₂ is aralkyl or heteroaralkyl.

In certain embodiments, the present invention relates to the aforementioned compound and any of the attendant definitions, wherein R₂ is aralkyl.

In certain embodiments, the present invention relates to the aforementioned compound and any of the attendant definitions, wherein R₂ is

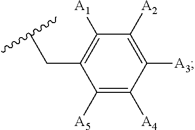

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to the aforementioned compound and any of the attendant definitions, wherein R₂ is

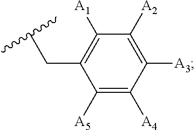

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₂ is

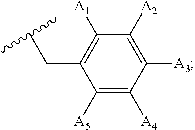

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₃ is alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₃ is heterocyclyl.

In certain embodiments, the present invention relates to the aforementioned compound and any of the attendant definitions, wherein R₄ is (heterocyclyl)alkyl.

In certain embodiments, the present invention relates to the aforementioned compound and any of the attendant definitions, wherein R₅ is alkyl.

In certain embodiments, the present invention relates to the aforementioned compound and any of the attendant definitions, wherein X₁ is absent or —O—; X₂ is absent; R₁ is OH; R₂ is aralkyl; R₃ is heterocyclyl; R₄ is alkyl, aryl or heteroaryl; and R₅ is alkyl.

Another aspect of the present invention relates to a compound, or a pharmaceutically acceptable salt thereof, of formula VI:

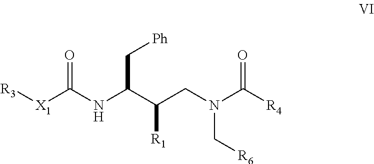

wherein, independently for each occurrence,

X₁ is absent or —O—;

R₁ is —OH or —NH₂;

R₃ is alkyl, cycloalkyl, heterocyclyl, aryl, heteroaryl, (heterocyclyl)alkyl, aralkyl or heteroaralkyl;

R₄ is aryl, amino, (amino)alkyl, amido, (amido)alkyl, heterocyclyl, (heterocyclyl)alkyl, heteroaryl, aralkyl or heteroaralkyl; and

R₆ is alkyl, cycloalkyl, or aryl;

provided that when X₁ is absent; R₃ is not

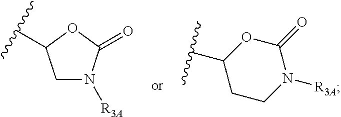

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

and provided that when X₂ is absent; R₄ is not

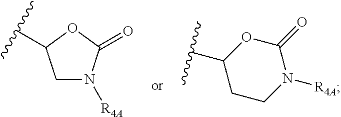

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₁ is —OH.

In certain embodiments, the present invention relates to the aforementioned compound and any of the attendant definitions, wherein R₁ is —NH₂.

In certain embodiments, the present invention relates to the aforementioned compound and any of the attendant definitions, wherein X₁ is —O—.

In certain embodiments, the present invention relates to the aforementioned compound and any of the attendant definitions, wherein R₃ is heterocyclyl.

In certain embodiments, the present invention relates to the aforementioned compound and any of the attendant definitions, wherein R₃ is

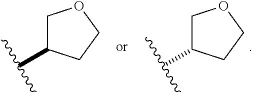

In certain embodiments, the present invention relates to the aforementioned compound and any of the attendant definitions, wherein X₁ is —O—; and R₃ is heterocyclyl.

In certain embodiments, the present invention relates to the aforementioned compound and any of the attendant definitions, wherein X₁ is —O—; and R₃ is

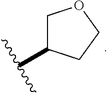

In certain embodiments, the present invention relates to the aforementioned compound and any of the attendant definitions, wherein R₄ is heterocyclyl.

In certain embodiments, the present invention relates to the aforementioned compound and any of the attendant definitions, wherein R₆ is alkyl.

n certain embodiments, the present invention relates to the aforementioned compound and any of the attendant definitions, wherein R₆ is —CH(CH₃)₂.

Another aspect of the present invention relates to a compound, or a pharmaceutically acceptable salt thereof, of formula VII:

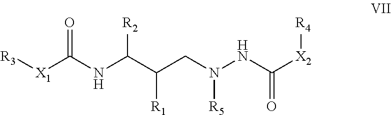

wherein, independently for each occurrence,

X₁ is absent, —O—, —S—, —NR— or

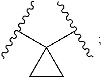

X₂ is absent, —O—, —S—, —NR— or

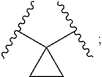

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, (cycloalkyl)alkyl, amino, (amino)alkyl, amido, (amido)alkyl, (keto)alkyl, cycloalkyl, heterocyclyl, aryl, heteroaryl, (heterocyclyl)alkyl, aralkyl or heteroaralkyl; and

the stereochemical configuration at any stereocenter is R, S, or a mixture of these configurations;

provided that when X₁ is absent; R₃ is not

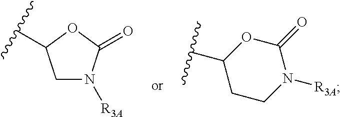

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

and provided that when X₂ is absent; R₄ is not

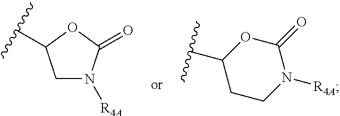

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein X₂ is absent.

In certain embodiments, the present invention relates to the aforementioned compound and any of the attendant definitions, wherein X₁ is absent; and X₂ is absent.

In certain embodiments, the present invention relates to the aforementioned compound and any of the attendant definitions, wherein R₁ is —OH or —NH₂.

In certain embodiments, the present invention relates to the aforementioned compound and any of the attendant definitions, wherein R₂ is aralkyl or heteroaralkyl.

In certain embodiments, the present invention relates to the aforementioned compound and any of the attendant definitions, wherein R₂ is aralkyl.

In certain embodiments, the present invention relates to the aforementioned compound and any of the attendant definitions, wherein R₂ is

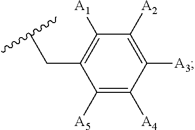

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to the aforementioned compound and any of the attendant definitions, wherein R₂ is

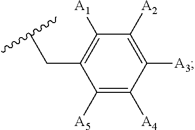

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₂ is

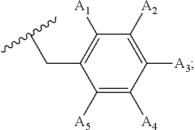

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to the aforementioned compound and any of the attendant definitions, wherein R₃ is alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein R₃ is (amido)alkyl or heterocyclyl.

In certain embodiments, the present invention relates to the aforementioned compound and any of the attendant definitions, wherein R₄ is (amido)alkyl or heterocyclyl.

In certain embodiments, the present invention relates to the aforementioned compound and any of the attendant definitions, wherein R₅ is alkyl or aryl.

In certain embodiments, the present invention relates to the aforementioned compound and any of the attendant definitions, wherein X₁ is absent; X₂ is absent; R₁ is —OH; R₂ is aralkyl; R₃ is (amido)alkyl or heterocyclyl; R₄ is (amido)alkyl or heterocyclyl; and R₅ is alkyl or aryl.

Another aspect of the present invention relates to a compound, or a pharmaceutically acceptable salt thereof, of formula VIII:

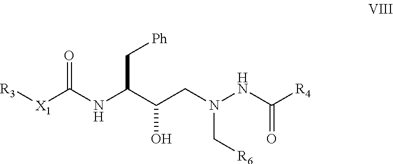

wherein, independently for each occurrence,

X₁ is absent or —O—;

R₃ is alkyl, alkenyl, amino, (amino)alkyl, amido, (amido)alkyl, cycloalkyl, heterocyclyl, aryl, heteroaryl, (heterocyclyl)alkyl, aralkyl or heteroaralkyl;

R₄ is aryl, heteroaryl, aralkyl or heteroaralkyl; and

R₆ is alkyl, cycloalkyl, heterocyclyl, aryl, heteroaryl, aralkyl or heteroaralkyl;

provided that when X₁ is absent; R₃ is not

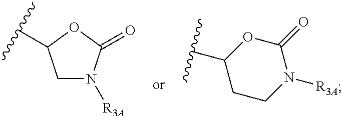

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;

and provided that when X₂ is absent; R₄ is not

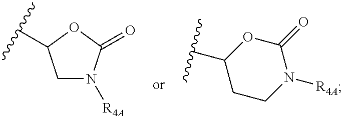

wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to the aforementioned compound and any of the attendant definitions, wherein X₁ is absent.

In certain embodiments, the present invention relates to the aforementioned compound and any of the attendant definitions, wherein R₃ is amino, (amino)alkyl, (amido)alkyl or heterocyclyl.

In certain embodiments, the present invention relates to the aforementioned compound and any of the attendant definitions, wherein R₃ is

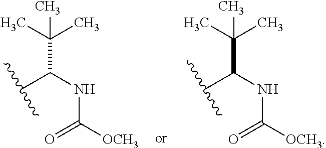

In certain embodiments, the present invention relates to the aforementioned compound and any of the attendant definitions, wherein R₄ is amino, (amino)alkyl, (amido)alkyl or heterocyclyl.

In certain embodiments, the present invention relates to the aforementioned compound and any of the attendant definitions, wherein R₄ is

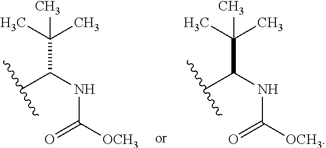

In certain embodiments, the present invention relates to the aforementioned compound and any of the attendant definitions, wherein R₆ is alkyl or aryl.

In certain embodiments, the present invention relates to the aforementioned compound and any of the attendant definitions, wherein R₆ is

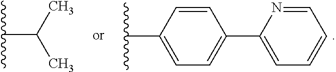

In certain embodiments, the present invention relates to the aforementioned compound and any of the attendant definitions, wherein R₃ is

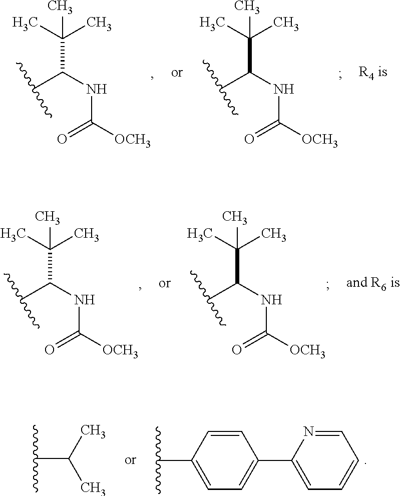

One aspect of the present invention relates to a compound, or a pharmaceutically acceptable salt thereof, of formula XI:

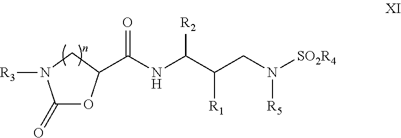

wherein, independently for each occurrence,

n is 1 or 2;

R₁ is —OH, —SH, or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, provided that when R₁ is —OH; R₂ is

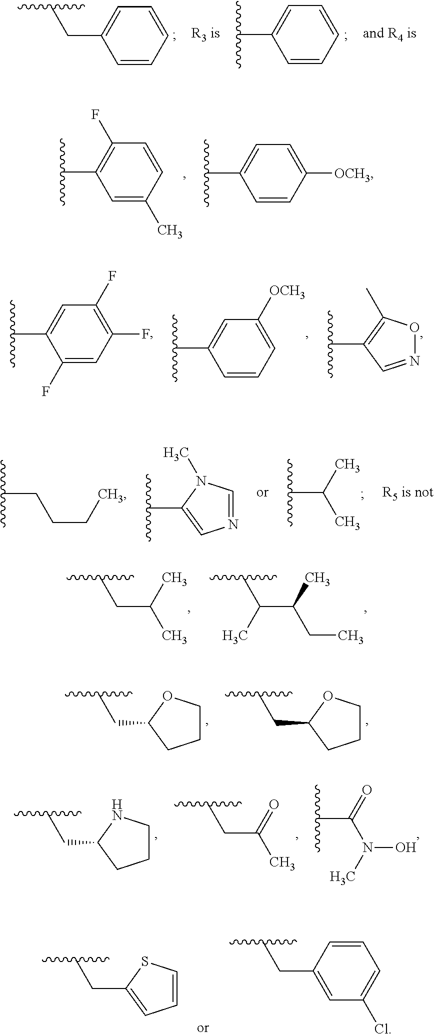

In certain embodiments, the present invention relates to any of the aforementioned compounds and any of the attendant definitions, provided that when R₁ is —OH; R₂ is

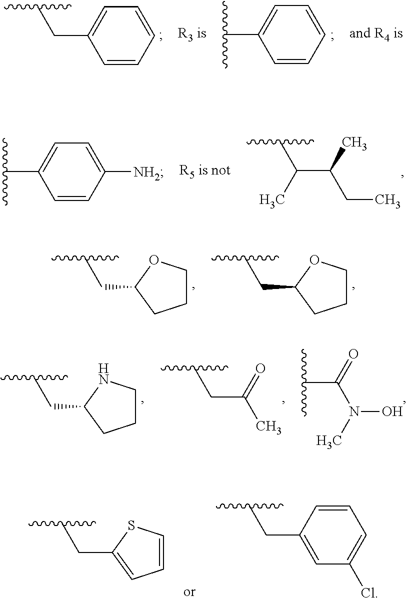

In certain embodiments, the present invention relates to any of the aforementioned compounds and any of the attendant definitions, provided that when R₁ is —OH; R₂ is

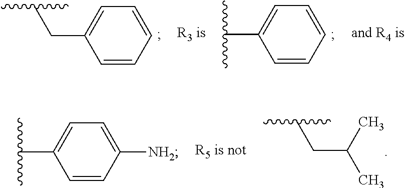

In certain embodiments, the present invention relates to any of the aforementioned compounds and any of the attendant definitions, provided that when R₁ is —OH; R₂ is

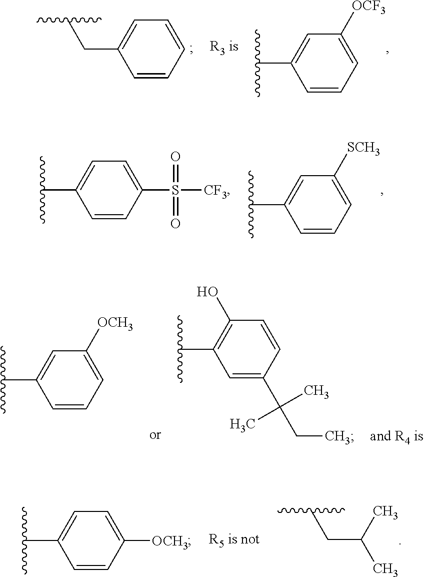

In certain embodiments, the present invention relates to any of the aforementioned compounds and any of the attendant definitions, provided that when R₁ is —OH; R₂ is

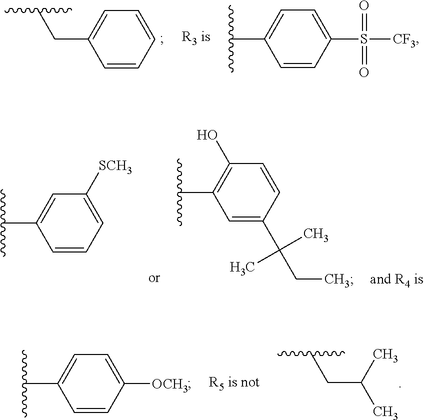

In certain embodiments, the present invention relates to the aformentioned compound and any of the attendant definitions, wherein n is 1.

In certain embodiments, the present invention relates to any of the aforementioned compounds and any of the attendant definitions, wherein R₁ is OH.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

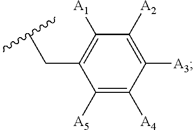

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

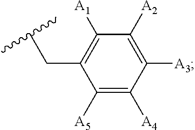

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₂ is

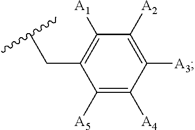

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₃ is aryl.

In certain embodiments, the present invention relates to any of the aforementioned compounds and any of the attendant definitions, wherein R₃ is

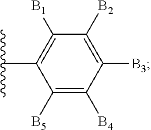

and B₁, B₂, B₃, B₄ and B₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₃ is

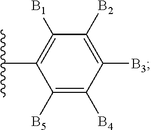

B₁, B₂, B₄ and B₅ are hydrogen; and B₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is

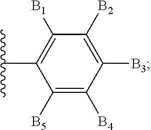

B₁, B₂, B₃ and B₅ are hydrogen; and B₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₄ is aryl.

In certain embodiments, the present invention relates to any of the aforementioned compounds and any of the attendant definitions, wherein R₄ is

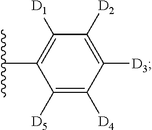

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₄ is

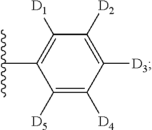

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is

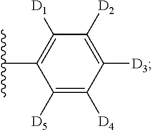

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₅ is alkyl, (heterocyclyl)alkyl, aralkyl or heteroaralkyl.

In certain embodiments, the present invention relates to any of the aforementioned compounds and any of the attendant definitions, wherein R₁ is OH; R₂ is aralkyl or heteroaralkyl; R₃ is aryl or heteroaryl; and R₄ is aryl or heteroaryl.

In certain embodiments, the present invention relates to any of the aforementioned compounds and any of the attendant definitions, wherein R₁ is OH; R₂ is aralkyl or heteroaralkyl; R₃ is aryl or heteroaryl; and R₅ is alkyl, (heterocyclyl)alkyl, aralkyl or heteroaralkyl.

In certain embodiments, the present invention relates to any of the aforementioned compounds and any of the attendant definitions, wherein R₁ is OH; R₂ is aralkyl or heteroaralkyl; R₄ is aryl or heteroaryl; and R₅ is alkyl, (heterocyclyl)alkyl, aralkyl or heteroaralkyl.

In certain embodiments, the present invention relates to any of the aforementioned compounds and any of the attendant definitions, wherein R₁ is OH; R₃ is aryl or heteroaryl; R₄ is aryl or heteroaryl; and R₅ is alkyl, (heterocyclyl)alkyl, aralkyl or heteroaralkyl.

In certain embodiments, the present invention relates to any of the aforementioned compounds and any of the attendant definitions, wherein R₁ is OH; R₂ is aralkyl; R₃ is aryl; R₄ is aryl; and R₅ is alkyl.

In certain embodiments, the present invention relates to any of the aforementioned compounds and any of the attendant definitions, wherein R₁ is OH; R₂ is aralkyl; R₃ is aryl; R₄ is aryl; and R₅ is aralkyl.

In certain embodiments, the present invention relates to any of the aforementioned compounds and any of the attendant definitions, wherein R₁ is OH; R₂ is aralkyl; R₃ is aryl; R₄ is aryl; and R₅ is heteroaralkyl.

In certain embodiments, the present invention relates to any of the aforementioned compounds and any of the attendant definitions, wherein R₁ is OH; R₂ is aralkyl; R₃ is aryl; R₄ is aryl; and R₅ is (heterocyclyl)alkyl.

Another aspect of the present invention relates to a compound, or a pharmaceutically acceptable salt thereof, of formula XII:

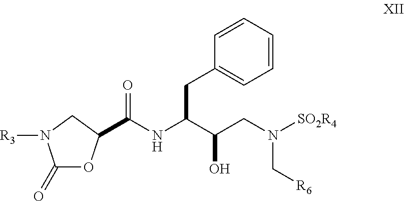

wherein, independently for each occurrence,

R₃ is alkyl, (amino)alkyl, (amido)alkyl, cycloalkyl, heterocyclyl, aryl, heteroaryl, (heterocyclyl)alkyl, aralkyl or heteroaralkyl;

R₄ is aryl, heteroaryl, aralkyl or heteroaralkyl; and

R₆ is alkyl, cycloalkyl, heterocyclyl, aryl, heteroaryl, aralkyl or heteroaralkyl.

Another aspect of the present invention relates to a compound, or a pharmaceutically acceptable salt thereof, of formula iso-XII:

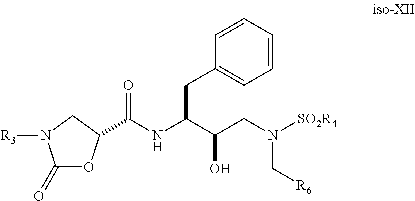

wherein, independently for each occurrence,

R₃ is alkyl, (amino)alkyl, (amido)alkyl, cycloalkyl, heterocyclyl, aryl, heteroaryl, (heterocyclyl)alkyl, aralkyl or heteroaralkyl;

R₄ is aryl, heteroaryl, aralkyl or heteroaralkyl; and

R₆ is alkyl, cycloalkyl, heterocyclyl, aryl, heteroaryl, aralkyl or heteroaralkyl.

In certain embodiments, the present invention relates to any of the aforementioned compounds and any of the attendant definitions, provided that when R₃ is

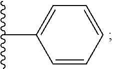

### and R₄ is

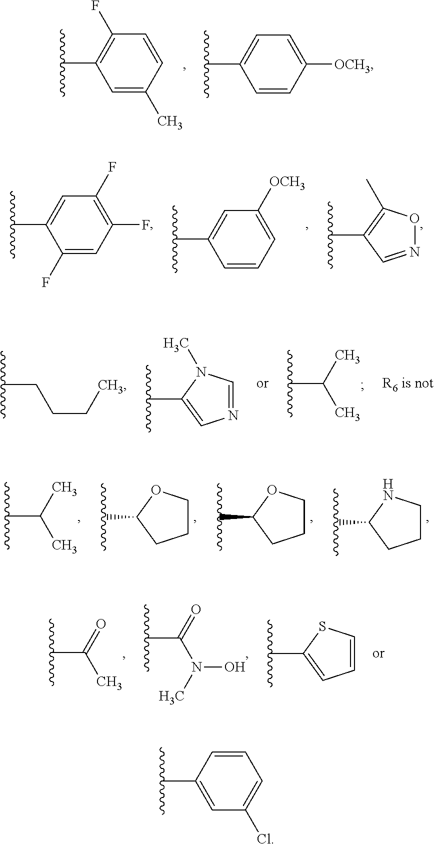

In certain embodiments, the present invention relates to any of the aforementioned compounds and any of the attendant definitions, provided that when R₃ is

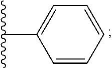

### and R₄ is

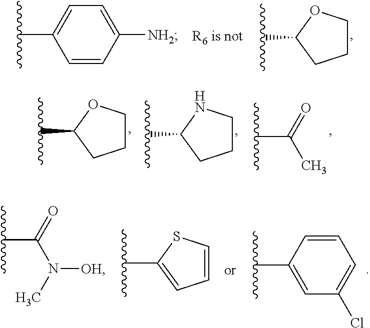

In certain embodiments, the present invention relates to any of the aforementioned compounds and any of the attendant definitions, provided that when R₃ is

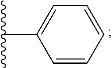

### and R₄ is

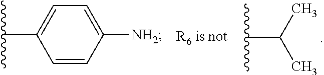

In certain embodiments, the present invention relates to any of the aforementioned compounds and any of the attendant definitions, provided that when R₃ is

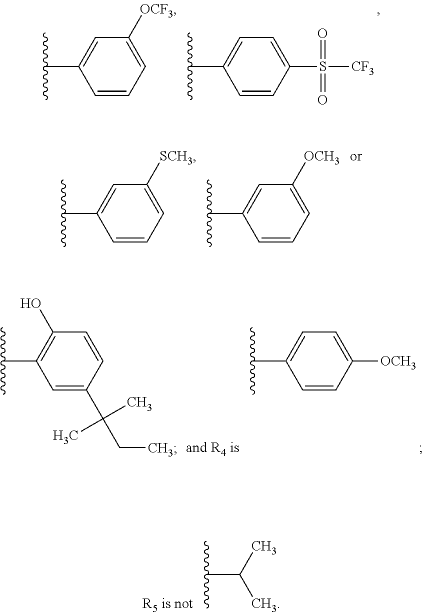

In certain embodiments, the present invention relates to any of the aforementioned compounds and any of the attendant definitions, provided that when R₃ is

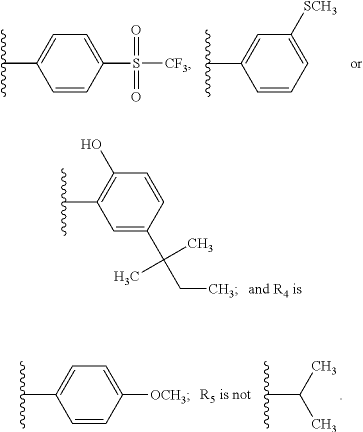

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₃ is aryl.

In certain embodiments, the present invention relates to any of the aforementioned compounds and any of the attendant definitions, wherein R₃ is

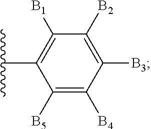

and B₁, B₂, B₃, B₄ and B₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₃ is

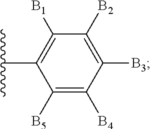

B₁, B₂, B₄ and B₅ are hydrogen; and B₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is

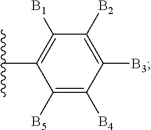

B₁, B₂, B₃ and B₅ are hydrogen; and B₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₄ is aryl.

In certain embodiments, the present invention relates to any of the aforementioned compounds and any of the attendant definitions, wherein R₄ is

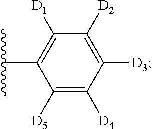

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₄ is

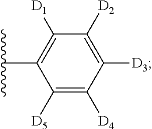

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is

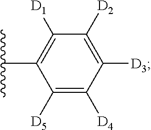

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₆ is alkyl, heterocyclyl, aryl or heteroaryl.

In certain embodiments, the present invention relates to any of the aforementioned compounds and any of the attendant definitions, wherein R₆ is alkyl.

In certain embodiments, the present invention relates to any of the aforementioned compounds and any of the attendant definitions, wherein R₃ is aryl or heteroaryl; and R₄ is aryl or heteroaryl.

In certain embodiments, the present invention relates to any of the aforementioned compounds and any of the attendant definitions, wherein R₃ is aryl or heteroaryl; and R₆ is alkyl, heterocyclyl, aryl or heteroaryl.

In certain embodiments, the present invention relates to any of the aforementioned compounds and any of the attendant definitions, wherein R₄ is aryl or heteroaryl; and R₆ is alkyl, heterocyclyl, aryl or heteroaryl.

In certain embodiments, the present invention relates to any of the aforementioned compounds and any of the attendant definitions, wherein R₃ is aryl or heteroaryl; R₄ is aryl or heteroaryl; and R₆ is alkyl, heterocyclyl, aryl or heteroaryl.

In certain embodiments, the present invention relates to any of the aforementioned compounds and any of the attendant definitions, wherein R₃ is aryl; and R₄ is aryl.

In certain embodiments, the present invention relates to any of the aforementioned compounds and any of the attendant definitions, wherein R₃ is aryl; and R₆ is alkyl.

In certain embodiments, the present invention relates to any of the aforementioned compounds and any of the attendant definitions, wherein R₄ is aryl; and R₆ is alkyl.

In certain embodiments, the present invention relates to any of the aforementioned compounds and any of the attendant definitions, wherein R₃ is aryl; R₄ is aryl; and R₆ is alkyl.

In certain embodiments, the present invention relates to any of the aforementioned compounds and any of the attendant definitions, wherein R₃ is

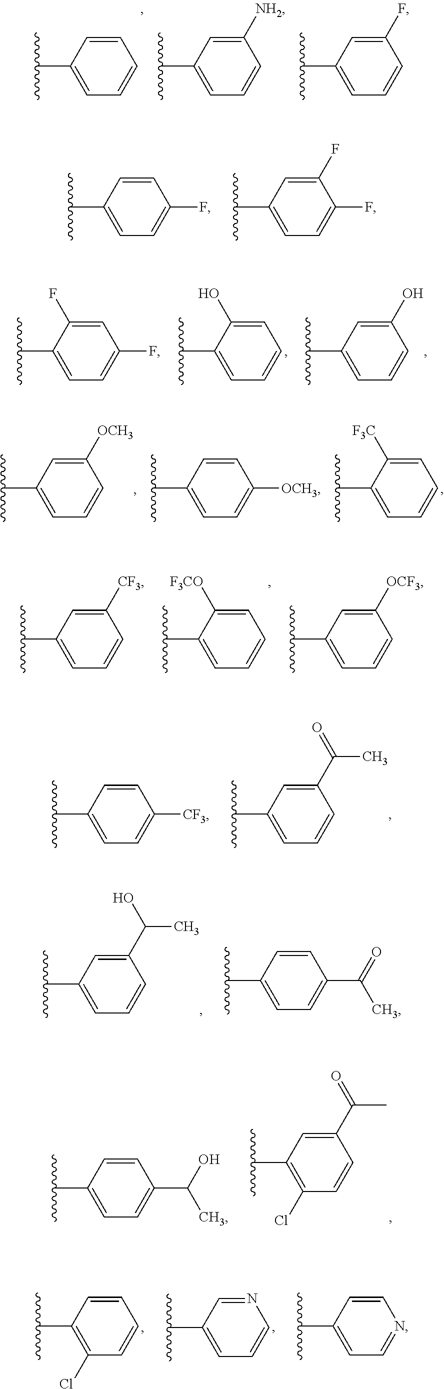

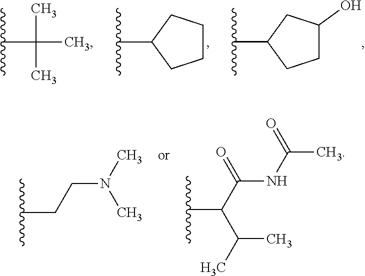

In certain embodiments, the present invention relates to any of the aforementioned compounds and any of the attendant definitions, wherein R₄ is

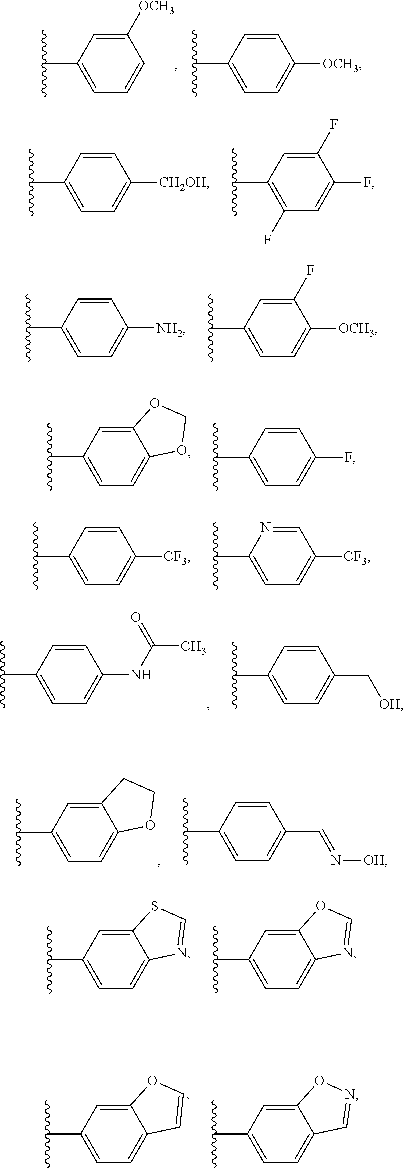

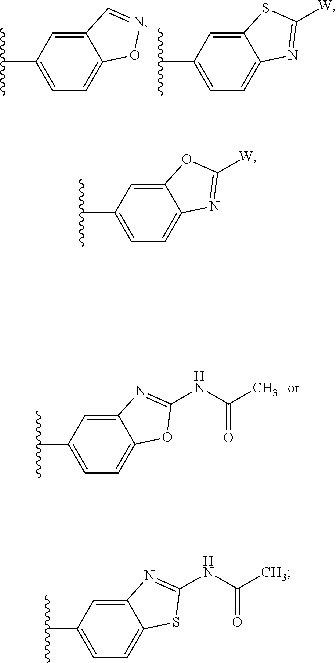

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

In certain embodiments, the present invention relates to any of the aforementioned compounds and any of the attendant definitions, wherein R₆ is

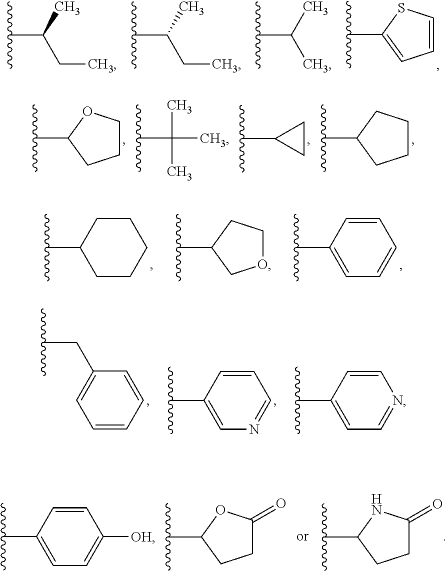

In certain embodiments, the present invention relates to any of the aforementioned compounds and any of the attendant definitions, wherein R₃ is

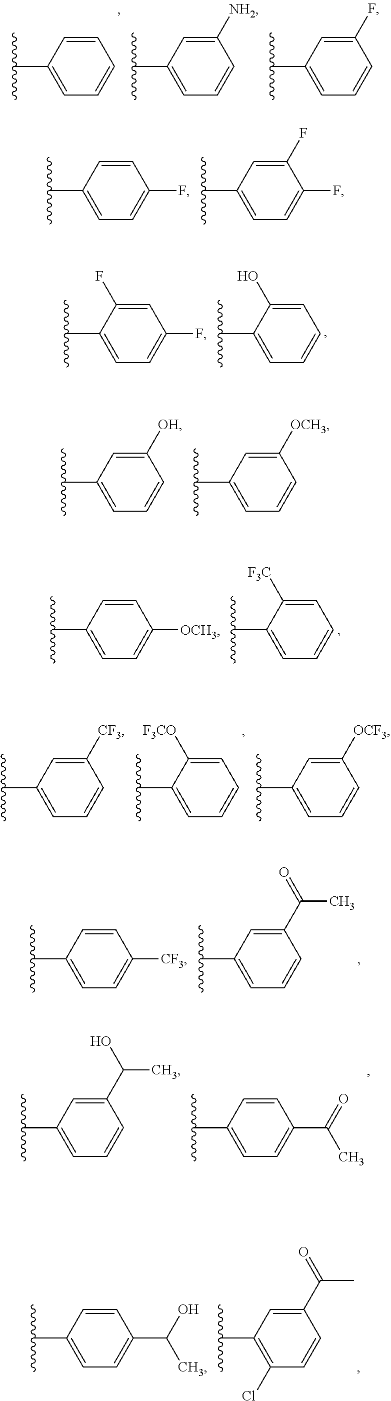

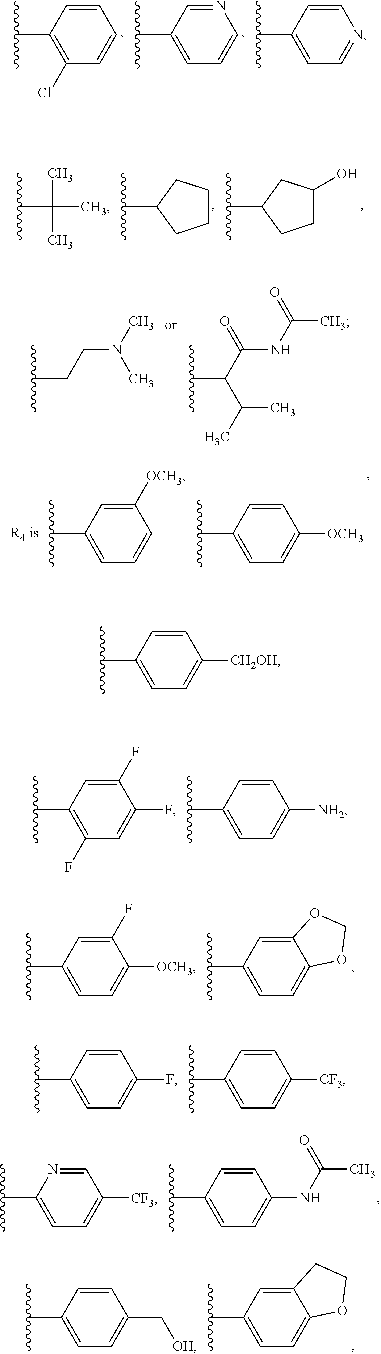

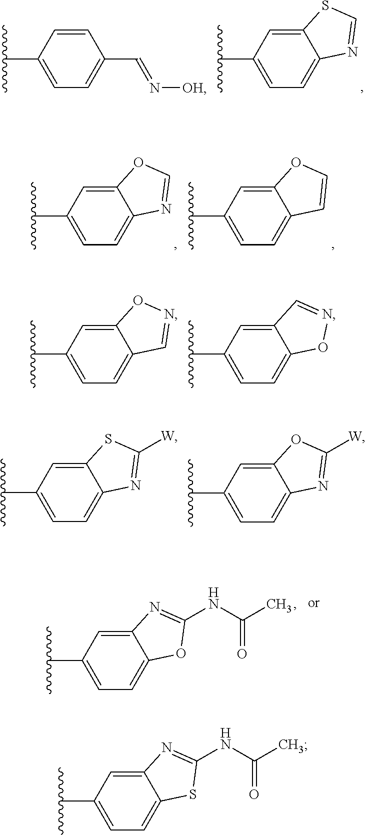

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

In certain embodiments, the present invention relates to any of the aforementioned compounds and any of the attendant definitions, wherein R₃ is

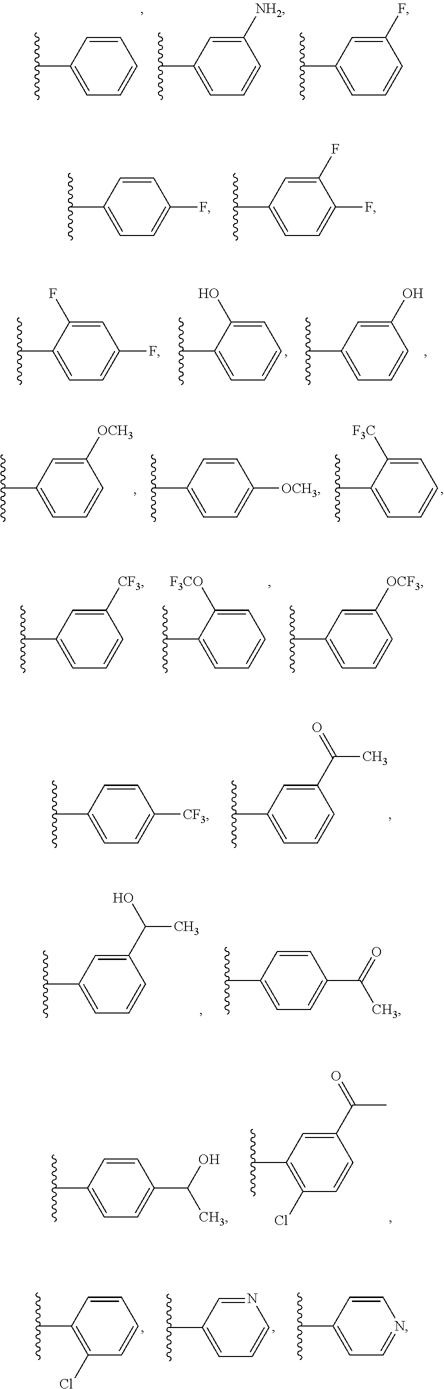

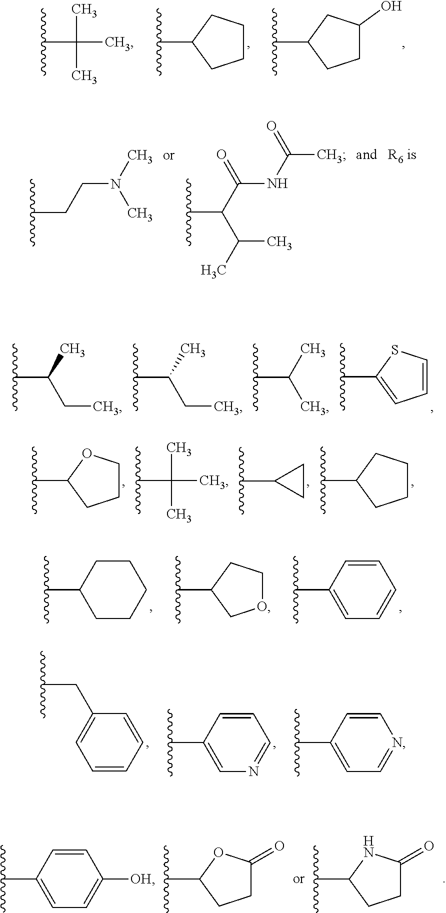

In certain embodiments, the present invention relates to any of the aforementioned compounds and any of the attendant definitions, wherein R₄ is

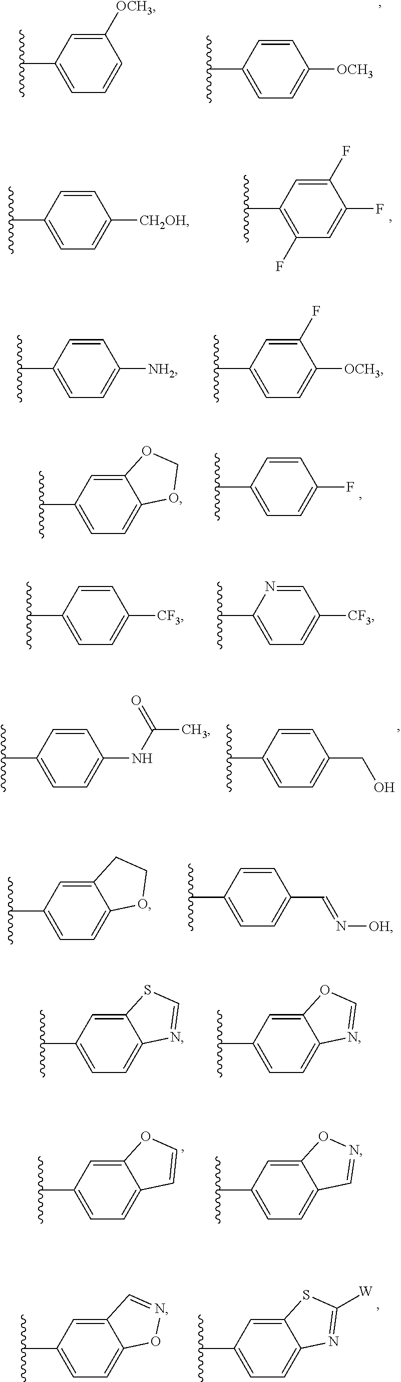

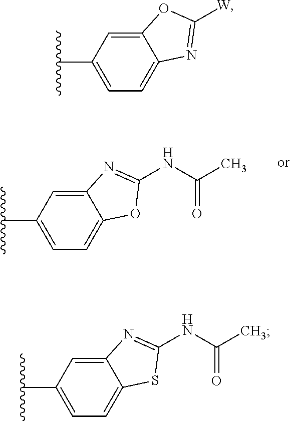

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; p is 1-10 inclusive; and R₆ is

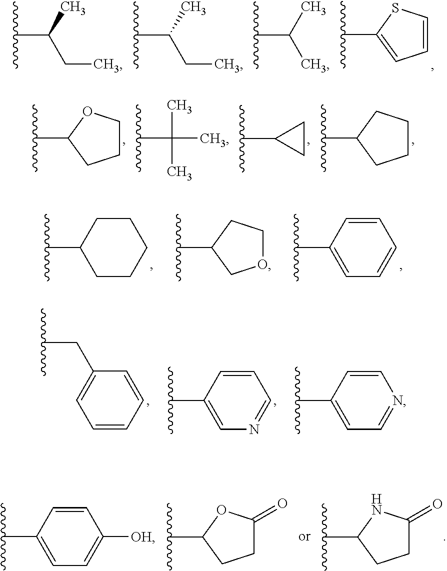

In certain embodiments, the present invention relates to any of the aforementioned compounds and any of the attendant definitions, wherein R₃ is

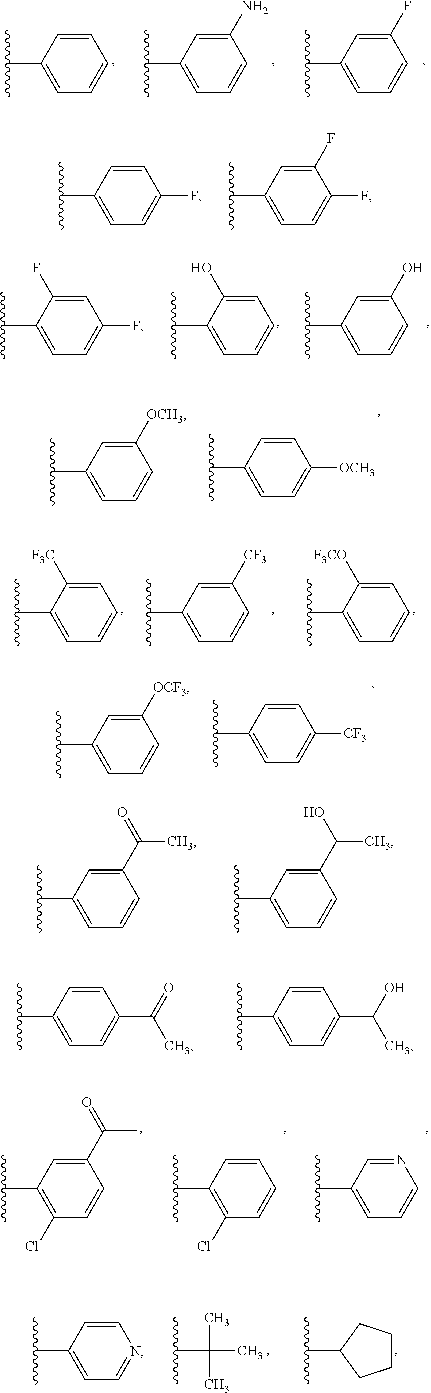

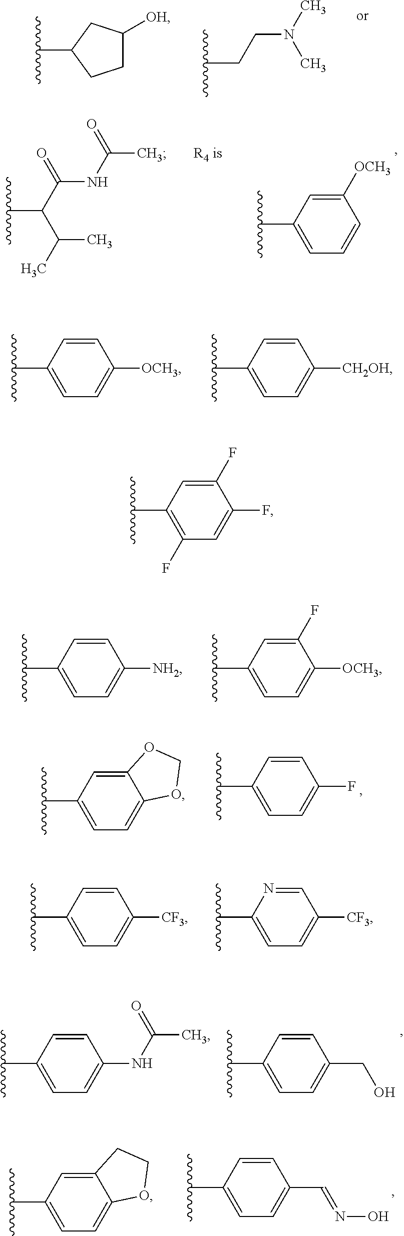

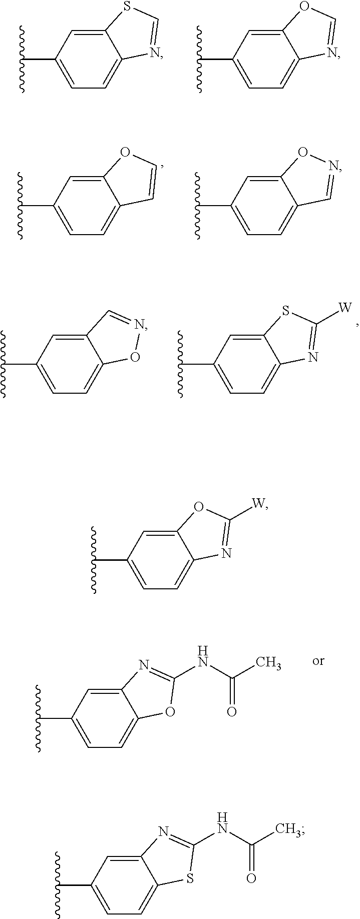

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; p is 1-10 inclusive; and R₆ is

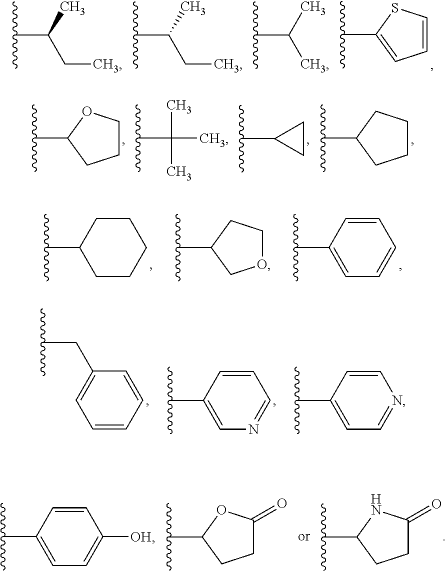

Another aspect of the present invention relates to a compound, or pharmaceutically acceptable salt thereof, selected from the group consisting of:

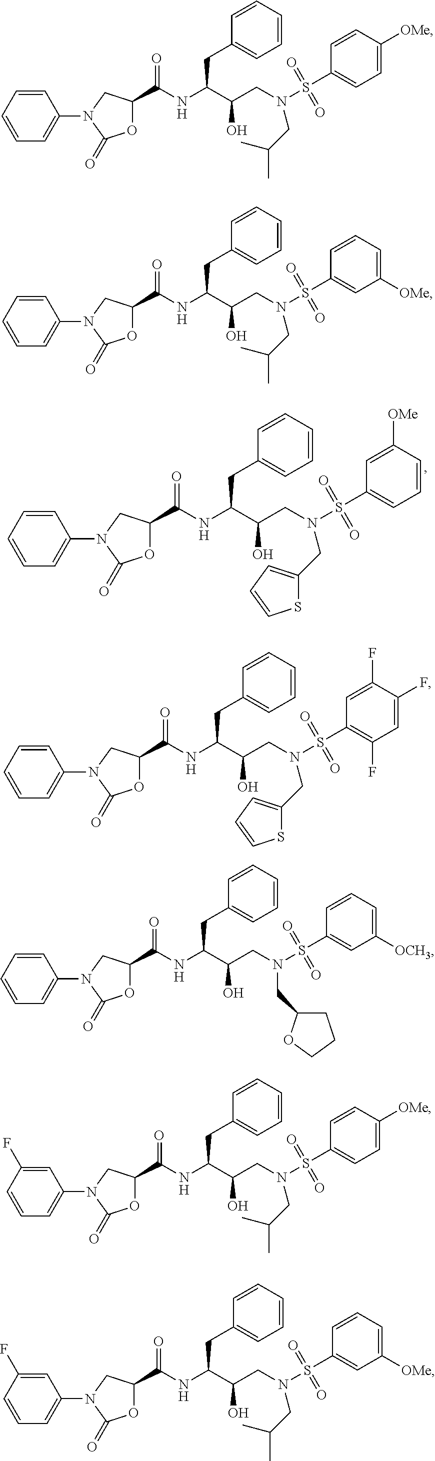

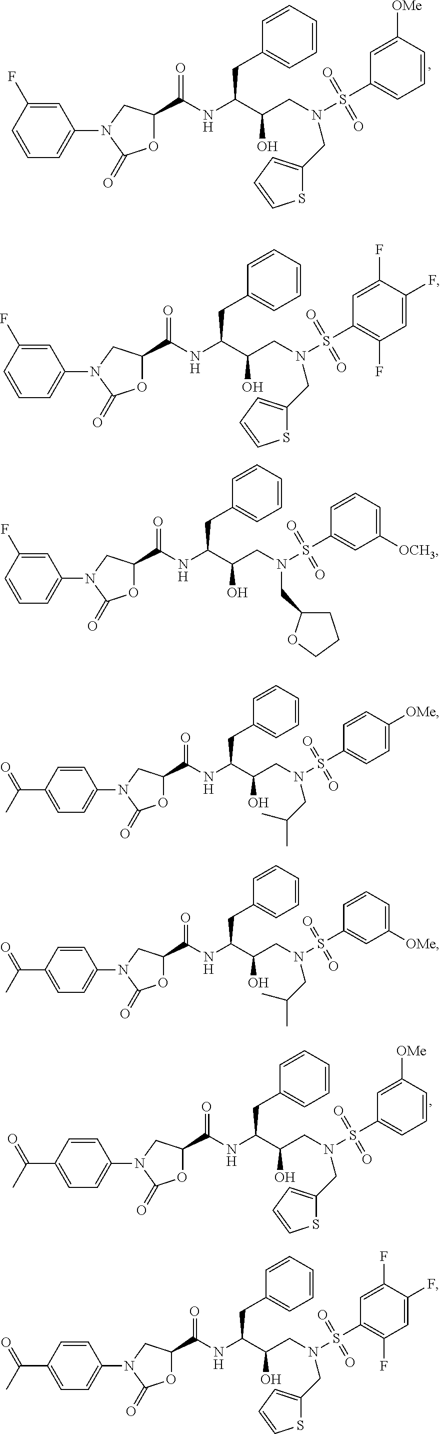

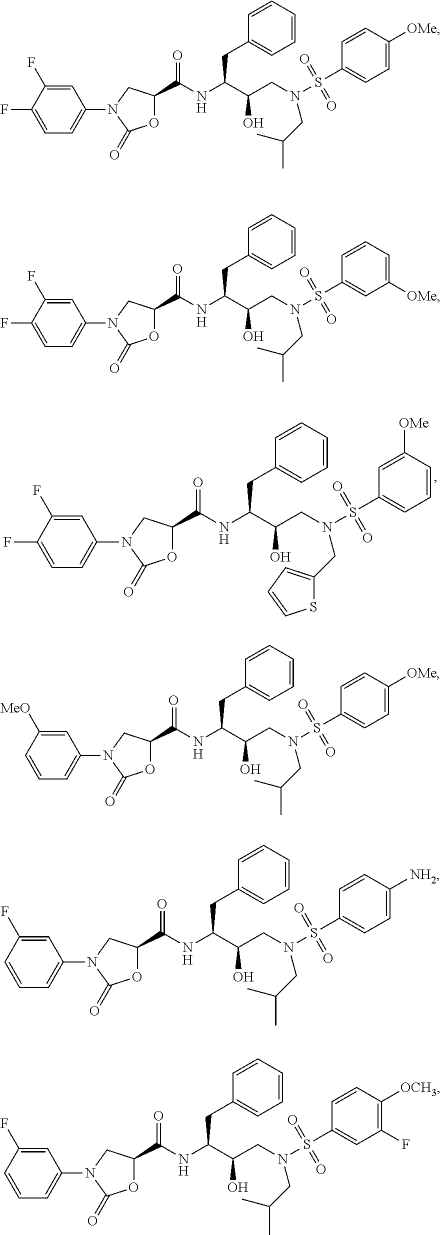

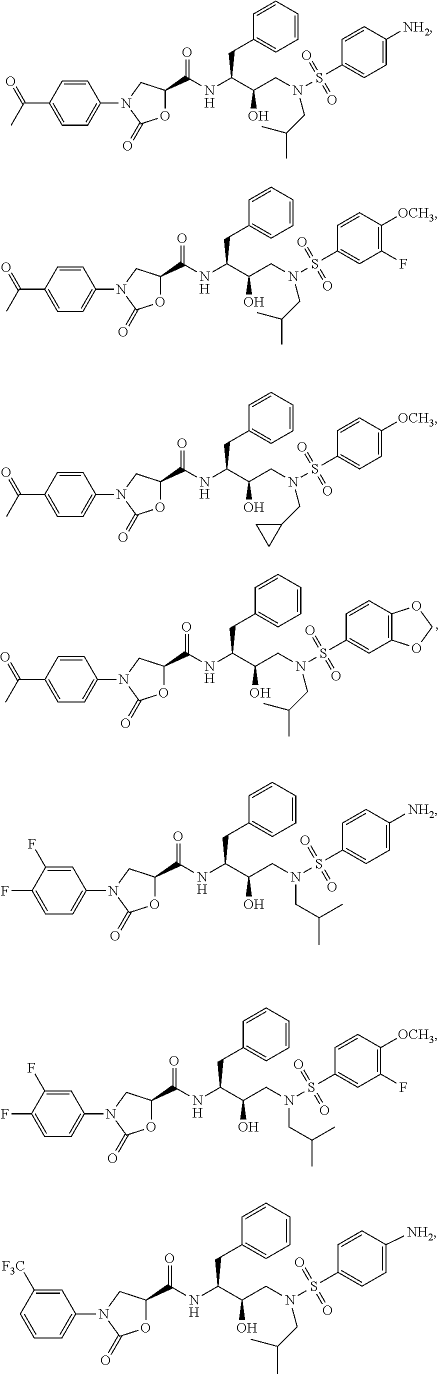

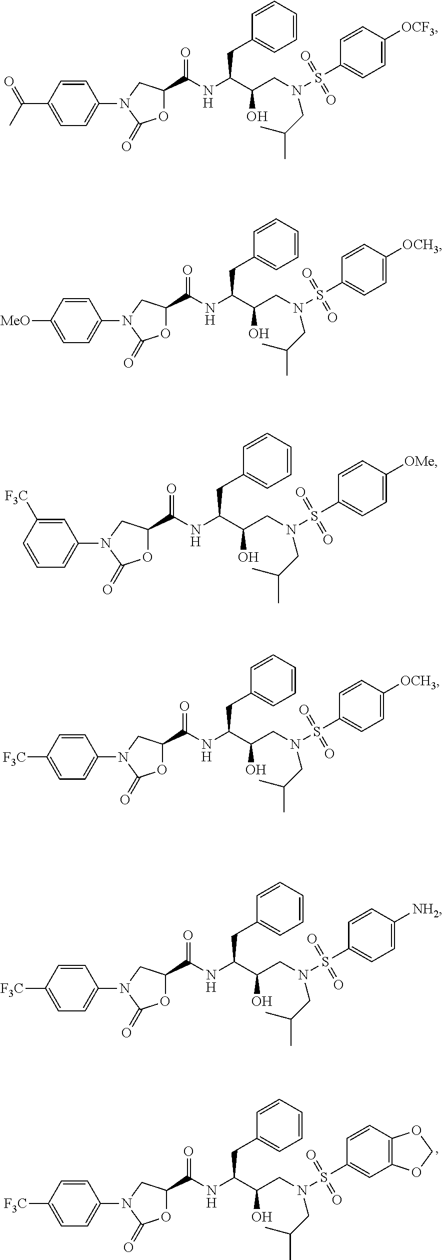

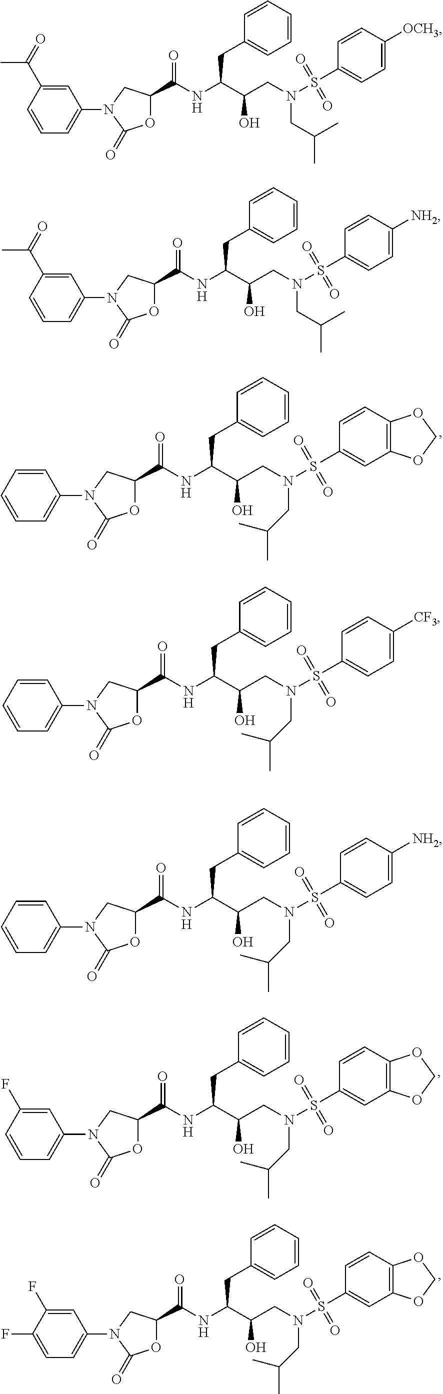

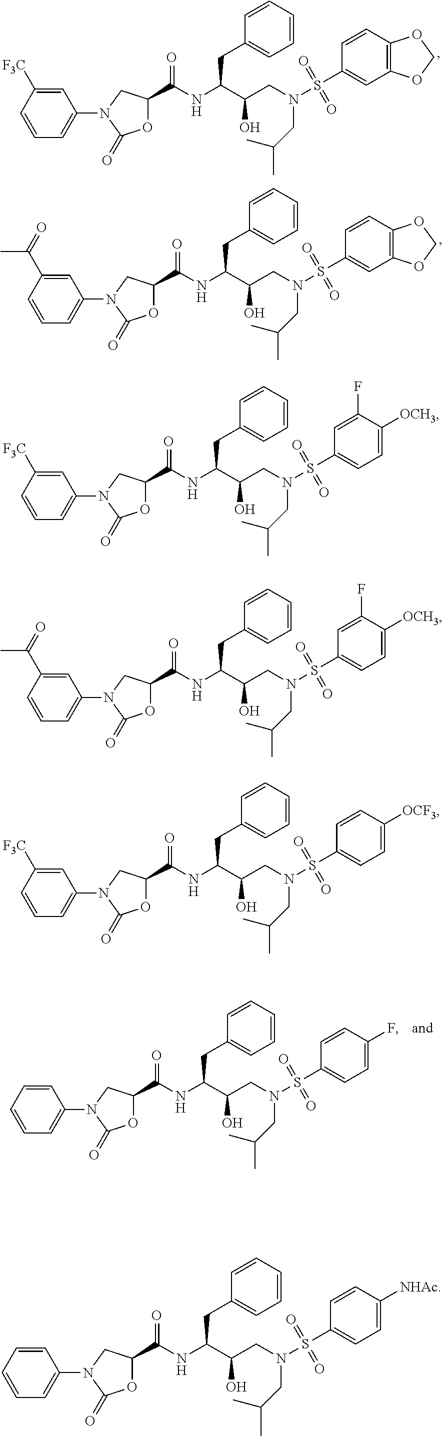

Another aspect of the present invention relates to a compound, or pharmaceutically acceptable salt thereof, selected from the group consisting of:

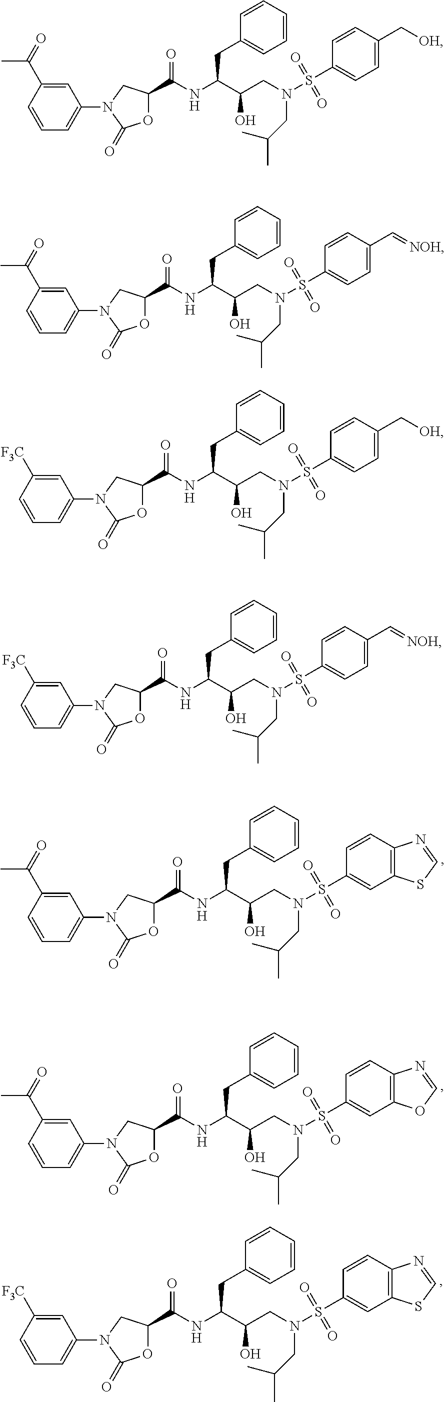

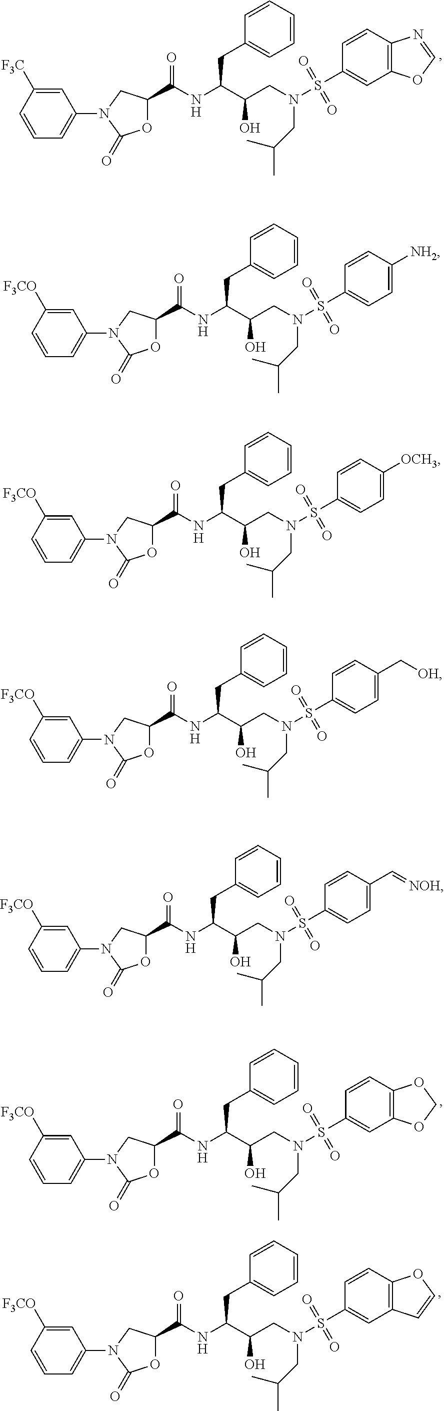

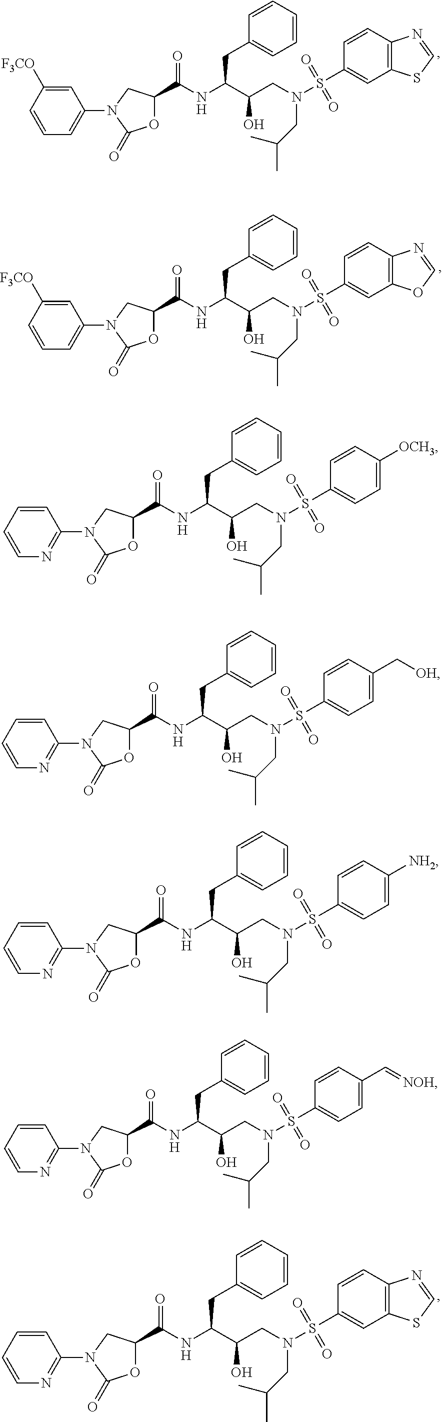

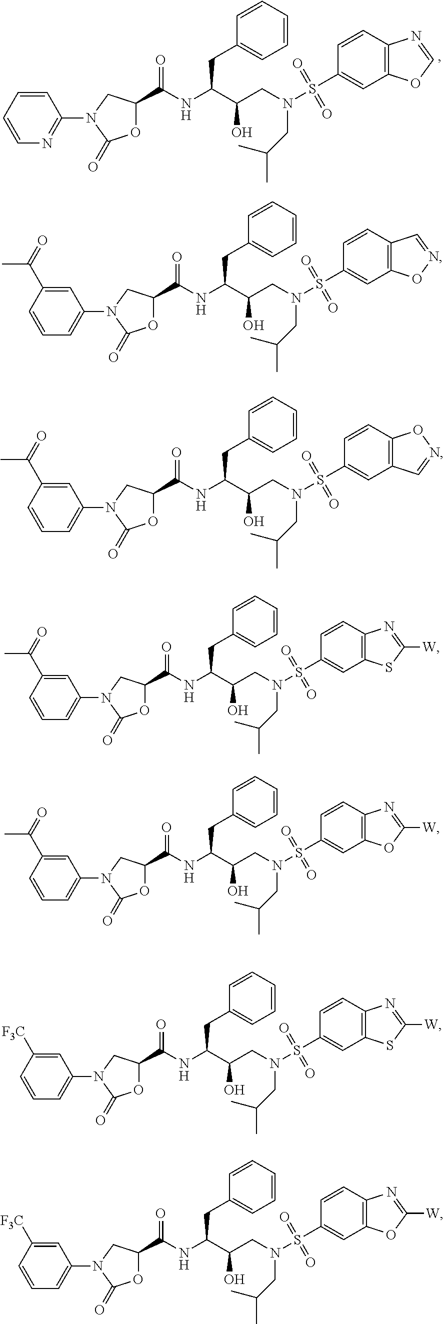

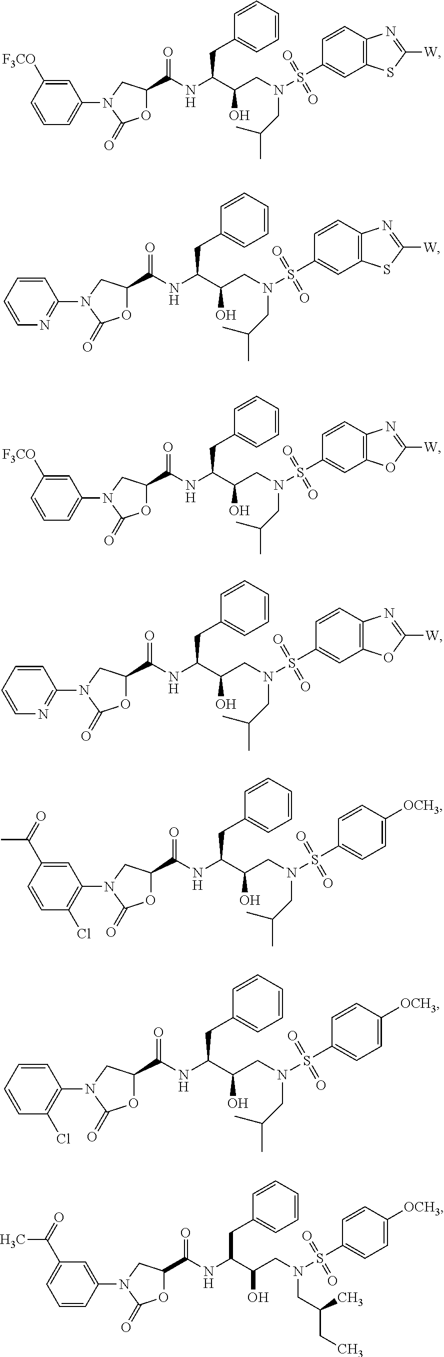

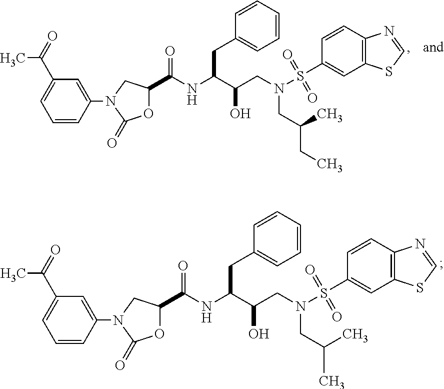

wherein W is selected, independently for each occurrence, from the group consisting of —NHR⁷ or —NHR(CH₂)ₚN(R⁷)₂; R⁷ is selected, independently for each occurrence, from the group consisting of hydrogen, alkyl, aralkyl, heteroaralkyl and acyl; and p is 1-10 inclusive.

In certain embodiments, the present invention relates to any of the aforementioned compounds and any of the attendant definitions, wherein W is —NHC(═O)-alkyl or —NHC(═O)-fluoroalkyl.

In certain embodiments, the present invention relates to any of the aforementioned compounds and any of the attendant definitions, wherein W is —NHC(═O)CH₃ or —NHC(═O)CF₃.

Another aspect of the present invention relates to a compound, or pharmaceutically acceptable salt thereof, selected from the group consisting of

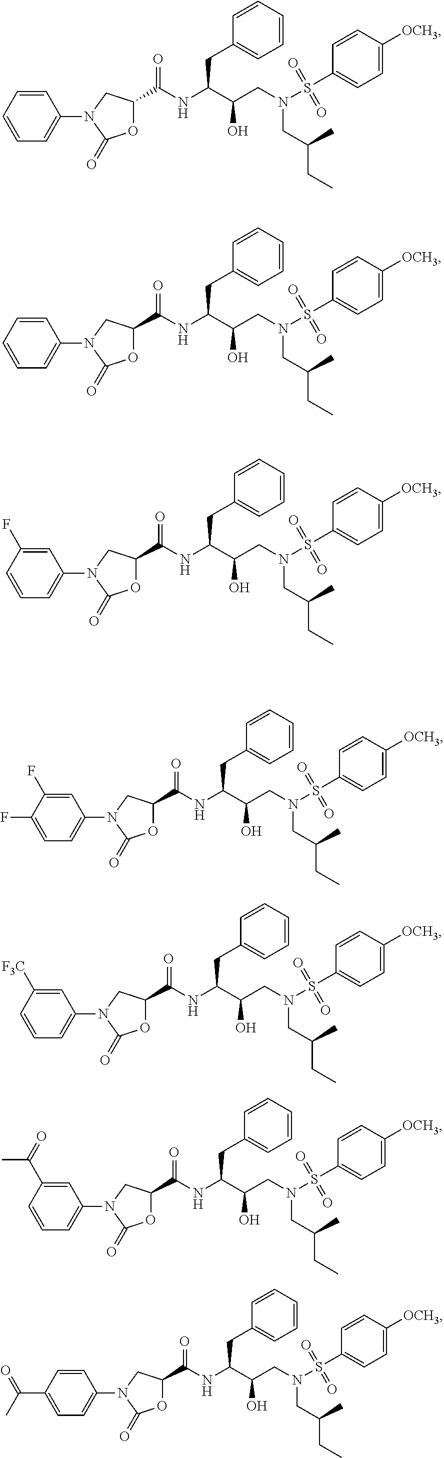

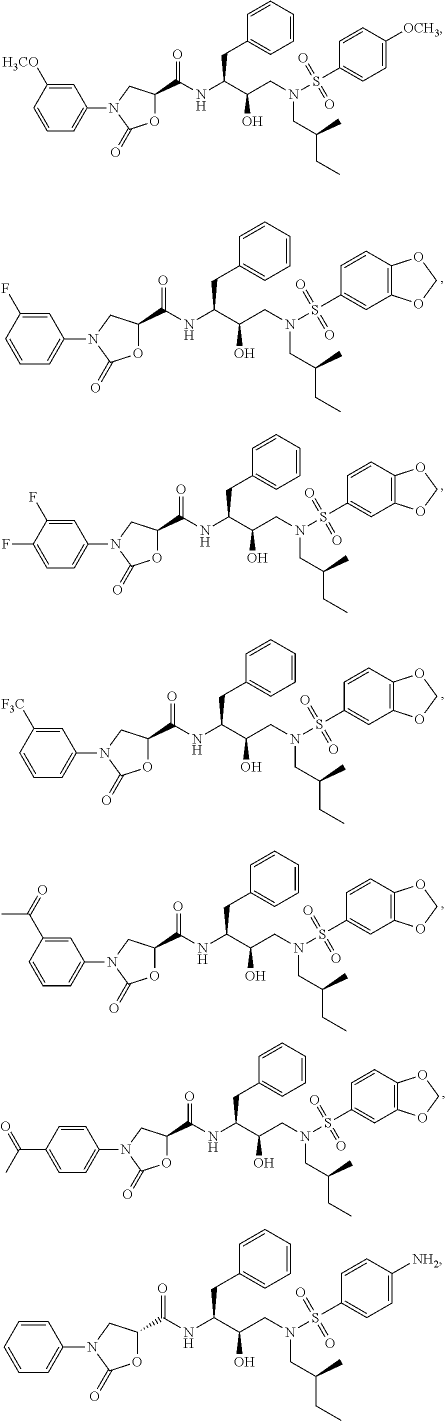

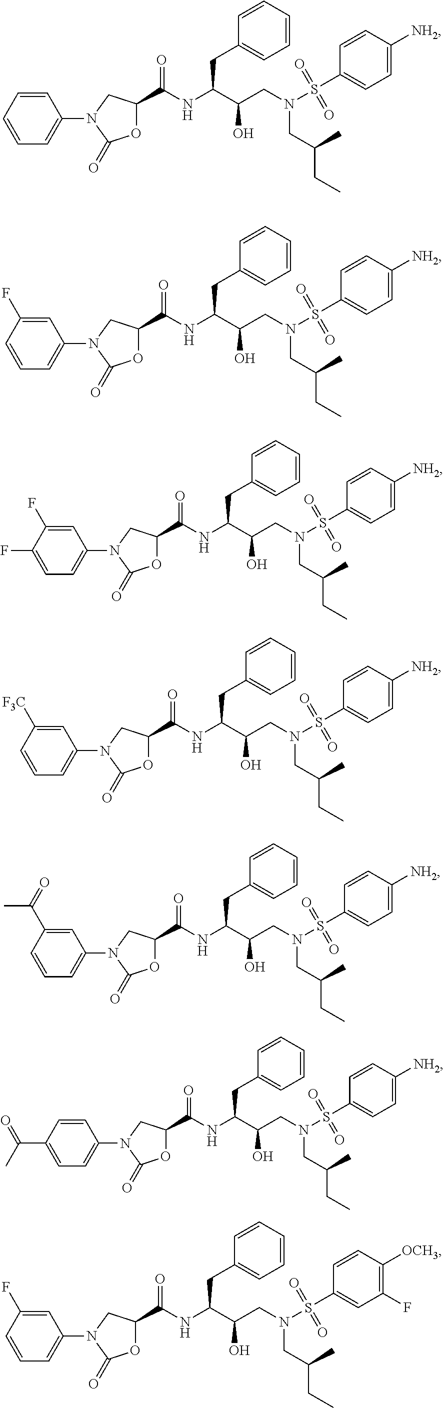

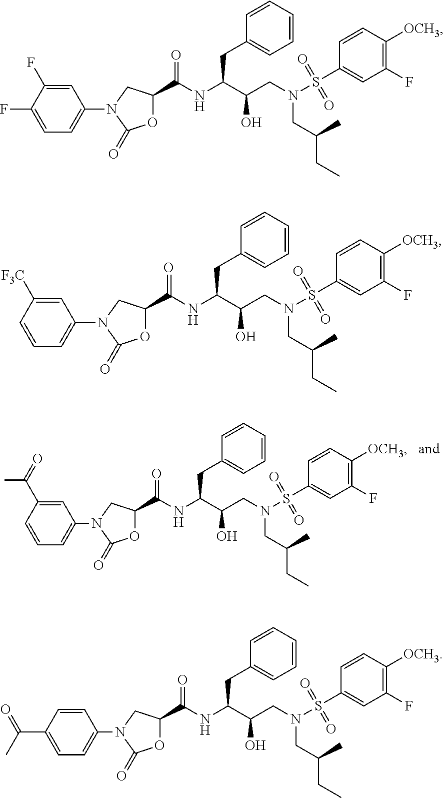

Another aspect of the present invention relates to a compound, or pharmaceutically acceptable salt thereof, selected from the group consisting of:

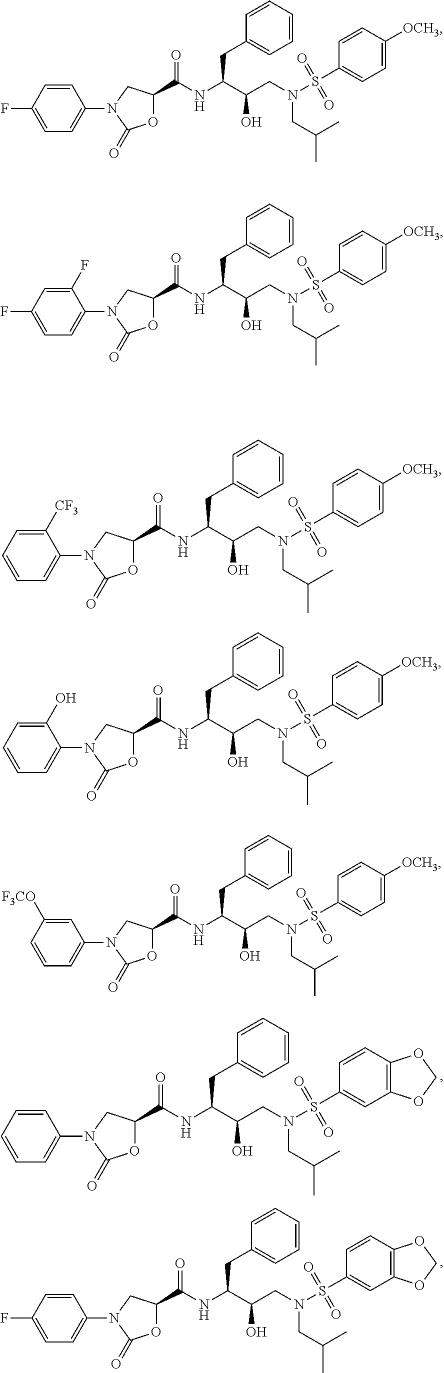

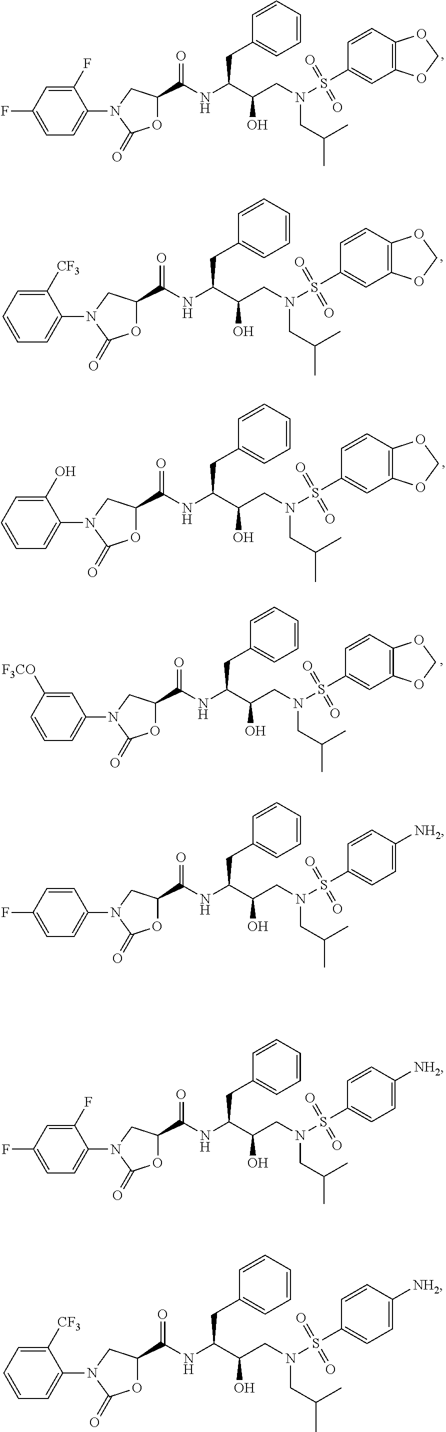

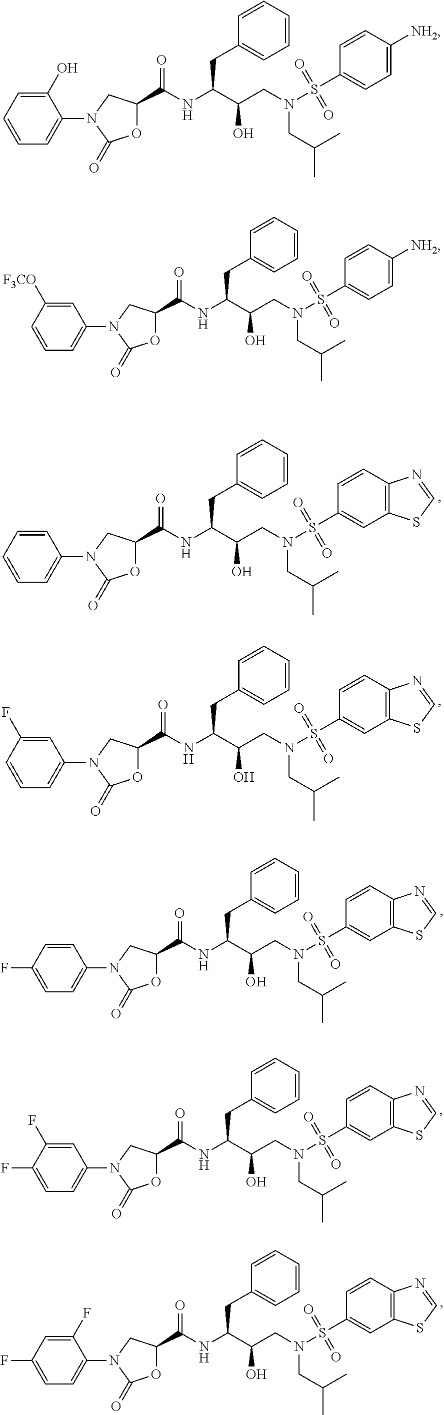

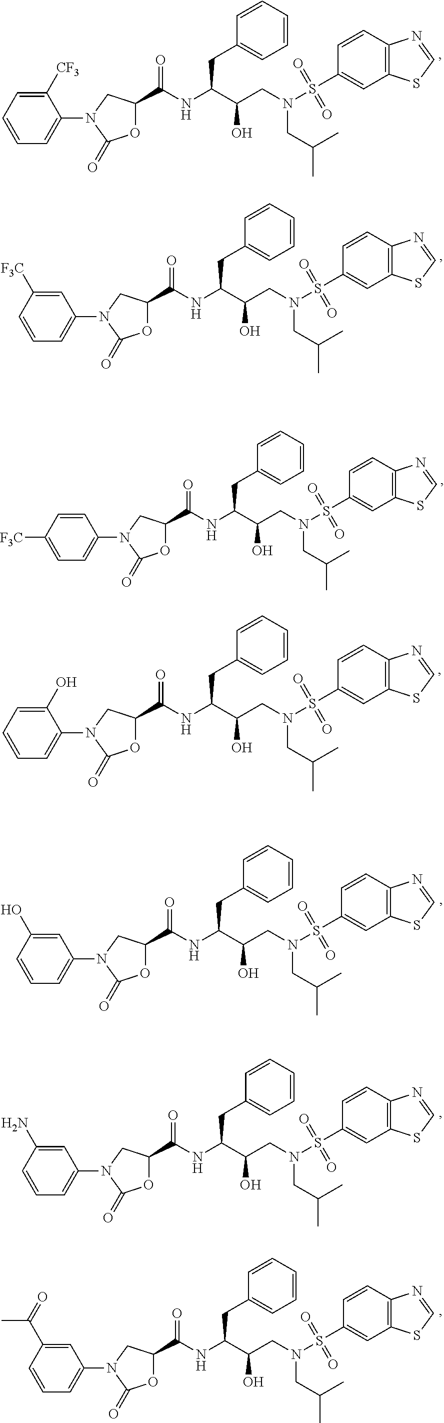

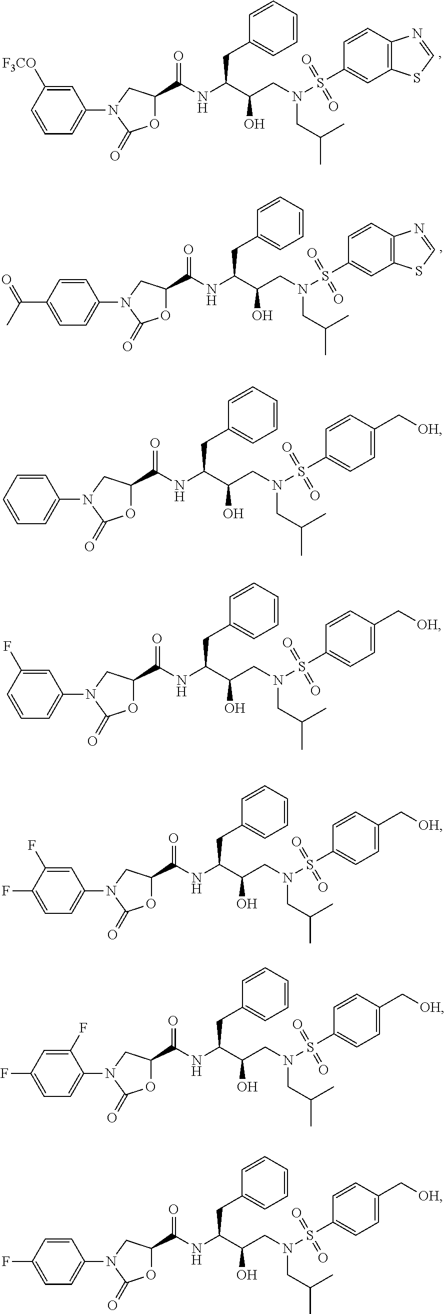

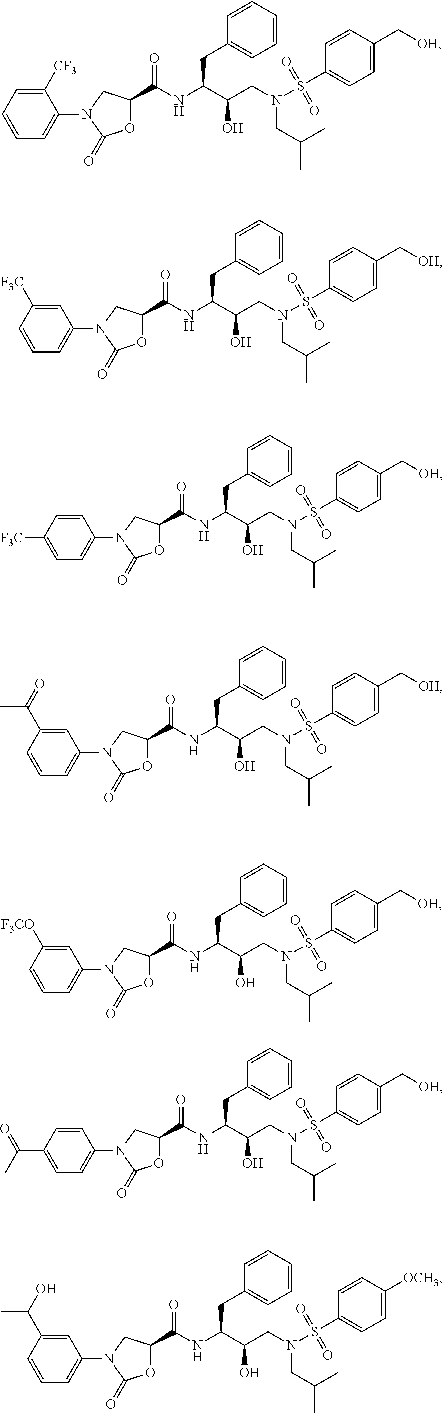

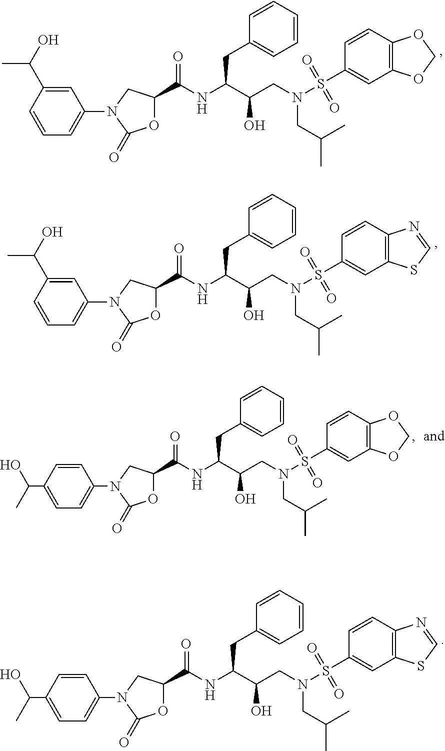

Another aspect of the present invention relates to a compound, or pharmaceutically acceptable salt thereof, selected from the group consisting of

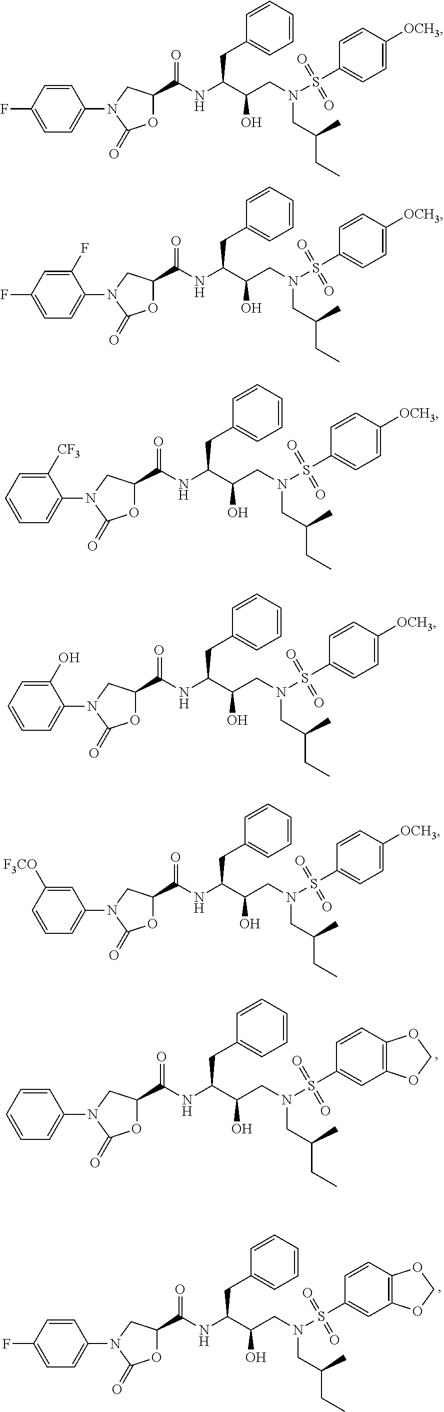

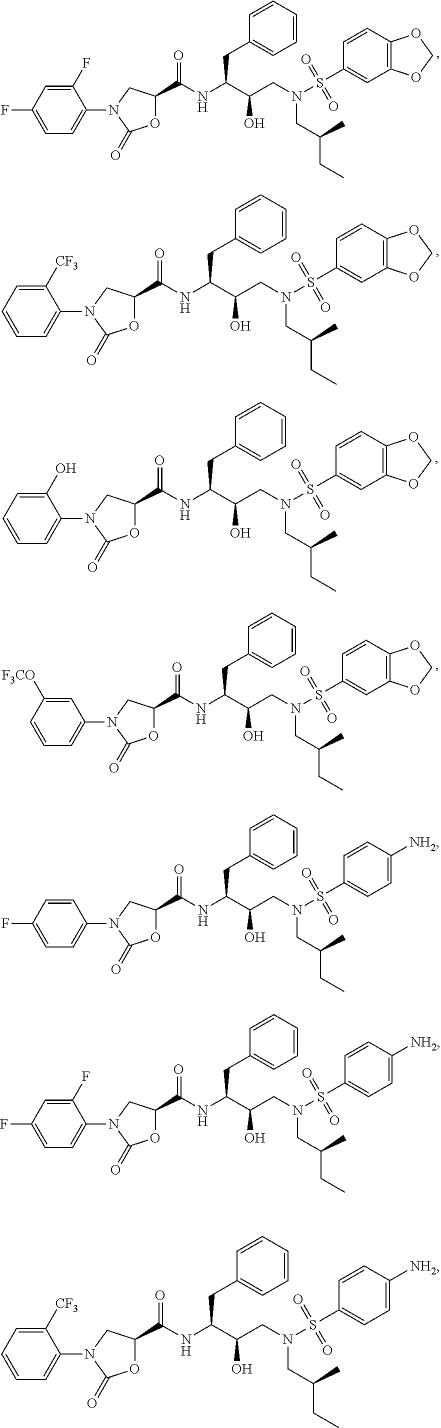

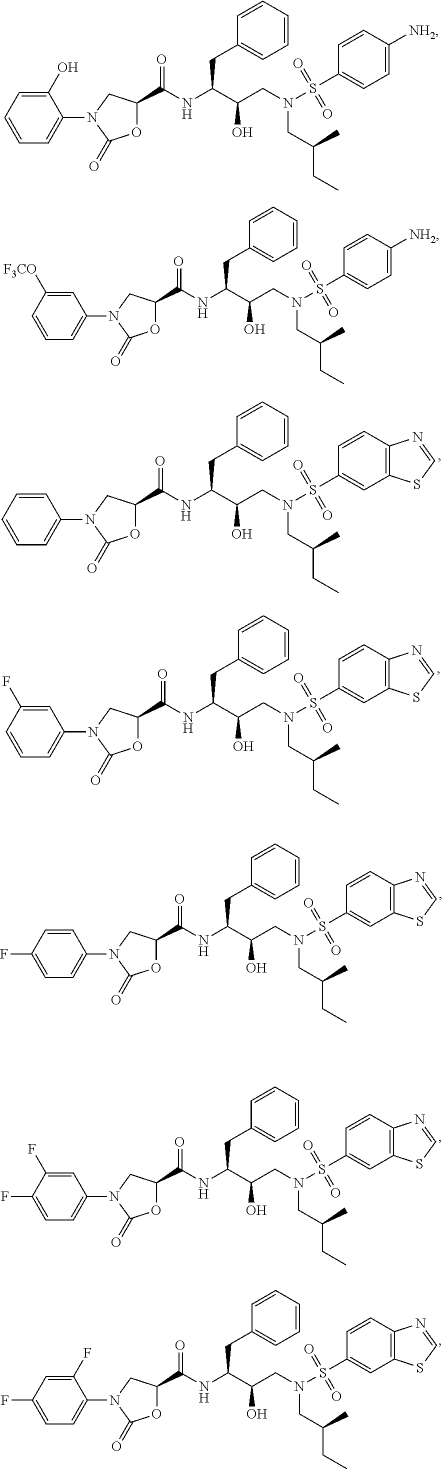

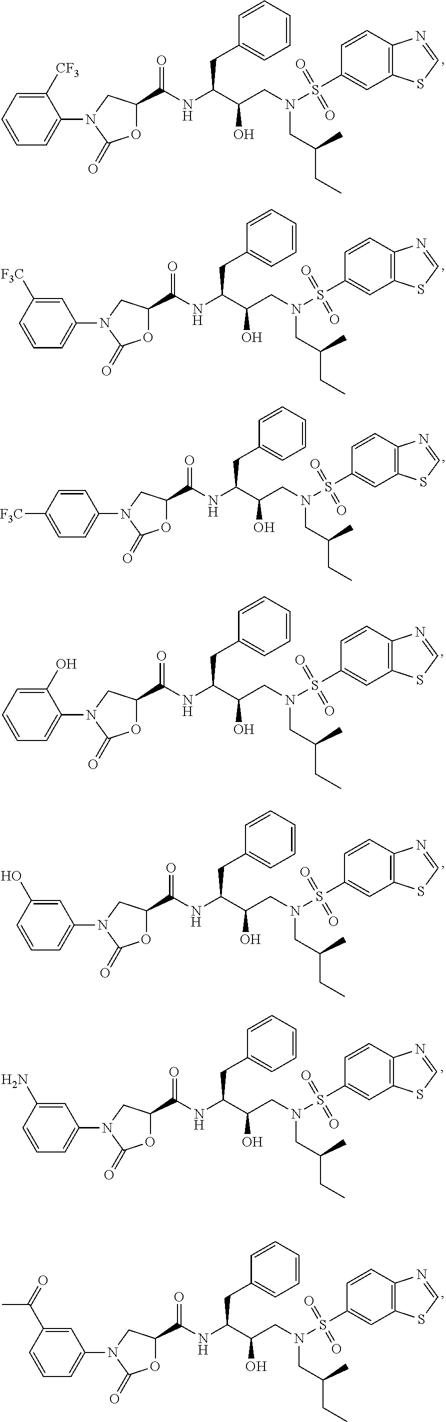

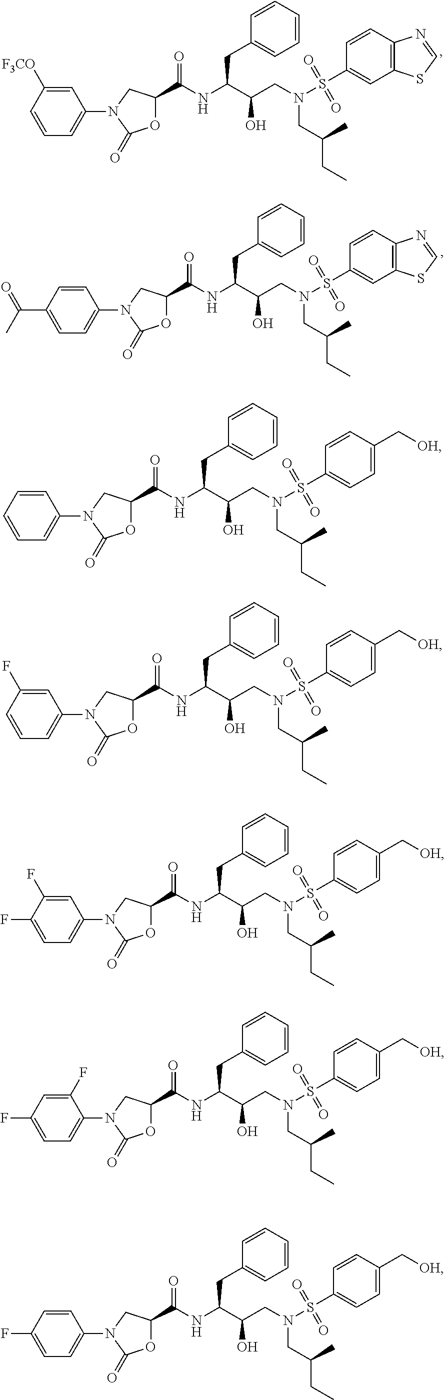

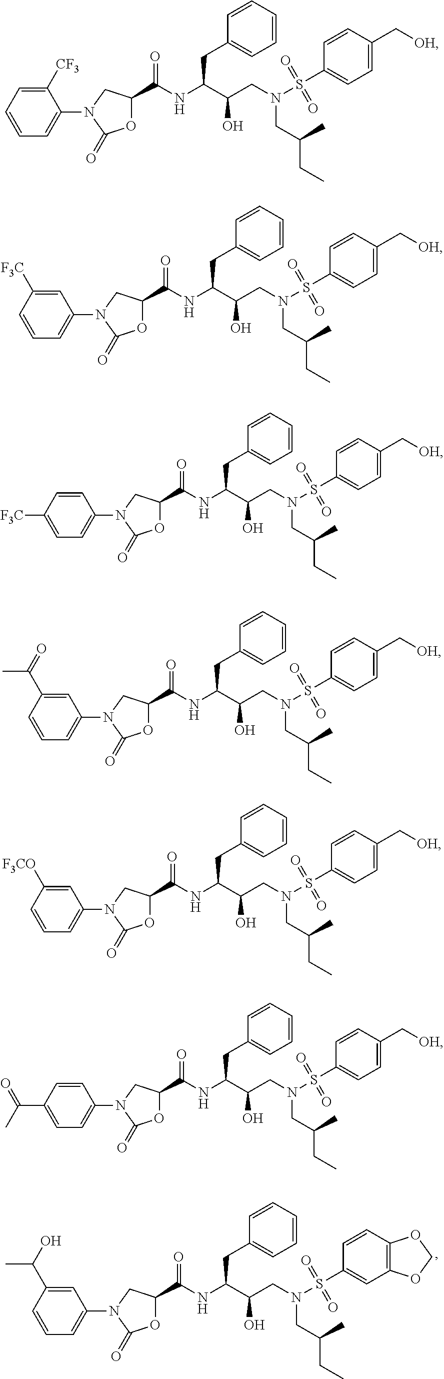

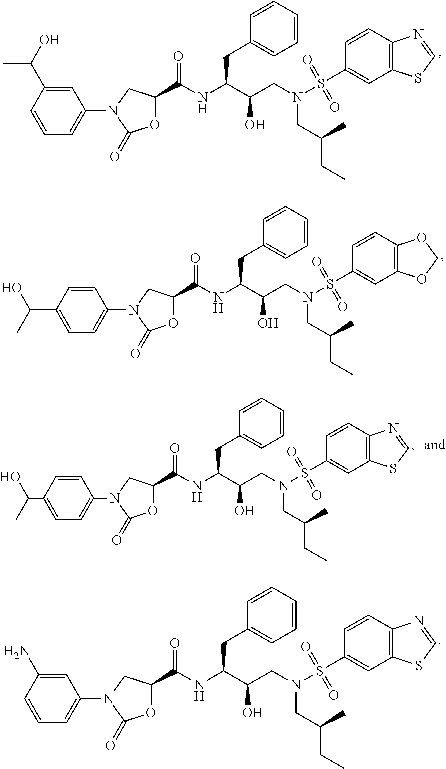

Another aspect of the present invention relates to a compound, or a pharmaceutically acceptable salt thereof, of formula XIIIA:

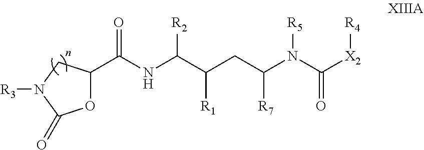

wherein, independently for each occurrence,

n is 1 or 2;

X₂ is absent, —O—, —S— or —NR—;

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, aralkyl, heteroaralkyl or acyl;

R₇ is hydrogen, alkyl, (cycloalkyl)alkyl, (amino)alkyl, (amido)alkyl, (keto)alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, provided that when n is 1; R₁ is —OH; R₂ is

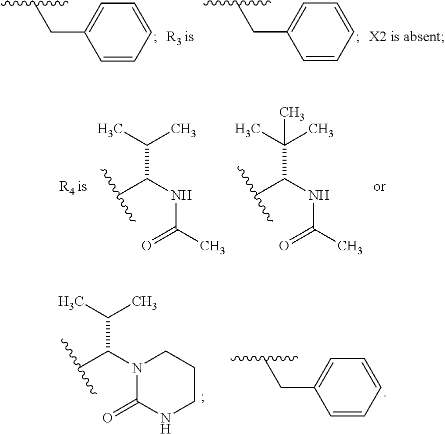

### and R₅ is H; R₇ is not

In certain embodiments, the present invention relates to any of the aforementioned compounds and any of the attendant definitions, wherein n is 1.

In certain embodiments, the present invention relates to any of the aforementioned compounds and any of the attendant definitions, wherein X₂ is absent.

In certain embodiments, the present invention relates to any of the aforementioned compounds and any of the attendant definitions, wherein R₁ is —OH.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

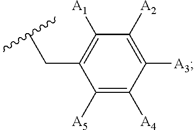

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

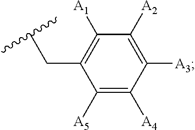

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₂ is

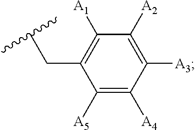

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₄ is alkyl, aryl or heteroaryl.

In certain embodiments, the present invention relates to any of the aforementioned compounds and any of the attendant definitions, wherein R₅ is hydrogen.

In certain embodiments, the present invention relates to any of the aforementioned compounds and any of the attendant definitions, wherein R₇ is

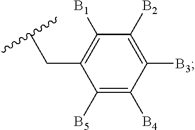

and B₁, B₂, B₃, B₄ and B₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₇ is

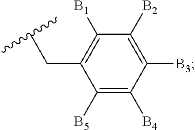

B₁, B₂, B₄ and B₅ are hydrogen; and B₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₇ is

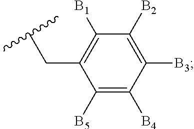

B₁, B₂, B₃ and B₅ are hydrogen; and B₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₇ is alkyl, (cycloalkyl)alkyl, (amino)alkyl, (amido)alkyl, (heterocyclyl)alkyl, aralkyl or heteroaralkyl.

In certain embodiments, the present invention relates to any of the aforementioned compounds and any of the attendant definitions, wherein R₇ is alkyl, (cycloalkyl)alkyl or aralkyl.

In certain embodiments, the present invention relates to any of the aforementioned compounds and any of the attendant definitions, wherein X₂ is absent; R₁ is —OH; R₂ is aralkyl; R₃ is aryl or heteroaryl; R₄ is alkyl, aryl or heteroaryl; R₅ is hydrogen; and R₇ is alkyl, (cycloalkyl)alkyl or aralkyl.

Another aspect of the present invention relates to a compound, or a pharmaceutically acceptable salt thereof, of formula XIIIB:

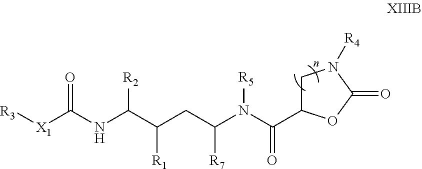

wherein, independently for each occurrence,

n is 1 or 2;

X₁ is absent, —O—, —S— or —NR—;

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is alkyl, cycloalkyl, heterocyclyl, aryl, heteroaryl, (heterocyclyl)alkyl, aralkyl or heteroaralkyl;

R₄ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₅ is hydrogen, alkyl, aralkyl, heteroaralkyl or acyl;

R₇ is hydrogen, alkyl, (cycloalkyl)alkyl, (amino)alkyl, (amido)alkyl, (keto)alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, provided that when n is 1; R₁ is —OH; R₂ is

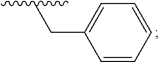

X₁ is absent; R₃ is

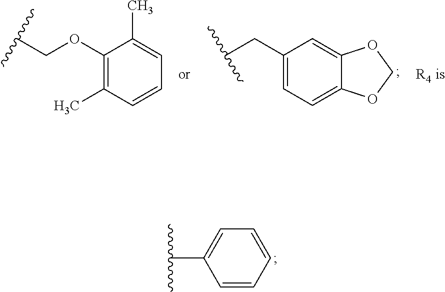

### and R₅ is H; R₇ is not

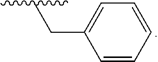

In certain embodiments, the present invention relates to any of the aforementioned compounds and any of the attendant definitions, provided that when n is 1; R₁ is —OH; R₂ is

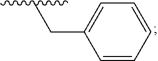

### X₁ is —O—; R₃ is

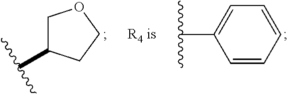

### and R₅ is H; R₇ is not

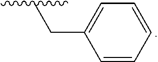

In certain embodiments, the present invention relates to any of the aforementioned compounds and any of the attendant definitions, wherein n is 1.

In certain embodiments, the present invention relates to any of the aforementioned compounds and any of the attendant definitions, wherein X₁ is absent.

In certain embodiments, the present invention relates to any of the aforementioned compounds and any of the attendant definitions, wherein R₁ is —OH.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

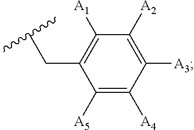

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

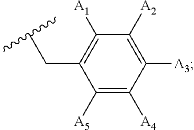

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is alkyl, aryl or heteroaryl.

In certain embodiments, the present invention relates to any of the aforementioned compounds and any of the attendant definitions, wherein R₂ is

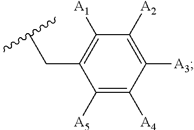

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₅ is hydrogen.

In certain embodiments, the present invention relates to any of the aforementioned compounds and any of the attendant definitions, wherein R₇ is

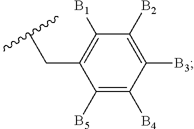

and B₁, B₂, B₃, B₄ and B₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₇ is

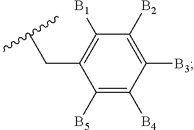

B₁, B₂, B₄ and B₅ are hydrogen; and B₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₇ is

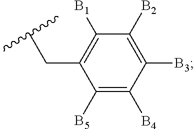

B₁, B₂, B₃ and B₅ are hydrogen; and B₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₇ is alkyl, (cycloalkyl)alkyl, (amino)alkyl, (amido)alkyl, (heterocyclyl)alkyl, aralkyl or heteroaralkyl.

In certain embodiments, the present invention relates to any of the aforementioned compounds and any of the attendant definitions, wherein R₇ is alkyl, (cycloalkyl)alkyl or aralkyl.

In certain embodiments, the present invention relates to any of the aforementioned compounds and any of the attendant definitions, wherein X₁ is absent; R₁ is —OH; R₂ is aralkyl; R₃ is aryl or heteroaryl; R₄ is alkyl, aryl or heteroaryl; R₅ is hydrogen; and R₇ is alkyl, (cycloalkyl)alkyl or aralkyl.

Another aspect of the present invention relates to a compound, or a pharmaceutically acceptable salt thereof, of formula XIVA:

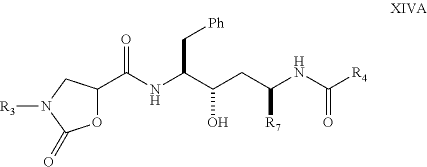

wherein, independently for each occurrence,

R₃ is alkyl, alkenyl, (amino)alkyl, (amido)alkyl, cycloalkyl, heterocyclyl, aryl, heteroaryl, (heterocyclyl)alkyl, aralkyl or heteroaralkyl;

R₄ is aryl, (amino)alkyl, (amido)alkyl, heterocyclyl, (heterocyclyl)alkyl, heteroaryl, aralkyl or heteroaralkyl;

R₇ is alkyl, cycloalkyl, (cycloalkyl)alkyl or aralkyl; and

the stereochemical configuration at any undefined stereocenter is R or S.

In certain embodiments, the present invention relates to any of the aforementioned compounds and any of the attendant definitions, provided that when R₃ is

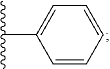

### and R₄ is

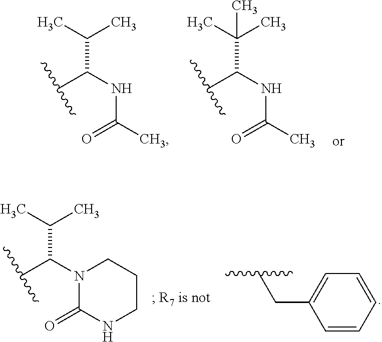

In certain embodiments, the present invention relates to any of the aforementioned compounds and any of the attendant definitions, wherein R₃ is aryl or heteroaralkyl.

In certain embodiments, the present invention relates to any of the aforementioned compounds and any of the attendant definitions, wherein R₄ is aryl, (amino)alkyl, (amido)alkyl, (keto)alkyl (heterocyclyl)alkyl or heterocyclyl.

In certain embodiments, the present invention relates to any of the aforementioned compounds and any of the attendant definitions, wherein R₇ is alkyl, cycloalkyl or (cycloalkyl)alkyl.

In certain embodiments, the present invention relates to any of the aforementioned compounds and any of the attendant definitions, R₃ is aryl or heteroaryl; R₄ is aryl, (amino)alkyl, (amido)alkyl, (keto)alkyl (heterocyclyl)alkyl or heterocyclyl; and R₇ is alkyl, cycloalkyl or (cycloalkyl)alkyl.

In certain embodiments, the present invention relates to any of the aforementioned compounds and any of the attendant definitions, wherein R₃ is

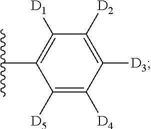

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₃ is

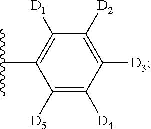

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is

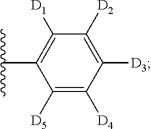

D₁, D₂, D₃ and D₅ are hydrogen; and D₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is -Ph.

In certain embodiments, the present invention relates to any of the aforementioned compounds and any of the attendant definitions, wherein R₇ is

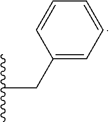

Another aspect of the present invention relates to a compound, or a pharmaceutically acceptable salt thereof, of formula XIVB:

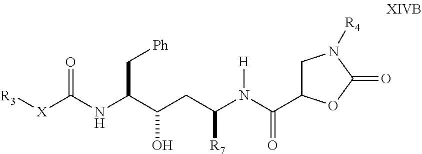

wherein, independently for each occurrence,

X is absent, —O—, —N(H)— or —S—;

R₃ is aryl, (amino)alkyl, (amido)alkyl, heterocyclyl, (heterocyclyl)alkyl, heteroaryl, aralkyl or heteroaralkyl;

R₄ is alkyl, alkenyl, (amino)alkyl, (amido)alkyl, cycloalkyl, heterocyclyl, aryl, heteroaryl, (heterocyclyl)alkyl, aralkyl or heteroaralkyl;

R₇ is alkyl, cycloalkyl, (cycloalkyl)alkyl or aralkyl; and

the stereochemical configuration at any undefined stereocenter is R or S.

In certain embodiments, the present invention relates to any of the aforementioned compounds and any of the attendant definitions, provided that when R₃ is

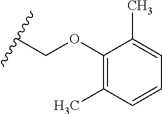

or

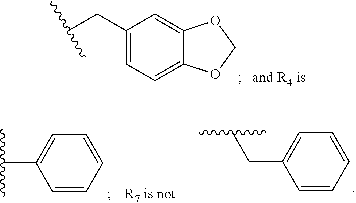

In certain embodiments, the present invention relates to any of the aforementioned compounds and any of the attendant definitions, provided that when R₃ is

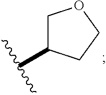

### and R₄ is

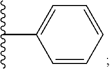

R₇ is not

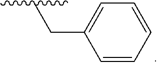

In certain embodiments, the present invention relates to any of the aforementioned compounds and any of the attendant definitions, wherein R₃ is (amino)alkyl, (amido)alkyl, (keto)alkyl, cycloalkyl, heterocyclyl, aryl, heteroaryl, (heterocyclyl)alkyl, aralkyl or heteroaralkyl.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₇ is alkyl, cycloalkyl or (cycloalkyl)alkyl.

In certain embodiments, the present invention relates to any of the aforementioned compounds and any of the attendant definitions, R₄ is aryl or heteroaryl; R₃ is aryl, (amino)alkyl, (amido)alkyl, (keto)alkyl (heterocyclyl)alkyl or heterocyclyl; and R₇ is alkyl, cycloalkyl or (cycloalkyl)alkyl.

In certain embodiments, the present invention relates to any of the aforementioned compounds and any of the attendant definitions, wherein R₄ is

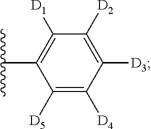

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₄ is

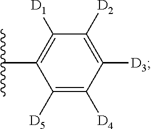

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is

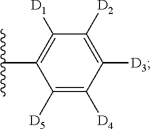

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is -Ph.

In certain embodiments, the present invention relates to any of the aforementioned compounds and any of the attendant definitions, wherein R₇ is

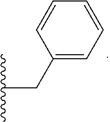

Another aspect of the present invention relates to a compound, or a pharmaceutically acceptable salt thereof, selected from the group consisting of

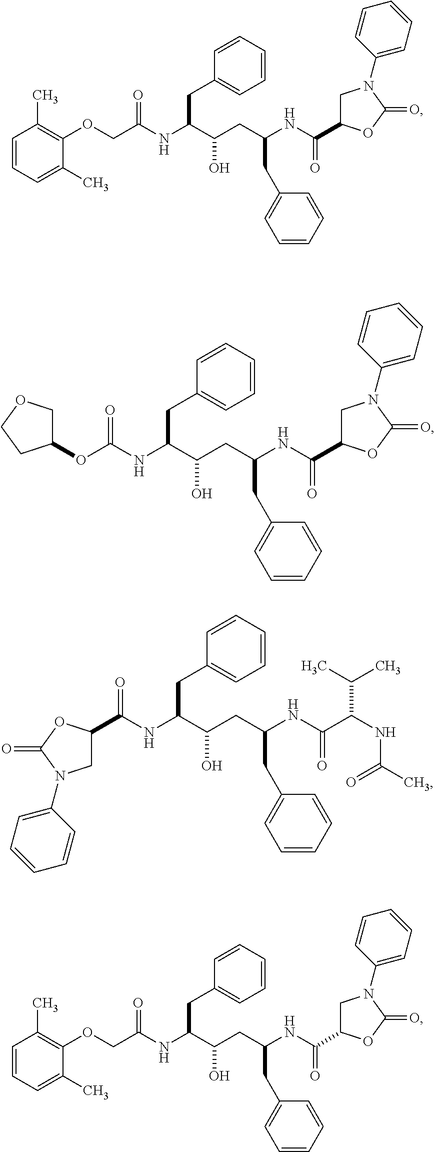

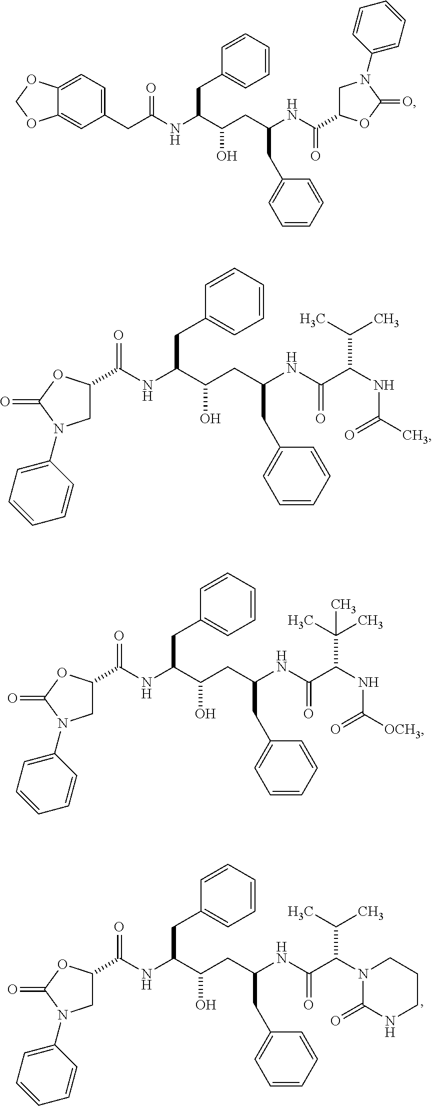

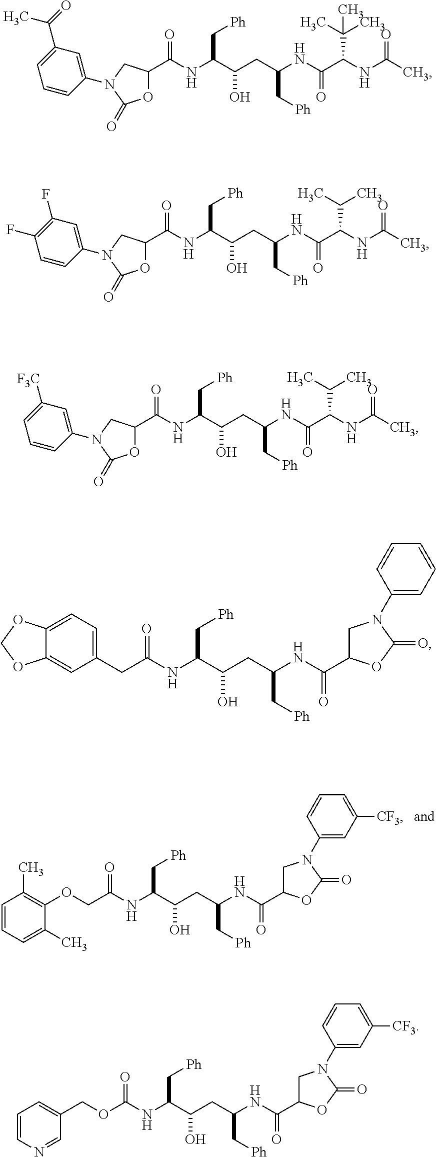

Another aspect of the present invention relates to a compound, or a pharmaceutically acceptable salt thereof, of formula XVA:

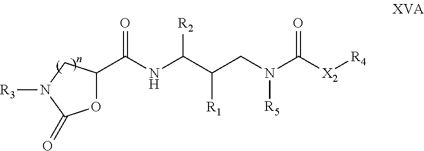

wherein, independently for each occurrence,

n is 1 or 2;

X₂ is absent, —O—, —S— or —NR—;

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, (cycloalkyl)alkyl, (amino)alkyl, (amido)alkyl, (keto)alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein X₂ is absent.

In certain embodiments, the present invention relates to any of the aforementioned compounds and any of the attendant definitions, wherein R₁ is —OH.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

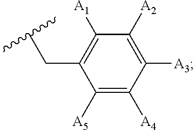

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

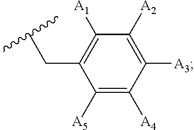

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₂ is

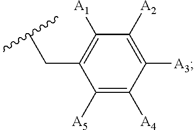

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₄ is (heterocyclyl)alkyl.

In certain embodiments, the present invention relates to any of the aforementioned compounds and any of the attendant definitions, wherein R₅ is alkyl.

In certain embodiments, the present invention relates to any of the aforementioned compounds and any of the attendant definitions, wherein X₂ is absent; R₁ is —OH; R₂ is aralkyl; R₃ is aryl; R₄ is alkyl, aryl or heteroaryl; and R₅ is alkyl.

Another aspect of the present invention relates to a compound, or a pharmaceutically acceptable salt thereof, of formula XVB:

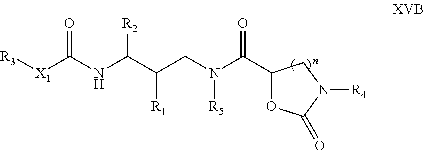

wherein, independently for each occurrence,

n is 1 or 2;

X₁ is absent, —O—, —S— or —NR—;

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is alkyl, cycloalkyl, heterocyclyl, aryl, heteroaryl, (heterocyclyl)alkyl, aralkyl or heteroaralkyl;

R₄ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₅ is hydrogen, alkyl, (cycloalkyl)alkyl, (amino)alkyl, (amido)alkyl, (keto)alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, provided that when n is 1; R₁ is —OH; R₂ is

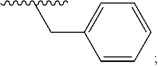

### X₁ is —O—; R₃ is

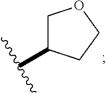

### and R₄ is

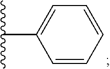

### R₅ is not

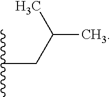

In certain embodiments, the present invention relates to any of the aforementioned compounds and any of the attendant definitions, wherein n is 1.

In certain embodiments, the present invention relates to any of the aforementioned compounds and any of the attendant definitions, wherein X₁ is absent.

In certain embodiments, the present invention relates to any of the aforementioned compounds and any of the attendant definitions, wherein R₁ is —OH.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

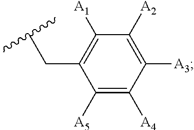

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

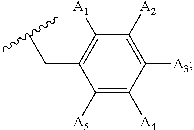

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₂ is

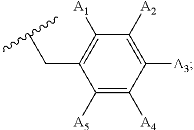

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₅ is alkyl.

In certain embodiments, the present invention relates to any of the aforementioned compounds and any of the attendant definitions, wherein X₁ is absent; R₁ is —OH; R₂ is aralkyl; R₄ is aryl; R₃ is alkyl, aryl or heteroaryl; and R₅ is alkyl.

Another aspect of the present invention relates to a compound, or a pharmaceutically acceptable salt thereof, of formula XVIA:

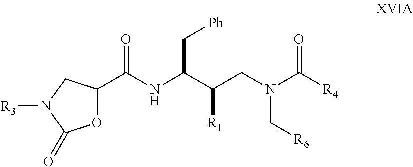

wherein, independently for each occurrence,

R₁ is —OH or —NH₂;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is aryl, (amino)alkyl, (amido)alkyl, heterocyclyl, (heterocyclyl)alkyl, heteroaryl, aralkyl or heteroaralkyl;

R₆ is alkyl, cycloalkyl, or aryl; and

the stereochemical configuration at any undefined stereocenter is R or S.

In certain embodiments, the present invention relates to any of the aforementioned compounds and any of the attendant definitions, wherein R₁ is —OH.

In certain embodiments, the present invention relates to any of the aforementioned compounds and any of the attendant definitions, wherein R₁ is —NH₂.

In certain embodiments, the present invention relates to any of the aforementioned compounds and any of the attendant definitions, wherein X₂ is —O—.

In certain embodiments, the present invention relates to any of the aforementioned compounds and any of the attendant definitions, wherein R₄ is heterocyclyl.

In certain embodiments, the present invention relates to any of the aforementioned compounds and any of the attendant definitions, wherein R₄ is

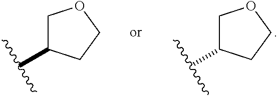

In certain embodiments, the present invention relates to any of the aforementioned compounds and any of the attendant definitions, wherein X₂ is —O—; and R₄ is heterocyclyl.

In certain embodiments, the present invention relates to any of the aforementioned compounds and any of the attendant definitions, wherein X₂ is —O—; and R₄ is

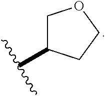

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₃ is

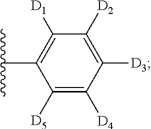

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₃ is

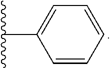

In certain embodiments, the present invention relates to any of the aforementioned compounds and any of the attendant definitions, wherein R₃ is

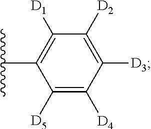

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is

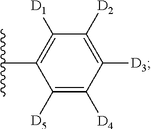

D₁, D₂, D₃ and D₅ are hydrogen; and D₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₆ is alkyl.

In certain embodiments, the present invention relates to any of the aforementioned compounds and any of the attendant definitions, wherein R₆ is —CH(CH₃)₂.

Another aspect of the present invention relates to a compound, or a pharmaceutically acceptable salt thereof, of formula XVIB:

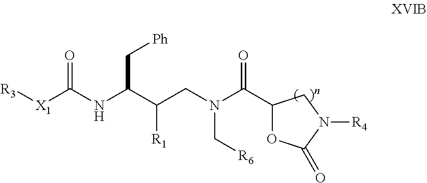

wherein, independently for each occurrence,

R₁ is —OH or —NH₂;

R₃ is aryl, (amino)alkyl, (amido)alkyl, heterocyclyl, (heterocyclyl)alkyl, heteroaryl, aralkyl or heteroaralkyl;

R₄ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₆ is alkyl, cycloalkyl, or aryl; and

the stereochemical configuration at any undefined stereocenter is R or S.

In certain embodiments, the present invention relates to any of the aforementioned compounds and any of the attendant definitions, provided that when n is 1; R₁ is —OH; X₁ is —O—; R₃ is

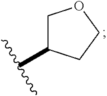

### and R₄ is

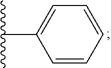

### R₆ is not

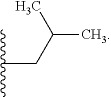

In certain embodiments, the present invention relates to any of the aforementioned compounds and any of the attendant definitions, wherein R₁ is —OH.

In certain embodiments, the present invention relates to any of the aforementioned compounds and any of the attendant definitions, wherein R₁ is —NH₂.

In certain embodiments, the present invention relates to any of the aforementioned compounds and any of the attendant definitions, wherein X₁ is —O—.

In certain embodiments, the present invention relates to any of the aforementioned compounds and any of the attendant definitions, wherein R₃ is heterocyclyl.

In certain embodiments, the present invention relates to any of the aforementioned compounds and any of the attendant definitions, wherein R₃ is

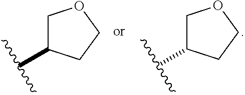

In certain embodiments, the present invention relates to any of the aforementioned compounds and any of the attendant definitions, wherein X₁ is —O—; and R₃ is heterocyclyl.

In certain embodiments, the present invention relates to any of the aforementioned compounds and any of the attendant definitions, wherein X₁ is —O—; and R₃ is

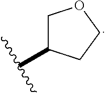

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₄ is

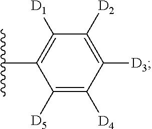

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₄ is

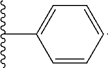

In certain embodiments, the present invention relates to any of the aforementioned compounds and any of the attendant definitions, wherein R₄ is

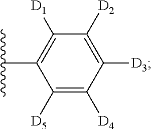

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is

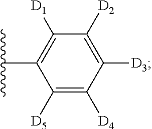

D₁, D₂, D₃ and D₅ are hydrogen; and D₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₆ is alkyl.

In certain embodiments, the present invention relates to any of the aforementioned compounds and any of the attendant definitions, wherein R₆ is —CH(CH₃)₂.

Another aspect of the present invention relates to a compound, or a pharmaceutically acceptable salt thereof, selected from the group consisting of

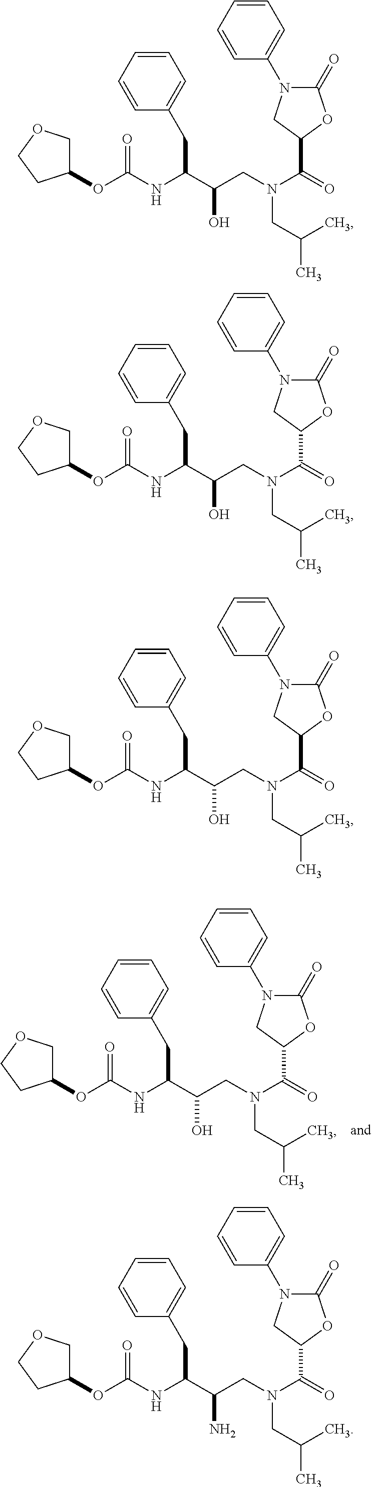

Another aspect of the present invention relates to a compound, or a pharmaceutically acceptable salt thereof, of formula XVIIA:

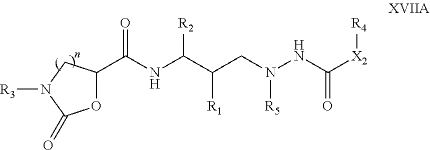

wherein, independently for each occurrence,

n is 1 or 2;

X₂ is absent, —O—, —S— or —NR—;

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is alkyl, cycloalkyl, heterocyclyl, aryl, heteroaryl, (heterocyclyl)alkyl, aralkyl or heteroaralkyl;

R₅ is hydrogen, alkyl, (cycloalkyl)alkyl, (amino)alkyl, (amido)alkyl, (keto)alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, provided that when n is 1; R₁ is —OH; R₂ is

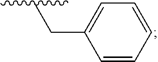

X₂ is absent; R₃ is

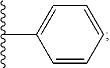

### and R₄ is

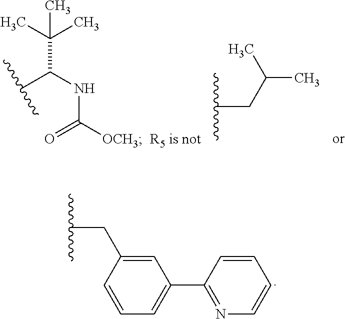

In certain embodiments, the present invention relates to any of the aforementioned compounds and any of the attendant definitions, wherein n is 1.

In certain embodiments, the present invention relates to any of the aforementioned compounds and any of the attendant definitions, wherein X₂ is absent.

In certain embodiments, the present invention relates to any of the aforementioned compounds and any of the attendant definitions, wherein R₁ is —OH or —NH₂.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

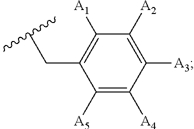

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

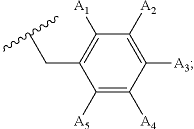

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₂ is

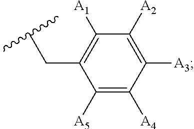

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₄ is (amido)alkyl or heterocyclyl.

In certain embodiments, the present invention relates to any of the aforementioned compounds and any of the attendant definitions, wherein R₅ is alkyl or aryl.

In certain embodiments, the present invention relates to any of the aforementioned compounds and any of the attendant definitions, wherein X₂ is absent; R₁ is —OH; R₂ is aralkyl; R₃ is aryl; R₄ is (amido)alkyl or heterocyclyl; and R₅ is alkyl or aryl.

Another aspect of the present invention relates to a compound, or a pharmaceutically acceptable salt thereof, of formula XVIIB:

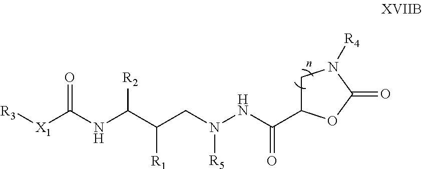

wherein, independently for each occurrence,

n is 1 or 2;

X₁ is absent, —O—, —S— or —NR—;

R₁ is —OH, —SH or —NHR;

R is hydrogen, alkyl, aralkyl, heteroaralkyl or acyl;

R₂ is hydrogen, alkyl, cycloalkyl, heterocyclyl, aryl, heteroaryl, (heterocyclyl)alkyl, aralkyl or heteroaralkyl;

R₃ is alkyl, cycloalkyl, heterocyclyl, aryl, heteroaryl, (heterocyclyl)alkyl, aralkyl or heteroaralkyl;

R₄ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₅ is hydrogen, alkyl, (cycloalkyl)alkyl, (amino)alkyl, (amido)alkyl, (keto)alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, provided that when n is 1; R₁ is —OH; R₂ is

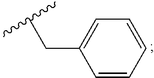

X₁ is absent; R₃

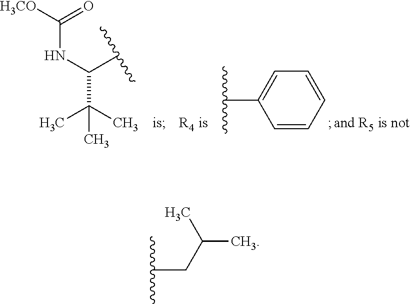

In certain embodiments, the present invention relates to any of the aforementioned compounds and any of the attendant definitions, wherein n is 1.

In certain embodiments, the present invention relates to any of the aforementioned compounds and any of the attendant definitions, wherein X₁ is absent.

In certain embodiments, the present invention relates to any of the aforementioned compounds and any of the attendant definitions, wherein R₁ is —OH or —NH₂.

In certain embodiments, the present invention relates to any of the aforementioned compounds and any of the attendant definitions, wherein R₂ is aralkyl or heteroaralkyl.

In certain embodiments, the present invention relates to any of the aforementioned compounds and any of the attendant definitions, wherein R₂ is aralkyl.

In certain embodiments, the present invention relates to any of the aforementioned compounds and any of the attendant definitions, wherein R₂ is

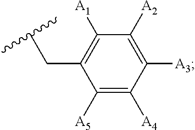

and A₁, A₂, A₃, A₄ and A₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₂ is

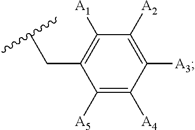

A₁, A₂, A₄ and A₅ are hydrogen; and A₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₂ is

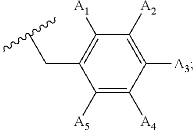

A₁, A₂, A₃ and A₅ are hydrogen; and A₄ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is (amido)alkyl or heterocyclyl.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₅ is alkyl or aryl.

In certain embodiments, the present invention relates to any of the aforementioned compounds and any of the attendant definitions, wherein X₁ is absent; R₁ is —OH; R₂ is aralkyl; R₄ is aryl; R₃ is (amido)alkyl or heterocyclyl; and R₅ is alkyl or aryl.

Another aspect of the present invention relates to a compound, or a pharmaceutically acceptable salt thereof, of formula XVIIIA:

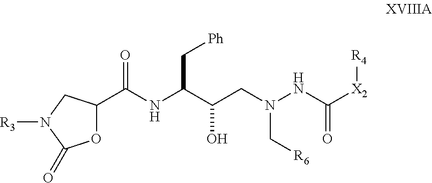

wherein, independently for each occurrence,

X₂ is absent or —O—;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is aryl, heteroaryl, aralkyl or heteroaralkyl;

R₆ is alkyl, cycloalkyl, heterocyclyl, aryl, heteroaryl, aralkyl or heteroaralkyl; and

the stereochemical configuration at any undefined stereocenter is R or S.

In certain embodiments, the present invention relates to any of the aforementioned compounds and any of the attendant definitions, provided that when X₂ is absent; R₃ is

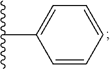

### and R₄ is

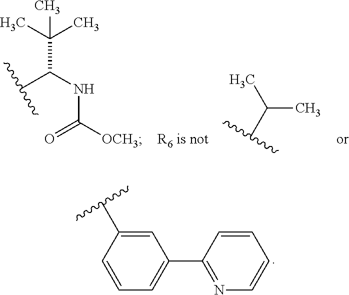

In certain embodiments, the present invention relates to any of the aforementioned compounds and any of the attendant definitions, wherein X₂ is absent.

In certain embodiments, the present invention relates to any of the aforementioned compounds and any of the attendant definitions, wherein R₃ is aryl or heteroaryl.

In certain embodiments, the present invention relates to any of the aforementioned compounds and any of the attendant definitions, wherein R₃ is

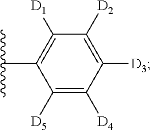

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₃ is

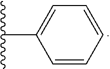

In certain embodiments, the present invention relates to any of the aforementioned compounds and any of the attendant definitions, wherein R₃ is

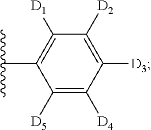

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is

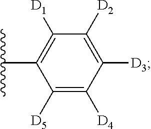

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is (amino)alkyl, (amido)alkyl or heterocyclyl.

In certain embodiments, the present invention relates to any of the aforementioned compounds and any of the attendant definitions, wherein R₄ is

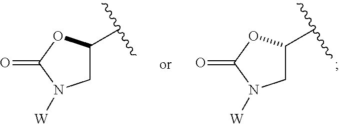

and W is aryl or heteroaryl.

In certain embodiments, the present invention relates to any of the aforementioned compounds and any of the attendant definitions, wherein W is

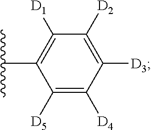

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein W is

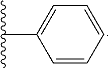

In certain embodiments, the present invention relates to any of the aforementioned compounds and any of the attendant definitions, wherein W is

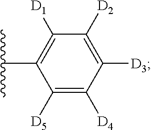

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein W is

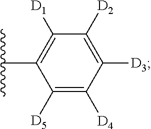

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₆ is alkyl or aryl.

In certain embodiments, the present invention relates to any of the aforementioned compounds and any of the attendant definitions, wherein R₆ is

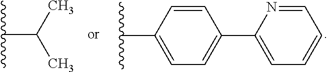

Another aspect of the present invention relates to a compound, or a pharmaceutically acceptable salt thereof, of formula XVIIIB:

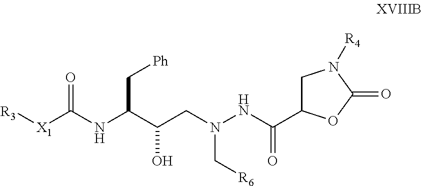

wherein, independently for each occurrence,

X₁ is absent or —O—;

R₃ is aryl, heteroaryl, aralkyl or heteroaralkyl;

R₄ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₆ is alkyl, cycloalkyl, heterocyclyl, aryl, heteroaryl, aralkyl or heteroaralkyl; and

the stereochemical configuration at any undefined stereocenter is R or S.

In certain embodiments, the present invention relates to any of the aforementioned compounds and any of the attendant definitions, provided that when n is 1; R₁ is —OH; R₂ is

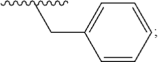

X₁ is absent; R₃ is

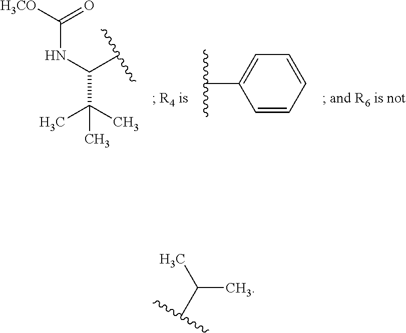

In certain embodiments, the present invention relates to any of the aforementioned compounds and any of the attendant definitions, wherein X₁ is absent.

In certain embodiments, the present invention relates to any of the aforementioned compounds and any of the attendant definitions, wherein R₄ is aryl or heteroaryl.

In certain embodiments, the present invention relates to any of the aforementioned compounds and any of the attendant definitions, wherein R₄ is

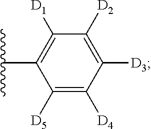

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein R₄ is

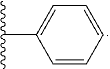

In certain embodiments, the present invention relates to any of the aforementioned compounds and any of the attendant definitions, wherein R₄ is

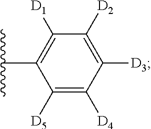

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₄ is

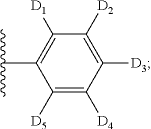

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₃ is (amino)alkyl, (amido)alkyl or heterocyclyl.

In certain embodiments, the present invention relates to any of the aforementioned compounds and any of the attendant definitions, wherein R₃ is

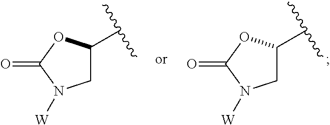

and W is aryl or heteroaryl.

In certain embodiments, the present invention relates to any of the aforementioned compounds and any of the attendant definitions, wherein W is

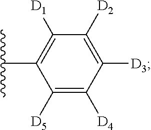

and D₁, D₂, D₃, D₄ and D₅ are independently selected from the group consisting of hydrogen, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl and cyano.

In certain embodiments, the present invention relates to any of the aforementioned compounds and any of the attendant definitions, wherein W is

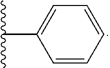

In certain embodiments, the present invention relates to any of the aforementioned compounds and any of the attendant definitions, wherein W is

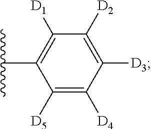

D₁, D₂, D₄ and D₅ are hydrogen; and D₃ is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein W is

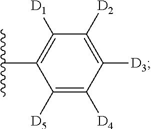

D₁, D₂, D₃ and D₅ are hydrogen; and D4 is halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aryl, heteroaryl, trifluoromethyl or cyano.

In certain embodiments, the present invention relates to any of the aforementioned compounds and any of the attendant definitions, wherein R₆ is alkyl or aryl.

In certain embodiments, the present invention relates to any of the aforementioned compounds and any of the attendant definitions, wherein R₆ is

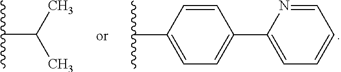

Another aspect of the present invention relates to a compound, or a pharmaceutically acceptable salt thereof, selected from the group consisting of

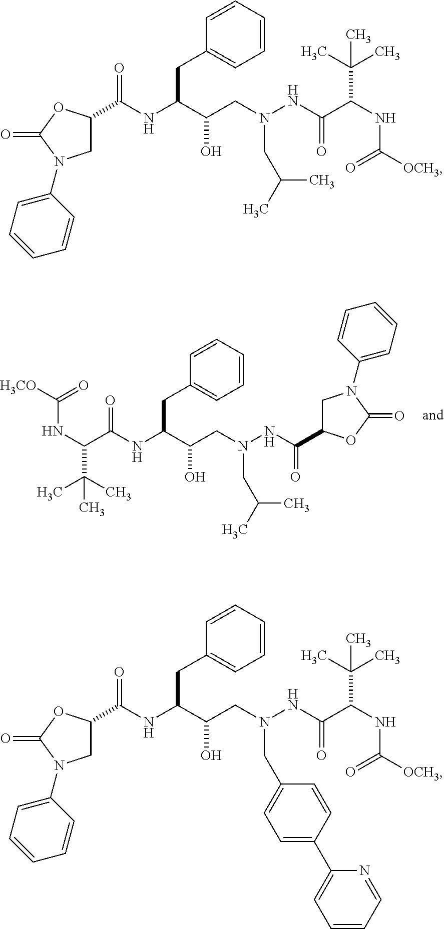

Another aspect of the present invention relates to a compound, or a pharmaceutically acceptable salt thereof, of formula XIX:

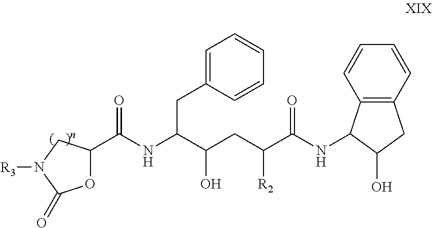

wherein, independently for each occurrence,

n is 1 or 2;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl; and the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein R₂ is

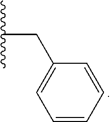

In certain embodiments, the present invention relates to any of the aforementioned compounds and any of the attendant definitions, wherein R₃ is

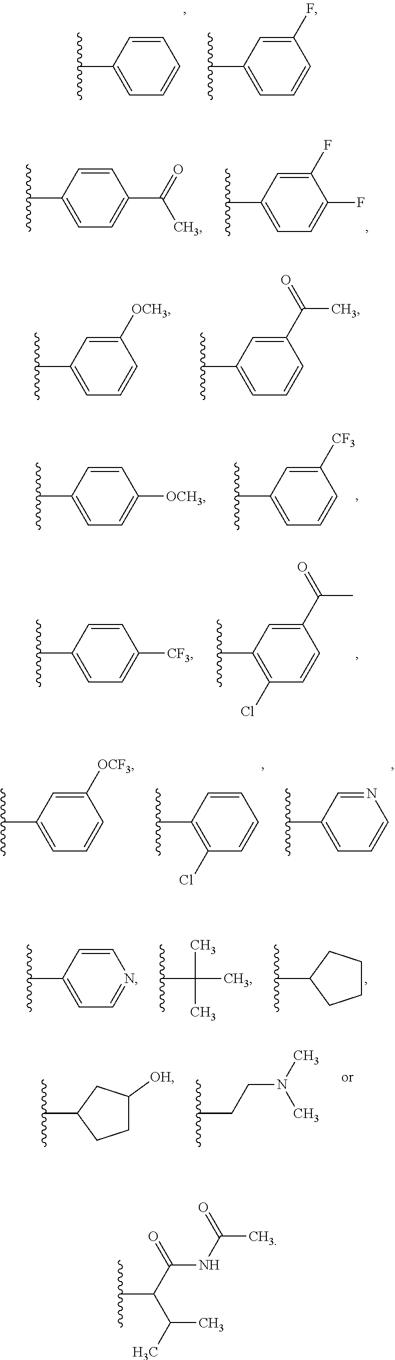

Another aspect of the present invention relates to a compound, or a pharmaceutically acceptable salt thereof, of formula XX:

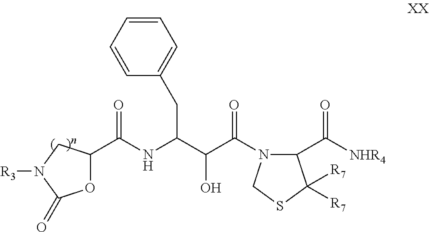

wherein, independently for each occurrence,

n is 1 or 2;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is hydrogen, alkyl, cycloalkyl, heterocyclyl, aryl, heteroaryl, (heterocyclyl)alkyl, aralkyl or heteroaralkyl;

R₇ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein R₃ is

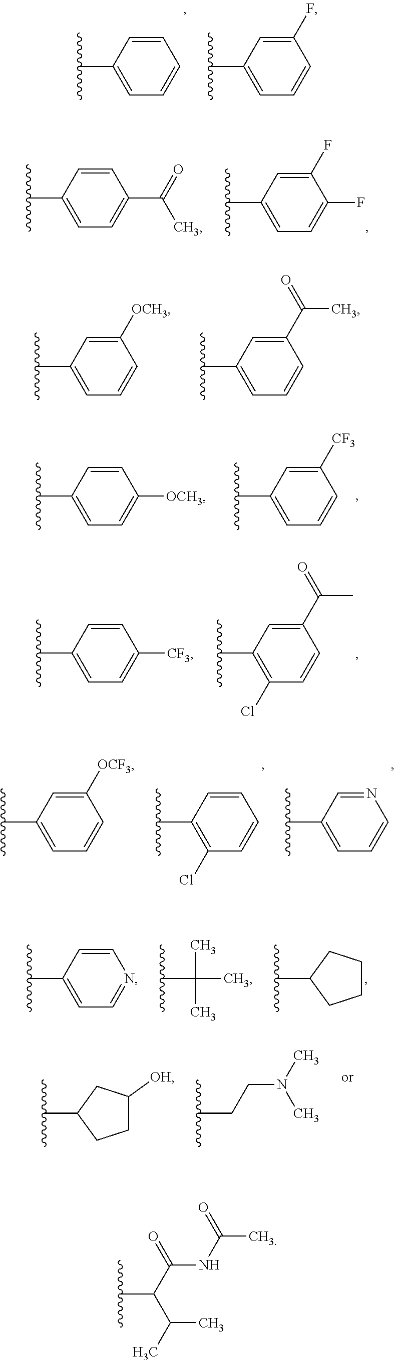

In certain embodiments, the present invention relates to any of the aforementioned compounds and any of the attendant definitions, wherein R₄ is

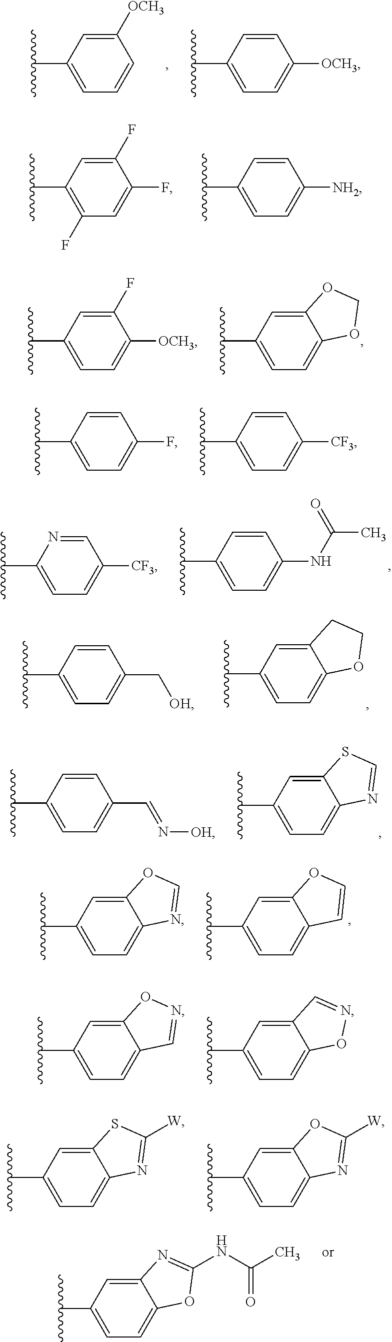

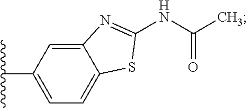

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

In certain embodiments, the present invention relates to any of the aforementioned compounds and any of the attendant definitions, wherein R₇ is hydrogen or alkyl.

In certain embodiments, the present invention relates to any of the aforementioned compounds and any of the attendant definitions, wherein R₇ is methyl.

In certain embodiments, the present invention relates to any of the aforementioned compounds and any of the attendant definitions, wherein R₇ is hydrogen.

Another aspect of the present invention relates to a compound, or a pharmaceutically acceptable salt thereof, of formula XXI:

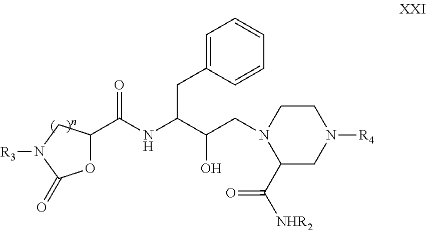

wherein, independently for each occurrence,

n is 1 or 2;

R₂ is hydrogen, alkyl, cycloalkyl, heterocyclyl, aryl, heteroaryl, (heterocyclyl)alkyl, aralkyl or heteroaralkyl;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein R₃ is

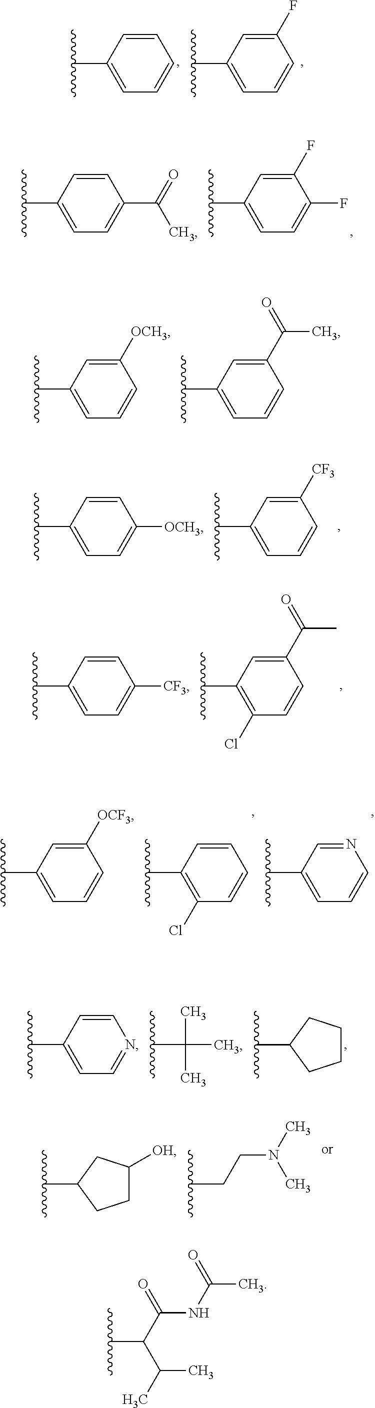

In certain embodiments, the present invention relates to any of the aforementioned compounds and any of the attendant definitions, wherein R₄ is

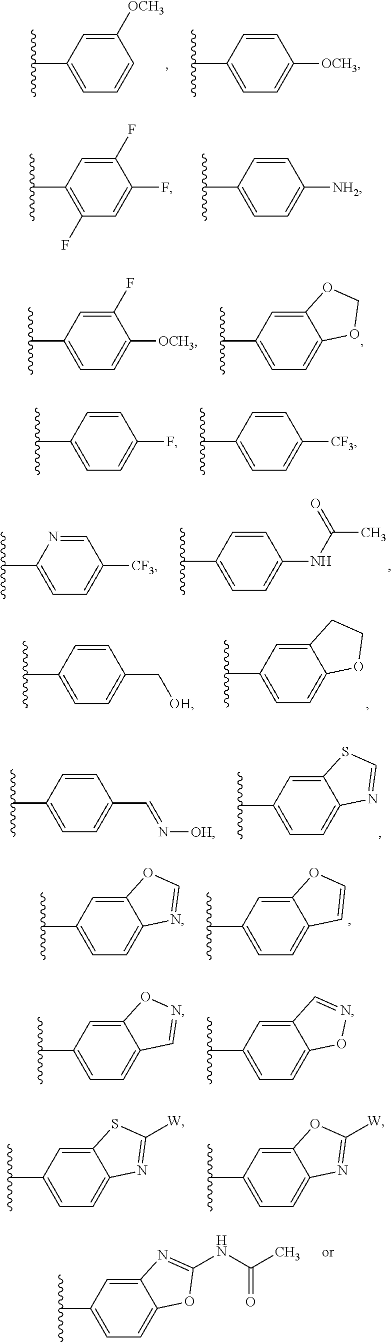

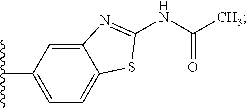

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

In certain embodiments, the present invention relates to any of the aforementioned compounds and any of the attendant definitions, wherein R₂ is hydrogen.

Another aspect of the present invention relates to a compound, or a pharmaceutically acceptable salt thereof, of formula XXII:

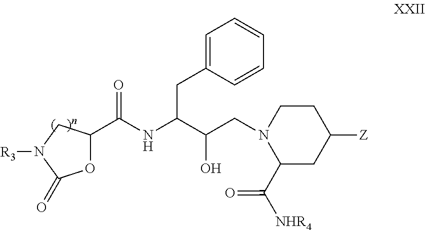

wherein, independently for each occurrence,

n is 1 or 2;

Z is hydrogen, R₄, or OR₄;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein Z is hydrogen.

In certain embodiments, the present invention relates to any of the aforementioned compounds and any of the attendant definitions, wherein Z is R₄.

In certain embodiments, the present invention relates to any of the aforementioned compounds and any of the attendant definitions, wherein Z is OR₄.

In certain embodiments, the present invention relates to any of the aforementioned compounds and any of the attendant definitions, wherein R₃ is

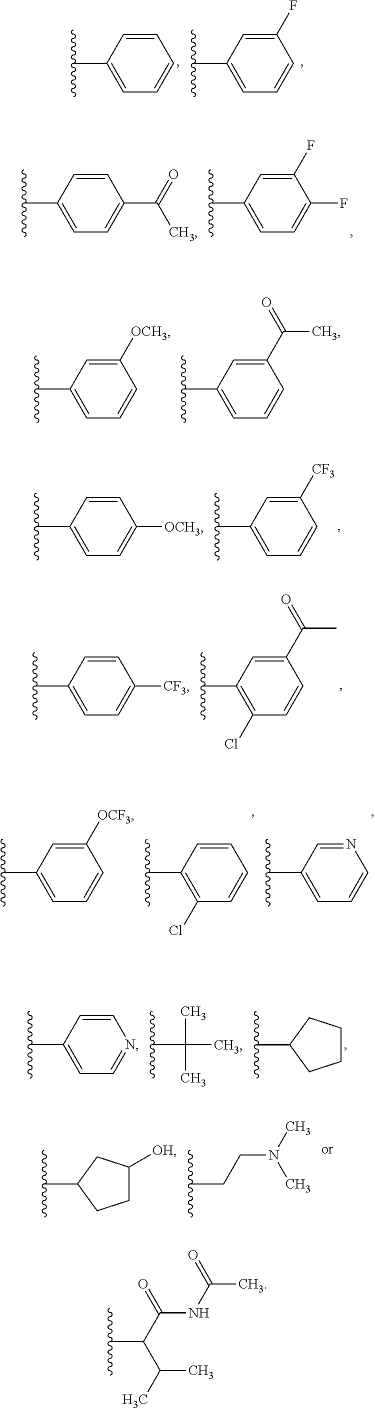

In certain embodiments, the present invention relates to any of the aforementioned compounds and any of the attendant definitions, wherein R₄ is

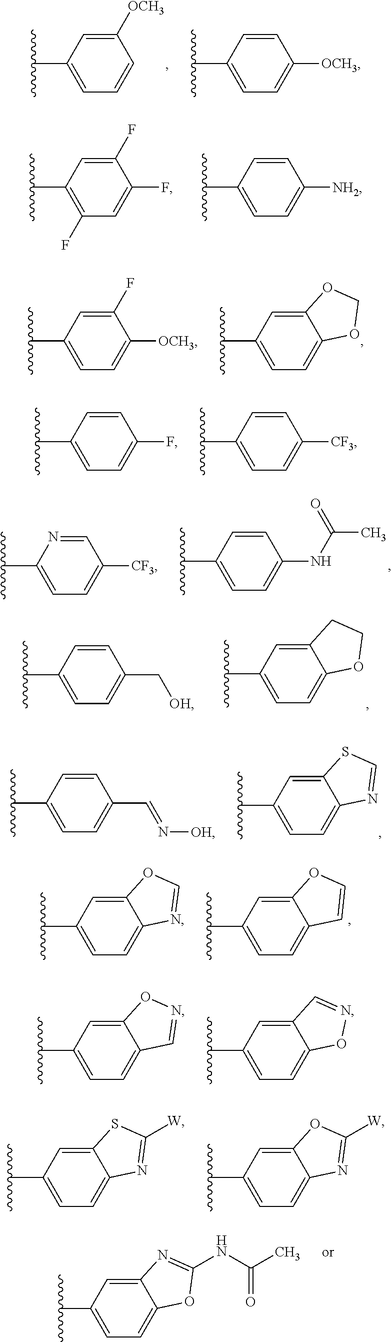

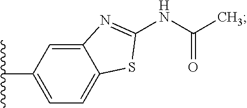

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

Another aspect of the present invention relates to a compound, or a pharmaceutically acceptable salt thereof, of formula XXIII:

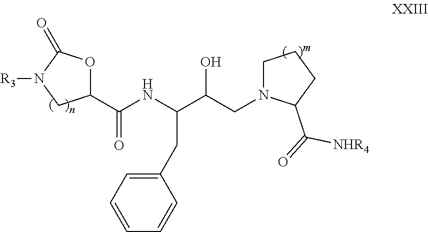

wherein, independently for each occurrence,

n is 1 or 2;

m is 1 or 2;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein m is 1.

In certain embodiments, the present invention relates to any of the aforementioned compounds and any of the attendant definitions, wherein R₃ is

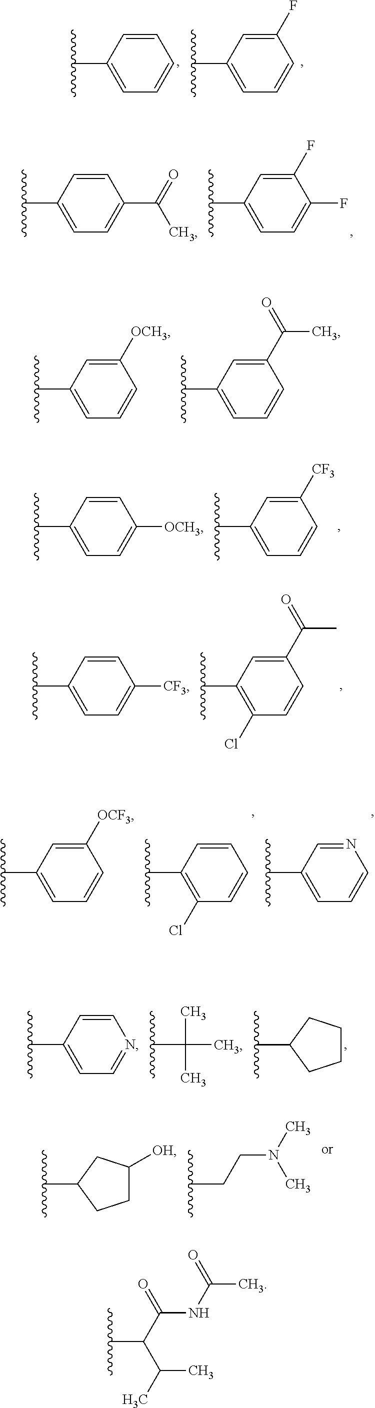

In certain embodiments, the present invention relates to any of the aforementioned compounds and any of the attendant definitions, wherein R₄ is

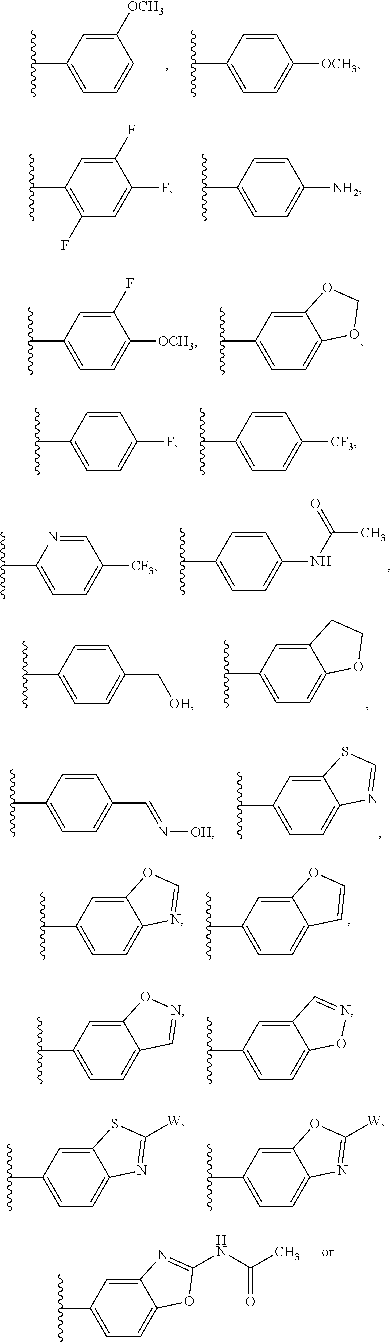

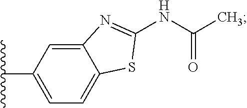

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

Another aspect of the present invention relates to a compound, or a pharmaceutically acceptable salt thereof, of formula XXIV:

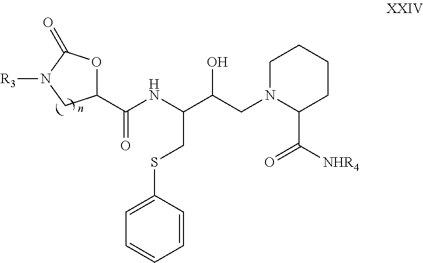

wherein, independently for each occurrence,

n is 1 or 2;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein R₃ is

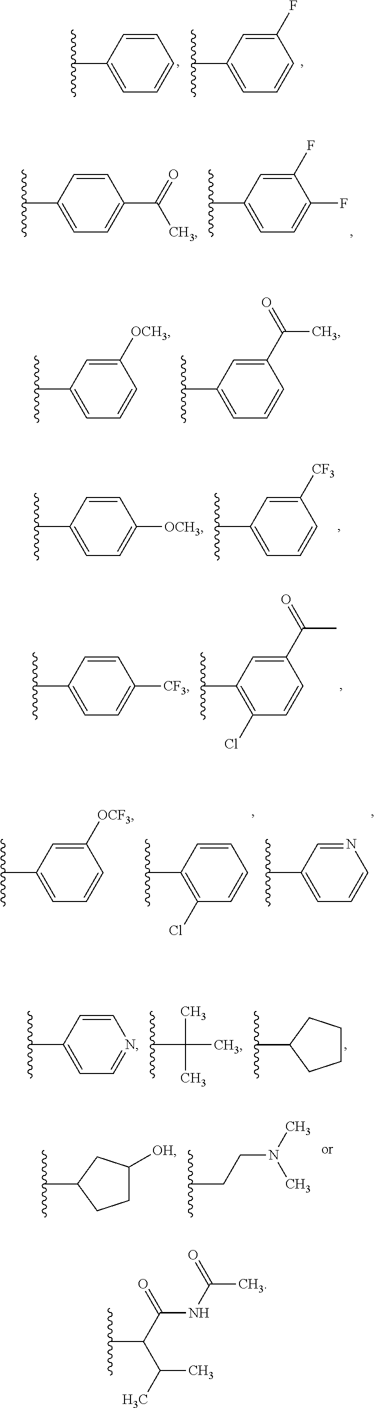

In certain embodiments, the present invention relates to any of the aforementioned compounds and any of the attendant definitions, wherein R₄ is

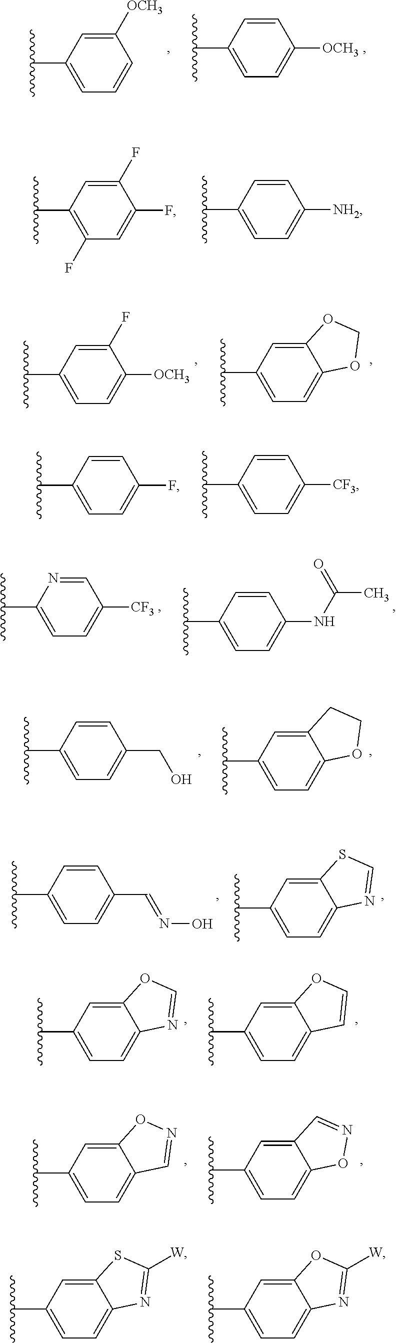

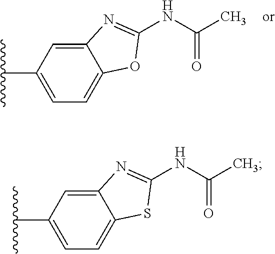

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

Another aspect of the present invention relates to a compound, or a pharmaceutically acceptable salt thereof, of formula XXV:

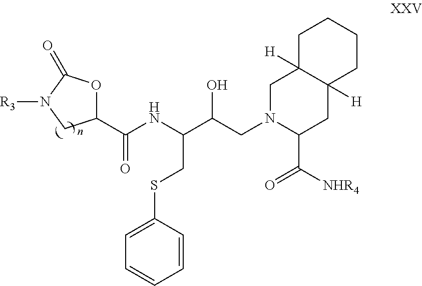

wherein, independently for each occurrence,

n is 1 or 2;

R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;

R₄ is hydrogen, alkyl, cycloalkyl, heterocyclyl, aryl, heteroaryl, (heterocyclyl)alkyl, aralkyl or heteroaralkyl; and

the stereochemical configuration at any undefined stereocenter is R, S, or a mixture of these configurations.

In certain embodiments, the present invention relates to any of the aforementioned compounds and any of the attendant definitions, wherein n is 1.

In certain embodiments, the present invention relates to any of the aforementioned compounds and any of the attendant definitions, wherein R₃ is

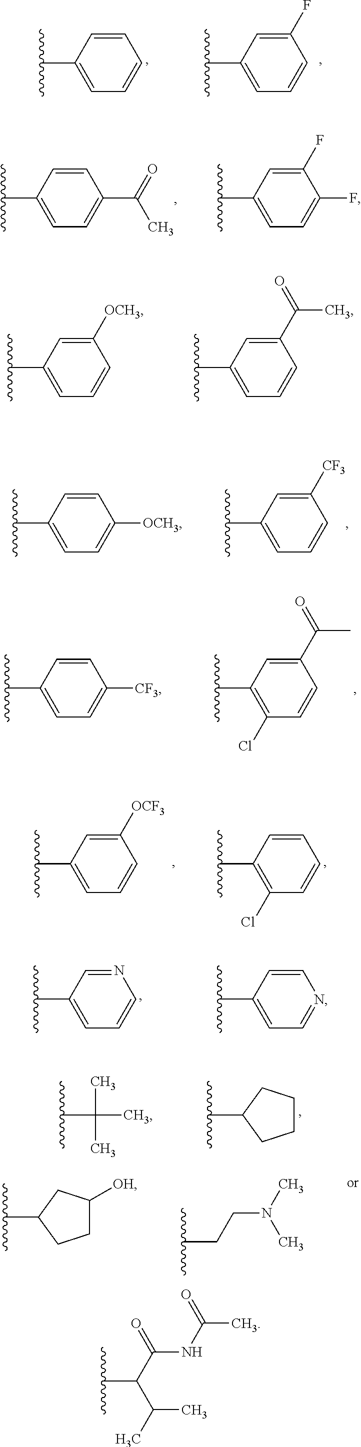

In certain embodiments, the present invention relates to any of the aforementioned compounds and any of the attendant definitions, wherein R₄ is

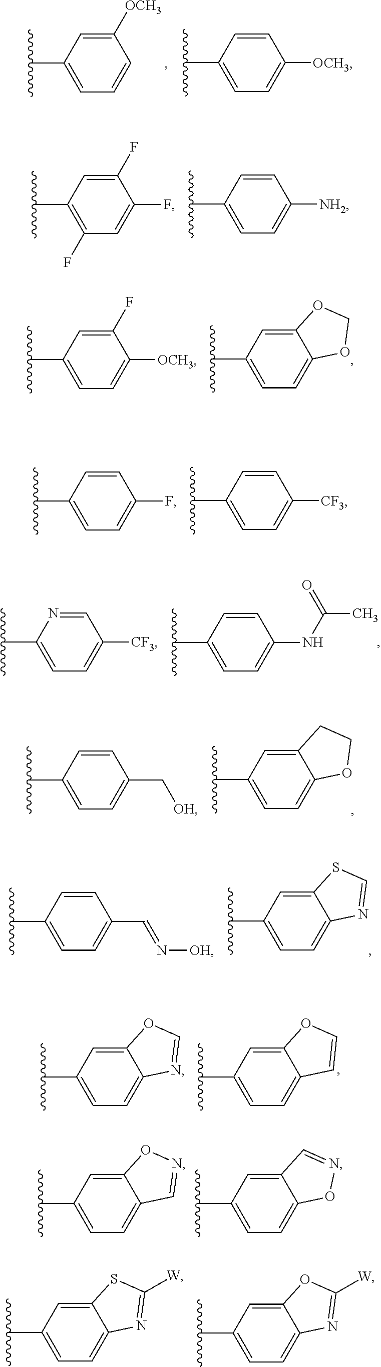

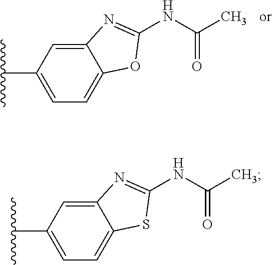

wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

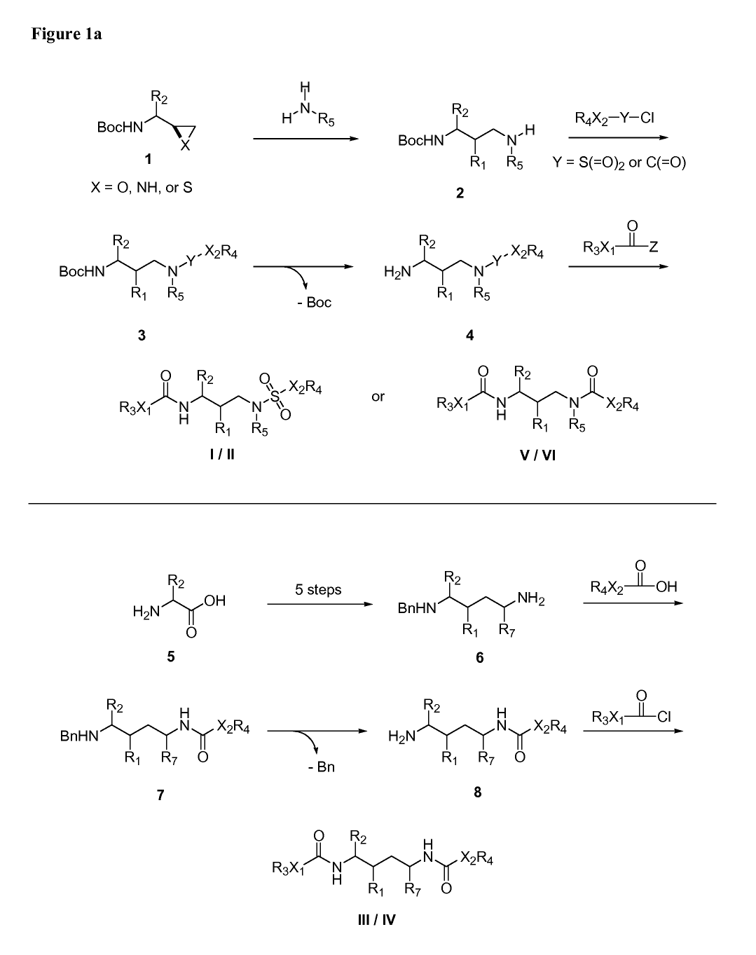

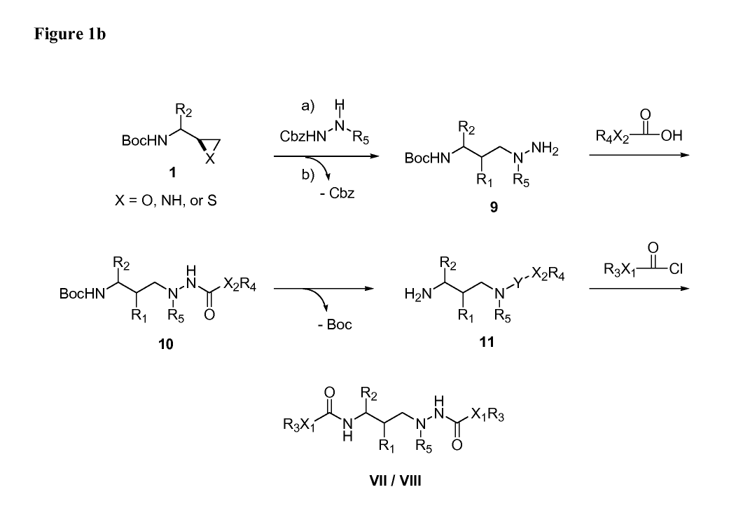

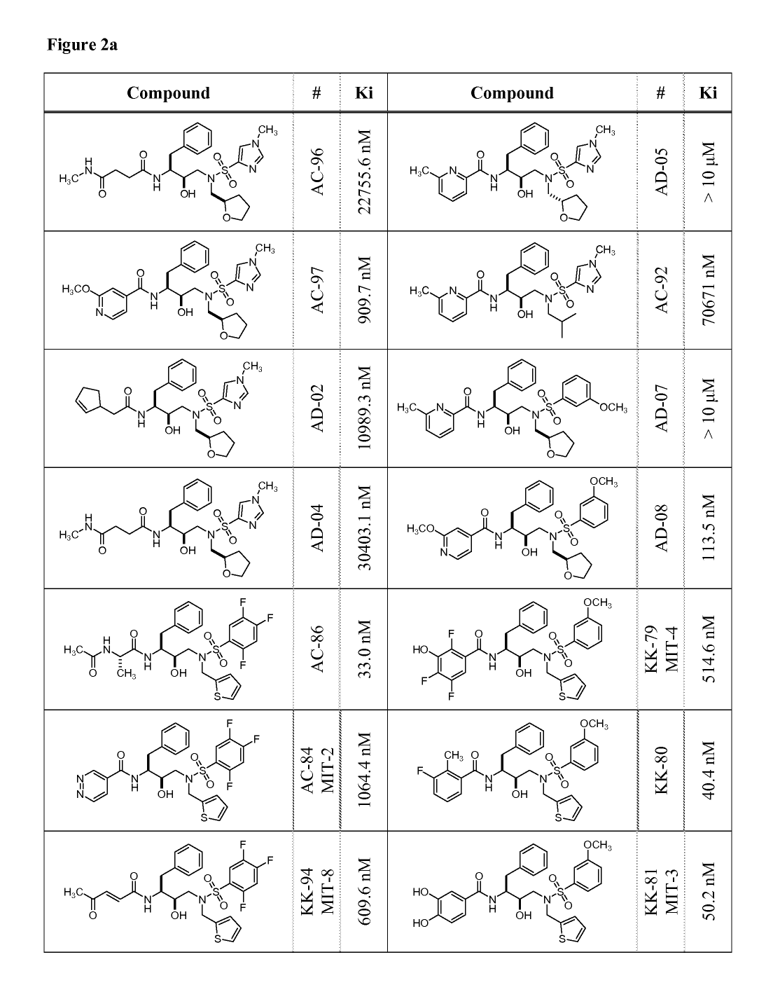

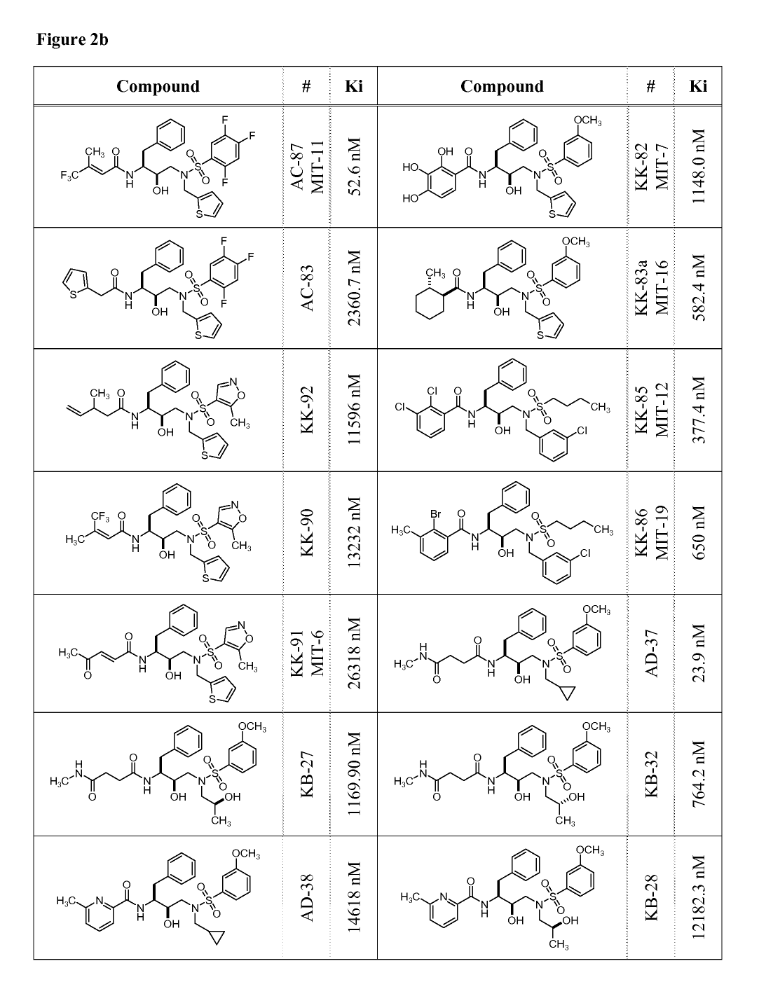

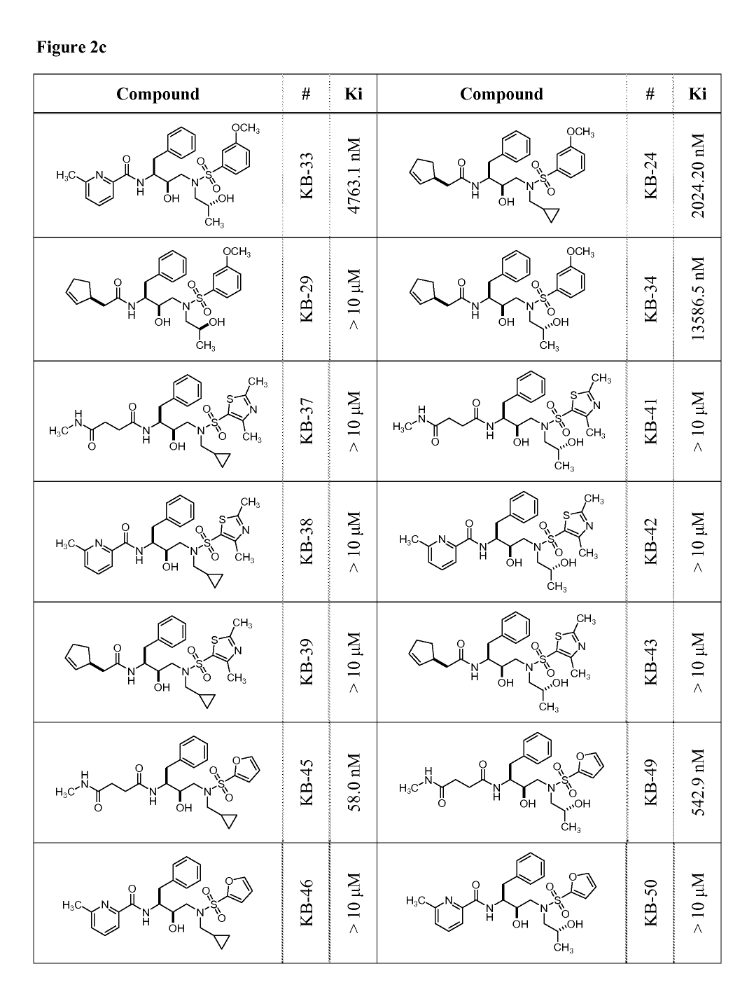

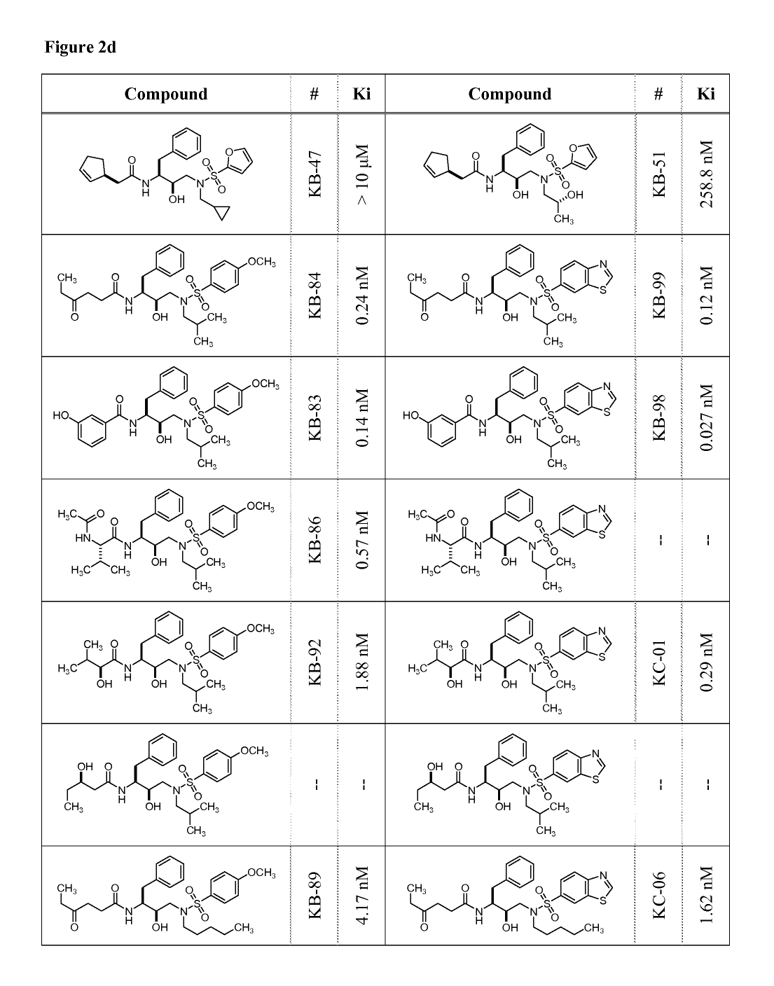

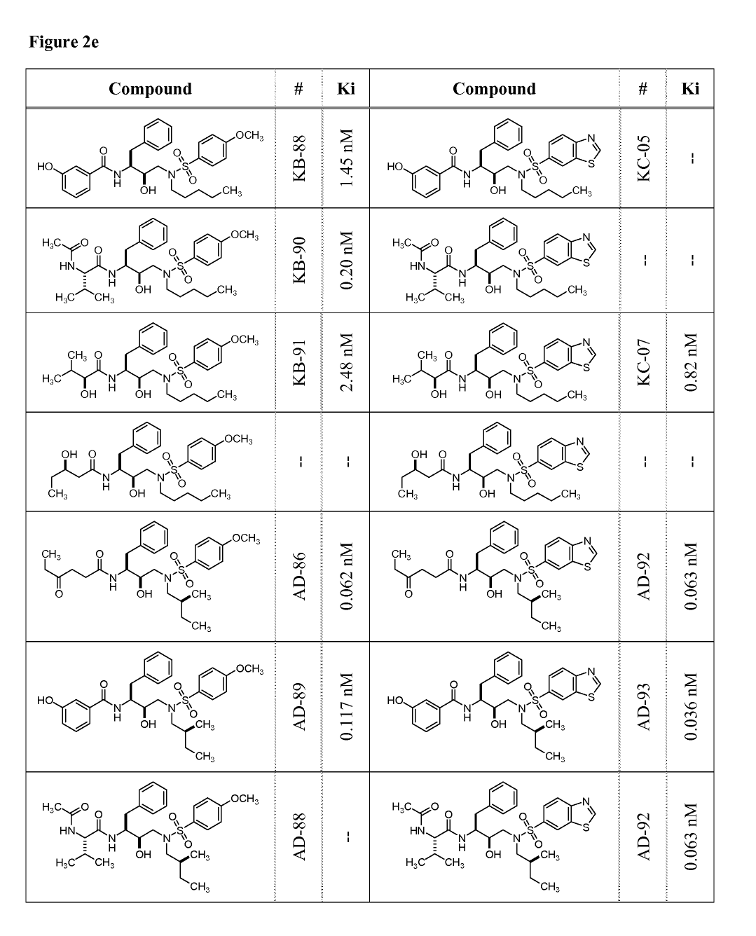

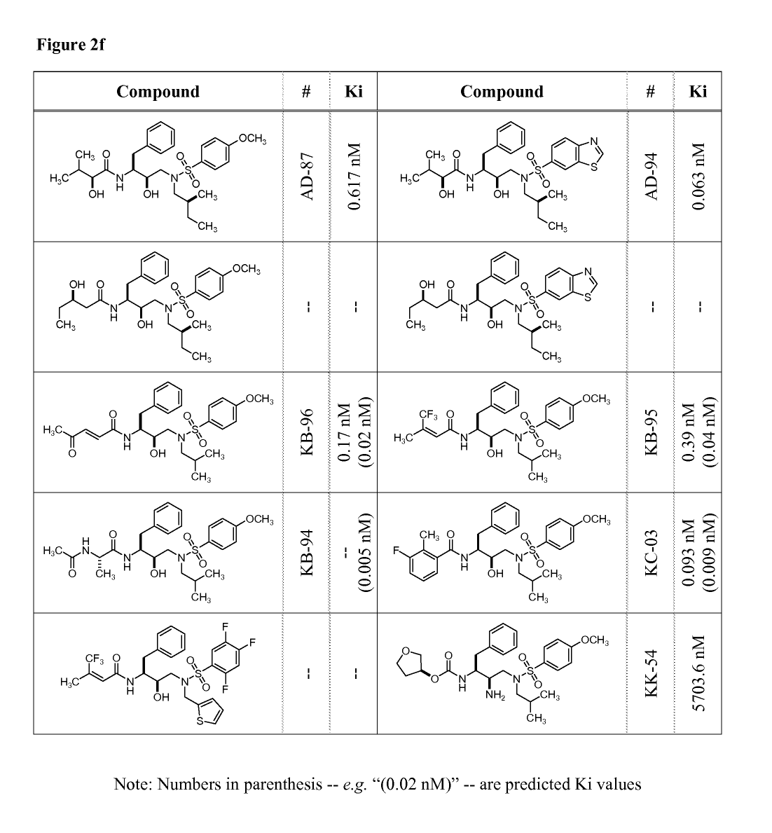

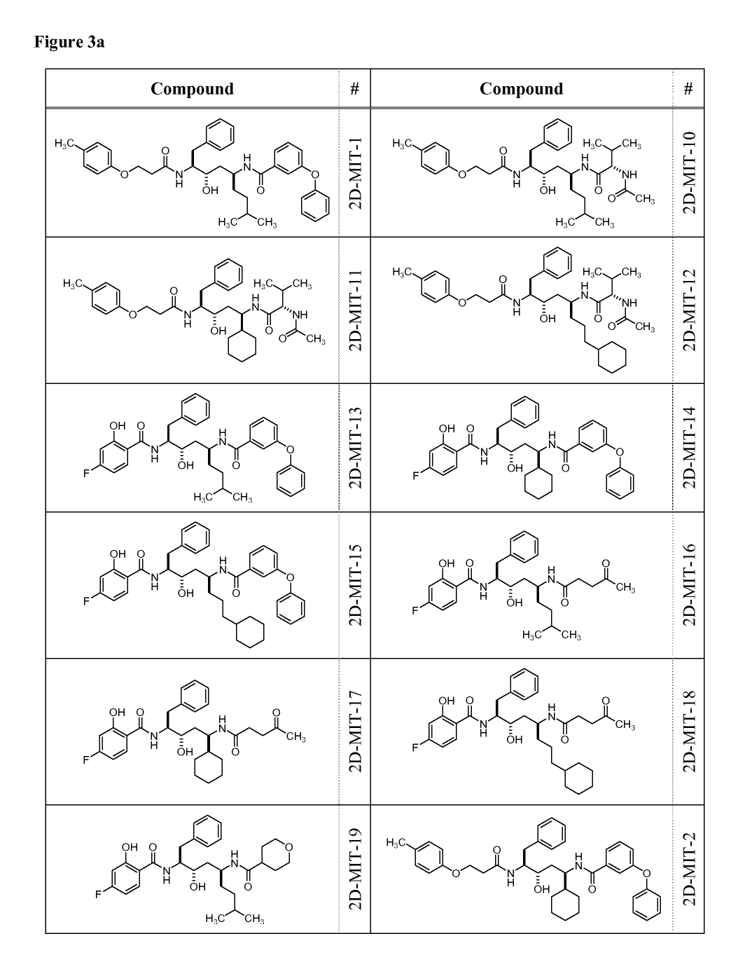

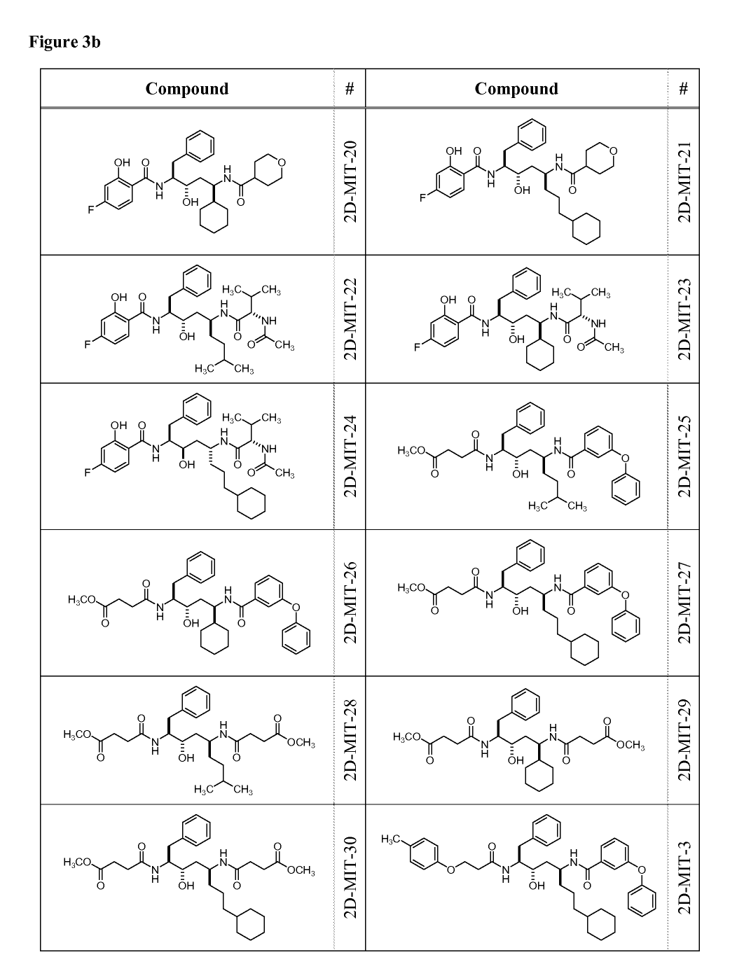

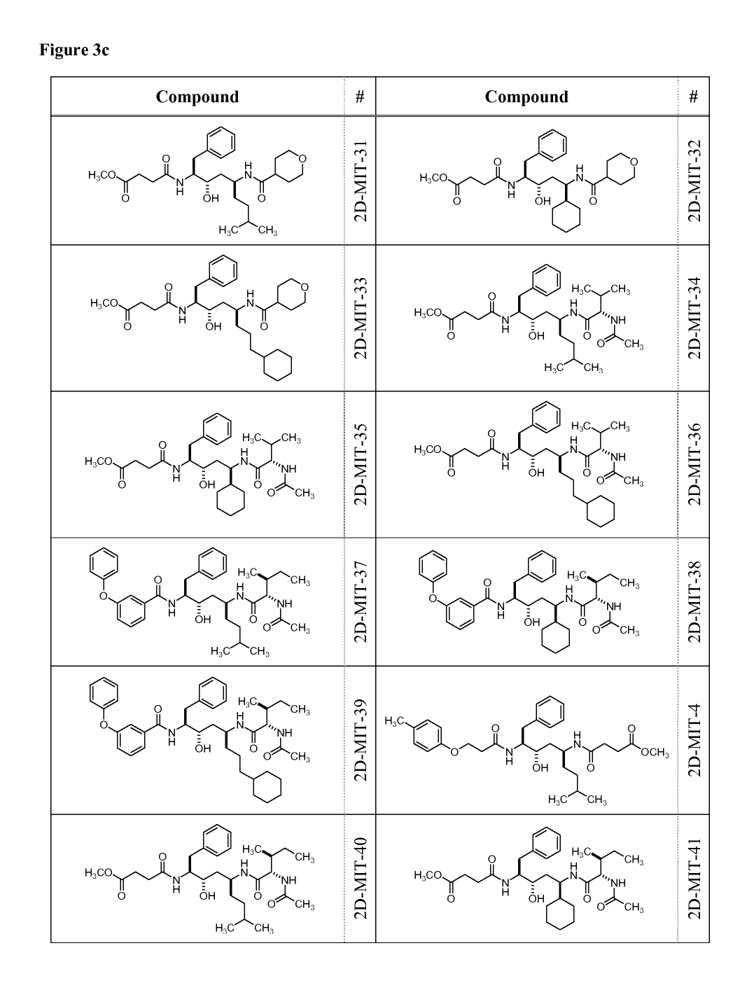

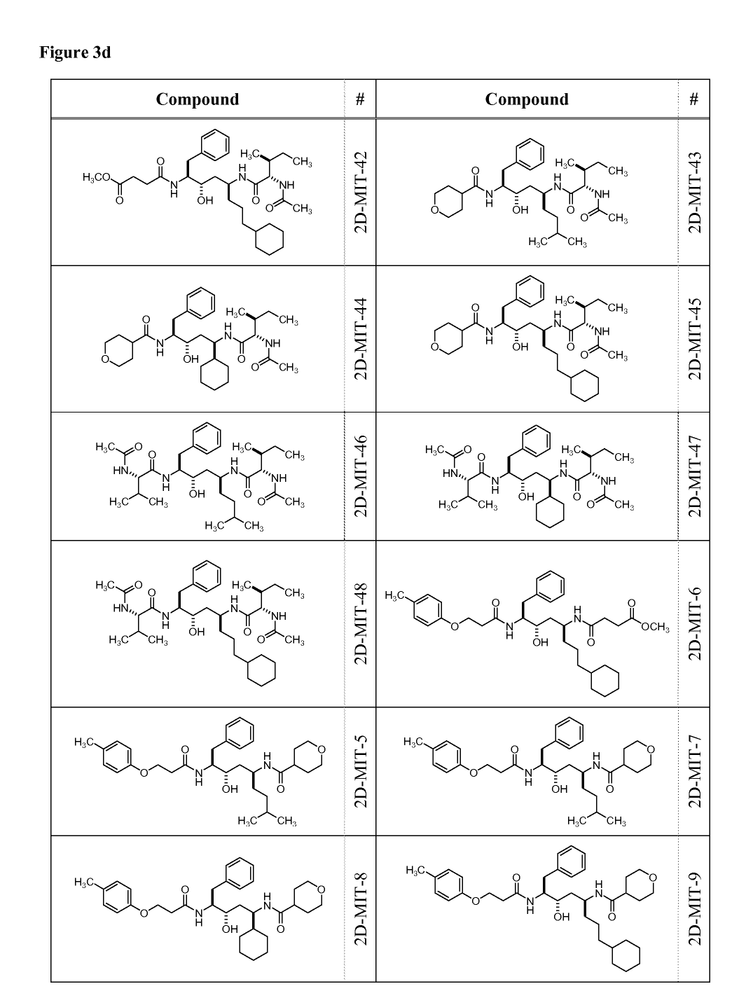

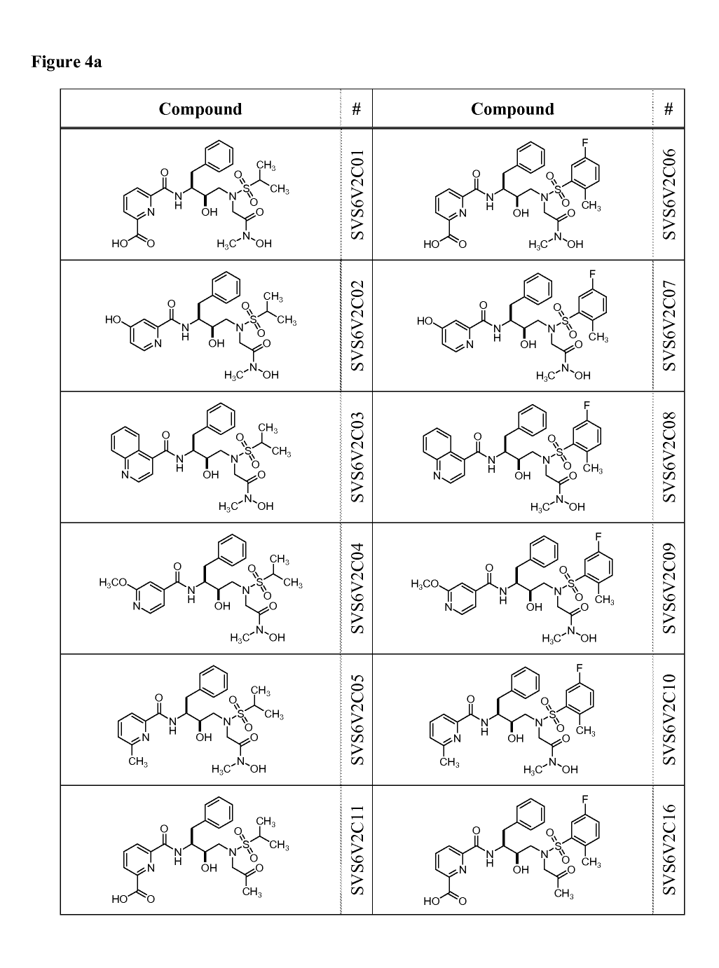

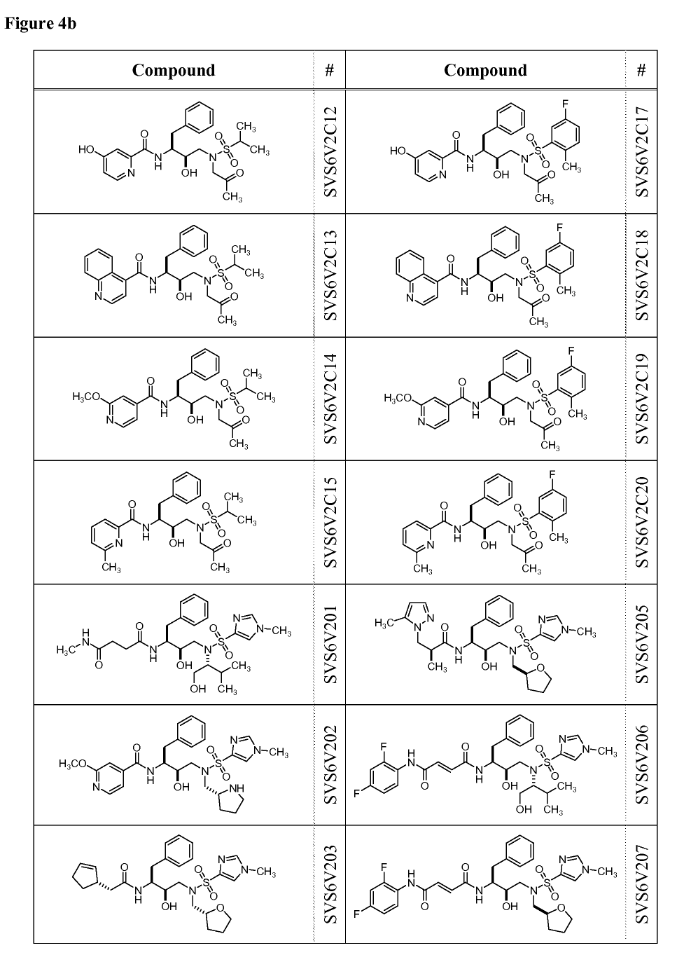

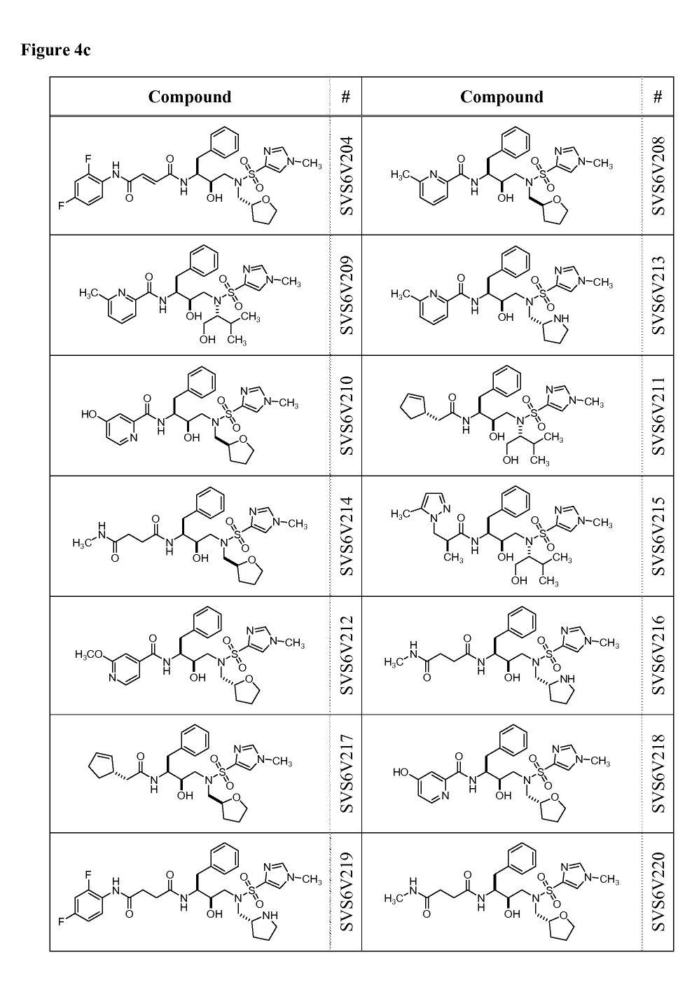

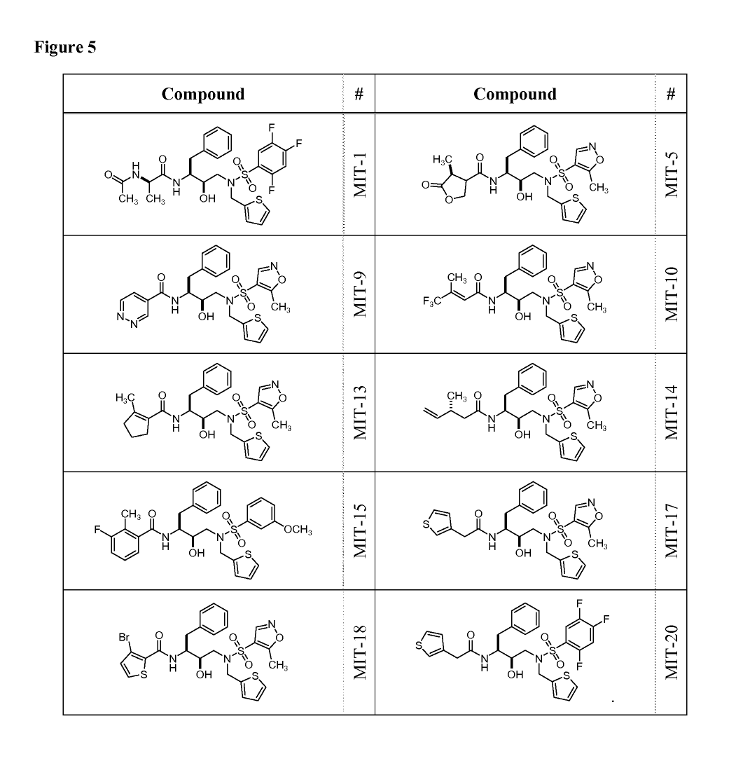

Synthesis of Selected Compounds of the Invention. The protease inhibitors I-VIII can be synthesized using the synthetic schemes outlined in FIGS. 1a-b. The definition of each of the variables may be the same as shown in formulae I-VIII above.

Protease inhibitors I, II, V and VI can be prepared using the synthetic scheme shown in FIG. 1a (top). As shown therein, an epoxide, for example, can be reacted with an amine in a stereoselective manner to yield amine 2. Amine 2 is reacted with sulfonyl chloride or an acyl chloride to yield 3. Deprotection followed by reaction with an acid chloride, for example, yields inhibitor I, II, V or VI.

Protease inhibitor III and IV can be prepared using the synthetic scheme shown in FIG. 1a (bottom). Amino acid 5 can be converted to amine 6 using standard synthetic procedures. Reaction with an acid yields amide 7. Deprotection followed by reaction with an acid chloride yields inhibitor III or IV.

Protease inhibitor IV can be prepared using the synthetic scheme in FIG. 1b. As shown in the scheme, an epoxide, for example, can be reacted with a protected hydrazine in a stereoselective manner to yield hydrazine 9, after deprotection. Hydrazine 9 is reacted with an acid to yield amide 10. Further deprotection yields amine 11 followed by reaction with acid chloride yields inhibitor VII or VIII.

As can be seen from FIGS. 1a and 1b, the R groups of the inhibitors are determined by choosing suitable reagents and starting material. Similarly, the stereochemistry of the inhibitors is determined by choosing appropriate starting material and reagents.

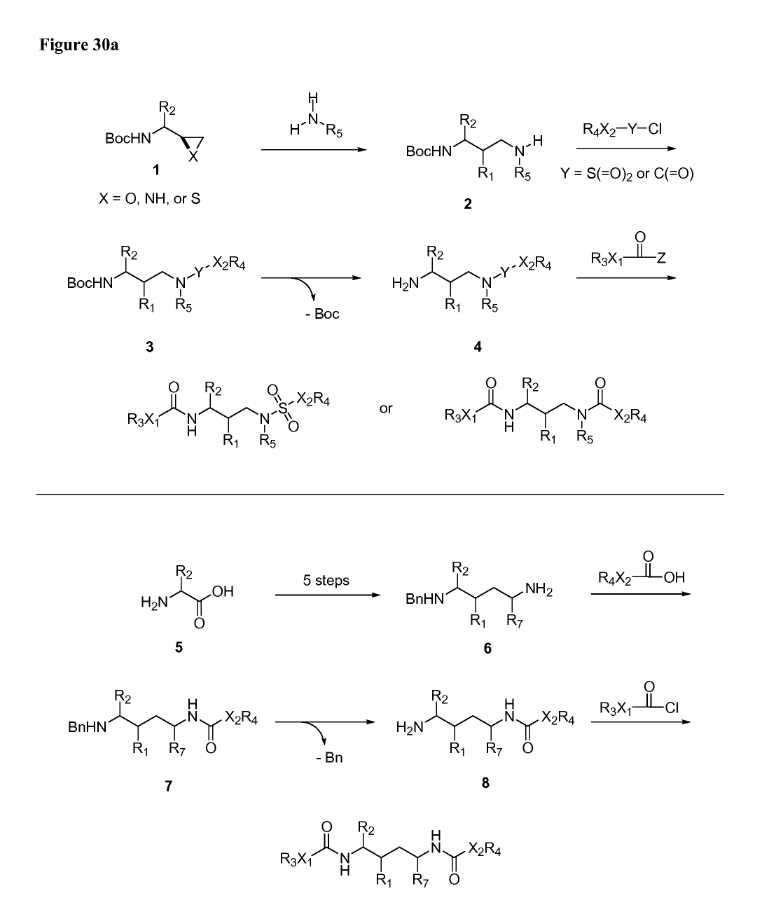

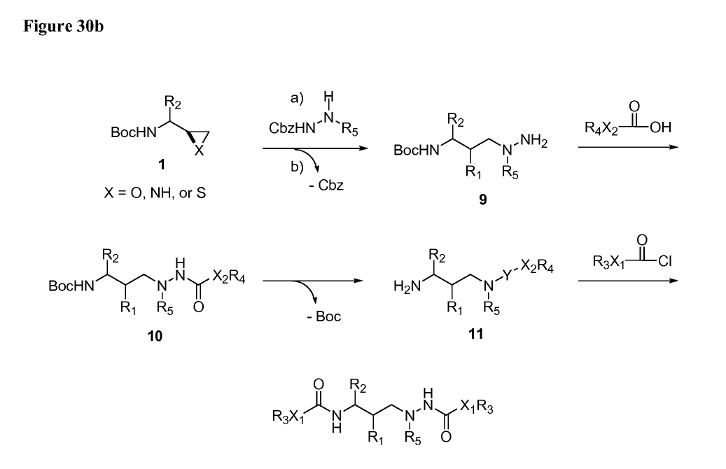

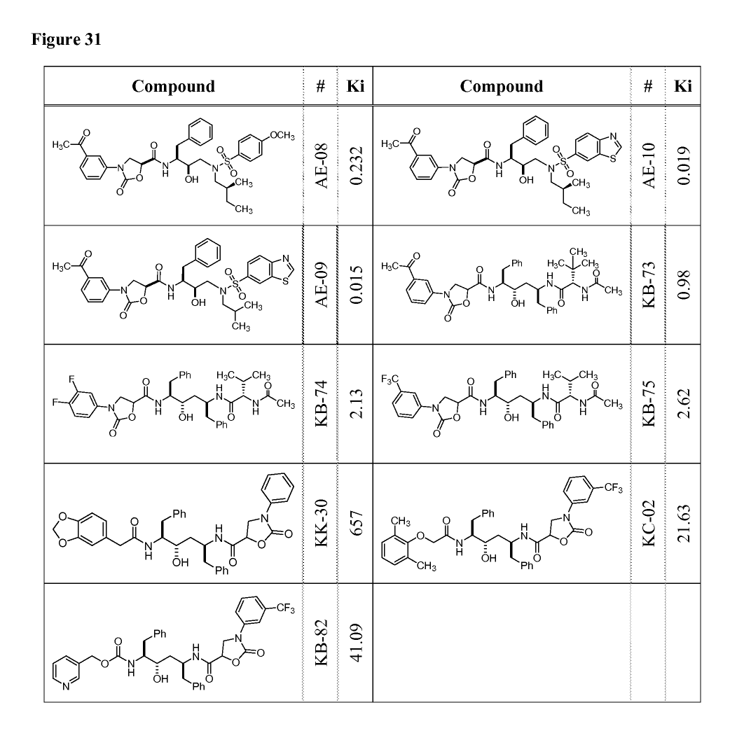

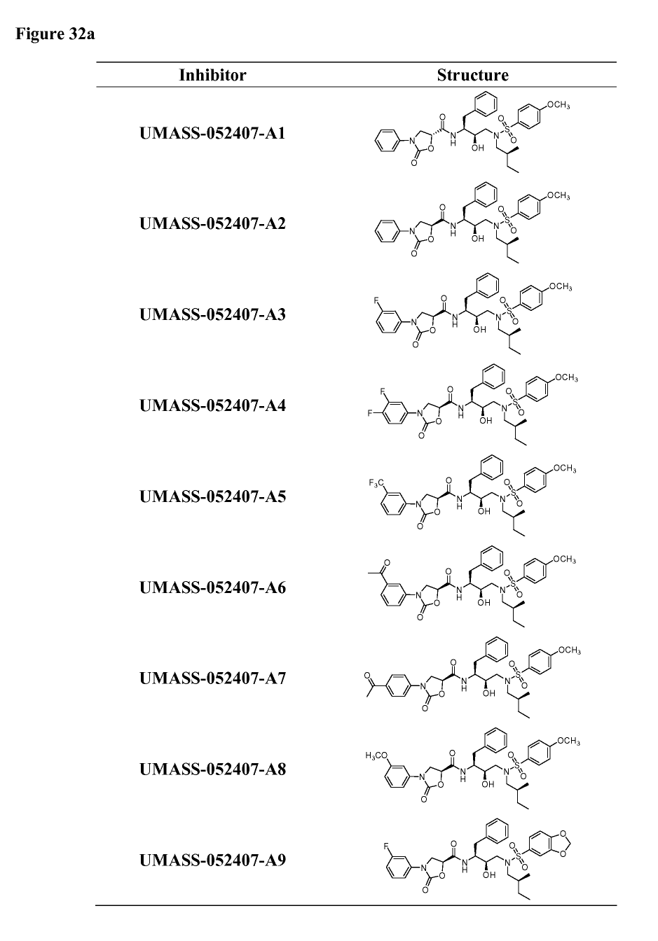

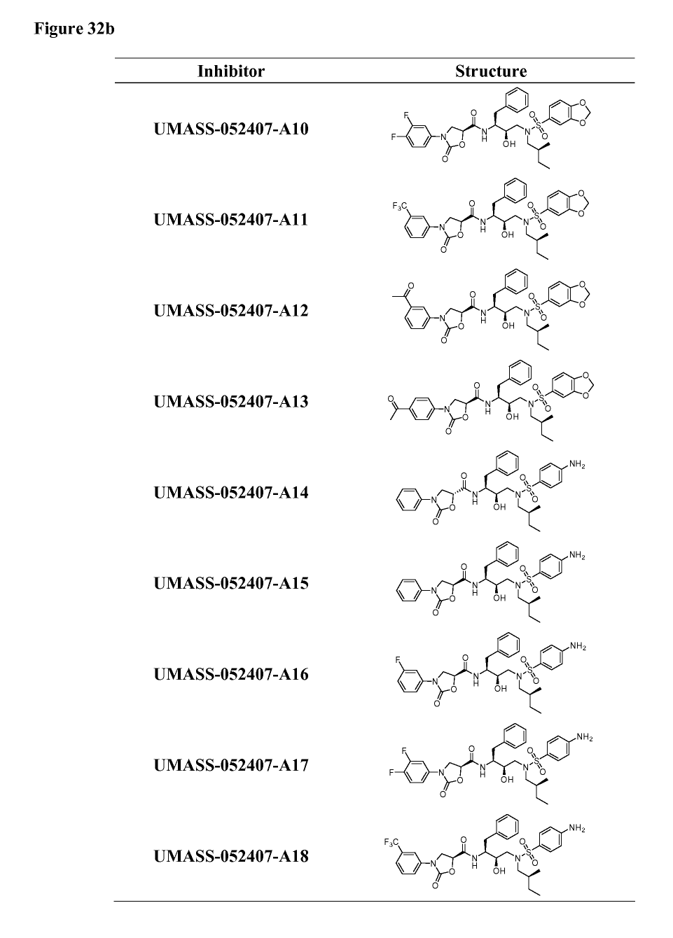

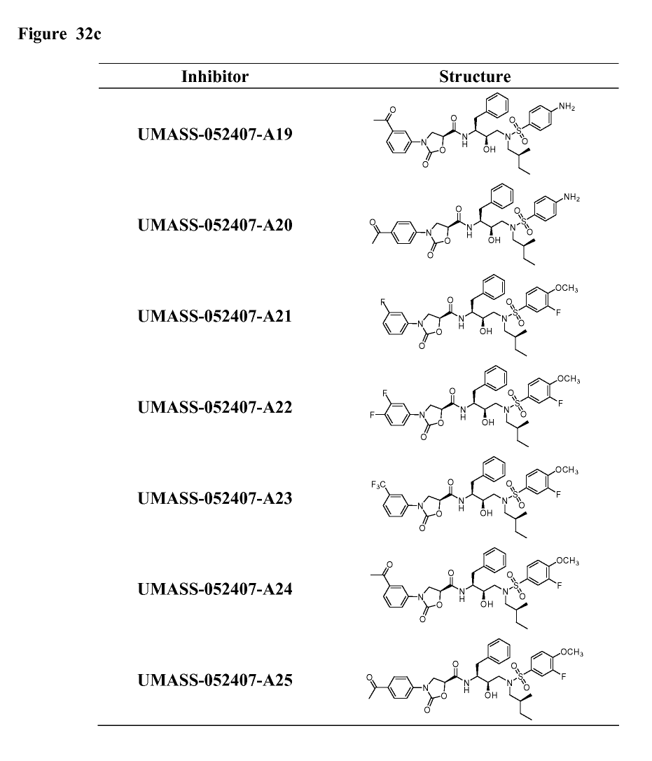

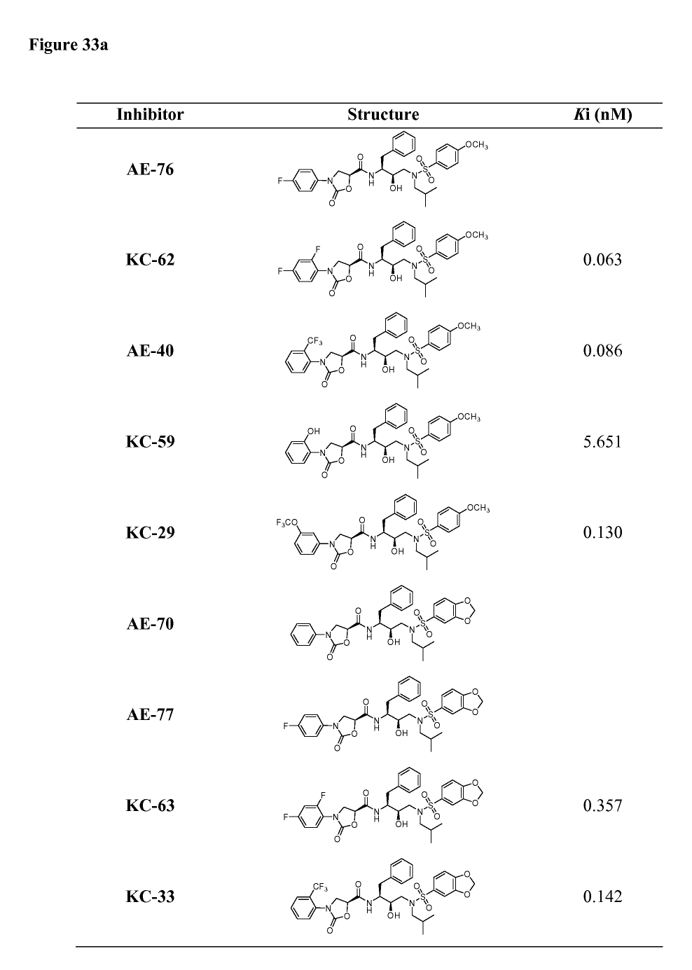

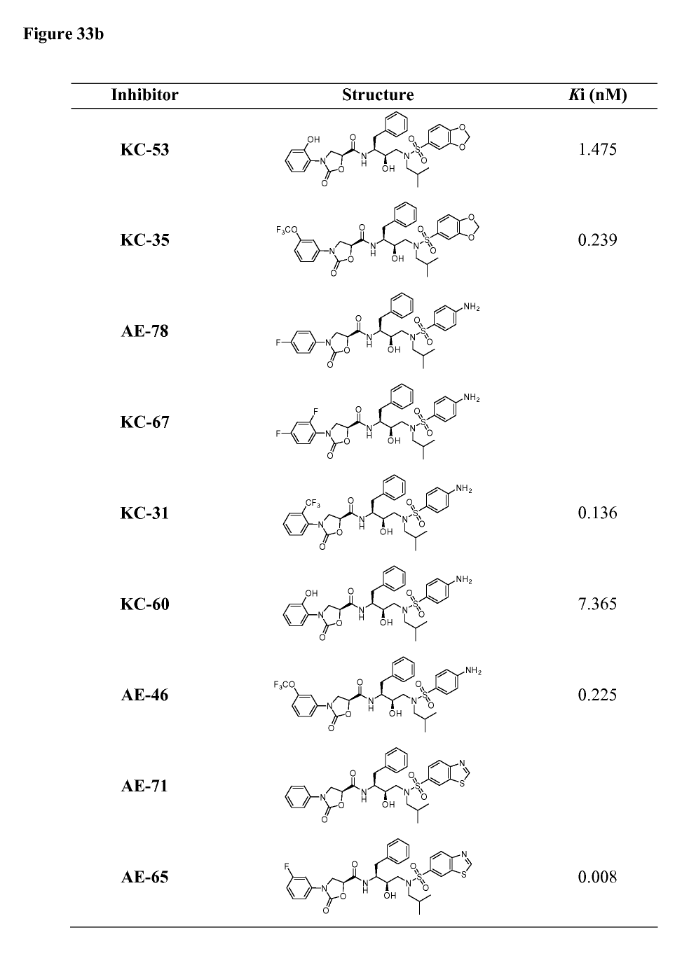

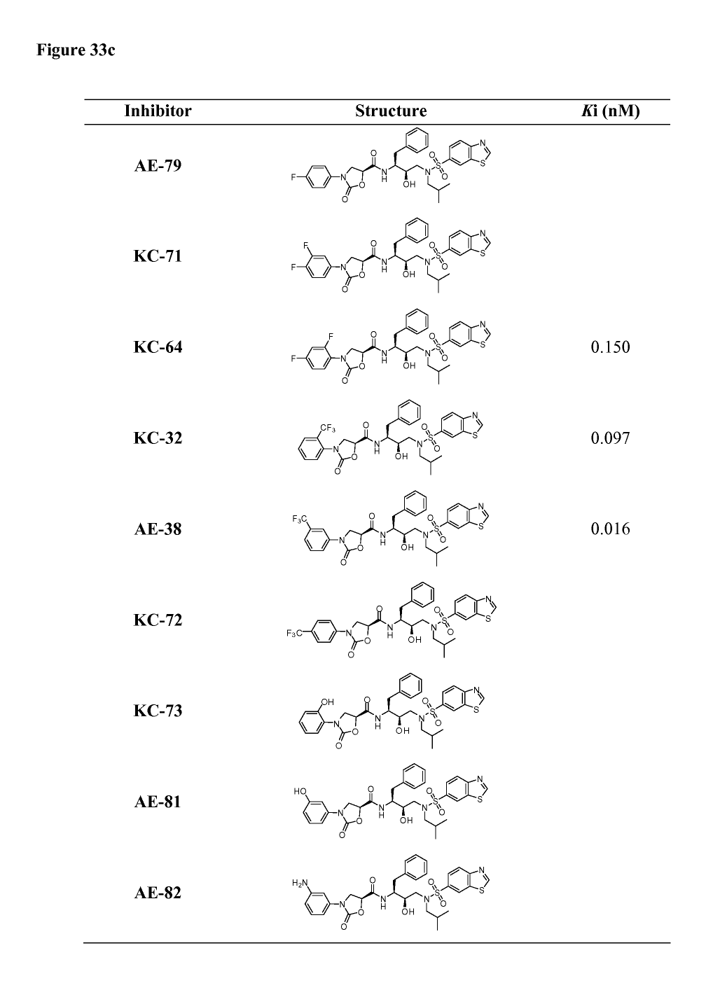

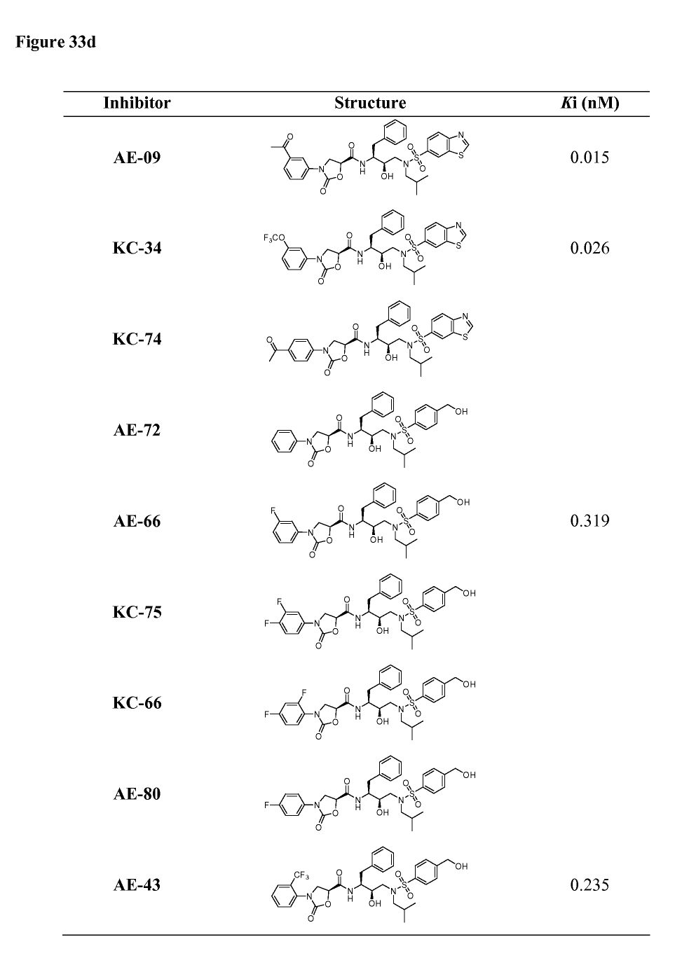

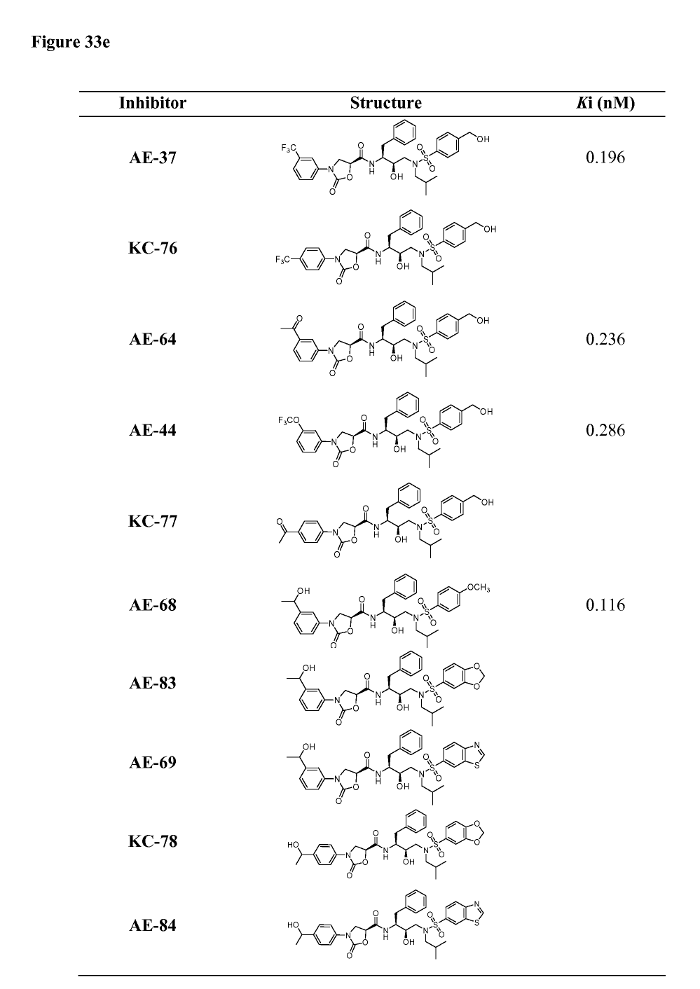

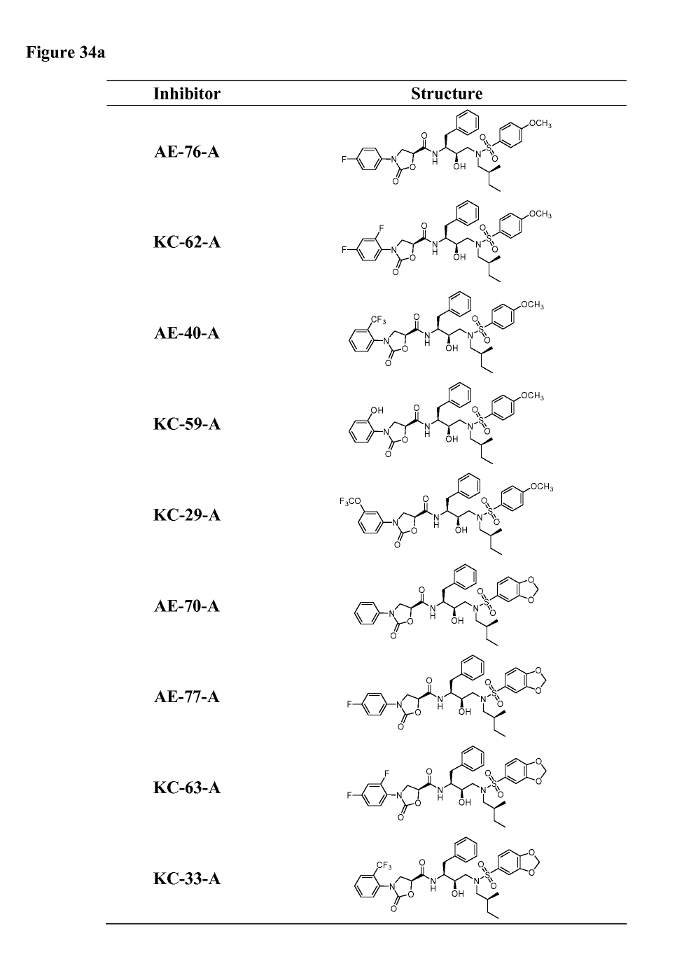

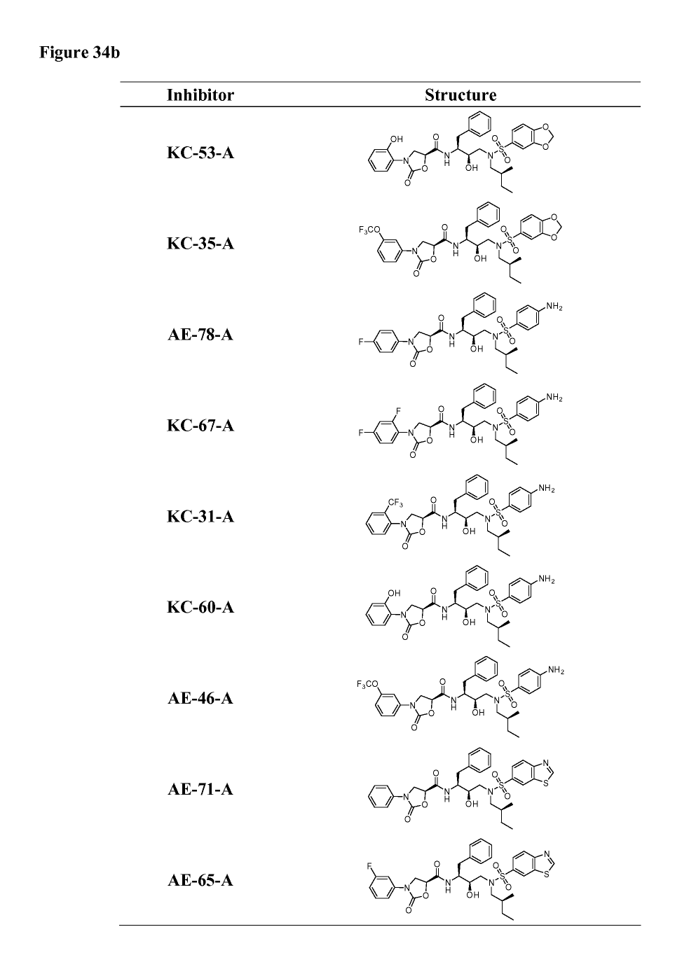

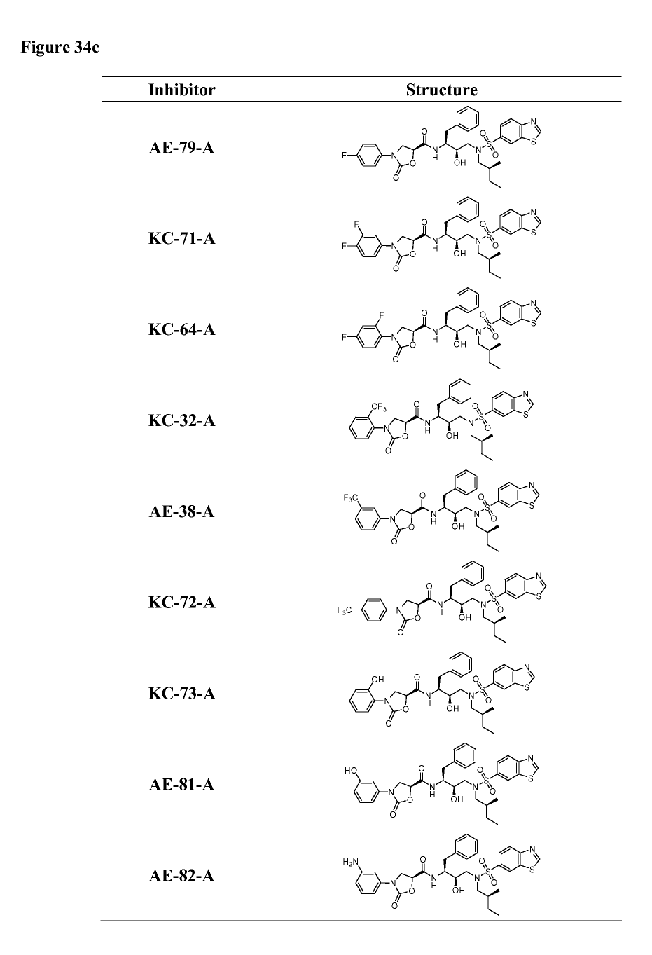

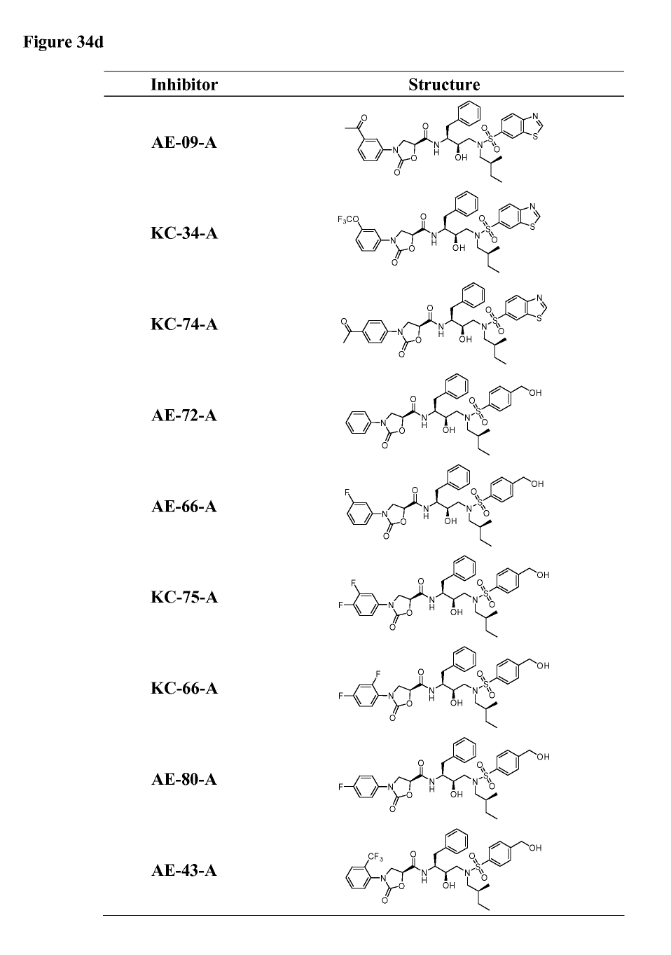

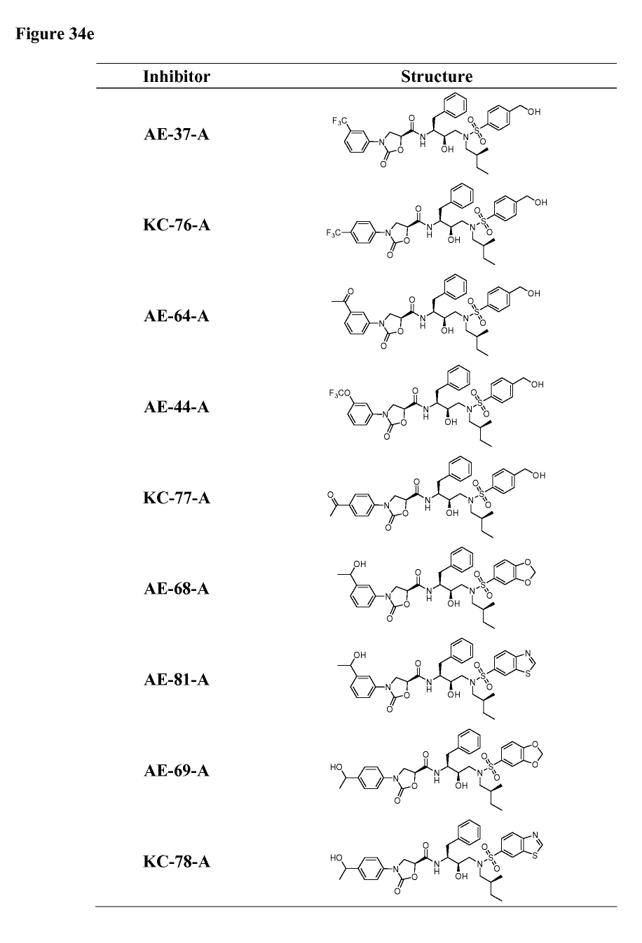

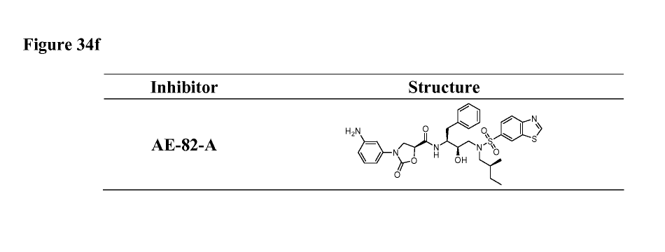

Protease inhibitors XI, XII, XV and XVI can be prepared using the synthetic scheme shown in FIG. 30a (top). As shown therein, an epoxide, for example, can be reacted with an amine in a stereoselective manner to yield amine 2. Amine 2 is reacted with sulfonyl chloride or an acyl chloride to yield 3. Deprotection followed by reaction with an acid chloride, for example, yields inhibitor XI, XII, XV or XVI.

Protease inhibitor XIII and XIV can be prepared using the synthetic scheme shown in FIG. 30a (bottom). Amino acid 5 can be converted to amine 6 using standard synthetic procedures. Reaction with an acid yields amide 7. Deprotection followed by reaction with an acid chloride yields inhibitor XIII or XIV.

Protease inhibitor XIV can be prepared using the synthetic scheme in FIG. 30b. As shown in the scheme, an epoxide, for example, can be reacted with a protected hydrazine in a stereoselective manner to yield hydrazine 9, after deprotection. Hydrazine 9 is reacted with an acid to yield amide 10. Further deprotection yields amine 11 followed by reaction with acid chloride yields inhibitor XVII or XVIII.

As can be seen from FIGS. 30a and 30b, the R groups of the inhibitors are determined by choosing suitable reagents and starting material. Similarly, the stereochemistry of the inhibitors is determined by choosing appropriate starting material and reagents.

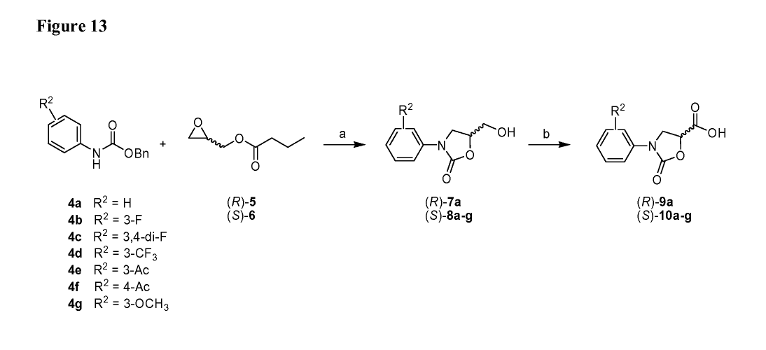

For example, chiral N-phenyloxazolidine-S-carboxylic acids 9 and 10 used in the synthesis of designed inhibitors, were prepared following the literature procedure as outlined in FIG. 13. (Brickner, S. J. et al. J. Med. Chem. 1996, 39, 673-679; Hester, J. B. WO 2003/006440, hereby incorporated by reference; and Thomas, R. C. et al. WO 2003/072553, hereby incorporated by reference). The intermediate chiral alcohols, 5-(hydroxymethyl)-3-aryl-oxazolidine-2-ones 7-8, were obtained from substituted anilines in two steps. The reaction of CBZ protected anilines 4a-g with either (R)- or (S)-enantiomer of glycidyl butyrate promoted by n-BuLi provided chiral alcohols 7a and 8a-g. This one pot, three step cascade reaction involves the initial ring opening of chiral epoxide with N-lithium species followed by an intramolecular cyclization and finally an in situ ester hydrolysis. (Brickner, S. J. et al. J. Med. Chem. 1996, 39, 673-679.) Oxidation of the resulting chiral alcohols using catalytic ruthenium chloride provided the desired N-phenyloxazolidine-S-carboxylic acids 9a and 10a-g (Scheme 1). In case of unsubstituted phenyloxazolidines, both (R)- and (S)-enantiomers, 9a and 10a respectively, were prepared from the corresponding chiral epoxide. All other compounds with substituted phenyl ring, 10b-g, were prepared only as (S)-enantiomers.

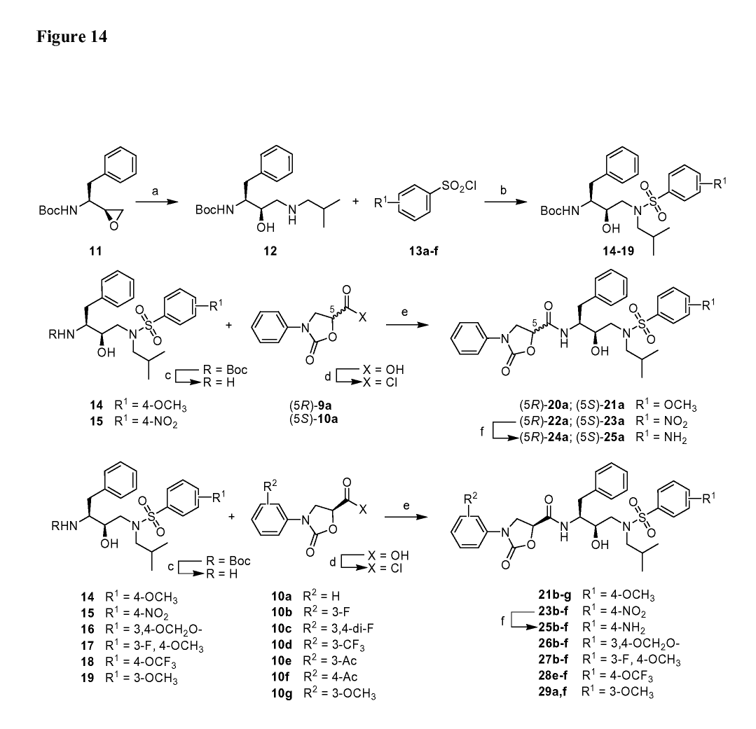

The synthetic route applied for the preparation of designed protease inhibitors is illustrated in FIG. 14. The Boc protected intermediate (R)-(hydroxyethylamino)sulfonamides 14-19 were prepared following literature procedure. (Koh, Y. et al. Antimicrob. Agents Chemother. 2003, 47, 3123-3129; and Surleraux, D. L. N. G. et al. J. Med. Chem. 2005, 48, 1813-1822.) Briefly, ring opening of commercially available chiral epoxide, (1S,2S)-(1-oxiranyl-2-phenylethyl)carbamic acid tert-butyl ester, 11 with isobutylamine provided the amino alcohol 12. Reactions of substituted phenylsulfonyl chlorides with 12 afforded the sulfonamides 14-19 that were coupled with phenyloxazolidine fragments. Initially, four compounds were synthesized using either unsubstituted (R)- or (S)-3-phenyloxazolidine-S-carboxylic acid 9a or 10a attached to the (R)-(hydroxyethylamino)-sulfonamide isostere at P2 position. Previously optimized phenylsulfonamides, 4-methoxyphenylsulfonamide and 4-aminophenylsulfonamide, were utilized as P2′ ligands. Thus, removal of the Boc protection of sulfonamides 14-15 followed by the reactions of the resulting amino alcohols with either (R)- or (S)-enantiomer of the activated carboxylic acids 9a or 10a provided the target compounds 20a-23a (FIG. 14). In the case of compounds 22a and 23a, the nitro group was reduced using tin chloride to afford the corresponding amino derivatives 24a and 25a. It has to be noted that attempts to use the standard amide coupling conditions, EDCI/HOBt/DIEA, were not very successful and resulted in poor yields mainly because of very slow reactions even with DMF as solvent. In all subsequent reactions the carboxylic acids 9 and 10 were converted to the corresponding acid chlorides using oxalyl chloride.

Series of inhibitors were synthesized using substituted (S)-phenyloxazolidines at P2 and different phenylsulfonamide groups at P2′ position for structure-activity relationship (SAR) studies. Following the deprotection of sulfonamide intermediates 14-19, the resulting amines were reacted with (S)—N-phenyloxazolidine-S-carbonyl chlorides obtained by the activation of the corresponding carboxylic acids 10b-g to afford the target compounds 21 and 25-29 (FIG. 14). The compounds 23b-f containing 4-nitrophenylsulfonamide group at P2′ position were transformed to the corresponding 4-aminophenylsulfonamide derivatives 25b-f by the reduction of the nitro group.

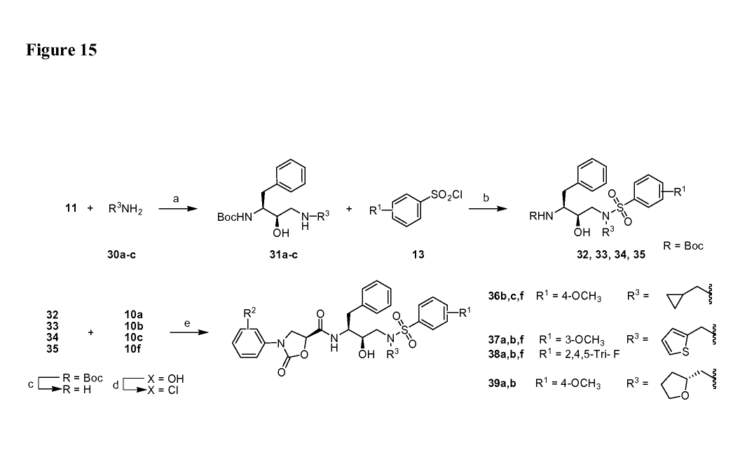

In addition to the compounds described above, series of compounds were prepared with variations at three different positions. The isobutyl group at P1′ position was replaced with three cyclic primary amines. Again, starting from commercially available chiral epoxide 11, the target compounds were synthesized using an analogous synthetic route (FIG. 15). In brief, ring opening of epoxide 11 with primary amines 30a-c provided amino alcohols 31a-c. Reactions of various substituted phenylsulfonyl chlorides with 31a-c provided sulfonylamides 32-35. After deprotection of intermediate compounds 32-35, the resulting amines were reacted with (S)—N-phenyloxazolidine-S-carbonyl chlorides prepared from the corresponding carboxylic acids 10 to afford the target compounds 36-39 (FIG. 15).

Pharmaceutical Compositions. The methods described herein include the manufacture and use of pharmaceutical compositions, which include the protease inhibitors described herein as active ingredients. Also included are the pharmaceutical compositions themselves. These compositions can be administered using routes of administration and dosages similar to those used for known HIV protease inhibitors.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describes pharmaceutically acceptable salts in detail in J. Pharmaceutical Sciences 1977, 66: 1-19, incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Methods of formulating pharmaceutical compositions are known in the art; see, e.g., Remington: The Science and Practice of Pharmacy, 20th Ed. (Baltimore, Md.: Lippincott Williams & Wilkins, 2000). Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with their intended route(s) of administration. Examples of routes of administration include parenteral, e.g., by intravenous, intradermal, or subcutaneous injection; or mucosal (e.g., by oral ingestion, inhalation, or rectal or vaginal administration) administration. Compositions intended for parenteral administration can include the following components: a sterile diluent, such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates and agents for the adjustment of tonicity, such as sodium chloride or dextrose. pH can be adjusted with acids or bases, such as hydrochloric acid or sodium hydroxide, as appropriate. A parenteral preparation can be enclosed in ampoules, disposable syringes or multiple dose vials made of glass or plastic.

Pharmaceutical compositions suitable for injectable use can include sterile aqueous solutions (where the active ingredient is water soluble) or dispersions and sterile powders for the preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent necessary to allow administration via syringe. It should be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating, such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols, such as mannitol, sorbitol, and/or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder, such as microcrystalline cellulose, gum tragacanth or gelatin; an excipient, such as starch or lactose, a disintegrating agent such as alginic acid, Primogel, or corn starch; a lubricant such as magnesium stearate or Sterotes; a glidant, such as colloidal silicon dioxide; a sweetening agent, such as sucrose or saccharin; or a flavoring agent, such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798; hereby incorporated by reference.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The pharmaceutical compositions can also be prepared in the form of suppositories (e.g., with conventional suppository bases, such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; hereby incorporated by reference.

The pharmaceutical compositions can be included in a container, kit, pack, or dispenser, optionally with instructions for administration. A kit may comprise one or more compounds described herein and/or one or more other therapeutic compounds and/or a device for their administration, e.g., a syringe.

Biological Evaluation of Selected Compounds of the Invention. HIV protease inhibitor activities were determined by fluorescence resonance energy transfer (FRET) method. (Matayoshi, E. D. et al. Science 1990, 247, 954-958.) Protease substrate (Arg-Glu(EDANS)-Ser-Gln-Asn-Tyr-Pro-Ile-Val-Gln-Lys(DABCYL)-Arg) was labeled with the energy transfer donor (EDANS) and acceptor (DABCYL) dyes at its two ends to perform FRET. Inhibitor binding dissociation constant (Ki value) was obtained by nonlinear regression fitting to the plot of initial velocity as a function of inhibitor concentration based on Morrison equation. (Greco, W. R. et al. J. Biol. Chem. 1979, 254, 12104-12109.) The activities of all the synthesized inhibitors against wild type HIV-1 protease (Q7K) were determined in triplicate. Chemical structures of inhibitors and their inhibitory activities (Ki values) are presented in the figures.

In addition, a small set of protease inhibitors with potent activities against wild type protease was studied against a panel of multidrug-resistant (MDR) mutants of HIV-1 proteases each representing different paradigms of resistance. The mutant variants were selected by examining the Stanford HIV-1 Drug Resistance Database (http://hivdb.stanford.edu) of HIV-1 infected-patient sequences of viral isolates. The three selected protease variants represent the pattern of resistance mutations that occur under the selective pressure of three or more currently prescribed protease inhibitors. (Wu, T. D. et al. J. Virol. 2003, 77, 4836-4847.) These MDR mutant variants are L10I, G48V, I54V, L63P, V82A (M1), D30N, L63P, N88D (M2), and L10I, L63P, A71V, G73S, I84V, L90M (M3). The inhibitory activities of selected protease inhibitors against M1-M3 mutant HIV-1 proteases were examined. For comparison two currently marketed drugs amprenavir (APV) and lopinavir (LPV) were also studied against the selected panel of mutant proteases.

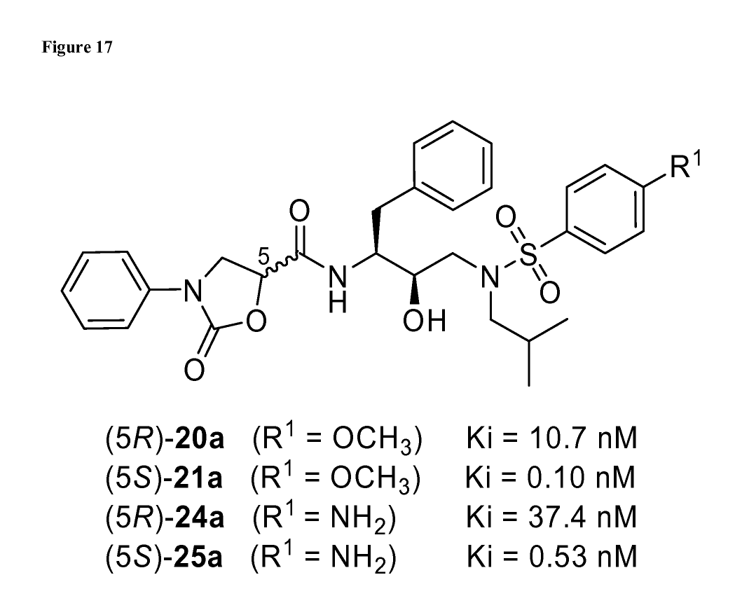

To test the design concept, protease inhibitors based on hydroxyethylamine isostere incorporating unsubstituted N-phenyloxazolidine-S-carboxamide were prepared and tested for their inhibitory activities against wild type HIV-1 protease. It was also critical to determine which stereoisomer of phenyloxazolidine based P2 ligand will bind more favorably to the protease. For these preliminary investigations, two pairs of compounds containing either (R)— or (S)—N-phenyloxazolidine-S-carboxylic acid, (5R)-9a or (5S)-10a, and two phenylsulfonamide groups, 4-methoxy- and 4-aminophenylsulfonamide, were prepared and tested (FIG. 17). The inhibitors 20a and 24a containing (5R)-9a attached at P2 and both 4-methoxy and 4-amino substituted phenylsulfonamides at P2′ position exhibited nanomolar inhibitory activities against wild type HIV-1 protease with Ki=10.7 and Ki=37.4 nM, respectively. When (5S)-10a was attached at P2 position, there was significant improvement in the inhibitory activities with both phenylsulfonamide groups. Interestingly, the compound 21a with 4-methoxybenzenesulfonamide as P2′ ligand was more potent (Ki=0.1 nM) than the 4-aminobenzenesulfonamide analog 25a (Ki=0.53 nM). The protease inhibitory activities of compounds 21a and 25a are comparable to that of structurally related drug amprenavir (APV) 1 with Ki=0.10 nM.

These results clearly show that hydroxyethylamine isostere based inhibitors incorporating N-phenyloxazolidine-S-carboxamides as P2 ligands exhibit potent HIV-1 protease inhibitory activities. These studies also suggest that compounds containing (5S)—N-phenyloxazolidine-5-carboxamides are more potent inhibitors of HIV-1 protease compared to the corresponding (5R)-inhibitors. Thus, in subsequent SAR studies only (S)—N-phenyloxazolidine-S-carboxylic acids, 10a-g, were used.

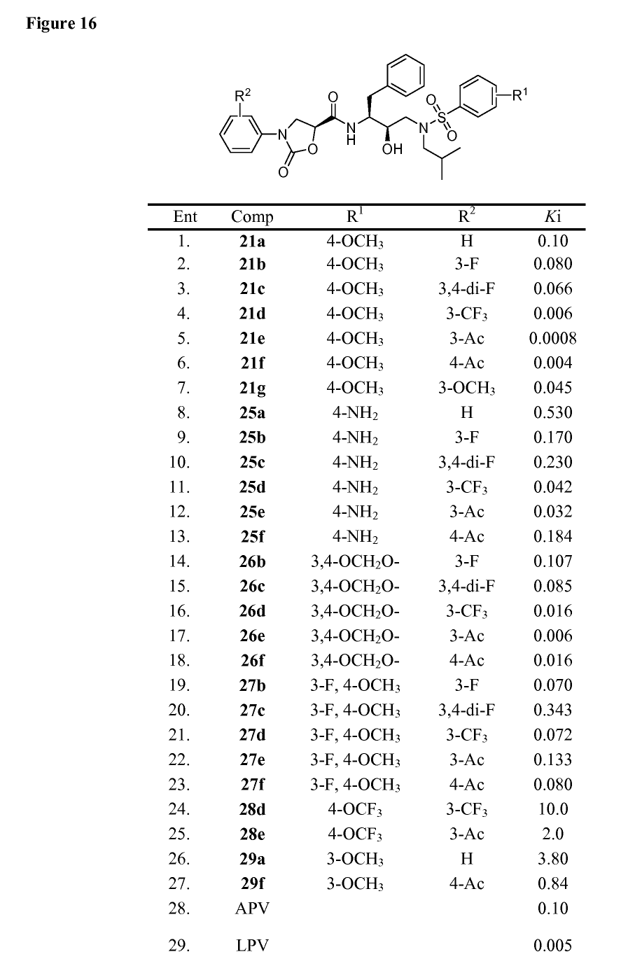

Structure-Activity Relationship Studies. To explore the possibilities of enhancing the potency of 21a and 25a, we synthesized and tested series of analogs with variations at both P2 and P2′ positions. The inhibitor series 21 and 25-29 were prepared using small set of mainly substituted (S)—N-phenyloxazolidine-S-carboxylic acids 10b-g, linked to the (R)-(hydroxyethylamino)sulfonamides 14-19 as P2 ligands (FIG. 14). All the compounds were evaluated for their activities against wild type HIV-1 protease and Ki values are presented in FIG. 16; Ki values of 21a and 25a are also included. The inhibitory activities of amprenavir (APV) and lopinavir (LPV) were also determined using the same assay conditions and Ki data is included in FIG. 16 for comparison.

In the first series of inhibitors, the 4-methoxybenzenesulfonamide group at P2′ was kept constant and substituted N-phenyloxazolidines 10b-g were attached to the (R)-(hydroxyethylamino)sulfonamide isostere 14 as P2 ligands. Compared to 21a, all the compounds with substituted phenyl ring 21b-g exhibited improved inhibitory activities against HIV-1 protease (FIG. 16, entries 1-7). For the inhibitors with 3-fluoro and 3,4-difluoro groups on the phenyl ring, 21b and 21c, the improvement was relatively minor; Ki values of 21b and 21c are 80 pM and 66 pM, respectively. Introduction of polar groups, trifluoromethyl, acetyl and methoxy, at positions 3 and 4 of the phenyl ring resulted in significant increase in the potency of the inhibitors. The Ki value of compound 21d with 3-trifluoromethyl group is 6 pM and the 3-methoxy analog 21g showed Ki value of 45 pM. Inhibitors 21e and 21f with 3- and 4-acetyl groups are the most potent HIV-1 protease inhibitors discovered in our studies with Ki values of 0.8 pM and 4 pM, respectively.

The protease inhibitors 25b-f with 4-aminobenzenesulfonamide at P2′ did not show much improvement in the protease inhibitory activities as was observed in the corresponding 4-methoxy analogs 21. Only the inhibitors with 3-trifluoromethyl and 3-acetyl substituted phenyl ring, 25d (Ki=42 pM) and 25e (Ki=32 pM), exhibited significant increase in potency compared to the unsubstituted analog 25a. Inhibitors with 3-fluoro (25b), 3,4-di-fluoro (25c) and 4-acetyl (25f) groups at the phenyl ring showed relatively small improvement in activities compared to 25a (FIG. 16, entries 9-10 & 13). Compounds 26b-f with another commonly used P2′ ligand, 1,3-[benzo]dioxolane sulfonamide, also showed very potent inhibitory activities. Again, inhibitors with 3-trifluoromethyl (26d), 3-acetyl (26e) and 4-acetyl (26f) groups at the phenyl ring of oxazolidine were the most potent against HIV-1 protease with Ki values of 16 pM, 6 pM and 16 pM, respectively.

The introduction of disubstituted 3-fluoro-4-methoxyphenylsulfonamide at P2′ (26b-f) showed mixed results with different substituted phenyloxazolidines at P2. It appears that the addition of 3-fluoro group on the phenyl ring at P2′ is not well tolerated. Relatively low Ki values were observed for derivatives with 3-fluoro (27b, Ki=70 pM), 3-trifluoromethyl (27d, Ki=72 pM), and 4-acetyl (27f, Ki=80 pM), groups compared to derivatives with 3,4-difluoro (27c, Ki=343 pM), and 3-acetyl (27e, Ki=133 pM) groups. The replacement of 4-methoxyphenyl with 4-trifluoromethoxyphenyl resulted in the complete loss of activity (inhibitors 28d-e). Changing the position of the methoxy group from 4- to 3-position in 29a and 29f also resulted in significant loss of protease inhibitory activity (FIG. 16, entries 26 & 27). These results suggest that inhibitors with electron donating substituents, methoxy, dioxolane and amino, at P2′ phenylsulfonamide group exhibit potent HIV-1 protease inhibitory activities. Other substitutions and modifications are not well tolerated and could result in significant loss of protease inhibitory activity.

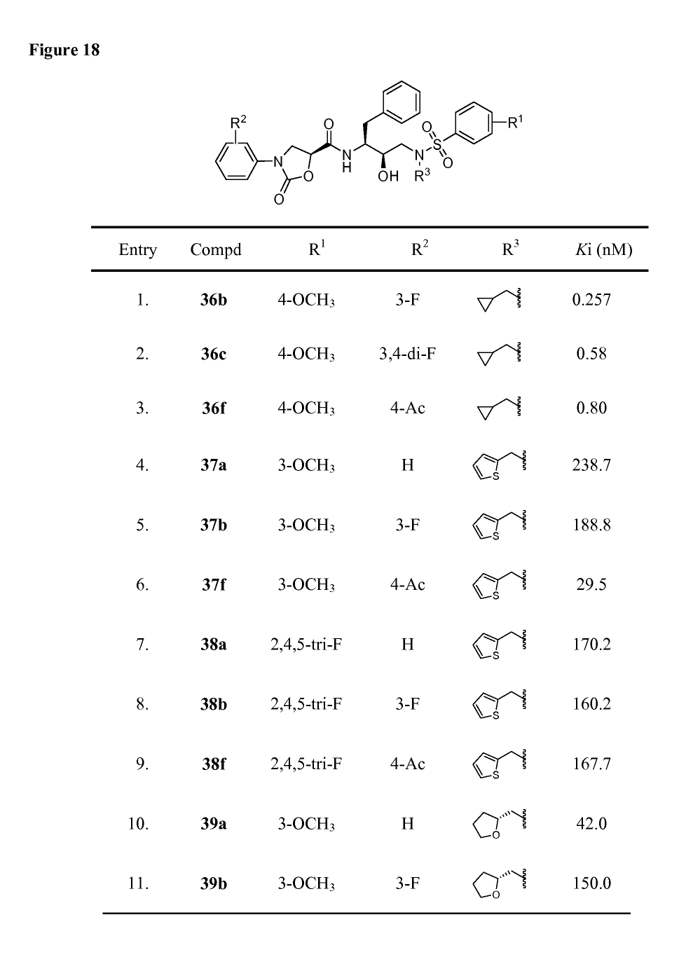

In addition to the variations at P2 and P2′ positions, SAR effort was extended to replace the isobutyl group at P1′ with cyclic groups. Selected primary amines 30a-c include small hydrophobic group cyclopropylmethylamine as well as polar saturated (S)-(2-tetrahydrofuranyl)methylamine and unsaturated 2-(thiophenyl)methylamine heterocyclic groups. Again, sulfonyl chlorides utilized at R¹ position are all substituted benzene derivatives containing functional groups at various positions. A small set of compounds with variations at all three R¹, R² and R³ positions, corresponding to P2′, P2 and P1′, were prepared and tested (FIG. 18). Suprisingly, replacing the isobutyl group at P1′ with cyclopropylmethyl resulted in significantly lower inhibitory activities. The Ki values of 36b (257 pM), 36c (580 pM) and 36f (80 pM) are about 3, 9 and 200 fold higher than the corresponding inhibitors with isobutyl group at P1′ position. Inhibitors 37-39 with (2-thiophenyl)methyl, and (S)-(2-tetrahydrofuranyl)methyl groups at P1′ position all showed very weak inhibitory activities against HIV-1 protease (Table 2 entries 4-11).

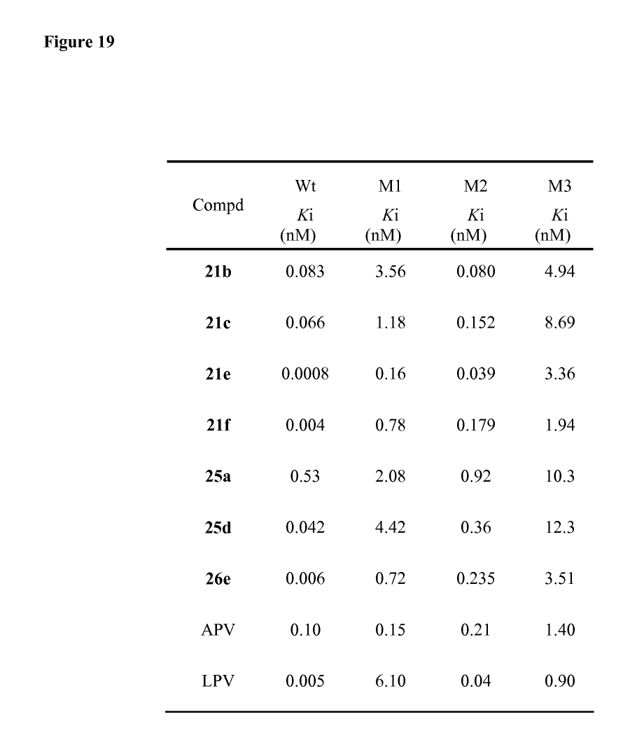

Selected Inhibitor Activities against MDR Mutants. Selected inhibitors that exhibited potent activities against wild type HIV-1 protease were further evaluated for their activities against a panel of MDR mutant variants. At least one compound was selected from each of the series incorporating different phenyloxazolidine based P2 ligands. In some cases, different functional group variations on benzenesulfonamide fragment were selected in preference to compounds with lowest Ki values in that series. The Ki values of selected inhibitors against M1-M3 MDR mutant protease are presented in FIG. 19. Two known drugs, amprenavir (APV, a structurally related compound) and lopinavir (LPV) were also studied for comparisons. The data show that all protease inhibitors loose affinity against mutant variants compared to the wild type protease. However, the relative loss of activity is different in different class of inhibitors. While LPV significantly loses activity against mutants M1 and M3 but, it still retains high affinity against M2 mutant. In case of APV, the relative loss of activity is not that significant against all mutants but, its Ki value for wild type is also relatively high. The protease inhibitors with oxazolidine at P2 showed some loss of activities against M1 mutant, significant loss against M3 mutant, but still retained potent inhibitory activities against M2 mutant. The activity of the most potent protease inhibitor from this series, 21e (Ki=0.8 pM against wild type) was less affected by drug resistance mutations and showed quite potent activity against M1 and M2 MDR enzymes and inhibited M1 and M2 MDR enzymes with a Ki values of 160 pM and 39 pM, respectively.

Methods of Treatment. The methods described herein include methods for the treatment or prevention of a viral infection, e.g., an HIV, infection and Acquired Immunodeficiency Syndrome (AIDS) or AIDS Related Complex (ARC). Generally, the methods include administering a therapeutically effective amount of a protease inhibitor described herein, to a subject (e.g., a human or other primate) in need thereof, or who has been determined to be in need of, such treatment, e.g., a subject who is (or is determined to be) infected with HIV. A subject who is likely to be infected with HIV, e.g., a person in a high risk group, may also be treated as indicated herein. Subjects also include women who are expecting a child (pregnant women) and in whom a treatment reduces the likelihood of transmission of HIV to the child.

In addition to HIV-1 infections, the methods described herein are also expected to be beneficial for treating or preventing HIV-2 infections. Among HIV-1 viruses, it is expected that the methods will be effective against any HIV-1 strain, such as those of group M, O and N, and subtypes A, B, C, D, E, F, G, H, I, J and K and “circulating recombinant forms” or CRFs thereof. The compounds described herein may also be used for treating any other viral infections in which the viral agent has a protease inhibitor that can be inhibited by the compounds described herein.

As used in this context, to “treat” means to ameliorate at least one clinical symptom or parameter of HIV infection or preventing it from worsening or preventing the transmission of HIV, e.g., from mother to child. For example, a treatment can result in a reduction in viral load, and/or an increase in number of CD4+ T cells (“CD4 count”). When a subject has achieved a reduction in viral load, and/or an increase in CD4 count, then treatment may also include maintaining the reduction in viral load, and/or the increased CD4 count, e.g., preventing a resurgence of viral load and/or a decrease in CD4 count. These, and other clinically relevant parameters, can be measured using methods known in the art. For example, viral load can be measured, e.g., using PCR or branched DNA (bDNA) assays known in the art. CD4 counts can be measured, e.g., using hematology, DYNAbeads™ (Dynal Biotech/Invitrogen Corp., Brown Deer, Wis.), flow cytometry (e.g., FACSCount™, BD Biosciences, Franklin Lakes, N.J.) or enzyme-linked immunosorbent assay (ELISA) methods (see, e.g., Lyamuya et al., J. Immunol. Methods 195(1-2):103-12 (1996); Paxton et al., Clin. Diagn. Lab. Immunol. 2(1):104-114 (1995); Saah et al. Arch. Pathol. Lab. Med. 121(9):960-2 (1997); Mwaba et al., Lancet 362 1459-60 (2003)). Healthy adults and teenagers generally have a CD4 count of at least 800 cells per cubic millimeter of blood; a CD4 count below 200 is associated with severe risk of illness (e.g., AIDS-related diseases, such as Kaposi's sarcoma or pneumocystic pneumonia). Current guidelines suggest treatment for HIV should be started when the CD4 count is less than about 350 and/or the viral load is greater than about 50,000.

A “therapeutically effective amount” is an amount sufficient to effect a desired therapeutic effect, e.g., a reduction in viral load, and/or an increase in number of CD4+ T cells. An effective amount can be administered in one or more administrations, applications or dosages. A therapeutically effective amount of a composition may depend on the composition selected. The compositions can be administered once, one or more times per day, and/or one or more times per week; including once every other day. In certain embodiments, the compositions will be administered two or three times per day. The skilled artisan will appreciate that certain factors may influence the dosage and timing required to treat effectively a subject, including but not limited to the severity of the disease or disorder, previous treatments, the general health and/or age of the subject, and any other indications present. Treatment of a subject with a therapeutically effective amount of a protease inhibitor described herein can include a single treatment or a series of treatments.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to select a dose and administration schedule that minimizes severe side effects while maximizing therapeutic efficacy.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in a method described herein, a therapeutically effective dosage range can be estimated initially from cell culture assays. A dose can be further formulated in animal models to achieve a circulating plasma concentration range that includes the IC50 (i.e., the concentration of the test compound that achieves a half-maximal inhibition of symptoms) as determined in cell culture. Such information can be used to determine more accurately useful doses in humans. Levels in plasma may be measured, for example, by high performance liquid chromatography. In some embodiments, a therapeutically effective amount of a new protease inhibitor described herein ranges from about 0.1 to 10 mg per day, or about 0.3 to 5 mg/day.

In some embodiments, one or more of the protease inhibitors described herein will be administered in combination with one or more other therapeutic agents, e.g., as part of a highly active antiretroviral therapy (HAART) regimen that includes one or more other anti-retroviral agents. For example, the methods may include administration of one or more of a non-nucleoside reverse transcriptase inhibitor (NNRTI), such as efavirenz (Sustiva™), nevirapine (Viramune™) and delavirdine (Rescriptor™), 8 and 9-Cl TIBO (tivirapine), loviride, TMC-125, dapivirine, MKC-442, UC 781, UC 782, Capravirine, DPC 961, DPC963, DPC082, DPC083, calanolide A, SJ-1366, TSAO, 4″-deaminated TSAO, MV150 and MV026048; a nucleoside reverse transcriptase inhibitor (NRTI), such as AZT (zidovudine, Retrovir™)/3TC (lamivudine, Epivir™), emtricitabine (Emtriva™) and d4T (stavudine, Zerit™)/3TC, and d-drugs (ddI [didanosine, Videx™/VidexEC™], ddC [zalcitabine, Hivid™], d4T), Abacavir, FTC, DAPD, dOTC, and DPC 817; a nucleotide reverse transcriptase inhibitor, such as tenofovir (Viread™) and PMEA; a fusion inhibitor, such as enfuvirtide (Fuzeon™), T20, T1249, 5-helix and D-peptide ADS-J1; an entry inhibitor; a co-receptor binding inhibitor, such as AMD 3100, AMD-3465, AMD7049, AMD3451 (Bicyclams), TAK 779; SHC-C (SCH351125), SHC-D, PRO-140RT inhibitors, such as foscarnet and prodrugs; an RNAse H inhibitor, such as SP1093V and PD126338; a TAT inhibitor, such as RO-5-3335, K12 and K37; an integrase inhibitor, such as L 708906, L 731988 and S-1360; another protease inhibitor, such as amprenavir and prodrug GW908, nelfinavir, saquinavir, indinavir, lopinavir, palinavir, BMS186316, atazanavir, DPC 681, DPC 684, tipranavir, AG1776, mozenavir, GS3333, KNI-413, KNI-272, L754394, L756425, LG-71350, PD161374, PD173606, PD177298, PD178390, PD178392, PNU 140135, TMC114 maslinic acid and U-140690; a glycosylation inhibitor, such as castanospermine, deoxynojirimycine; or a binding inhibitor, such as dextran sulfate, suramine, polyanions, soluble CD4, PRO-542 and BMS-806. Other drugs include those set forth at http://aidsinfo.nih.gov/, hereby incorporated by reference.

Other therapeutic agents that may be coadministered with one or more agents described herein are agents that inhibit metabolic enzymes, e.g., inhibitors of cytochrome P450 (CYP450) enzymes. For example, a compound described herein may be administered, simultaneously or not, with an inhibitor of CYP3A4, e.g., Ritonavir, or an inhibitor of CYP2C19, CYP1A2, CYP2D6, or CYP2C9. Exemplary inhibitors of 2C9 are described, e.g., in U.S. publication No. 2006.0069042, hereby incorporated by reference.

The compounds of the present invention may also be administered in combination with immunomodulators (e.g., bropirimine, anti-human alpha interferon antibody, IL-2, methionine enkephalin, interferon alpha, HE-2000 and naltrexone), antibiotics (e.g., pentamidine isothiorate), cytokines (e.g. Th2), modulators of cytokines, chemokines or the receptors thereof (e.g. CCR5) or hormones (e.g. growth hormone), to ameliorate, combat, or eliminate HIV infection and its symptoms.

In some embodiments, the methods further comprise administering a second therapeutic agent, wherein the second therapeutic agent is selected from the group consisting of amprenavir (Agenerase®; APV), tipranavir (Aptivus®; TPV), indinavir (Crixivan®; IDV), saquinavir (Invirase®; SQV), lopinavir and ritonavir (Kaletra®; LPV), fosamprenavir (Lexiva®; FPV), ritonavir (Norvir®; RTV), atazanavir (Reyataz®; ATZ), nelfinavir (Viracept®; NFV), brecanavir, and darunavir.

In some embodiments, the methods further comprise administering a second therapeutic agent, wherein the second therapeutic agent is ritonavir (Kaletra®; LPV).

In some embodiments, the methods further comprise administering a second therapeutic agent, wherein the second therapeutic agent is selected from the group consisting of zidovudine (AZT; Azidothymidine; Retrovir®), didanosine (Dideoxyinosine; ddI; Videx®), zalcitabine (Dideoxycytidine; ddC; Hivid®), lamivudine (3TC; Epivir®), stavudine (2′,3′-didehydro-3′-deoxythymidine; D4T; Zerit®), abacavir succinate (1592U89 succinate; Ziagen® ABC), Combivir® (lamivudine & zidovudine; (−)-3TC & AZT), and Trizivir® (abacavir & lamivudine & zidovudine; ABC & (−)-3TC & AZT).

In some embodiments, the methods further comprise administering a second therapeutic agent, wherein the second therapeutic agent is selected from the group consisting of nevirapine (BI-RG-587; Viramune®), delavirdine (BHAP; U-90152; Rescriptor®), and (efavirenz; DMP-266; Sustiva®).

In some embodiments, the methods further comprise administering a second therapeutic agent, wherein the second therapeutic agent is T-20 (Fuzeon®; Enfuvirtide; DP-178; Pentafuside; GP41 127-162 AA).

In some embodiments, the methods further comprise administering a second therapeutic agent, wherein the second therapeutic agent is TMCC114, or TMCC114 in combination with a reverse transcriptase inhibitor. In some embodiments, the methods further comprise administering a second therapeutic agent, wherein the second therapeutic agent is Lipinavir, or Lupanivir in combination with a reverse transcriptase inhibitor.

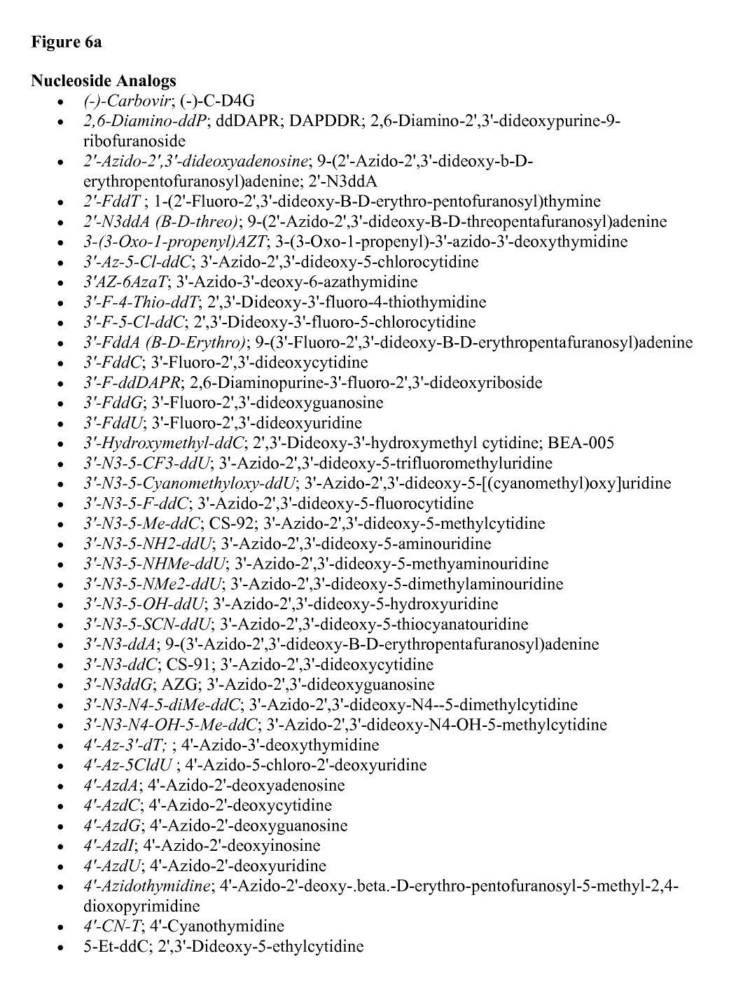

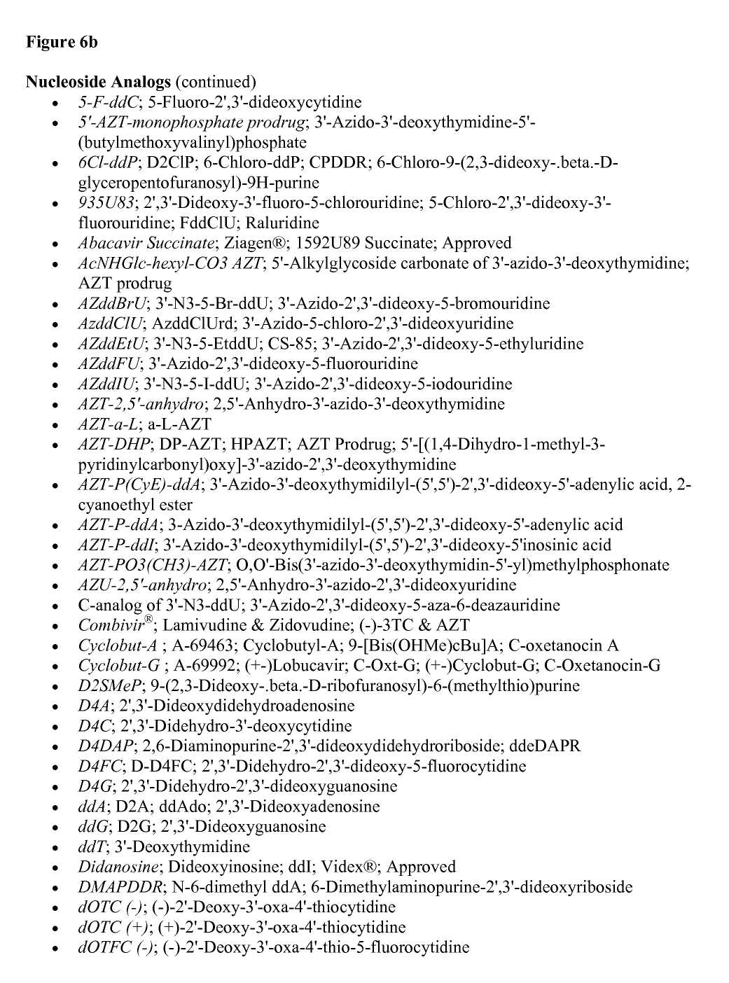

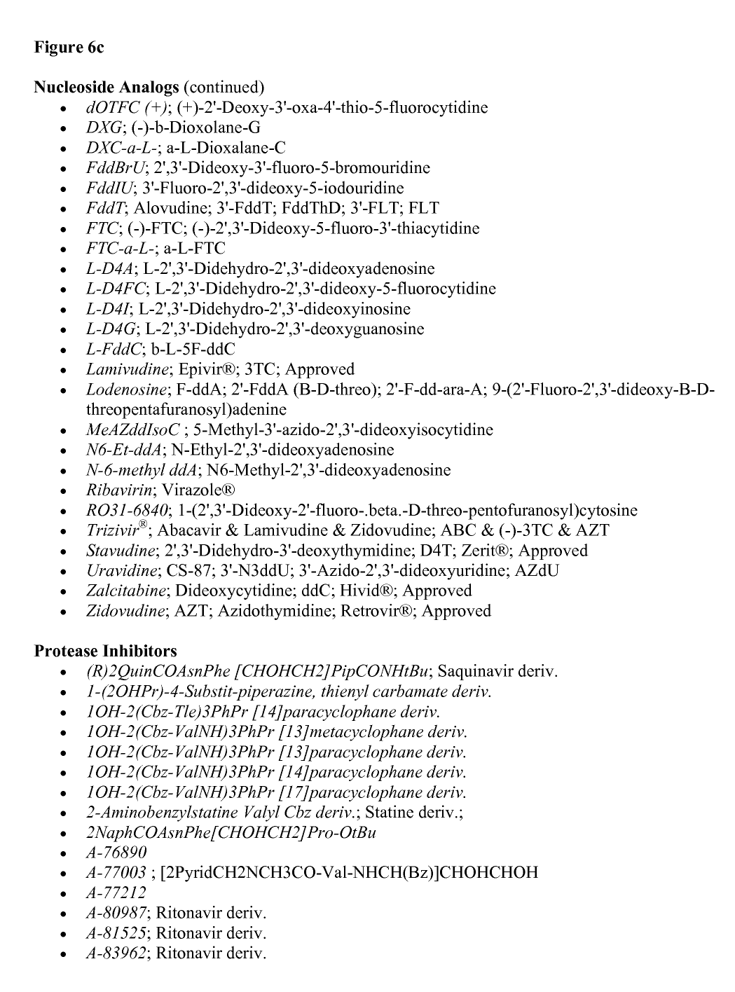

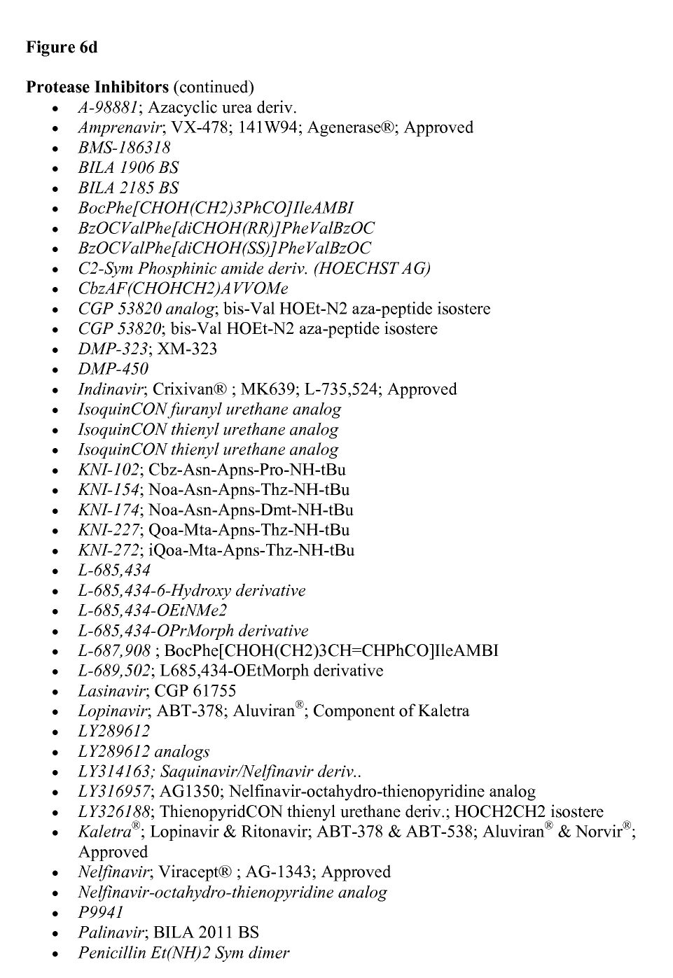

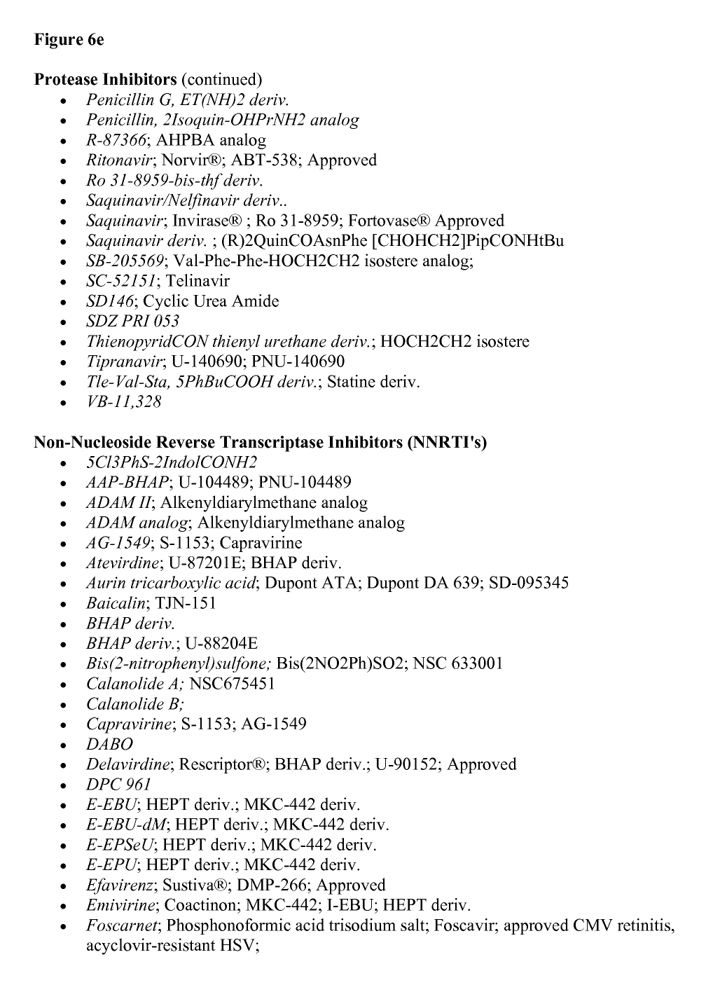

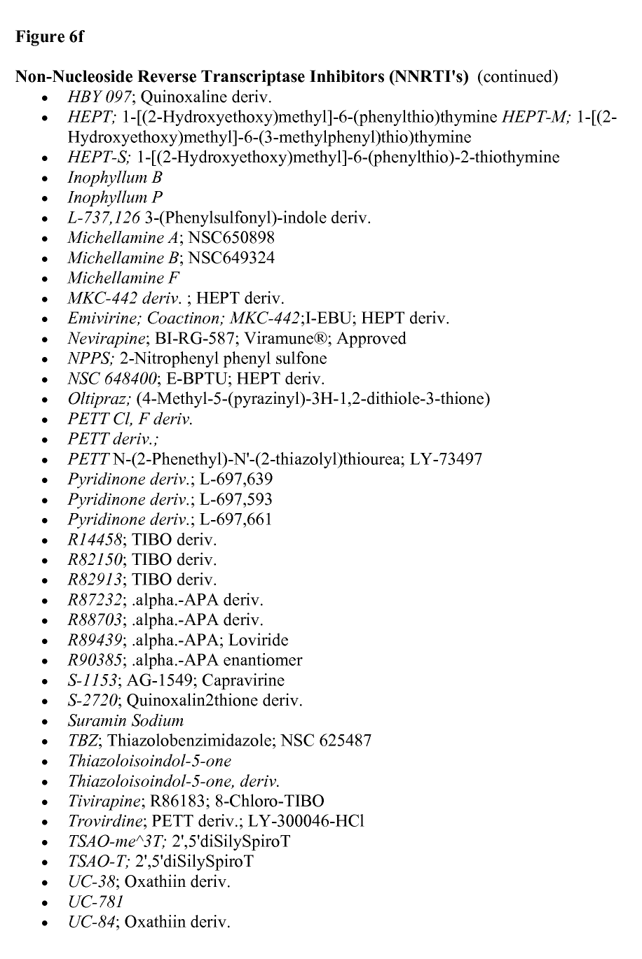

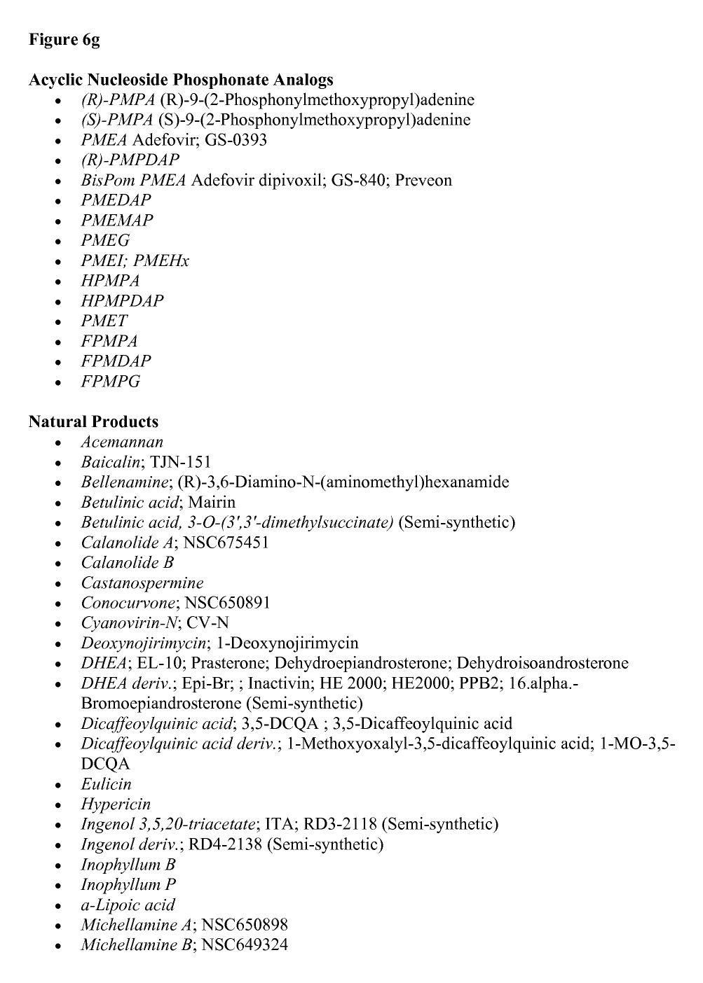

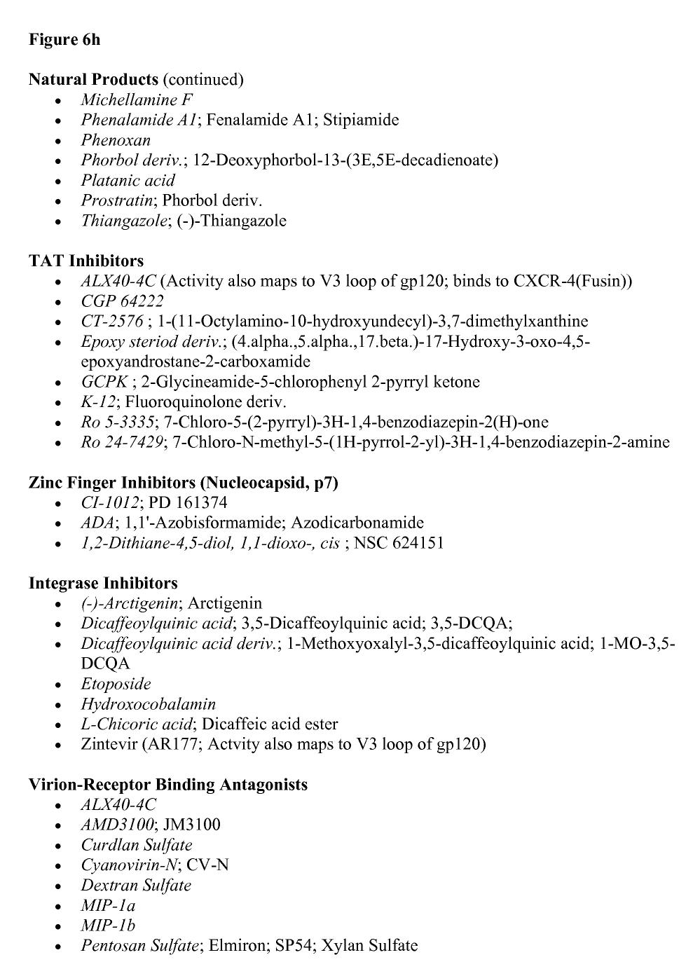

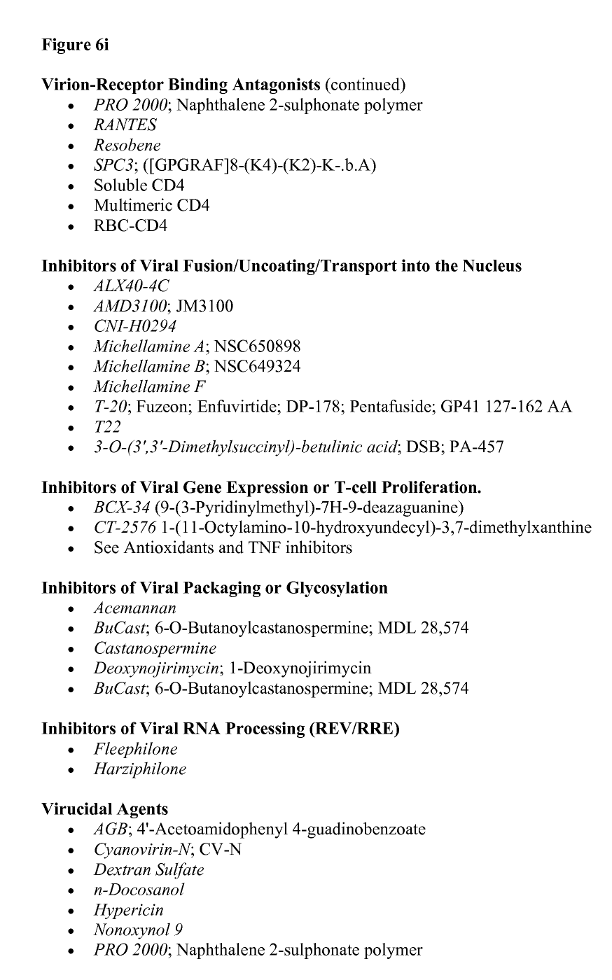

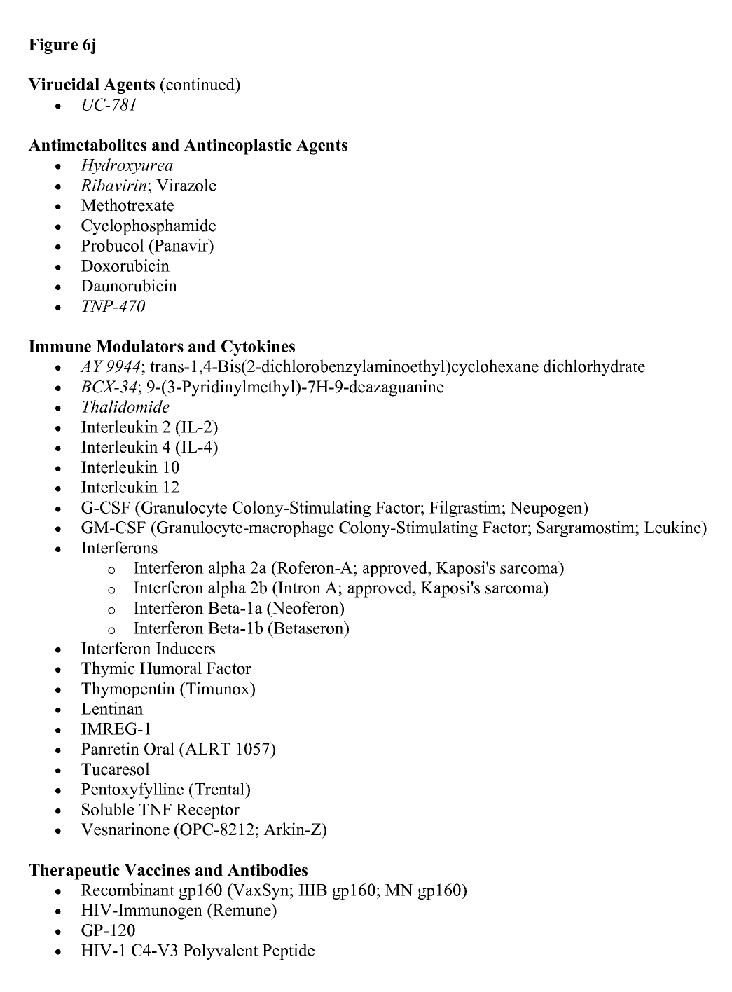

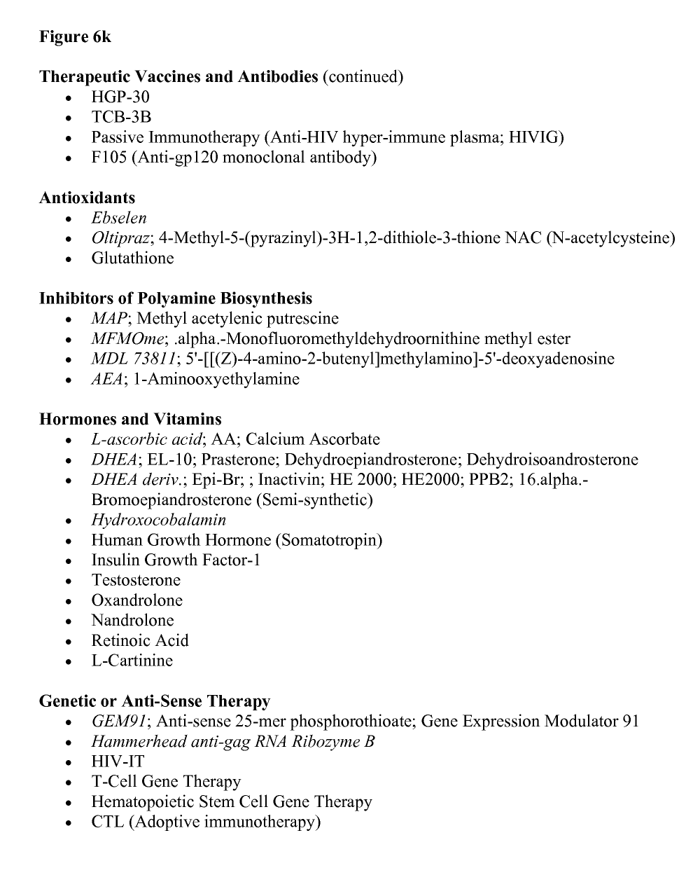

Combination therapy in different formulations may be administered simultaneously, separately or sequentially. Alternatively, such combination may be administered as a single formulation, whereby the active ingredients are released from the formulation simultaneously or separately. Compositions comprising at least two inhibitors described herein and/or one or more other protease inhibitors and/or other therapeutic agents are also provided herein. In certain embodiments the compounds of the invention can be combined with one or more of any anti-HIV compounds (e.g. those listed in FIGS. 6a-k). Additional compounds which may be combined with one or more of the inventive compounds, and further discussion of combination therapy can be found in Yeni, P. G. et al. JAMA 2004, 292(2), 251-265; Pozniak, A. et al. Business Briefing Clinical Virology & Infectious Disease 2004, 1-7; and Chittick, G. E. et al. Antimicrobial Agents and Chemotherapy 2006, 1304-1310; all of which are hereby incorporated by reference.

Definitions. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The term “HIV” is known to one skilled in the art to refer to Human Immunodeficiency Virus. There are two types of HIV: HIV-1 and HIV-2. There are many different strains of HIV-1. The strains of HIV-1 can be classified into three groups: the “major” group M, the “outlier” group 0 and the “new” group N. These three groups may represent three separate introductions of simian immunodeficiency virus into humans. Within the M-group there are at least ten subtypes or clades: e.g., Glade A, B, C, D, E, F, G, H, I, J, and K. A “Glade” is a group of organisms, such as a species, whose members share homologous features derived from a common ancestor. Any reference to HIV in this application includes all of these tupes and strains.

As known to one skilled in the art, “retroviruses” are diploid positive-strand RNA viruses that replicate through an integrated DNA intermediate (proviral DNA). In particular, upon infection by the RNA virus, the lentiviral genome is reverse-transcribed into DNA by a virally encoded reverse transcriptase that is carried as a protein in each retrovirus. The viral DNA is then integrated pseudo-randomly into the host cell genome of the infecting cell, forming a “provirus” which is inherited by daughter cells. The retrovirus genome contains at least three genes: gag codes for core and structural proteins of the virus; pol codes for reverse transcriptase, protease and integrase; and env codes for the virus surface proteins. Within the retrovirus family, HIV is classified as a lentivirus, having genetic and morphologic similarities to animal lentiviruses such as those infecting cats (feline immunodeficiency virus), sheep (visna virus), goats (caprine arthritis-encephalitis virus), and non-human primates (simian immunodeficiency virus).

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroa toms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on page 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: —N(R51)(R50) or [—N(R50)(R52)(R53)]⁺, wherein R50, R51, R52 and R53 each independently represent a hydrogen, an alkyl, an alkenyl, —(CH₂)ₘ—R61, or R50 and R51 or R52, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: —N(R50)-C(═O)R54, wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: —C(═O)N(R50)(R51), wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas: —C(═O)—X50-R55 or —X50-C(═O)—R56, wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula: —C(R75)(═NOR), wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: —S(═O)₂OR57, in which R57 is an electron pair, hydrogen, alkyl, cycloalkyl, or aryl.

The term “sulfate” is art recognized and includes a moiety that may be represented by the general formula: —OS(═O)₂OR57, in which R57 is as defined above.

The term “sulfonamido” is art recognized and includes a moiety that may be represented by the general formula: —N(R50)-S(═O)₂OR56, in which R50 and R56 are as defined above.

The term “sulfamoyl” is art-recognized and refers to a moiety that may be represented by the general formula: —S(═O)₂N(R50)(R51), in which R50 and R51 are as defined above.

The term “sulfonyl” is art-recognized and refers to a moiety that may be represented by the general formula: —S(═O)₂R58, in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: —S(═O)R58, in which R58 is defined above.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g., alkyl, m, n, and the like, when it occurs more than once in any structure, is intended to be independent of its definition elsewhere in the same structure.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, “Handbook of Chemistry and Physics”, 67th Ed., 1986-87, inside cover.

### EXEMPLIFICATION

The invention now being generally described, it will be more readily understood by reference to the following examples which are included merely for purposes of illustration of certain aspects and embodiments of the present invention, and are not intended to limit the invention

General Experimental Procedures. ¹H and ¹³C NMR spectra were recorded on a Varian 400 MHz NMR spectrometer, operating at 400 MHz for ¹H and 100 MHz for ¹³C. Chemical shifts are reported in ppm relative to the solvent signal, and coupling constant (J) values are reported in Hertz (Hz). Thin-layer chromatography (TLC) was performed on E. Merck silica gel 60-F-254 plates and spots were visualized with UV light. Flash column chromatography was performed using 230-400 mesh silica gels (E-Merck). High resolution mass spectra (HRMS) were recorded on Waters Q-TOF Premier mass spectrometer by direct infusion of solutions of each compound using electrospray ionization (ESI) in positive mode. Low resolution mass spectra were obtained using Waters Alliance HT/Micromass ZQ system (ESI). Tetrahydrofuran was distilled from sodium/benzophenone Anhydrous Dichloromethane, N,N-dimethylformamide, benzene, and toluene were purchased from Aldrich and used as such. All reagents and chemicals were purchased from commercial vendors and used as received.

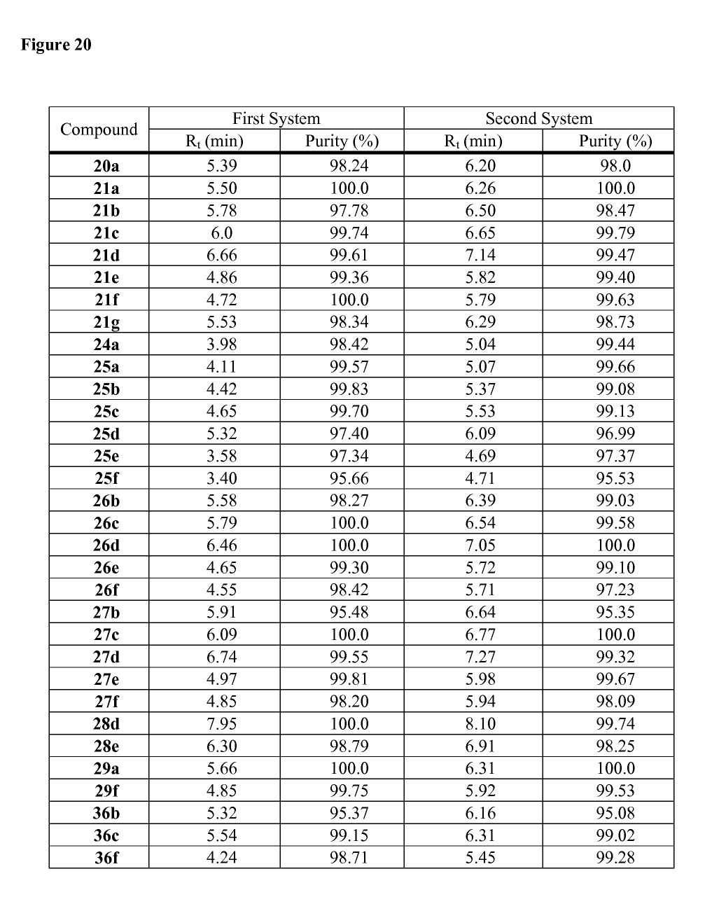

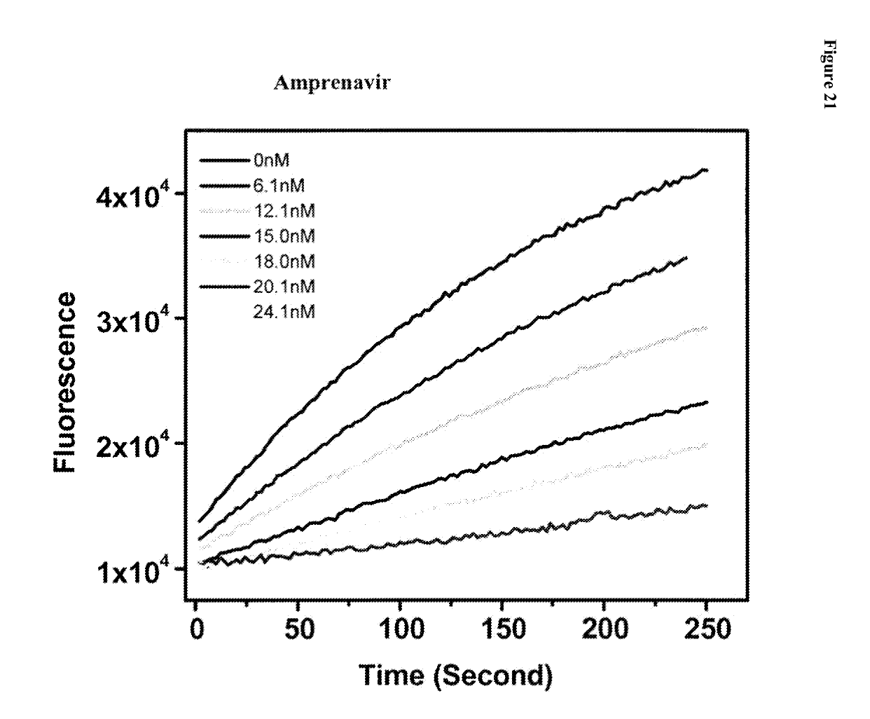

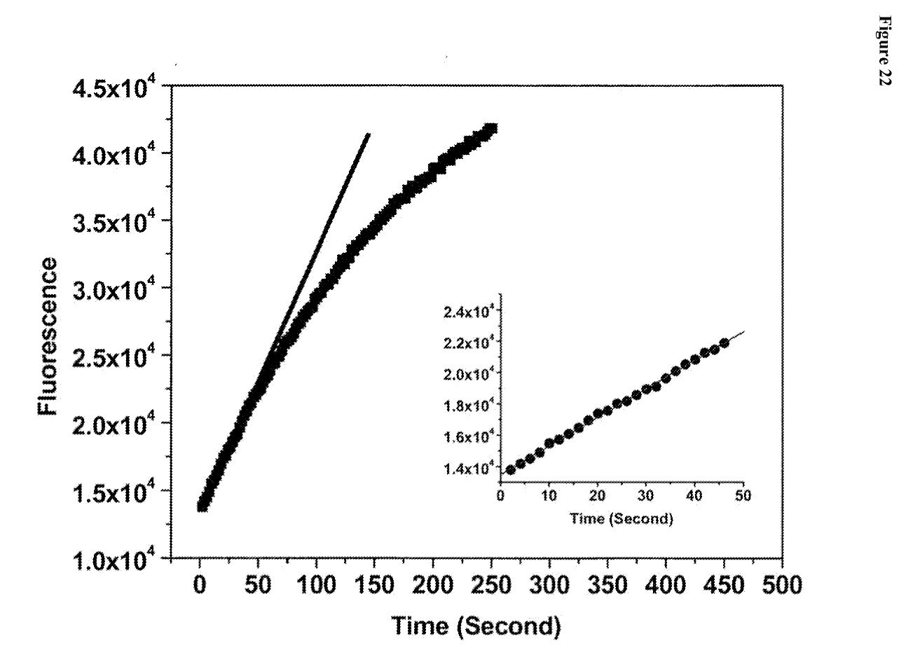

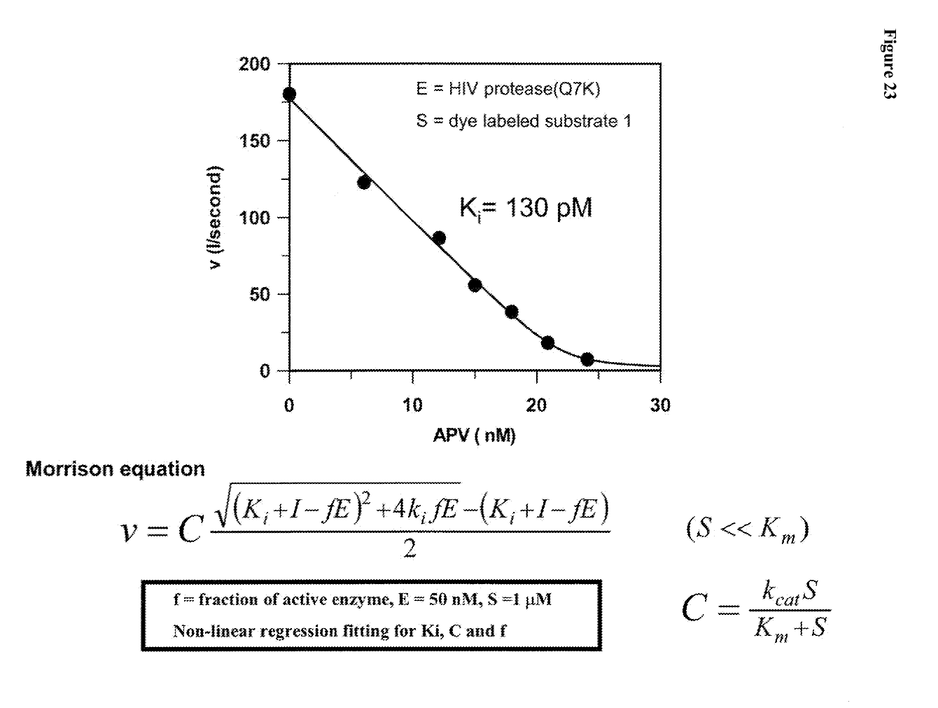

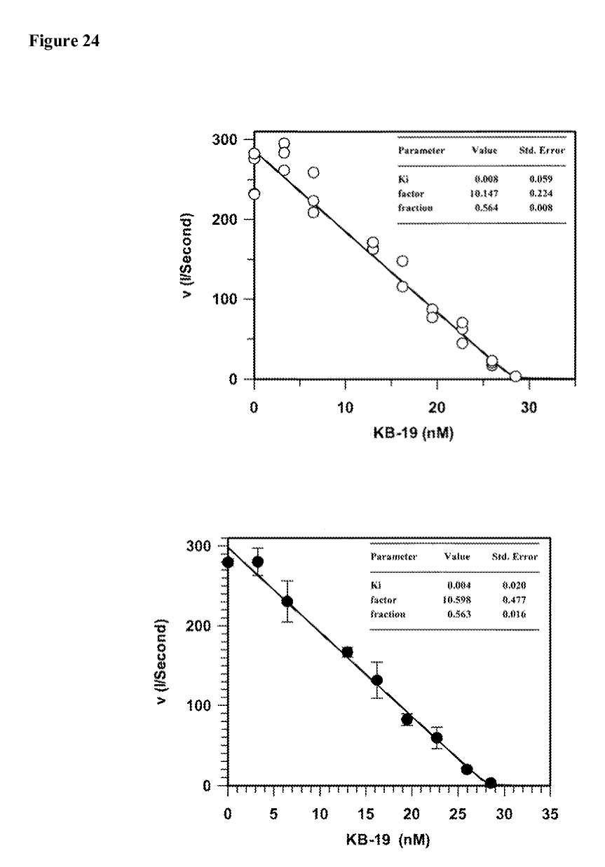

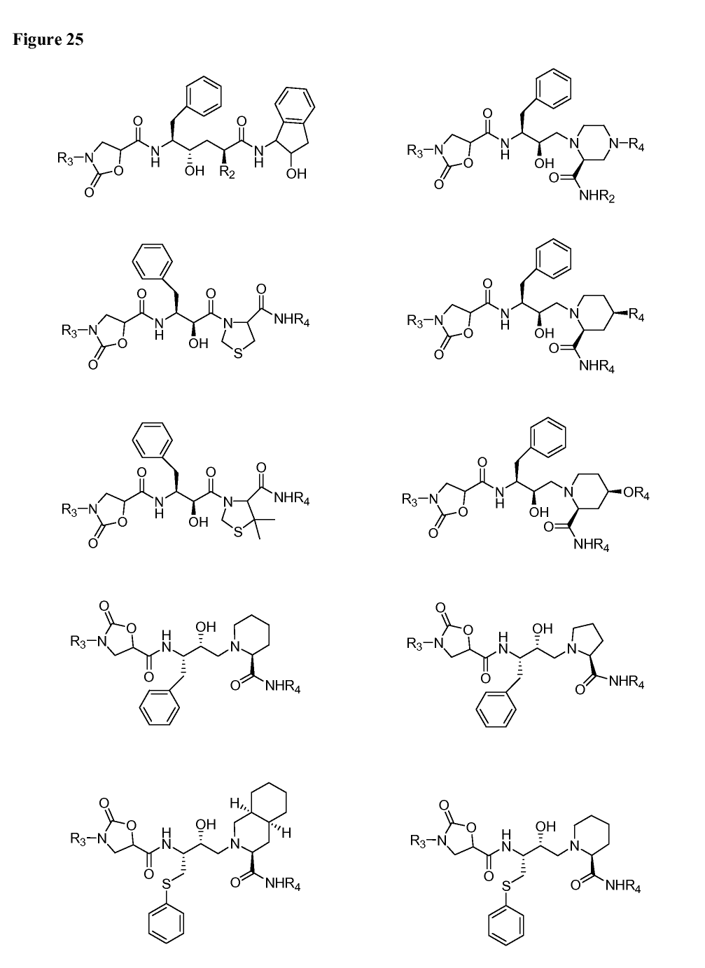

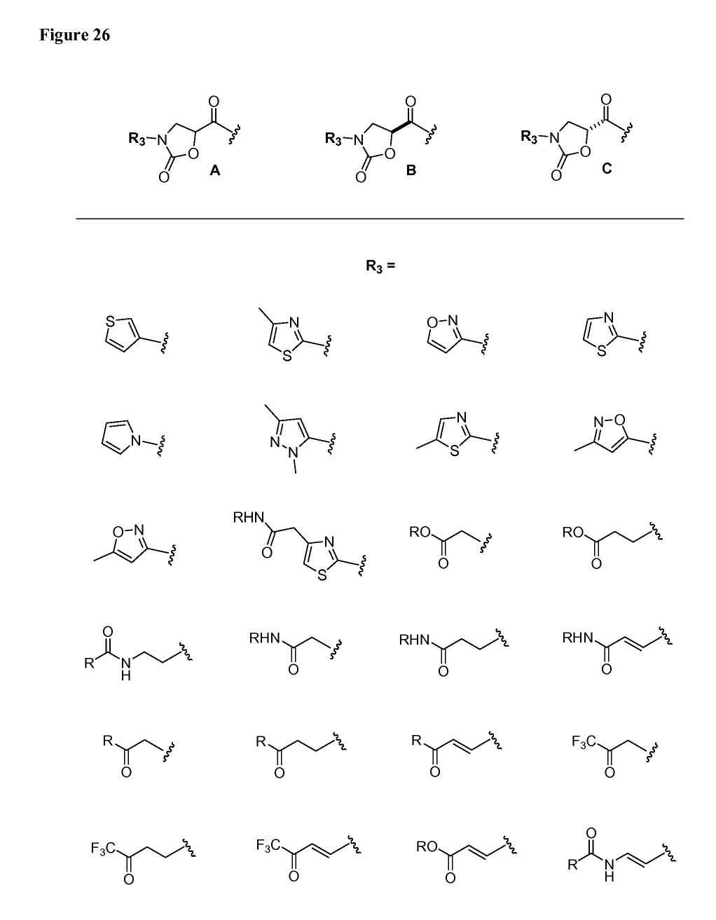

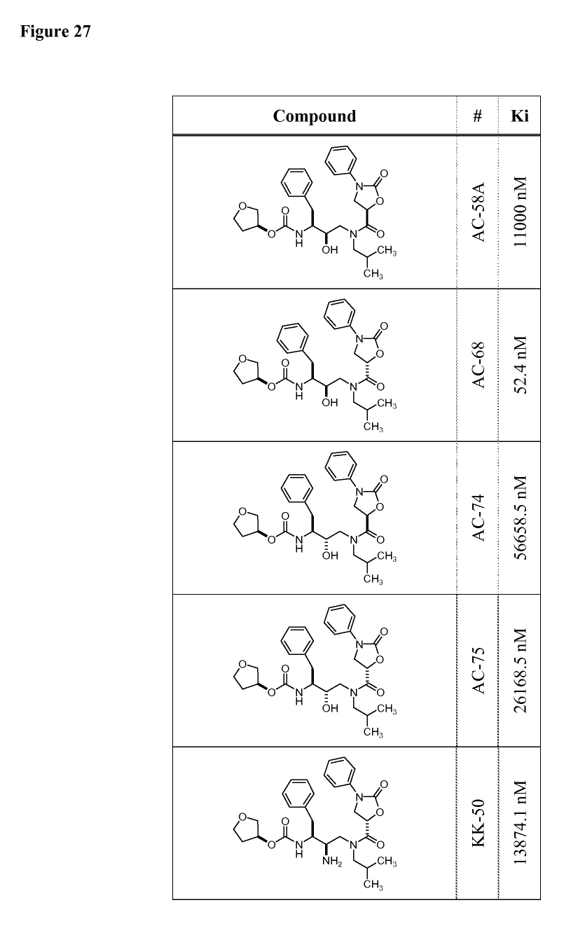

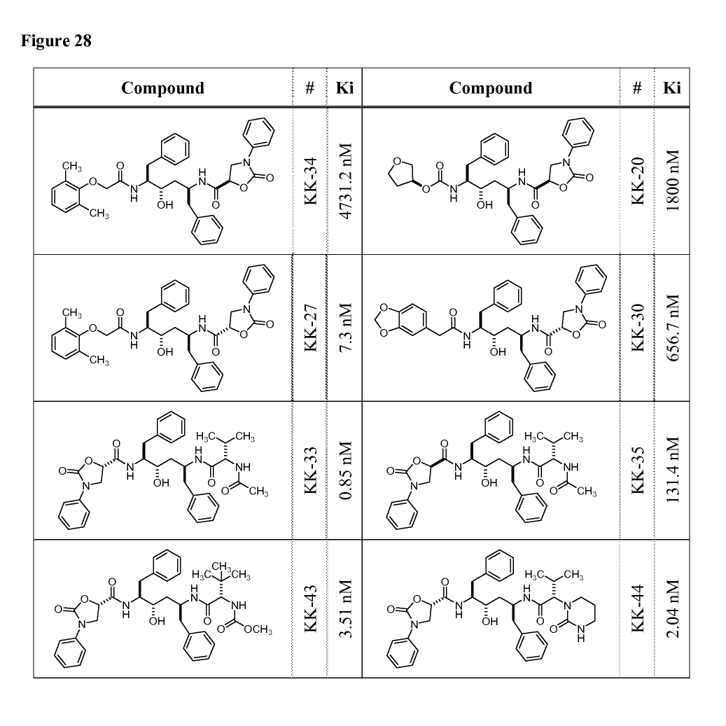

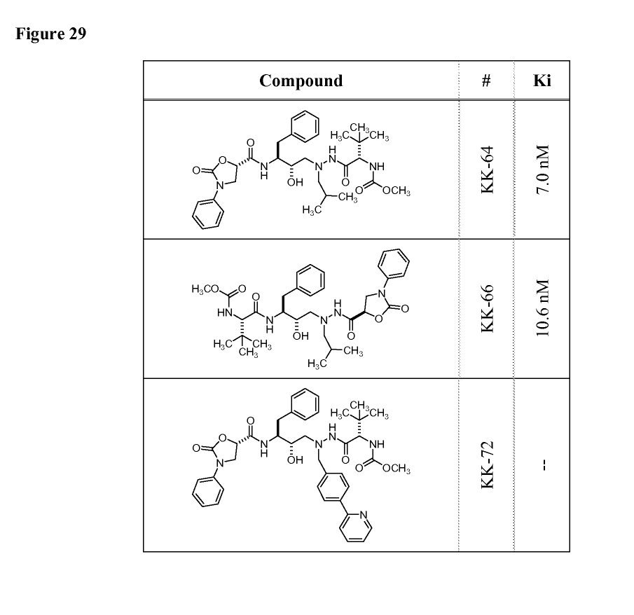

Analytical reversed-phase high performance liquid chromatography (HPLC) was performed on a Waters Separation Module 2695 system equipped with an auto sampler and a Waters 996 photodiode array detector. Purity of the final compounds was determined using two different chromatographic systems. First system: column, Waters Nova-Pak RP-C18 (4 μm, 3.9 mm×150 mm); mobile phase A, 10 mM ammonium acetate in water; mobile phase B, acetonitrile. Using a flow rate of 0.8 mL/min, gradient elution was performed from 50% B to 100% B over 10 min. Second system: column, Agilent Zorbax 300SB-C8 (5 □m, 4.6 mm×250 mm); mobile phase A, 0.1% trifluoroacetic acid in water; mobile phase B, 0.1% trifluoroacetic acid in acetonitrile. Gradient elution was performed from 50% B to 100% B over 10 min at a flow rate of 1 mL/min. A table containing the retention time and purity of selecyed compound is shown in FIG. 20.

### Example 1

### General Procedure for the CBZ Protection of Substituted Anilines

Solid NaHCO₃ (32.65 g, 388.5 mmols) was added to an ice-cooled solution of aniline derivative (185 mmols) in acetone-water mixture (2:1) (300 mL) followed by the slow addition of benzyl chloroformate (27 mL, 190 mmols). The resulting slurry was warmed to ambient temperature and stirred overnight. Reaction mixture was poured onto ice and the resulting precipitate was filtered, washed with water and dried. Product was purified by recrystallization from a mixture of hexanes and ethylacetate to provide the pure product as crystalline solid. Compounds 4a-g were prepared following this general procedure.

### Example 2

### General Procedure for the Synthesis of 5-(Hydroxymethyl)-Oxazolidinones 7 and 8

A solution of CBZ protected aniline derivative 4 (34.7 mmols) in dry THF (150 mL) was cooled to −78° C. under dry N₂ atmosphere. A solution of n-BuLi (1.6 M in hexanes; 25 mL, 40 mmols) was slowly added keeping the temperature below −70° C. After stirring the reaction mixture at −78° C. for 45 minutes, a solution of chiral glycidyl butyrate (5 g, 34.7 mmols) in dry THF was slowly added. The resulting mixture was stirred at −78° C. for 2 hours and then slowly warmed to room temperature and stirred overnight. Reaction was quenched by the addition of saturated aqueous NH₄Cl solution. Ethyl acetate and water were added and layers separated. The aqueous layer was further extracted with ethyl acetate (3 times). Combined organic extract was washed with saturated aqueous NaCl solution, dried (Na₂SO₄), filtered and evaporated to yield a pale yellow solid. This solid was triturated with a mixture of chloroform and hexanes and filtered to provide pure alcohol as off white solid. Compounds 8a-g were prepared following this general procedure.

(R)—S-(Hydroxymethyl)-3-phenyloxazolidine-2-one (7a). ¹H NMR (400 MHz, CD₃OD) δ 7.55 (m, 2H), 7.35 (m, 2H), 7.12 (m, 1H), 4.71 (m, 1H), 4.10 (t, J=9.2 Hz, 1H), 3.91 (dd, J=8.8, 6.8 Hz, 1H), 3.82 (dd, J=12.8, 3.6 Hz, 1H), 3.67 (dd, J=12.4, 4.0 Hz, 1H); ¹³C NMR (100 MHz, CD₃OD) [symbol] 139.76, 129.97, 125.14, 119.72, 95.67, 75.05, 63.29, 47.78.

### Example 3

### General Procedure for the Synthesis of Phenylloxazolidinone-5-Carboxylic Acids 9 and 10

To an ice-cooled solution of NaIO₄ (35 mmols) in water (75 mL) was added a solution of the alcohol 7 or 8 (10 mmols) in a mixture of CH₃CN and CCl₄ (1:1) (100 mL). Solid RuCl₃.H₂O (0.5 mmol) was added and the reaction mixture was stirred at 0° C. for 30 minutes, warmed to room temperature and stirred for 4-6 hours. Reaction was quenched by adding CH₂Cl₂ and layers were separated. The aqueous layer was further extracted with CH₂Cl₂, combined organic extract was dried (Na₂SO₄) and evaporated to provide a gummy solid. Crude product was purified by column chromatography on silica gel using a mixture of 25% CH₃CN in CH₂Cl₂+1% HCO₂H as eluent. This method provided the desired phenyloxazolidine-S-carboxylic acids as solids in excellent yields. The following compounds were prepared by this general procedure:

(R)-2-Oxo-3-phenyloxazolidine-S-carboxylic acid (9a). ¹H NMR (400 MHz, CDCl₃) δ 7.48 (m, 2H), 7.36 (m, 2H), 7.15 (m, 1H), 5.89 (br. s, 1H), 5.09 (dd, J=10.0, 5.2 Hz, 1H), 4.33 (t, J=9.6 Hz, 1H), 4.16 (dd, J=8.8, 4.8 Hz, 1H).

(S)-2-Oxo-3-phenyloxazolidine-S-carboxilic acid (10a). ¹H NMR (400 MHz, CDCl₃) δ 7.50 (m, 2H), 7.36 (m, 2H), 7.16 (m, 1H), 5.80 (br. s, 1H), 5.07 (dd, J=10.0, 5.2 Hz, 1H), 4.32 (t, J=9.6 Hz, 1H), 4.15 (dd, J=9.2, 4.4 Hz, 1H).

(S)-3-(3-Fluorophenyl)-2-oxo-oxazolidine-S-carboxylic acid (10b). ¹H NMR (400 MHz, CD₃OD) δ 7.51 (m, 1H), 7.37 (m, 1H), 7.27 (dd, J=8.4, 2.4 Hz, 1H), 6.88 (dt, J=8.0, 2.4 Hz, 1H), 5.16 (dd, J=10.0, 5.6 Hz, 1H), 4.38 (t, J=9.6 Hz, 1H), 4.14 (dd, J=9.2, 5.2 Hz, 1H).

(S)-3-(3,4-Difluorophenyl)-2-oxo-oxazolidine-S-carboxylic acid (10c). ¹H NMR (400 MHz, CD₃OD) δ7.72-7.66 (m, 1H), 7.27-7.24 (m, 2H), 5.16 (dd, J=10.0, 5.6 Hz, 1H), 4.37 (t, J=9.6 Hz, 1H), 4.14 (dd, J=9.2, 5.6 Hz, 1H).

(S)-2-Oxo-3-[(3-trifluoromethyl)phenyl]oxazolidine-S-carboxylic acid (10d). ¹H NMR (400 MHz, CDCl₃) δ7.75 (d, J=8.0 Hz, 1H), 7.72 (s, 1H), 7.50 (t, J=8.0 Hz, 1H), 7.41 (d, J=8.0 Hz, 1H), 6.24 (br. s 1H), 5.15 (dd, J=9.6, 5.2 Hz, 1H), 4.38 (t, J=9.6 Hz, 1H), 4.21 (dd, J=9.2, 5.2 Hz, 1H).

(S)-3-(3-Acetylphenyl)-2-oxo-oxazolidine-S-carboxylic acid (10e). ¹H NMR (400 MHz, CD₃OD) δ8.15 (s, 1H), 7.83 (dd, J=8.0, 2.4 Hz, 1H), 7.78 (d, J=8.0 Hz, 1H), 7.52 (t, J=8.4 Hz, 1H), 5.19 (dd, J=9.6, 5.2 Hz, 1H), 4.45 (t, J=9.2 Hz, 1H), 4.20 (dd, J=9.6, 5.6 Hz, 1H), 2.61 (s, 3H).

(S)-3-(4-Acetylphenyl)-2-oxo-oxazolidine-S-carboxylic acid (100. ¹H NMR (400 MHz, DMSO) 87.97 (dd, J=7.6, 1.6 Hz, 2H), 7.69 (dd, J=7.6, 1.6 Hz, 2H), 5.20 (dd, J=10.0, 5.6 Hz, 1H), 4.39 (t, J=9.6 Hz, 1H), 4.16 (dd, J=9.2, 5.2 Hz, 1H), 2.53 (s, 3H).

(S)-3-(3-Methoxyphenyl)-2-oxo-oxazolidine-S-carboxylic acid (10g). ¹H NMR (400 MHz, CDCl₃) δ7.27 (t, J=8.0 Hz, 1H), 7.21 (t, J=2.4 Hz, 1H), 6.98 (dd, J=8.8, 2.8 Hz, 1H), 6.71 (dd, J=8.4, 2.4 Hz, 1H), 5.09 (dd, J=10.0, 5.6 Hz, 1H), 4.33 (t, J=9.6 Hz, 1H), 4.18-4.07 (m, 2H), 3.81 (s, 3H).

### Example 4

### General Procedure for the Ring Opening of Epoxide with Amines

A solution of the chiral epoxide 11 (1-Oxiranyl-2-phenylethyl)carbamic acid tert-butyl ester) (10 mmol) in EtOH (75 mL) was added to isobutyl amine (10 mL; large excess) and the mixture was heated at 80° C. for 3 hours. After cooling to room temperature, solvents were removed under reduced pressure. Product was purified by recrystallization from ethyl acetate-hexanes mixture to provide the product as white solid in excellent yield.

### Example 5

### General Procedure for the Synthesis of (Hydroxyethylamino)-Sulfonamides

To an ice-cooled solution of the secondary amine 12 (5 mmol) in CH₂Cl₂ (20 mL) was added an aqueous solution of Na₂CO₃ (8 mmol in 5 mL H₂O) followed by the slow addition of sulfonyl chloride (5 mmol) solution in CH₂Cl₂ (5 mL). After 15 minutes the reaction mixture was warmed to ambient temperature and stirred till no starting material was detected by tlc. Reaction mixture was diluted with CH₂Cl₂ and layers were separated. Organic extract was washed with saturated aqueous NaCl solution, dried (Na₂SO₄), filtered and evaporated. Product was purified by flash chromatography on silica gel using mixture of ethyl acetate and hexanes as eluent to afford pure product.

The following compounds were prepared by this general procedure: N-[(1S,2R)-1-Benzyl-2-hydroxy-3-[isobutyl[(4-methoxyphenyl)sulfonyl]amino]propyl]carbamic acid tert-butyl ester (14); N-[(1S,2R)-1-Benzyl-2-hydroxy-3-[isobutyl[(4-nitrophenyl)sulfonyl]-amino]propyl]carbamic acid tert-butyl ester (15); N-[(1S,2R)-3-[[(Benzo-[1,3]-dioxole-5-sulfonyl)](isobutyl)amino]-1-benzyl-2-hydroxypropyl]carbamic acid tert-butyl ester (16); N-[(1S,2R)-1-Benzyl-3-[[(3-fluoro-4-methoxyphenyl)sulfonyl](isobutyl)amino]-2-hydroxypropyl]-carbamic acid tert-butyl ester (17); N-[(1S,2R)-1-Benzyl-2-hydroxy-3-[isobutyl[[(4-trifluoro-methoxy)phenyl]sulfonyl]amino]propyl]carbamic acid tert-butyl ester (18); N-[(1S,2R)-1-Benzyl-2-hydroxy-3-[isobutyl[(3-methoxyphenyl)sulfonyl]amino]propyl]carbamic acid tert-butyl ester (19); N-[(1S,2R)-1-Benzyl-3-[(cyclopropylmethyl)[(3-methoxyphenyl)sulfonyl]-amino]-2-hydroxypropyl]carbamic acid tert-butyl ester (32); N-[(1S,2R)-1-Benzyl-2-hydroxy-3-[[(3-methoxyphenyl)sulfonyl](2-thiophenylmethyl)]amino]propyl]carbamic acid tert-butyl ester (33); N-[(1S,2R)-1-Benzyl-2-hydroxy-3-[(2-thiophenylmethyl)[(2,4,5-trifluorophenyl)sulfonyl]-amino]propyl]carbamic acid tert-butyl ester (34); and N-[(1S,2R)-1-Benzyl-2-hydroxy-3-[[(3-methoxyphenyl)sulfonyl][(R)-(tetrahydro-2-furanyl)methyl]amino]propyl]carbamic acid tert-butyl ester (35).

### Example 6

### General Procedure for the Coupling Reaction

Excess oxalyl chloride was added to solid phenyloxazolidinone-S-carboxylic acid (0.5 mmols) and the resulting mixture was stirred at room temperature overnight. Oxalyl chloride was removed by distillation under reduced pressure and residue dried under high vacuum for 30 minutes. A solution of the resulting acid chloride in dry THF (5 mL) was used in the coupling reaction.

To an ice-cooled mixture of the Boc deprotected amine (0.5 mmols) in dry THF (5 mL) was added Et₃N (1.1 mmols) followed by slow addition of the acid chloride solution. After 15 minutes the reaction mixture was warmed to room temperature and stirred until reaction was complete (monitored by tlc). Small amount of water and ethyl acetate were added and layers were separated. The organic extract was washed with saturated aqueous NaCl solution, dried (Na₂SO₄), filtered and evaporated. Flash chromatography on silica gel using mixture of ethyl acetate and hexanes (in some cases, methanol/chloroform mixture) as eluent, provided the target compound as solid.

### Example 7

### General Procedure for the Reduction of the Nitro Group

A mixture of the nitro compound (0.4 mmols) and SnCl₂.2H₂O (0.45 g, 2 mmols) in ethyl acetate (10 mL) was heated at 80° C. for 2-3 hours. Reaction mixture was allowed to cool to ambient temperature and treated with saturated aqueous NaHCO₃ solution (10 m). It was diluted with ethyl acetate and layers were separated, aqueous layer was further extracted with ethyl acetate (2×). Combined organic extract was washed with saturated aqueous NaCl solution, dried (Na₂SO₄) and evaporated to yield a foamy solid. Flash chromatography on silica gel using mixture of methanol in chloroform as eluent, provided the target compound as solid.

### Example 8

### Selected Spectral Data for Designed Protease Inhibitor

General experimental procedures for the synthesis of intermediates, phenyloxazolidines (9-10) and (R)-(hydroxyethylamino)sulfonamides (14-19 and 32-35) and for the coupling reactions to provide the target compounds (20-29 and 36-39) are provided above.

(5R)—N-[(1S,2R)-1-Benzyl-2-hydroxy-3-[isobutyl[(4-methoxyphenyl)sulfonyl]amino]-propyl]-2-oxo-3-phenyloxazolidine-S-carboxamide (20a). ¹H NMR (400 MHz, CDCl₃) δ 7.67 (d, J=8.8 Hz, 2H), 7.46 (d, J=8.4 Hz, 2H), 7.35 (t, J=7.6 Hz, 2H), 7.24-7.13 (m, 6H), 6.92 (d, J=8.8 Hz, 2H), 6.75 (d, J=8.4 Hz, 1H), 4.71 (dd, J=10.0, 6.0 Hz, 1H), 4.17 (m, 2H), 4.07 (dd, J=9.2, 6.0 Hz, 1H), 3.91 (m, 1H), 3.84 (m, 1H), 3.82 (s, 3H), 3.10-2.77 (m, 6H), 1.76 (m, 1H), 0.84 (d, J=6.4 Hz, 3H), 0.79 (d, J=6.8 Hz, 3H); ¹³C NMR (100 MHz, CDCl₃) δ 168.98, 163.23, 153.06, 137.49, 137.46, 129.91, 129.67 (2C), 129.44 (2C), 129.41 (2C), 128.79 (2C), 126.96, 124.95, 118.55 (2C), 114.54 (2C), 72.28, 70.03, 58.90, 55.80, 54.41, 53.56, 48.19, 35.05, 27.46 20.25, 20.11. HRMS (ESI) m/z: C₃₁H₃₈N₃O₇S (M+H)⁺ calcd, 596.2430; found, 596.2421.

(5S)—N-[(1S,2R)-1-Benzyl-2-hydroxy-3-[isobutyl[(4-methoxyphenyl)sulfonyl]amino]propyl]-2-oxo-3-phenyloxazolidine-S-carboxamide (21a). ¹H NMR (400 MHz, CDCl₃) δ7.74 (d, J=8.8 Hz, 2H), 7.45-7.38 (m, 4H), 7.19 (t, J=7.2 Hz, 1H), 7.12 (d, J=7.2 Hz, 2H), 7.04-6.98 (m, 4H), 6.89 (t, J=7.6 Hz, 1H), 6.69 (d, J=9.6 Hz, 1H), 4.77 (dd, J=9.6, 5.6 Hz, 1H), 4.22 (m, 1H), 4.04 (t, J=9.6 Hz, 1H), 3.90 (m, 1H), 3.87 (s, 3H), 3.78 (d, J=2.4 Hz, 1H), 3.36 (dd, J=9.2, 6.0 Hz, 1H), 3.23 (dd, J=15.2, 9.2 Hz, 1H), 3.11 (dd, J=13.6, 4.4 Hz, 1H), 3.02 (dd, J=13.6, 8.8 Hz, 1H), 2.96 (dd, J=15.2, 2.8 Hz, 1H), 2.81 (dd, J=13.6, 6.8 Hz, 1H), 2.73 (dd, J=14.0, 10.8 Hz, 1H), 1.84 (m, 1H), 0.96 (d, J=6.8 Hz, 3H), 0.90 (d, J=6.4 Hz, 3H); ¹³C NMR (100 MHz, CDCl₃) [symbol] 168.86, 163.25, 153.12, 137.52, 137.49, 129.94, 129.68 (2C), 129.50 (2C), 129.26 (2C), 128.50 (2C), 126.61, 124.76, 118.47 (2C), 114.56 (2C), 72.46, 69.87, 58.91, 55.79, 53.78, 53.52, 48.29, 35.64, 27.44, 20.31, 20.07. HRMS (ESI) m/z: C₃₁H₃₈N₃O₇S (M+H)⁺ calcd, 596.2430; found, 596.2438.

(5S)—N-[(1S,2R)-1-Benzyl-2-hydroxy-3-[isobutyl[(4-methoxyphenyl)sulfonyl]amino]-propyl]-3-(3-fluorophenyl)-2-oxo-oxazolidine-S-carboxamide (21b). ¹H NMR (400 MHz, CDCl₃) δ7.74 (d, J=8.8 Hz, 2H), 7.39-7.33 (m, 2H), 7.10 (m, 3H), 7.04-6.98 (m, 4H), 6.93-6.85 (m, 2H), 6.68 (d, J=9.6 Hz, 1H), 4.78 (dd, J=10.2, 6.0 Hz, 1H), 4.23 (m, 1H), 4.01 (t, J=9.6 Hz, 1H), 3.91 (m, 1H), 3.88 (s, 3H), 3.68 (br. s, 1H), 3.32 (dd, J=9.2, 6.0 Hz, 1H), 3.23 (dd, J=14.8, 9.2 Hz, 1H), 3.10 (dd, J=13.6, 4.4 Hz, 1H), 3.03 (dd, J=13.2, 8.8 Hz, 1H), 2.97 (dd, J=15.2, 2.4 Hz, 1H), 2.81 (dd, J=13.6, 6.8 Hz, 1H), 2.73 (dd, J=14.0, 10.4 Hz, 1H), 1.84 (m, 1H), 0.96 (d, J=6.4 Hz, 3H), 0.90 (d, J=6.4 Hz, 3H); ¹³C NMR (100 MHz, CDCl₃) [symbol] 168.60, 163.29, 163.18 (d, J=244.2 Hz), 152.82, 138.98 (d, J=10.6 Hz), 137.48, 130.48 (d, J=9.1 Hz), 129.91, 129.70 (2C), 129.50 (2C), 128.49 (2C), 126.60, 114.58 (2C), 113.52 (d, J=2.3 Hz), 111.49 (d, J=21.2 Hz), 106.06 (d, J=27.3 Hz), 72.47, 69.82, 58.95, 55.82, 53.79, 53.44, 48.20, 35.56, 27.46, 20.31, 20.08. HRMS (ESI) m/z: C₃₁H₃₇FN₃O₇S (M+H)⁺ calcd, 614.2336; found, 614.2357.

(5S)—N-[(1S,2R)-1-Benzyl-2-hydroxy-3-[isobutyl[(4-methoxyphenyl)sulfonyl]amino]-propyl]-3-(3,4-difluorophenyl)-2-oxo-oxazolidinone-S-carboxamide (21c). ¹H NMR (400 MHz, CDCl₃) δ7.74 (m, 2H), 7.54-7.48 (m, 1H), 7.19 (dd, J=18.4, 8.8 Hz, 1H), 7.12 (d, J=6.8 Hz, 2H), 7.05-6.97 (m, 5H), 6.89 (t, J=7.6 Hz, 1H), 6.75 (d, J=10.0 Hz, 1H), 4.78 (dd, J=9.6, 5.6 Hz, 1H), 4.25 (m, 1H), 3.99 (t, J=9.2 Hz, 1H), 3.92 (m, 1H), 3.87 (s, 3H), 3.69 (d, J=3.2 Hz, 1H), 3.31 (dd, J=9.2, 6.0 Hz, 1H), 3.22 (dd, J=15.2, 9.6 Hz, 1H), 3.12-2.97 (m, 3H), 2.82 (dd, J=13.2, 6.8 Hz, 1H), 2.74 (dd, J=14.0, 11.2 Hz, 1H), 1.84 (m, 1H), 0.95 (d, J=6.4 Hz, 3H), 0.90 (d, J=6.8 Hz, 3H); ¹³C NMR (100 MHz, CDCl₃) [symbol] 168.45, 163.35, 152.78, 150.49 (dd, J=246.4, 13.6 Hz), 147.41 (dd, J=245.0, 12.1 Hz), 137.46, 134.03 (m), 129.87, 129.72 (2C), 129.55 (2C), 128.53 (2C), 126.61, 117.62 (d, J=18.2 Hz), 114.62 (2C), 113.85 (q, J=3.0 Hz), 108.37 (dd, J=22.8 Hz), 72.47, 69.70, 59.05, 55.84, 53.86, 53.39, 48.25, 35.51, 27.54, 20.34, 20.08. HRMS (ESI) m/z: C₃₁H₃₆F₂N₃O₇S (M+H)⁺ calcd, 632.2242; found, 632.2251.

(5S)—N-[(1S,2R)-1-Benzyl-2-hydroxy-3-[isobutyl[(4-methoxyphenyl)sulfonyl]amino]-propyl]-2-oxo-3-[(3-trifluoromethyl)phenyl]oxazolidine-S-carboxamide (21d). ¹H NMR (400 MHz, CDCl₃) [symbol] 7.74 (dd, J=10.0, 2.4 Hz, 2H), 7.70 (dd, J=8.8, 1.6 Hz, 1H), 7.64 (s, 1H), 7.54 (t, J=8.0 Hz, 1H), 7.46 (d, J=8.0 Hz, 1H), 7.12 (dd, J=8.4, 1.6 Hz, 2H), 7.01-6.96 (m, 4H), 6.83 (t, J=7.2 Hz, 1H), 6.79 (d, J=10.0 Hz, 1H), 4.81 (dd, J=10.0, 6.0 Hz, 1H), 4.27 (m, 1H), 4.04 (t, J=9.6 Hz, 1H), 3.94 (m, 1H), 3.87 (s, 3H), 3.69 (br. s, 1H), 3.33 (dd, J=9.2, 5.6 Hz, 1H), 3.22 (dd, J=15.6, 9.6 Hz, 1H), 3.10 (dd, J=14.0, 4.4 Hz, 1H), 3.04-2.98 (m, 2H), 2.83 (dd, J=13.2, 6.8 Hz, 1H), 2.74 (dd, J=14.0, 10.8 Hz, 1H), 1.85 (m, 1H), 0.95 (d, J=6.4 Hz, 3H), 0.90 (d, J=6.8 Hz, 3H); ¹³C NMR (100 MHz, CDCl₃) [symbol] 168.49, 163.34, 152.83, 138.19, 137.43, 131.76 (d, J=32.6 Hz), 129.93, 129.87, 129.72 (2C), 129.53 (2C), 128.52 (2C), 126.58, 121.40, 121.24 (q, J=3.8 Hz), 115.02 (q, J=3.8 Hz), 114.61 (2C), 72.50, 69.86, 59.03, 55.83, 53.84, 53.36, 48.10, 35.56, 27.52, 20.34, 20.08. HRMS (ESI) m/z: C₃₂H₃₇F₃N₃O₇S (M+H)⁺ calcd, 664.2304; found, 664.2316.

(5S)-3-(3-Acetylphenyl)-N-[(1S,2R)-1-benzyl-2-hydroxy-3-[isobutyl[(4-methoxyphenyl)-sulfonyl]amino]propyl]-2-oxo-oxazolidine-S-carboxamide (21e). ¹H NMR (400 MHz, CDCl₃) δ 7.89 (t, J=1.6 Hz, 1H), 7.80-7.74 (m, 2H), 7.71 (d, J=9.2 Hz, 2H), 7.50 (t, J=8.0 Hz, 1H), 7.10 (d, J=7.2 Hz, 2H), 6.98 (m, 4H), 6.83 (m, 2H), 4.78 (dd, J=10.0, 6.0 Hz, 1H), 4.23 (m, 1H), 4.05 (t, J=9.6 Hz, 1H), 3.93 (m, 1H), 3.84 (s, 3H), 3.46 (s, 1H), 3.36 (dd, J=9.2, 5.6 Hz, 1H), 3.19 (dd, J=15.2, 9.6 Hz, 1H), 3.09 (dd, J=14.0, 4.4 Hz, 1H), 3.01-2.95 (m, 2H), 2.82 (dd, J=13.2, 6.8 Hz, 1H), 2.73 (dd, J=14.0, 10.8 Hz, 1H), 2.63 (s, 3H), 1.84 (m, 1H), 0.92 (d, J=6.4 Hz, 3H), 0.88 (d, J=6.4 Hz, 3H); ¹³C NMR (100 MHz, CDCl₃) [symbol] 197.59, 168.59, 163.31, 153.07, 138.10, 138.04, 137.52, 129.91, 129.71 (2C), 129.65, 129.55 (2C), 128.53 (2C), 126.62, 124.71, 123.01, 117.58, 114.60 (2C), 72.54, 69.93, 59.01, 55.82, 53.85, 53.44, 48.23, 35.63, 27.50, 26.90, 20.33, 20.08. HRMS (ESI) m/z: C₃₃H₄₀N₃O₈S (M+H)¹ calcd, 638.2536; found, 638.2544.

(5S)-3-(4-Acetylphenyl)-N-[(1S,2R)-1-benzyl-2-hydroxy-3-[isobutyl[(4-methoxyphenyl)-sulfonyl]amino]propyl]-2-oxo-oxazolidine-S-carboxamide (21f). ¹H NMR (400 MHz, CDCl₃) δ 7.99 (d, J=9.2 Hz, 2H), 7.72 (d, J=8.8 Hz, 2H), 7.53 (d, J=9.2 Hz, 2H), 7.10 (d, J=7.2 Hz, 2H), 6.98-6.93 (m, 4H), 6.86 (d, J=10.0 Hz, 1H), 6.80 (t, J=7.6 Hz, 1H), 4.80 (dd, J=10.0, 6.0 Hz, 1H), 4.24 (m, 1H), 4.05 (t, J=9.6 Hz, 1H), 3.95 (m, 1H), 3.85 (s, 3H), 3.33 (dd, J=9.6, 6.0 Hz, 1H), 3.19 (dd, J=14.8, 8.8 Hz, 1H), 3.10-2.94 (m, 3H), 2.83 (dd, J=13.2, 6.4 Hz, 1H), 2.73 (dd, J=14.0, 11.2 Hz, 1H), 2.60 (s, 3H), 1.84 (m, 1H), 0.92 (d, J=6.4 Hz, 3H), 0.88 (d, J=6.8 Hz, 3H); ¹³C NMR (100 MHz, CDCl₃) δ196.99, 168.46, 163.29, 152.75, 141.55, 137.51, 133.13, 129.86, 129.78 (2C), 129.68 (2C), 129.52 (2C), 128.44 (2C), 126.58, 117.57 (2C), 114.58 (2C), 72.47, 69.85, 58.95, 55.81, 53.78, 53.43, 48.0, 35.49, 27.46, 26.65, 20.31, 20.07. HRMS (ESI) m/z: C₃₃H₄₀N₃O₈S (M+H)⁺ calcd, 638.2536; found, 638.2545.

(5S)—N-[(1S,2R)-1-Benzyl-2-hydroxy-3-[isobutyl[(4-methoxyphenyl)sulfonyl]-amino]propyl]-3-(3-methoxyphenyl)-2-oxo-oxazolidine-S-carboxamide (21g). ¹H NMR (400 MHz, CDCl₃) δ7.73 (m, 2H), 7.29 (t, J=8.8 Hz, 1H), 7.14 (d, J=2 Hz, 1H), 7.11 (m, 2H), 7.03 (t, J=7.2 Hz, 2H), 6.98 (m, 2H), 6.92-6.88 (m, 2H), 6.73 (ddd, J=8.4, 2.8, 0.8 Hz, 1H), 6.70 (d, J=9.6 Hz, 1H), 4.75 (dd, J=10.4, 6.4 Hz, 1H), 4.21 (m, 1H), 4.01 (t, J=9.6 Hz, 1H), 3.89 (m, 1H), 3.87 (s, 3H), 3.84 (s, 3H), 3.68 (br. s, 1H), 3.32 (dd, J=9.6, 6.4 Hz, 1H), 3.21 (dd, J=15.2, 9.2 Hz, 1H), 3.10 (dd, J=14.0, 4.8 Hz, 1H), 3.01 (dd, J=13.2, 8.8 Hz, 1H), 2.96 (dd, J=15.2, 2.4 Hz, 1H), 2.80 (dd, J=13.6, 6.4 Hz, 1H), 2.73 (dd, J=13.6, 10.4 Hz, 1H), 1.83 (m, 1H), 0.95 (d, J=6.4 Hz, 3H), 0.89 (d, J=6.8 Hz, 3H); ¹³C NMR (100 MHz, CDCl₃) [symbol] 168.86, 163.37, 160.45, 152.94, 138.72, 137.39, 130.08, 129.87, 129.76 (2C), 129.55 (2C), 128.65 (2C), 126.79, 114.66 (2C), 110.61, 110.33, 104.73, 72.49, 69.77, 59.12, 55.89, 55.65, 53.97, 53.52, 48.41, 35.76, 27.60, 20.40, 20.11. HRMS (ESI) m/z: C₃₂H₄₀N₃O₈S (M+H)⁺ calcd, 626.2536; found, 626.2546.

(5R)—N-[(1S,2R)-3-[[(4-Aminophenyl)sulfonyl](isobutyl)amino]-1-benzyl-2-hydroxypropyl]-2-oxo-3-phenyloxazolidine-S-carboxamide (24a). ¹H NMR (400 MHz, CDCl₃) δ7.52-7.47 (m, 4H), 7.38 (t, J=8.4 Hz, 2H), 7.24-7.15 (m, 7H), 6.86 (d, J=8.8 Hz, 1H), 6.61 (d, J=8.8 Hz, 2H), 4.72 (dd, J=9.6, 6.4 Hz, 1H), 4.23-4.11 (m, 2H), 4.05 (dd, J=9.2, 6.0 Hz, 1H), 3.95 (m, 1H), 3.87 (br. s, 1H), 3.14-2.96 (m, 3H), 2.89-2.80 (m, 3H), 1.78 (m, 1H), 0.86 (d, J=6.8 Hz, 3H), 0.82 (d, J=6.4 Hz, 3H); ¹³C NMR (100 MHz, CDCl₃) δ 168.79, 153.29, 150.81, 137.68, 137.56, 129.61 (2C), 129.43 (2C), 129.34 (2C), 128.65 (2C), 126.79, 126.20, 124.83, 118.61 (2C), 114.32 (2C), 72.29, 70.12, 58.80, 54.30, 53.53, 48.18, 35.19, 27.33, 20.23, 20.12. HRMS (ESI) m/z: C₃₀H₃₇N₄O₆S (M+H)⁺ calcd, 581.2434; found, 581.2430.

(5S)—N-[(1S,2R)-3-[[(4-Aminophenyl)sulfonyl](isobutyl)amino]-1-benzyl-2-hydroxypropyl]-2-oxo-3-phenyloxazolidine-S-carboxamide (25a). ¹H NMR (400 MHz, CDCl₃) δ 7.56 (d, J=8.4 Hz, 2H), 7.43-7.37 (m, 5H), 7.18 (t, J=6.8 Hz, 1H), 7.10 (d, J=7.6 Hz, 2H), 6.98 (m, 3H), 6.87 (t, J=7.6 Hz, 1H), 6.69 (d, J=8.0 Hz, 2H), 4.77 (dd, J=9.6, 6.0 Hz, 1H), 4.23 (m, 1H), 4.02 (t, J=9.6 Hz, 1H), 3.93 (m, 1H), 3.31 (dd, J=9.2, 6.4 Hz, 1H), 3.19-3.0 (m, 3H), 2.93 (dd, J=13.6, 8.8 Hz, 1H), 2.82 (dd, J=13.2, 6.8 Hz, 1H), 2.71 (t, J=13.2 Hz, 1H), 1.83 (m, 1H), 0.91 (d, J=6.4 Hz, 3H), 0.88 (d, J=6.4 Hz, 3H); ¹³C NMR (100 MHz, CDCl₃) δ 168.82, 153.25, 150.64, 137.60, 137.47, 129.68 (2C), 129.51 (2C), 129.26 (2C), 128.46 (2C), 126.55, 126.41, 124.77, 118.49 (2C), 114.57 (2C), 72.45, 69.91, 58.89, 53.74, 53.50, 48.32, 35.63, 27.42, 20.32, 20.10. HRMS (ESI) m/z: C₃₀H₃₇N₄O₆S (M+H)⁺ calcd, 581.2434; found, 581.2438.

(5S)—N-[(1S,2R)-3-[[(4-Aminophenyl)sulfonyl](isobutyl)amino]-1-benzyl-2-hydroxypropyl]-3-(3-fluorophenyl)-2-oxo-oxazolidine-S-carboxamide (25b). ¹H NMR (400 MHz, CDCl₃) δ7.58 (d, J=8.8 Hz, 2H), 7.38-7.33 (m, 2H), 7.12-7.08 (m, 3H), 7.01 (t, J=8.0 Hz, 2H), 6.93-6.85 (m, 2H), 6.70 (d, J=8.8 Hz, 3H), 4.78 (dd, J=10.0, 6.0 Hz, 1H), 4.22 (m, 2H), 4.01 (t, J=9.2 Hz, 1H), 3.91 (m, 1H), 3.73 (br. s, 1H), 3.30 (dd, J=9.2, 5.6 Hz, 1H), 3.22 (dd, J=14.8, 9.2 Hz, 1H), 3.07 (dd, J=13.6, 4.4 Hz, 1H), 3.0 (dd, J=13.6, 8.8 Hz, 1H), 2.95 (dd, J=14.8, 2.4 Hz, 1H), 2.79 (dd, J=13.6, 6.8 Hz, 1H), 2.72 (dd, J=14.0, 10.8 Hz, 1H), 1.83 (m, 1H), 1.60 (br. s, 1H), 0.95 (d, J=6.4 Hz, 3H), 0.89 (d, J=6.4 Hz, 3H); ¹³C NMR (100 MHz, CDCl₃) [symbol] 168.51, 163.22 (d, J=244.2 Hz), 152.72, 150.92, 139.0 (d, J=10.6 Hz), 137.43, 130.50 (d, J=9.10 Hz), 129.77 (2C), 129.53 (2C), 128.54 (2C), 126.64, 126.33, 114.37 (2C), 113.50 (d, J=3.0 Hz), 111.52 (d, J=21.2 Hz), 106.06 (d, J=27.3 Hz), 72.46, 69.75, 59.11, 53.92, 53.40, 48.17, 35.62, 27.58, 20.38, 20.10. HRMS (ESI) m/z: C₃₀H₃₆FN₄O₆S (M+H)⁺ calcd, 599.2339; found, 599.2340.

(5S)—N-[(1S,2R)-3-[[(4-Aminophenyl)sulfonyl](isobutyl)amino]-1-benzyl-2-hydroxypropyl]-3-(3,4-difluorophenyl)-2-oxo-oxazolidine-S-carboxamide (25c). ¹H NMR (400 MHz, CDCl₃) δ7.56 (d, J=8.4 Hz, 2H), 7.54-7.48 (m, 1H), 7.19 (q, J=9.2 Hz, 1H), 7.11 (d, J=7.6 Hz, 2H), 7.02-6.96 (m, 3H), 6.90-6.85 (m, 2H), 6.68 (d, J=8.8 Hz, 2H), 4.78 (dd, J=10.0, 5.6 Hz, 1H), 4.28-4.21 (m, 2H), 3.98 (t, J=9.6 Hz, 1H), 3.94 (m, 1H), 3.76 (br. s, 1H), 3.27 (dd, J=9.2, 6.0 Hz, 1H), 3.17 (dd, J=15.2, 8.8 Hz, 1H), 3.08 (dd, J=14.0, 4.4 Hz, 1H), 2.99 (dd, J=14.8, 2.4 Hz, 1H), 2.95 (dd, J=13.6, 8.4 Hz, 1H), 2.81 (dd, J=13.2, 6.8 Hz, 1H), 2.73 (dd, J=14.0, 10.8 Hz, 1H), 1.83 (m, 1H), 0.92 (d, J=6.4 Hz, 3H), 0.89 (d, J=6.8 Hz, 3H); ¹³C NMR (100 MHz, CDCl₃) [symbol] 168.41, 152.92, 151.02, 150.44 (dd, J=246.5, 13.6 Hz), 147.36 (dd, J=245.0, 12.4 Hz), 137.59, 134.06 (m), 129.72 (2C), 129.55 (2C), 128.45 (2C), 126.52, 126.14, 117.59 (d, J=18.2 Hz), 114.32 (2C), 113.88 (q, J=3.8 Hz), 108.36 (d, J=22.0 Hz), 72.491, 69.75, 59.01, 53.81, 53.36, 48.27, 35.48, 27.48, 20.34, 20.10. HRMS (ESI) m/z: C₃₀H₃₅F₂N₄O₆S (M+H)⁺ calcd, 617.2245; found, 617.2246.

(5S)—N-[(1S,2R)-3-[[(4-Aminophenyl)sulfonyl](isobutyl)amino]-1-benzyl-2-hydroxypropyl]-2-oxo-3-[(3-trifluoromethyl)phenyl]oxazolidine-S-carboxamide (25d). ¹H NMR (400 MHz, CDCl₃) δ7.68 (d, J=8.0 Hz, 1H), 7.61 (s, 1H), 7.55 (d, J=8.8 Hz, 2H), 7.52 (t, J=8.0 Hz, 1H), 7.45 (d, J=7.6 Hz, 1H), 7.09 (d, J=7.2 Hz, 2H), 6.96 (t, J=7.6 Hz, 2H), 6.81 (t, J=7.6 Hz, 1H), 6.77 (d, J=10.4 Hz, 1H), 6.67 (d, J=8.8 Hz, 2H), 4.78 (dd, J=10.4, 6.0 Hz, 1H), 4.24 (m, 1H), 4.16 (br. s, 1H), 4.02 (t, J=9.6 Hz, 1H), 3.91 (m, 1H), 3.73 (br. s, 1H), 3.30 (dd, J=9.2, 6.0 Hz, 1H), 3.19 (dd, J=15.2, 9.2 Hz, 1H), 3.07 (dd, J=14.0, 4.4 Hz, 1H), 3.0-2.93 (m, 2H), 2.78 (dd, J=13.2, 6.8 Hz, 1H), 2.70 (dd, J=13.6, 10.8 Hz, 1H), 1.82 (m, 1H), 0.93 (d, J=6.8 Hz, 3H), 0.88 (d, J=6.8 Hz, 3H); ¹³C NMR (100 MHz, CDCl₃) [symbol] 168.44, 152.86, 150.98, 138.13, 137.50, 131.77 (d, J=32.6 Hz), 129.94, 129.77 (2C), 129.55 (2C), 128.50 (2C), 126.55, 126.24, 121.40, 121.23 (q, J=3.8 Hz), 115.02 (q, J=3.8 Hz), 114.35 (2C), 72.50, 69.85, 59.09, 53.89, 53.32, 51.06, 48.10, 35.57, 27.56, 20.37, 20.11. HRMS (ESI) m/z: C₃₁H₃₆F₃N₄O₆S (M+H)⁺ calcd, 649.2307; found, 649.2291.

(5S)-3-(3-Acetylphenyl)-N-[(1S,2R)-3-[[(4-aminophenyl)sulfonyl](isobutyl)amino]-1-benzyl-2-hydroxypropyl]-2-oxo-oxazolidine-S-carboxamide (25e). ¹H NMR (400 MHz, CDCl₃) δ7.91 (t, J=2.0 Hz, 1H), 7.80-7.74 (m, 2H), 7.54 (d, J=8.8 Hz, 2H), 7.50 (t, J=7.6 Hz, 1H), 7.10 (d, J=7.2 Hz, 2H), 6.97 (t, J=7.2 Hz, 2H), 6.92 (d, J=10.0 Hz, 1H), 6.83 (t, J=7.2 Hz, 1H), 6.65 (d, J=8.8 Hz, 2H), 4.78 (dd, J=10.0, 6.0 Hz, 1H), 4.22 (m, 2H), 4.03 (t, J=9.6 Hz, 1H), 3.94 (m, 1H), 3.79 (br. s, 1H), 3.34 (dd, J=9.2, 6.0 Hz, 1H), 3.15 (dd, J=15.2, 9.2 Hz, 1H), 3.08 (dd, J=14.0, 4.4 Hz, 1H), 3.01-2.90 (m, 2H), 2.80 (dd, J=13.6, 6.8 Hz, 1H), 2.72 (dd, J=13.6, 11.2 Hz, 1H), 2.62 (s, 3H), 1.82 (m, 1H), 0.91 (d, J=6.4 Hz, 3H), 0.87 (d, J=6.4 Hz, 3H); ¹³C NMR (100 MHz, CDCl₃) [symbol] 197.64, 168.54, 153.15, 151.03, 138.12, 138.01, 137.62, 129.72 (2C), 129.63, 129.56 (2C), 128.47 (2C), 126.55, 126.14, 124.69, 123.0, 117.61, 114.30 (2C), 72.55, 69.94, 59.02, 53.84, 53.41, 48.23, 35.65, 27.47, 26.90, 20.34, 20.10. HRMS (ESI) m/z: C₃₂H₃₉N₄O₇S (M+H)⁺ calcd, 623.2539; found, 623.2543.

(5S)-3-(4-Acetylphenyl)-N-[(1S,2R)-3-[[(4-aminophenyl)sulfonyl](isobutyl)amino]-1-benzyl-2-hydroxypropyl]-2-oxo-oxazolidine-S-carboxamide (25f). ¹H NMR (400 MHz, CDCl₃) δ8.04-8.01 (m, 2H), 7.59-7.53 (m, 4H), 7.11 (d, J=6.8 Hz, 2H), 6.98 (t, J=7.6 Hz, 2H), 6.81 (t, J=7.2 Hz, 1H), 6.72-6.66 (m, 3H), 4.80 (dd, J=9.6, 5.6 Hz, 1H), 4.24 (m, 1H), 4.16 (br. s, 2H), 4.06 (t, J=9.6 Hz, 1H), 3.91 (m, 1H), 3.74 (br. s, 1H), 3.35 (dd, J=9.6, 6.4 Hz, 1H), 3.24 (dd, J=15.2, 9.6 Hz, 1H), 3.08 (dd, J=14.0, 4.8 Hz, 1H), 3.01 (dd, J=13.6, 8.8 Hz, 1H), 2.94 (dd, J=14.8, 2.4 Hz, 1H), 2.79 (dd, J=13.2, 6.0 Hz, 1H), 2.73 (dd, J=14.0, 11.2 Hz, 1H), 2.63 (s, 3H), 1.83 (m, 1H), 0.96 (d, J=6.8 Hz, 3H), 0.90 (d, J=6.4 Hz, 3H); ¹³C NMR (100 MHz, CDCl₃) [symbol] 197.0, 168.39, 152.73, 151.0, 141.58, 137.52, 133.17, 129.81 (2C), 129.75 (2C), 129.55 (2C), 128.46 (2C), 126.60, 126.22, 117.58 (2C), 114.34 (2C), 72.47, 69.84, 59.08, 53.88, 53.40, 48.0, 35.54, 27.55, 26.68, 20.37, 20.11. HRMS (ESI) m/z: C₃₂H₃₉N₄O₇S (M+H)⁺ calcd, 623.2539; found, 623.2532.

(5S)—N-[(1S,2R)-3-[[(Benzo-[1,3]-dioxole-S-sulfonyl)](isobutyl)amino]-1-benzyl-2-hydroxypropyl]-3-(3-fluorophenyl)-2-oxo-oxazolidine-S-carboxamide (26b). ¹H NMR (400 MHz, CDCl₃) δ7.38-7.30 (m, 3H), 7.20 (s, 1H), 7.13-7.08 (m, 3H), 7.02 (t, J=8.0 Hz, 2H), 6.92-6.86 (m, 3H), 6.75 (d, J=9.6 Hz, 1H), 6.09 (s, 2H), 4.78 (dd, J=10.0, 6.0 Hz, 1H), 4.23 (m, 1H), 4.02 (t, J=9.6 Hz, 1H), 3.93 (m, 1H), 3.64 (br. s, 1H), 3.32 (dd, J=8.8, 5.6 Hz, 1H), 3.20 (dd, J=15.2, 9.2 Hz, 1H), 3.10 (dd, J=14.0, 4.4 Hz, 1H), 3.03-2.98 (m, 2H), 2.83 (dd, J=13.6, 6.4 Hz, 1H), 2.74 (dd, J=14.0, 10.8 Hz, 1H), 1.85 (m, 1H), 0.96 (d, J=6.8 Hz, 3H), 0.91 (d, J=6.0 Hz, 3H); ¹³C NMR (100 MHz, CDCl₃) [symbol] 168.63, 163.19 (d, J=243.4 Hz), 152.81, 151.77, 148.56, 139.01 (d, J=10.6 Hz), 137.45, 131.57, 130.48 (d, J=9.1 Hz), 129.49 (2C), 128.52 (2C), 126.63, 123.35, 113.52 (d, J=3.1 Hz), 111.50 (d, J=21.3 Hz), 108.58, 107.71, 106.06 (d, J=27.3 Hz), 102.56, 72.52, 69.81, 59.04, 53.87, 53.47, 48.20, 35.58, 27.48, 20.31, 20.07. HRMS (ESI) m/z: C₃₁H₃₅FN₃O₈S (M+H)⁺ calcd, 628.2129; found, 628.2127.

(5S)—N-[(1S,2R)-3-[[(Benzo-[1,3]-dioxole-S-sulfonyl)](isobutyl)amino]-1-benzyl-2-hydroxypropyl]-3-(3,4-difluorophenyl)-2-oxo-oxazolidine-S-carboxamide (26c). ¹H NMR (400 MHz, CDCl₃) δ7.54-7.48 (m. 1H), 7.36 (dd, J=8.0, 1.6 Hz, 1H), 7.22-7.11 (m, 4H), 7.04-6.98 (m, 3H), 6.89 (t, J=4.0 Hz, 2H), 6.81 (d, J=9.6 Hz, 1H), 6.08 (s, 2H), 4.79 (dd, J=10.0, 5.6 Hz, 1H), 4.25 (m, 1H), 4.0 (t, J=9.6 Hz, 1H), 3.95 (m, 1H), 3.64 (br. s, 1H), 3.30 (dd, J=9.2, 6.0 Hz, 1H), 3.17 (dd, J=15.2, 9.2 Hz, 1H), 3.10 (dd, J=13.6, 4.4 Hz, 1H), 3.05-2.95 (m, 2H), 2.85 (dd, J=13.6, 6.4 Hz, 1H), 2.74 (dd, J=13.6, 10.8 Hz, 1H), 1.85 (m, 1H), 0.94 (d, J=6.0 Hz, 3H), 0.90 (d, J=6.8 Hz, 3H). ¹³C NMR (100 MHz, CDCl₃) [symbol] 168.49, 152.84, 151.79, 150.45 (dd, J=246.3, 12.9 Hz), 148.57, 147.39 (dd, J=245.0, 12.9 Hz), 137.49, 134.05 (m), 131.53, 129.53 (2C), 128.51 (2C), 126.59, 123.36, 117.60 (d, J=18.2 Hz), 113.86 (d, J=3.8 Hz), 108.59, 108.37 (d, J=22.0 Hz), 107.71, 102.58, 72.54, 69.74, 59.07, 53.87, 53.42, 48.26, 35.50, 27.49, 20.31, 20.07. HRMS (ESI) m/z: C₃₁H₃₄F₂N₃O₈S (M+H)⁺ calcd, 646.2034; found, 646.2037.

(5S)—N-[(1S,2R)-3-[[(Benzo-[1,3]-dioxole-S-sulfonyl)](isobutyl)amino]-1-benzyl-2-hydroxypropyl]-2-oxo-3-[(3-trifluoromethyl)phenyl]oxazolidine-S-carboxamide (26d). ¹H NMR (400 MHz, CDCl₃) δ7.70 (dd, J=8.0, 1.2 Hz, 1H), 7.63 (s, 1H), 7.54 (t, J=8.4 Hz, 1H), 7.46 (d, J=7.6 Hz, 1H), 7.37 (dd, J=8.4, 2.0 Hz, 2H), 7.20 (d, J=2.0 Hz, 1H), 7.11 (dd, J=8.4, 1.6 Hz, 1H), 6.99 (t, J=7.2 Hz, 2H), 6.90 (d, J=8.0 Hz, 1H), 6.85-6.80 (m, 2H), 6.09 (s, 2H), 4.81 (dd, J=10.0, 5.6 Hz, 1H), 4.26 (m, 1H), 4.05 (t, J=9.6 Hz, 1H), 3.94 (m, 1H), 3.65 (br. s, 1H), 3.34 (dd, J=9.6, 6.4 Hz, 1H), 3.20 (dd, J=14.8, 8.8 Hz, 1H), 3.11 (dd, J=13.6, 4.4 Hz, 1H), 3.04-2.97 (m, 2H), 2.84 (dd, J=13.6, 7.2 Hz, 1H), 2.73 (dd, J=13.6, 11.2 Hz, 1H), 1.86 (m, 1H), 0.95 (d, J=6.8 Hz, 3H), 0.91 (d, J=6.8 Hz, 3H); ¹³C NMR (100 MHz, CDCl₃) [symbol] 168.54, 152.83, 151.82, 148.60, 138.11, 137.42, 131.78 (d, J=32.6 Hz), 131.52, 129.94, 129.52 (2C), 129.54 (2C), 126.61, 123.38, 121.40, 121.27 (q, J=3.8 Hz), 115.02 (q, J=3.8 Hz), 108.62, 107.73, 102.59, 72.57, 69.86, 59.14, 53.94, 53.39, 51.05, 48.11, 35.58, 27.54, 20.34, 20.08. HRMS (ESI) m/z: C₃₂H₃₅F₃N₃O₈S (M+H)⁺ calcd, 678.2097; found, 678.2101.

(5S)-3-(3-Acetylphenyl)-N-[(1S,2R)-3-[[(benzo-[1,3]-dioxole-5-sulfonyl)](isobutyl)amino]-1-benzyl-2-hydroxypropyl]-2-oxo-oxazolidine-S-carboxamide (26e). ¹H NMR (400 MHz, CDCl₃) δ7.95 (s, 1H), 7.77 (dd, J=7.6, 1.6 Hz, 2H), 7.52 (t, J=8.0 Hz, 1H), 7.36 (dd, J=8.0, 2.0 Hz, 1H), 7.19 (d, J=2.0 Hz, 1H), 7.13 (d, J=6.8 Hz, 2H), 6.99 (t, J=7.6 Hz, 3H), 6.89-6.83 (m, 2H), 6.07 (d, J=2 Hz, 2H), 4.82 (dd, J=10.0, 6.0 Hz, 1H), 4.24 (m, 1H), 4.07 (t, J=9.6 Hz, 1H), 3.98 (m, 1H), 3.77 (br. s, 1H), 3.38 (dd, J=9.2, 6.4 Hz, 1H), 3.20-3.11 (m, 2H), 3.06 (dd, J=15.2, 4.6 Hz, 1H), 2.97 (dd, J=13.2, 8.0 Hz, 1H), 2.87 (dd, J=13.2, 6.8 Hz, 1H), 2.75 (dd, J=14.0, 11.0 Hz, 1H), 2.64 (s, 3H), 1.87 (m, 1H), 0.93 (d, J=6.8 Hz, 3H), 0.90 (d, J=6.4 Hz, 3H); ¹³C NMR (100 MHz, CDCl₃) [symbol] 197.64, 168.68, 153.16, 151.72, 148.52, 138.10, 138.01, 137.61, 131.59, 129.62, 129.54 (2C), 128.50 (2C), 126.58, 124.71, 123.33, 123.0, 117.64, 108.56, 107.72, 102.55, 72.65, 69.96, 59.02, 53.86, 53.49, 48.25, 35.67, 27.44, 26.89, 20.29, 20.08. HRMS (ESI) m/z: C₃₃H₃₈N₃O₉S (M+H)⁺ calcd, 652.2329; found, 652.2324.

(5S)-3-(4-Acetylphenyl)-N-[(1S,2R)-3-[[(benzo-[1,3]-dioxole-S-sulfonyl)](isobutyl)-amino]-1-benzyl-2-hydroxypropyl]-2-oxo-oxazolidine-S-carboxamide (26f). ¹H NMR (400 MHz, CDCl₃) δ8.01 (d, J=8.8 Hz, 2H), 7.54 (d, J=8.8 Hz, 2H), 7.36 (dd, J=8.4, 2.0 Hz, 1H), 7.19 (d, J=1.6 Hz, 1H), 7.11 (d, J=7.2 Hz, 2H), 6.98 (t, J=7.6 Hz, 2H), 6.89 (d, J=8.0 Hz, 1H), 6.83-6.79 (m, 2H), 6.08 (s, 2H), 4.81 (dd, J=9.6, 5.6 Hz, 1H), 4.24 (m, 1H), 4.07 (t, J=9.6 Hz, 1H), 3.95 (m, 1H), 3.64 (br. s, 1H), 3.35 (dd, J=9.2, 6.0 Hz, 1H), 3.19 (dd, J=15.2, 9.2 Hz, 1H), 3.10 (dd, J=13.6, 4.4 Hz, 1H), 3.05-2.96 (m, 2H), 2.84 (dd, J=13.2, 6.4 Hz, 1H), 2.73 (dd, J=13.6, 10.8 Hz, 1H), 2.62 (s, 3H), 1.86 (m, 1H), 0.94 (d, J=6.8 Hz, 3H), 0.90 (d, J=6.8 Hz, 3H); ¹³C NMR (100 MHz, CDCl₃) [symbol] 196.97, 168.48, 152.71, 151.79, 148.57, 141.55, 137.46, 133.17, 131.53, 129.80 (2C), 129.52 (2C), 128.49 (2C), 126.64, 123.36, 117.58 (2C), 108.60, 107.71, 102.58, 72.53, 69.84, 59.09, 53.90, 53.46, 48.0, 35.53, 27.51, 26.66, 20.32, 20.08. HRMS (ESI) m/z: C₃₃H₃₈N₃O₉S (M+H)⁺ calcd, 652.2329; found, 652.2325.

(5S)—N-[(1S,2R)-1-Benzyl-3-[[(3-fluoro-4-methoxyphenyl)sulfonyl](isobutyl)amino]-2-hydroxypropyl]-3-(3-fluorophenyl)-2-oxo-oxazolidine-S-carboxamide (27b). ¹H NMR (400 MHz, CDCl₃) δ7.58 (m, 1H), 7.51 (dd, J=10.4, 2.4 Hz, 1H), 7.38-7.32 (m, 2H), 7.13-7.05 (m, 4H), 7.02 (t, J=7.6 Hz, 2H), 6.92-6.86 (m, 2H), 6.83 (d, J=10.0 Hz, 1H), 4.80 (dd, J=10.0, 6.0 Hz, 1H), 4.24 (m, 1H), 4.03 (t, J=9.6 Hz, 1H), 3.95 (s, 3H, overlapping signal), 3.94 (m, 1H, overlapping signal), 3.61 (d, J=3.2 Hz, 1H), 3.32 (dd, J=9.6, 6.0 Hz, 1H), 3.20 (dd, J=15.2, 9.2 Hz, 1H), 3.11 (dd, J=13.6, 4.4 Hz, 1H), 3.05 (dd, J=15.2, 2.8 Hz, 1H), 2.99 (dd, J=13.2, 8.4 Hz, 1H), 2.86 (dd, J=13.2, 6.4 Hz, 1H), 2.74 (dd, J=14.0, 10.8 Hz, 1H), 1.86 (m, 1H), 0.93 (d, J=6.4 Hz, 3H), 0.90 (d, J=6.4 Hz, 3H); ¹³C NMR (100 MHz, CDCl₃) [symbol] 168.67, 163.21 (d, J=244.2 Hz), 152.80, 151.95 (d, J=250.2 Hz), 151.83 (d, J=9.8 Hz), 139.0 (d, J=10.6 Hz), 137.37, 130.51 (d, J=9.1 Hz), 130.34 (d, J=4.5 Hz), 129.49 (2C), 128.56 (2C), 126.69, 124.89 (d, J=3.8 Hz), 115.60 (d, J=20.5 Hz), 113.53 (d, J=3 Hz), 113.19, 111.54 (d, J=21.2 Hz), 106.08 (d, J=26.6 Hz), 72.48, 69.81, 58.91, 56.63, 53.78, 53.50, 48.21, 35.59, 27.46, 20.29, 20.06. HRMS (ESI) m/z: C₃₁H₃₆F₂N₃O₇S (M+H)⁺ calcd, 632.2242; found, 632.2258.

(5S)—N-[(1S,2R)-1-Benzyl-3-[[(3-fluoro-4-methoxyphenyl)sulfonyl](isobutyl)amino]-2-hydroxypropyl]-3-(3,4-difluorophenyl)-2-oxo-oxazolidine-S-carboxamide (27c). ¹H NMR (400 MHz, CDCl₃) δ7.59-7.55 (m, 1H), 7.54-7.48 (m, 2H), 7.18 (q, J=9.2 Hz, 1H), 7.12 (d, J=7.2 Hz, 2H), 7.07-6.97 (m, 4H), 6.90 (t, J=7.2 Hz, 1H), 6.86 (d, J=10.0 Hz, 1H), 4.80 (dd, J=10.0, 6.0 Hz, 1H), 4.26 (m, 1H), 4.0 (t, J=9.2 Hz, 1H), 3.96 (m, 1H, overlapping signal), 3.95 (s, 3H), 3.62 (d, J=3.6 Hz, 1H), 3.31 (dd, J=9.2, 6.0 Hz, 1H), 3.18 (dd, J=15.2, 9.2 Hz, 1H), 3.21-3.04 (m, 2H), 2.97 (dd, J=13.6, 8.0 Hz, 1H), 2.87 (dd, J=13.6, 6.8 Hz, 1H), 2.74 (dd, J=13.6, 10.8 Hz, 1H), 1.86 (m, 1H), 0.92 (d, J=6.4 Hz, 3H), 0.89 (d, J=6.4 Hz, 3H); ¹³C NMR (100 MHz, CDCl₃) [symbol] 168.55, 152.88, 151.92 (d, J=250.2 Hz), 151.82 (d, J=10.6 Hz), 150.45 (dd, J=246.4, 12.9 Hz), 147.39 (dd, J=245.7, 12.9 Hz), 137.46, 134.03 (m), 130.32 (d, J=3.8 Hz), 129.51 (2C), 128.52 (2C), 126.61, 124.88 (d, J=3.8 Hz), 117.61 (d, J=18.2 Hz), 115.58 (d, J=21.2 Hz), 113.88 (q, J=3.8 Hz), 113.18, 108.37 (d, J=22.0 Hz), 72.50, 69.76, 58.86, 56.62, 53.72, 53.47, 48.28, 35.49, 27.42, 20.27, 20.06. HRMS (ESI) m/z: C₃₁H₃₅F₃N₃O₇S (M+H)⁺ calcd, 650.2148; found, 650.2151.

(5S)—N-[(1S,2R)-1-Benzyl-3-[[(3-fluoro-4-methoxyphenyl)sulfonyl](isobutyl)amino]-2-hydroxypropyl]-2-oxo-3-[(3-trifluoromethyl)phenyl)oxazolidine-S-carboxamide (27d). ¹H NMR (400 MHz, CDCl₃) δ 7.69 (d, J=8 Hz, 1H), 7.64 (br. s, 1H), 7.59-7.49 (m 3H), 7.45 (d, J=7.6 Hz, 1H), 7.12 (d, J=6.8 Hz, 2H), 7.05 (t, J=8.4 Hz, 1H), 6.99 (t, J=7.6 Hz, 2H), 6.87-6.82 (m, 2H), 4.81 (dd, J=10.0, 5.6 Hz, 1H), 4.27 (m, 1H), 4.06 (t, J=10.0 Hz, 1H), 3.96 (m, 1H, overlapping signal), 3.95 (s, 3H), 3.61 (br. s, 1H), 3.35 (dd, J=9.2, 5.6 Hz, 1H), 3.20 (dd, J=15.2, 9.2 Hz, 1H), 3.11 (dd, J=14.0, 4.4 Hz, 1H), 3.05 (dd, J=15.2, 3.2 Hz, 1H), 2.98 (dd, J=13.2, 8.0 Hz, 1H), 2.87 (dd, J=13.2, 6.8 Hz, 1H), 2.74 (dd, J=14, 10.8 Hz, 1H), 1.87 (m, 1H), 0.94 (d, J=6.4 Hz, 3H), 0.90 (d, J=6.4 Hz, 3H); ¹³C NMR (100 MHz, CDCl₃) [symbol] 168.59, 152.88, 151.95 (d, J=250.9 Hz), 151.84 (d, J=10.7 Hz), 138.09, 137.40, 131.76 (d, J=32.5 Hz), 130.30, 129.94, 129.50 (2C), 128.54 (2C), 126.62, 124.89 (d, J=3.8 Hz), 121.40, 121.25 (q, J=3.8 Hz), 115.60 (d, J=20.5 Hz), 115.04 (q, J=3.8 Hz), 113.19, 72.53, 69.88, 58.92, 56.63, 53.78, 53.43, 48.13, 35.56, 27.46, 20.29, 20.06. HRMS (ESI) m/z: C₃₂H₃₆F₄N₃O₇S (M+H)⁺ calcd, 682.2210; found, 682.2203.

(S)-3-(3-Acetylphenyl)-N-[(1S,2R)-1-Benzyl-3-[[(3-fluoro-4-methoxyphenyl)-sulfonyl](isobutyl)amino]-2-hydroxypropyl]-2-oxo-oxazolidine-S-carboxamide (27e). ¹H NMR (400 MHz, CDCl₃) δ 7.96 (m, 1H), 7.78-7.73 (m, 2H), 7.59-7.48 (m, 3H), 7.13 (d, J=7.2 Hz, 2H), 7.07-6.98 (m, 4H), 6.85 (t, J=8.0 Hz, 1H), 4.82 (dd, J=9.6, 5.6 Hz, 1H), 4.25 (m, 1H), 4.07 (t, J=9.6 Hz, 1H), 3.99 (br. s, 1H), 3.94 (s, 3H), 3.75 (m, 1H), 3.41-3.37 (m, 1H), 3.21-3.06 (m, 3H), 3.0-2.86 (m, 2H), 2.76 (dd, J=14.0, 11.2 Hz, 1H), 2.64 (s, 3H), 1.88 (m, 1H), 0.92 (d, J=6.8 Hz, 3H), 0.89 (d, J=6.4 Hz, 3H); ¹³C NMR (100 MHz, CDCl₃) δ197.65, 168.71, 153.18, 151.90 (d, J=251.0 Hz), 151.74 (d, J=9.9 Hz), 138.09, 138.01, 137.59, 130.46 (d, J=4.5 Hz), 129.62, 129.53 (2C), 128.51 (2C), 126.61, 124.86 (d, J=3.8 Hz), 124.74, 122.98, 117.64, 115.58 (d, J=20.4 Hz), 113.17, 72.60, 69.98, 58.82, 56.60, 53.69, 53.55, 48.26, 35.68, 27.38, 26.88, 20.25, 20.06. HRMS (ESI) m/z: C₃₃H₃₉FN₃O₈S (M+H)⁺ calcd, 656.2442; found, 656.2441.

(S)-3-(4-Acetylphenyl)-N-[(1S,2R)-1-Benzyl-3-[[(3-fluoro-4-methoxyphenyl)-sulfonyl](isobutyl)amino]-2-hydroxypropyl]-2-oxo-oxazolidine-S-carboxamide (27f). ¹H NMR (400 MHz, CDCl₃) δ8.01 (d, J=8.8 Hz, 2H), 7.58-7.52 (m, 3H), 7.50 (dd, J=10.4, 2.4 Hz, 1H), 7.11 (d, J=8.0 Hz, 2H), 7.04 (t, J=8.4 Hz, 1H), 6.97 (t, J=7.6 Hz, 2H), 6.92 (d, J=9.6 Hz, 1H), 6.82 (t, J=7.2 Hz, 1H), 4.82 (dd, J=9.6, 5.6 Hz, 1H), 4.26 (m, 1H), 4.08 (t, J=10.0 Hz, 1H), 3.98 (m, 1H), 3.94 (s, 3H), 3.68 (br. s, 1H), 3.35 (dd, J=9.6, 6.4 Hz, 1H), 3.18 (dd, J=15.2, 8.8 Hz, 1H), 3.13-3.06 (m, 2H), 2.97 (dd, J=13.2, 8.0 Hz, 1H), 2.89 (dd, J=13.6, 6.8 Hz, 1H), 2.74 (dd, J=14.0, 11.2 Hz, 1H), 2.61 (s, 3H), 1.87 (m, 1H), 0.91 (d, J=6.4 Hz, 3H), 0.89 (d, J=6.4 Hz, 3H); ¹³C NMR (100 MHz, CDCl₃) [symbol] 197.0, 168.55, 152.78, 151.90 (d, J=251.0 Hz), 151.78 (d, J=10.7 Hz), 141.54, 137.46, 133.15, 130.31, 129.78 (2C), 129.50 (2C), 128.47 (2C), 126.63, 124.86 (d, J=3.0 Hz), 117.58 (2C), 115.56 (d, J=20.5 Hz), 113.18, 72.49, 69.88, 58.83, 56.60, 53.69, 53.50, 48.01, 35.49, 27.39, 26.64, 20.26, 20.06. HRMS (ESI) m/z: C₃₃H₃₉FN₃O₈S (M+H)⁺ calcd, 656.2442; found, 656.2448.

(5S)—N-[(1S,2R)-1-Benzyl-2-hydroxy-3-[isobutyl[[(4-trifluoromethoxy)phenyl]sulfonyl]-amino]propyl]-2-oxo-3-[(3-trifluoromethyl)phenyl]oxazolidine-S-carboxamide (28d). ¹H NMR (400 MHz, CDCl₃) δ7.88-7.85 (m, 2H), 7.70 (d, J=8.0 Hz, 1H), 7.63 (s, 1H), 7.54 (dt, J=8.0, 2.4 Hz, 1H), 7.46 (d, J=8.0 Hz, 1H), 7.37 (dd, J=7.6, 1.2 Hz, 2H), 7.12 (dd, J=7.6, 1.2 Hz, 2H), 7.03-6.98 (m, 2H), 6.85 (dt, J=8.0, 2.4 Hz, 1H), 6.70 (d, J=8.0 Hz, 1H), 4.81 (dd, J=9.6, 6.0 Hz, 1H), 4.26 (m, 1H), 4.06 (t, J=10.0 Hz, 1H), 3.94 (m, 1H), 3.54 (br. s, 1H), 3.37 (dd, J=10.0, 6.0 Hz, 1H), 3.25 (dd, J=15.6, 9.2 Hz, 1H), 3.13-3.02 (m, 3H), 2.87 (dd, J=13.6, 6.0 Hz, 1H), 2.71 (dd, J=13.6, 9.6 Hz, 1H), 1.87 (m, 1H), 0.96 (d, J=6.4 Hz, 3H), 0.91 (d, J=6.4 Hz, 3H); ¹³C NMR (100 MHz, CDCl₃) [symbol] 168.61, 152.78, 152.57, 138.06, 137.26, 136.82, 131.81 (d, J=32.6 Hz), 129.96 129.71 (2C), 129.48 (2C), 128.61 (2C), 126.69, 121.40, 121.33, 121.27 (2C), 115.02 (m), 72.50, 69.84, 58.90, 53.79, 53.47, 51.07, 48.10, 35.56, 27.49, 20.28, 20.02. HRMS (ESI) m/z: C₃₂H₃₄F₆N₃O₇S (M+H)⁺ calcd, 718.2021; found, 718.2028.

(5S)-3-(3-Acetylphenyl)-N-[(1S,2R)-1-benzyl-2-hydroxy-3-[isobutyl[[(4-trifluoro-methoxy)phenyl]sulfonyl]amino]propyl]-2-oxo-oxazolidine-S-carboxamide (28e). ¹H NMR (400 MHz, CDCl₃) δ7.90-7.77 (m, 5H), 7.53 (t, J=8.0 Hz, 1H), 7.36 (d, J=8.8 Hz, 2H), 7.13 (d, J=8.0 Hz, 2H), 7.03 (t, J=8.0 Hz, 2H), 6.87 (t, J=7.6 Hz, 1H), 6.83 (d, J=9.6 Hz, 1H), 4.82 (dd, J=9.6, 5.6 Hz, 1H), 4.25 (m, 1H), 4.09 (t, J=10.0 Hz, 1H), 3.94 (m, 1H), 3.59 (d, J=3.2 Hz, 1H), 3.42 (dd, J=9.6, 6.0 Hz, 1H), 3.25 (dd, J=15.2, 9.2 Hz, 1H), 3.13 (dd, J=14.4, 4.8 Hz, 1H), 3.07-3.02 (m, 2H), 2.88 (dd, J=13.2, 6.4 Hz, 1H), 2.75 (dd, J=13.6, 10.8 Hz, 1H), 2.65 (s, 3H), 1.87 (m, 1H), 0.95 (d, J=6.8 Hz, 3H), 0.90 (d, J=6.8 Hz, 3H); ¹³C NMR (100 MHz, CDCl₃) [symbol] 197.53, 168.68, 152.95, 152.55, 138.06, 137.27, 136.85, 129.72 (2C), 129.68, 129.50 (2C), 128.65 (2C), 126.77, 124.79, 123.01, 121.27 (2C), 117.53, 72.52, 69.89, 58.93, 53.84, 53.54, 48.21, 35.65, 27.51, 26.90, 20.29, 20.03. HRMS (ESI) m/z: C₃₃H₃₇F₃N₃O₈S (M+H)⁺ calcd, 692.2253; found, 692.2244.

(5S)—N-[(1S,2R)-1-Benzyl-2-hydroxy-3-[isobutyl[(3-methoxyphenyl)sulfonyl]amino]-propyl]-2-oxo-3-phenyloxazolidine-S-carboxamide (29a). ¹H NMR (400 MHz, CDCl₃) δ7.46-7.36 (m, 6H), 7.31 (t, J=2.8 Hz, 1H), 7.20 (m, 1H), 7.12 (m, 3H), 7.02 (t, J=7.2 Hz, 2H), 6.88 (t, J=7.6 Hz, 1H), 6.77 (d, J=10 Hz, 1H), 4.78 (dd, J=10.0, 6.0 Hz, 1H), 4.22 (m, 1H), 4.04 (t, J=9.6 Hz, 1H), 3.93 (m, 1H), 3.86 (s, 3H), 3.64 (br. s, 1H), 3.36 (dd, J=8.8, 5.6 Hz, 1H), 3.25 (dd, J=15.6, 9.6 Hz, 1H), 3.12 (dd, J=14.0, 4.4 Hz, 1H), 3.07-3.01 (m, 2H), 2.87 (dd, J=13.6, 6.4 Hz, 1H), 2.73 (dd, J=14.0, 10.8 Hz, 1H), 1.86 (m, 1H), 0.96 (d, J=6.4 Hz, 3H), 0.90 (d, J=6.4 Hz, 3H); ¹³C NMR (100 MHz, CDCl₃) [symbol] 168.89, 160.23, 153.02, 139.53, 137.49, 137.37, 130.51, 129.49 (2C), 129.31 (2C), 128.59 (2C), 126.71, 124.82, 119.65, 119.16, 118.47 (2C), 112.65, 72.47, 69.82, 59.03, 55.91, 53.91, 53.56, 48.29, 35.70, 27.53, 20.31, 20.06. HRMS (ESI) m/z: C₃₁H₃₈N₃O₇S (M+H)⁺ calcd, 596.2430; found, 596.2435.

(5S)-3-(4-Acetylphenyl)-N-[(1S,2R)-1-benzyl-2-hydroxy-3-[isobutyl[(3-methoxyphenyl)-sulfonyl]amino]propyl]-2-oxo-oxazolidine-S-carboxamide (29f). ¹H NMR (400 MHz, CDCl₃) δ 8.0 (d, J=8.8 Hz, 2H), 7.54 (d, J=8.8 Hz, 2H), 7.43 (t, J=7.6 Hz, 1H), 7.36 (d, J=8.8 Hz, 1H), 7.30 (t, J=2.4 Hz, 1H), 7.10 (d, J=7.6 Hz, 3H), 6.97 (t, J=7.6 Hz, 2H), 6.85 (d, J=10.0 Hz, 1H), 6.80 (t, J=7.6 Hz, 1H), 4.81 (dd, J=9.6, 5.6 Hz, 1H), 4.25 (m, 1H), 4.06 (t, J=9.6 Hz, 1H), 3.96 (m, 1H), 3.85 (s, 3H), 3.68 (br. s, 1H), 3.34 (dd, J=9.2, 6.0 Hz, 1H), 3.23 (dd, J=15.4, 9.2 Hz, 1H), 3.12-3.05 (m, 2H), 3.02 (dd, J=13.6, 8.4 Hz, 1H), 2.89 (dd, J=13.6, 7.2 Hz, 1H), 2.74 (dd, J=14.0, 11.2 Hz, 1H), 2.61 (s, 3H), 1.87 (m, 1H), 0.93 (d, J=6.4 Hz, 3H), 0.90 (d, J=6.8 Hz, 3H); ¹³C NMR (100 MHz, CDCl₃) [symbol] 196.97, 168.49, 160.19, 152.72, 141.54, 139.49, 137.46, 133.14, 130.50, 129.78 (2C), 129.50 (2C), 128.46 (2C), 126.61, 119.61, 119.06, 117.57 (2C), 112.70, 72.47, 69.84, 58.97, 55.89, 53.81, 53.47, 47.99, 35.49, 27.46, 26.65, 20.28, 20.05. HRMS (ESI) m/z: C₃₃H₄₀N₃O₈S (M+H)⁺ calcd, 638.2536; found, 638.2538.

(5S)—N-[(1S,2R)-1-Benzyl-3-[(cyclopropylmethyl)[(3-methoxyphenyl)sulfonyl]amino]-2-hydroxypropyl]-3-(3-fluorophenyl)-2-oxo-oxazolidine-S-carboxamide (36b). ¹H NMR (400 MHz, CDCl₃) δ7.74 (d, J=9.2 Hz, 2H), 7.38-7.32 (m, 1H), 7.13 (d, J=6.8 Hz, 2H), 7.09 (dd, J=8.8, 2.0 Hz, 1H), 7.04-6.97 (m, 4H), 6.92-6.86 (m, 2H), 6.80 (d, J=10.0 Hz, 1H), 4.79 (dd, J=10.0, 5.6 Hz, 1H), 4.28 (m, 1H), 4.02 (t, J=9.2 Hz, 2H), 3.87 (s, 3H), 3.62 (br. s, 1H), 3.36-3.10 (m, 5H), 2.96 (dd, J=14.0, 7.2 Hz, 1H), 2.78 (dd, J=14.0, 11.2 Hz, 1H), 0.87 (m, 1H), 0.56-0.53 (m, 2H), 0.18 (m, 2H); ¹³C NMR (100 MHz, CDCl₃) δ168.63, 163.26, 163.20 (d, J=244.2 Hz), 152.77, 139.03 (d, J=10.6 Hz), 137.49, 130.53, 130.44, 129.57 (2C), 129.51 (2C), 128.54 (2C), 126.63, 114.61 (2C), 113.52 (d, J=3.1 Hz), 111.51 (d, J=21.2 Hz), 106.07 (d, J=27.3 Hz), 72.36, 69.81, 55.82, 55.04, 53.40, 52.15, 48.21, 35.57, 10.07, 4.72, 4.26. HRMS (ESI) m/z: C₃₁H₃₅FN₃O₇S (M+H)⁺ calcd, 612.2179; found, 612.2180.

(5S)—N-[(1S,2R)-1-Benzyl-3-[(cyclopropylmethyl)[(3-methoxyphenyl)sulfonyl]amino]-2-hydroxypropyl]-3-(3,4-difluorophenyl)-2-oxo-oxazolidine-S-carboxamide (36c). ¹H NMR (400 MHz, CDCl₃) δ7.74 (d, J=8.8 Hz, 2H), 7.54-7.48 (m, 1H), 7.19 (q, J=9.6 Hz, 1H), 7.13 (d, J=7.6 Hz, 2H), 7.05-6.96 (m, 5H), 6.90 (t, J=7.6 Hz, 1H), 6.82 (d, J=10.0 Hz, 1H), 4.80 (dd, J=10.0, 5.6 Hz, 1H), 4.29 (m, 1H), 4.04 (m, 1H), 4.0 (t, J=9.2 Hz, 1H), 3.87 (s, 3H), 3.60 (br. s, 1H) 3.35-3.24 (m, 3H), 3.21-3.10 (m, 2H), 2.96 (dd, J=14.4, 7.6 Hz, 1H), 2.79 (dd, J=14.0, 10.8 Hz, 1H), 0.91-0.83 (m, 1H), 0.54 (m, 2H), 0.18 (m, 2H); ¹³C NMR (100 MHz, CDCl₃) [symbol] 168.51, 163.27, 152.83, 150.46 (d, J=246.4 Hz), 147.38 (d, J=245.6 Hz), 137.57, 134.05 (m), 130.42, 129.56 (2C), 129.55 (2C), 128.51 (2C), 126.57, 117.59 (d, J=17.4 Hz), 114.60 (2C), 113.87 (d, J=3.8 Hz), 108.38 (d, J=22.7 Hz), 72.38, 69.76, 55.82, 55.03, 53.37, 52.12, 48.28, 35.48, 10.05, 4.71, 4.26. HRMS (ESI) m/z: C₃₁H₃₄F₂N₃O₇S (M+H)⁺ calcd, 630.2086; found, 630.2077.

(5S)-3-(4-Acetylphenyl)-N-[(1S,2R)-1-Benzyl-3-[(cyclopropylmethyl)[(3-methoxy-phenyl)sulfonyl]amino]-2-hydroxypropyl]-2-oxo-oxazolidine-S-carboxamide (36f). ¹H NMR (400 MHz, CDCl₃) δ8.02 (d, J=9.2 Hz, 2H), 7.74 (d, J=8.8 Hz, 2H), 7.55 (d, J=8.8 Hz, 2H), 7.12 (d, J=7.6 Hz, 2H), 7.02-6.97 (m, 4H), 6.83 (t, J=6.8 Hz, 1H), 6.69 (d, J=9.6 Hz, 1H), 4.81 (dd, J=10.4, 6.0 Hz, 1H), 4.28 (m, 1H), 4.08 (t, J=9.6 Hz, 1H), 4.02 (m, 1H), 3.87 (s, 3H), 3.54 (d, J=2.4 Hz, 1H), 3.39 (dd, J=9.2, 5.6 Hz, 1H), 3.30 (dd, J=15.2, 8.8 Hz, 1H), 3.23-3.18 (m, 2H), 3.12 (dd, J=14.4, 4.8 Hz, 1H), 2.94 (dd, J=14.0, 7.2 Hz, 1H), 2.78 (dd, J=13.6, 10.4 Hz, 1H), 2.62 (s, 3H), 0.88 (m, 1H), 0.56 (d, J=7.6 Hz, 2H), 0.23-0.15 (m, 2H); ¹³C NMR (100 MHz, CDCl₃) [symbol] 196.97, 168.50, 163.24, 152.74, 141.57, 137.58, 133.13, 130.44, 129.78 (2C), 129.54 (4C), 128.46 (2C), 126.58, 117.58 (2C), 114.59 (2C), 72.38, 69.89, 55.80, 55.0, 53.40, 52.09, 48.01, 35.49, 26.65, 10.02, 4.69, 4.26. HRMS (ESI) m/z: C₃₃H₃₈N₃O₈S (M+H)⁺ calcd, 636.2379; found, 636.2369.

(5S)—N-[(1S,2R)-1-Benzyl-2-hydroxy-3-[[(3-methoxyphenyl)sulfonyl](2-thiophenyl-methyl)]amino]propyl]-2-oxo-3-phenyloxazolidine-S-carboxamide (37a). ¹H NMR (400 MHz, CDCl₃) δ7.45-7.38 (m, 6H), 7.32 (d, J=2.0 Hz, 1H), 7.22-7.17 (m, 2H), 7.13 (m, 1H), 7.08 (d, J=8.0 Hz, 2H), 7.02 (t, J=7.6 Hz, 2H), 6.93-6.88 (m, 3H), 6.57 (d, J=9.6 Hz, 1H), 4.72 (dd, J=10.0, 6.0 Hz, 1H), 4.65 (AB, d, J=15.6 Hz, 1H), 4.59 (AB, d, J=15.6 Hz, 1H), 4.10 (m, 1H), 4.05 (t, J=9.6 Hz, 1H), 3.85 (s, 3H), 3.66 (dd, J=11.2, 6.4 Hz, 1H), 3.48 (br. s, 1H), 3.43 (dd, J=9.2, 6.0 Hz, 1H), 3.24 (m, 2H), 3.04 (dd, J=14.0, 4.0 Hz, 1H), 2.68 (dd, J=14.0, 10.4 Hz, 1H); ¹³C NMR (100 MHz, CDCl₃) [symbol] 168.97, 160.30, 153.01, 139.91, 138.26, 137.49, 137.29, 130.59, 129.50 (2C), 129.31 (2C), 128.59 (2C), 128.34, 127.21, 127.07, 126.67, 124.82, 119.68, 119.59, 118.47 (2C), 112.34, 71.96, 69.78, 55.91, 53.36, 51.68, 48.27, 48.20, 35.61. HRMS (ESI) m/z: C₃₂H₃₄N₃O₇S₂ (M+H)⁺ calcd, 636.1838; found, 636.1864.

(5S)—N-[(1S,2R)-1-Benzyl-2-hydroxy-3-[[(3-methoxyphenyl)sulfonyl](2-thiophenyl-methyl)]amino]propyl]-3-(3-fluorophenyl)-2-oxo-oxazolidine-S-carboxamide (37b). ¹H NMR (400 MHz, CDCl₃) δ7.47-7.34 (m, 4H), 7.32 (t, J=2.0 Hz, 1H), 7.24 (t, J=3.2 Hz, 1H), 7.14 (m, 1H), 7.10-7.01 (m, 5H), 6.93-6.87 (m, 4H), 6.43 (d, J=9.2 Hz, 1H), 4.72 (dd, J=10.0, 5.6 Hz, 1H), 4.65 (AB d, J=15.6 Hz, 1H), 4.58 (AB d, J=15.6 Hz, 1H), 4.11 (m, 1H), 4.02 (t, J=10.0 Hz, 1H), 3.86 (s, 3H), 3.63 (m, 1H), 3.38 (dd, J=9.2, 5.6 Hz, 1H), 3.29-3.17 (m, 2H), 3.04 (dd, J=14.0, 4.4, Hz, 1H), 2.67 (dd, J=13.6, 10.4, Hz, 1H); ¹³C NMR (100 MHz, CDCl₃) [symbol] 168.70, 163.19 (d, J=244.1 Hz), 160.28, 152.76, 139.91, 139.01 (d, J=10.6 Hz), 138.24, 137.32, 130.59, 130.51 (d, J=9.8 Hz), 129.49 (2C), 128.53 (2C), 128.35, 127.18, 127.04, 126.62, 119.66, 119.56, 113.51 (d, J=2.2 Hz), 112.37, 111.52 (d, J=21.3 Hz), 106.05 (d, J=26.5 Hz), 72.04, 69.74, 55.91, 53.30, 51.58, 48.16, 48.15, 35.56. HRMS (ESI) m/z: C₃₂H₃₃FN₃O₇S₂ (M+H)⁺ calcd, 654.1744; found, 654.1766.

(S)-3-(4-Acetylphenyl)-N-[(2S,3R)-1-benzyl-2-hydroxy-3-[[(3-methoxyphenyl)-sulfonyl](2-thiophenylmethyl)]amino]propyl]-2-oxo-oxazolidine-S-carboxamide (37f). ¹H NMR (400 MHz, CDCl₃) δ8.02 (d, J=8.8 Hz, 2H), 7.55 (d, J=9.2 Hz, 2H), 7.45 (t, J=7.6 Hz, 1H), 7.40 (d, J=7.6 Hz, 1H), 7.32 (t, J=2.4 Hz, 1H), 7.23 (dd, J=4.4, 2.8 Hz, 1H), 7.13 (m, 1H), 7.07 (d, J=7.2 Hz, 2H), 6.99 (t, J=7.6 Hz, 2H), 6.91 (m, 2H), 6.84 (t, J=7.6 Hz, 1H), 6.54 (d, J=9.6 Hz, 1H), 4.76 (dd, J=10.0, 6.0 Hz, 1H), 4.65 (AB d, J=15.2 Hz, 1H), 4.59 (AB d, J=15.2 Hz, 1H), 4.14 (m, 1H), 4.07 (t, J=9.6 Hz, 1H), 3.85 (s, 3H), 3.68 (m, 1H), 3.41 (m, 2H), 3.24 (m, 2H), 3.04 (dd, J=14.0, 4.4, Hz, 1H), 2.67 (dd, J=13.6, 10.4, Hz, 1H), 2.62 (s, 3H); ¹³C NMR (100 MHz, CDCl₃) [symbol] 196.98, 168.56, 160.31, 152.63, 141.54, 139.81, 138.25, 137.27, 133.20, 130.63, 129.84 (2C), 129.51 (2C), 128.52 (2C), 128.34, 127.22, 127.10, 126.66, 119.67, 119.58, 117.57 (2C), 112.41, 72.01, 69.75, 55.93, 53.28, 51.73, 48.29, 47.97, 35.58, 26.68. HRMS (ESI) m/z: C₃₄H₃₆N₃O₈S₂ (M+H)⁺ calcd, 678.1944; found, 678.1953.

(5S)—N-[(1S,2R)-1-Benzyl-2-hydroxy-3-[(2-thiophenylmethyl)[(2,4,5-trifluorophenyl)-sulfonyl]amino]propyl]-2-oxo-3-phenyloxazolidine-S-carboxamide (38a). ¹H NMR (400 MHz, CDCl₃) δ7.76-7.70 (m, 1H), 7.43-7.36 (m, 4H), 7.19-7.16 (m, 2H), 7.08-7.01 (m, 5H), 6.93-6.86 (m, 3H), 6.67 (d, J=9.2 Hz, 1H), 4.79-4.67 (m, 3H), 4.12 (m, 1H), 4.06 (t, J=9.6 Hz, 1H), 3.79 (m, 1H), 3.49-3.33 (m, 4H), 3.02 (dd, J=14.0, 4.4 Hz, 1H), 2.69 (dd, J=14.0, 10.4 Hz, 1H); ¹³C NMR (100 MHz, CDCl₃) [symbol] 169.21, 154.8 (dd, J=253.2, 10.6 Hz), 153.45 (dt, J=257.0, 13.6 Hz), 153.05, 146.48 (dd, J=253.2, 13.2 Hz), 137.57, 137.44, 137.14, 129.43 (2C), 129.34 (2C), 128.68 (2C), 127.20, 127.14, 126.81, 124.89, 119.64 (d, J=22.0 Hz), 118.51 (2C), 107.72 (dd, J=27.3, 21.2 Hz), 72.10, 69.81, 53.78, 51.05, 51.02, 48.29, 47.63, 35.45, 29.88. HRMS (ESI) m/z: C₃₁H₂₉F₃N₃O₆S₂ (M+H)⁺ calcd, 660.1450; found, 660.1462.

(5S)—N-[(1S,2R)-1-Benzyl-2-hydroxy-3-[(2-thiophenylmethyl)[(2,4,5-trifluorophenyl)-sulfonyl]amino]propyl]-3-(3-fluorophenyl)-2-oxo-oxazolidine-S-carboxamide (38b). ¹H NMR (400 MHz, CDCl₃): δ 7.78-7.72 (m, 1H), 7.39-7.32 (m, 2H), 7.22 (d, J=4.8, 1.2 Hz, 1H), 7.11-7.03 (m, 6H), 6.95-6.87 (m, 4H), 6.55 (d, J=9.2 Hz, 1H), 4.79-4.69 (m, 3H), 4.14 (m, 1H), 4.05 (t, J=9.6 Hz, 1H), 3.78 (m, 1H), 3.47-3.35 (m, 3H), 3.18 (d, J=4.0 Hz, 1H), 3.04 (dd, J=14.0, 4.8 Hz, 1H), 2.72 (dd, J=14.0, 11.2 Hz, 1H); ¹³C NMR (100 MHz, CDCl₃) δ 168.94, 164.42, 161.98, 154.75 (dd, J=250.0, 10.6 Hz), 153.43 (dt, J=248.0, 10.4 Hz)), 152.83, 146.32 (dd, J=248.2, 10.4 Hz), 138.97 (d, J=10.6 Hz), 137.51, 137.21, 130.52 (d, J=9.5 Hz), 129.43 (2C), 128.72, 128.62 (2C), 127.17, 127.09, 126.74, 119.61 (d, J=22.0 Hz), 113.55, 111.58 (d, J=21.2 Hz), 107.69 (dd, J=27.3, 21.3 Hz), 106.10 (d, J=27.3 Hz), 72.24, 69.81, 53.71, 50.94, 48.21, 47.55, 35.40. HRMS (ESI) m/z: C₃₁H₂₈F₄N₃O₆S₂ (M+H)⁺ calcd, 678.1355; found, 678.1377.

(S)-3-(4-Acetylphenyl)-N-[(2S,3R)-1-Benzyl-2-hydroxy-3-[(2-thiophenylmethyl)[(2,4,5-trifluorophenyl)sulfonyl]amino]propyl]-2-oxo-oxazolidine-S-carboxamide (38f). ¹H NMR (400 MHz, CDCl₃) δ8.02 (d, J=9.2 Hz, 2H), 7.78-7.72 (m, 1H), 7.55 (d, J=9.2 Hz, 2H), 7.21 (dd, J=5.2, 1.2 Hz, 1H), 7.10-6.99 (m, 5H), 6.93-6.85 (m, 3H), 6.67 (d, J=8.8 Hz, 1H), 4.82-4.70 (m, 3H), 4.17 (m, 1H), 4.10 (t, J=10 Hz, 1H), 3.84 (m, 1H), 3.50-3.36 (m, 3H), 3.30 (d, J=4.0 Hz, 1H), 3.04 (dd, J=14.0, 4.4 Hz, 1H), 2.71 (dd, J=14.0, 10.8 Hz, 1H), 2.62 (s, 3H); ¹³C NMR (100 MHz, CDCl₃) [symbol] 196.97, 168.80, 154.50 (dd, J=252.3, 10.8 Hz), 153.4 (dt, J=255.6, 12.2 Hz), 152.68, 146.28 (dd, J=252.4, 10.5 Hz), 141.51, 137.53, 137.17, 133.25, 129.84 (2C), 129.45 (2C), 128.71, 128.62 (2C), 127.21, 127.16, 126.78, 119.76 (d, J=22.0 Hz), 117.61 (2C), 107.74 (dd, J=27.3, 21.2 Hz), 72.22, 69.81, 53.69, 51.09, 51.06, 48.0, 47.66, 35.43, 26.67. HRMS (ESI) m/z: C₃₃H₃₁F₃N₃O₇S₂ (M+H)⁺ calcd, 702.1555; found, 702.1561.

(5S)—N-[(1S,2R)-1-Benzyl-2-hydroxy-3-[[(3-methoxyphenyl)sulfonyl][(R)-(tetrahydro-2-furanyl)methyl]amino]propyl]-2-oxo-3-phenyloxazolidine-S-carboxamide (39a). ¹H NMR (400 MHz, CDCl₃) δ7.47-7.38 (m, 5H), 7.35 (d, J=7.6 Hz, 1H), 7.29 (d, J=1.8 Hz, 1H), 7.19 (t, J=7.2 Hz, 2H), 7.11 (m, 3H), 7.0 (t, J=7.6 Hz, 2H), 6.89 (t, J=7.6 Hz, 1H), 6.83 (d, J=10 Hz, 1H), 5.27 (br. s, 1H), 4.81 (dd, J=10.4, 6.0 Hz, 1H), 4.31-4.18 (m, 2H), 4.05 (t, J=9.6 Hz, 1H), 4.0 (t, J=10.0 Hz, 1H), 3.92-3.80 (m, 2H, overlapping signal), 3.88 (s, 3H, overlapping signal), 3.66 (dt, J=15.2, 2.4 Hz, 1H), 3.43 (dd, J=9.2, 6.0 Hz, 1H), 3.05 (dd, J=14.0, 4.4 Hz, 1H), 2.88 (dd, J=9.2, 4.8 Hz, 1H), 2.84 (dd, J=9.6, 4.8 Hz, 1H), 2.72 (dd, J=13.2, 10.0 Hz, 1H), 2.04 (m, 1H), 1.92 (m, 2H), 1.47 (m, 1H); ¹³C NMR (100 MHz, CDCl₃) [symbol] 168.53, 160.22, 153.13, 139.78, 137.65, 137.57, 130.53, 129.67 (2C), 129.28 (2C), 128.44 (2C), 126.55, 124.67, 119.45, 119.02, 118.44 (2C), 112.68, 80.49, 73.81, 69.87, 68.48, 56.48, 56.27, 55.91, 52.90, 48.29, 35.96, 29.22, 25.46. HRMS (ESI) m/z: C₃₂H₃₈N₃O₈S (M+H)⁺ calcd, 624.2379; found, 624.2390.

(5S)—N-[(1S,2R)-1-Benzyl-2-hydroxy-3-[[(3-methoxyphenyl)sulfonyl][(R)-(tetrahydro-2-furanyl)methyl]amino]propyl]-3-(3-fluorophenyl)-2-oxo-oxazolidine-S-carboxamide (39b). ¹H NMR (400 MHz, CDCl₃) δ7.42 (t, J=7.6 Hz, 1H), 7.38-7.32 (m, 2H), 7.28 (t, J=2.4 Hz, 1H), 7.11 (m, 4H), 7.0 (t, J=7.2 Hz, 2H), 6.91-6.86 (m, 2H), 6.84 (d, J=9.6 Hz, 1H), 5.28 (d, J=2.8 Hz, 1H), 4.82 (dd, J=10.0, 5.6 Hz, 1H), 4.31-4.19 (m, 2H), 4.02 (m, 2H), 3.92-3.80 (m, 2H, overlapping signal), 3.85 (s, 3H, overlapping signal), 3.69-3.64 (m, 2H), 3.39 (dd, J=9.2, 5.6 Hz, 1H), 3.05 (dd, J=14.0, 4.4 Hz, 1H), 2.89-2.82 (m, 2H), 2.71 (dd, J=14.0, 10.4 Hz, 1H), 2.04 (m, 1H), 1.92 (m, 2H), 1.46 (m, 1H); ¹³C NMR (100 MHz, CDCl₃) [symbol] 168.29, 163.21 (d, J=243.4 Hz), 160.22, 152.86, 139.75, 139.17 (d, J=10.6 Hz), 137.59, 130.54, 130.46 (d, J=9.1 Hz), 129.67 (2C), 128.40 (2C), 126.50, 119.43, 118.99, 113.48 (d, J=3.1 Hz), 112.71, 111.38 (d, J=20.4 Hz), 106.03 (d, J=27.3 Hz), 80.51, 73.85, 69.84, 68.49, 56.53, 56.31, 55.90, 52.87, 48.20, 35.92, 29.23, 25.46. HRMS (ESI) m/z: C₃₂H₃₇FN₃O₈S (M+H)⁺ calcd, 642.2285; found, 642.2289.

### Example 9

### Biological Evaluation of HIV-1 Protease Inhibitors

HIV-1 protease inhibitor activities were determined by fluorescence resonance energy transfer (FRET) method (Matayoshi, E. D.; Wang, G. T.; Krafft, G. A.; Erickson, J. Novel fluorogenic substrates for assaying retroviral proteases by resonance energy transfer. Science 1990, 247, 954-958.) Protease substrate (Arg-Glu(EDANS)-Ser-Gln-Asn-Tyr-Pro-Ile-Val-Gln-Lys(DABCYL)-Arg) was purchased from Molecular Probe. The energy transfer donor (EDANS) and acceptor (DABCYL) were labeled at its two ends respectively to perform FRET. Fluorescence measurements were carried out on PTI fluorescence spectrophotometer (Photon Technology International) at 30° C. Excitation and emission wavelengths were set at 340 nm and 490 nm, respectively. Each reaction was recorded for about 10 min. Wide type HIV-1 protease (Q7K) and its MDR mutants M1 (L10I, G48V, I54V, L63P, V82A), M2 (D30N, L63P, N88D), and M3 (L10I, L63P, A71V, G73S, I84V, L90M) were desalted through PD-10 columns (Amersham Biosciences). Sodium Acetate (20 mM, pH 5) was used as elution buffer. Apparent protease concentrations were around 50 nM estimated by UV spectrophotometer (Shimadzu) at 280 nm. All inhibitors were dissolved in DMSO and diluted to appropriate concentrations. Protease (2 μL) and inhibitor (2 μL) or DMSO were mixed and incubated for 20-30 min at room temperature before initialing substrate cleavage reaction. Throughout this work, 150 μL of 1 μM substrate was used. Substrate buffer is composite of 0.1 M sodium acetate, 1 M sodium chloride, 1 mM ethylenediaminetetraacetic acid (EDTA), 1 mM dithiothreitol (DTT), 2% dimethylsulfoxide (DMSO) and 1 mg/mL bovine serum albumin (BSA) with an adjusted pH 4.7. Inhibitor binding dissociation constant (Ki) was obtained by nonlinear regression fitting (GraFit 5, Erithacus software) to the plot of initial velocity as a function of inhibitor concentrations based on Morrison equation. (Greco, W. R.; Hakala, M. T. Evaluation of methods for estimating the dissociation constant of tight binding enzyme inhibitors. J. Biol. Chem. 1979, 254, 12104-12109.) The initial velocities were derived from the linear range of reaction curves.

### Example 10

### Synthesis of Protease Inhibitors Containing a Hydroxyethylamine (HEA) Core

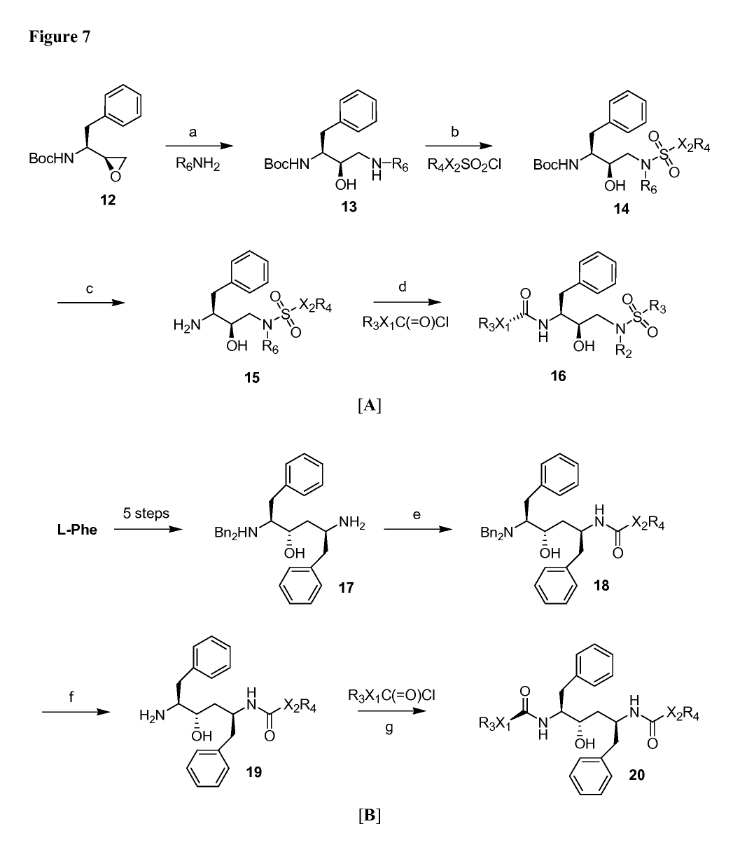

The designed inhibitors with a hydroxyethylamine (HEA) core isostere can be synthesized in four steps starting with commercially available chiral epoxide (1S,2S enantiomer) 12. Ring opening of epoxide 12 with various primary and secondary amines provided compounds 13. Reaction of 13 with various sulfonyl chlorides gave compounds 14. After deprotecting the Boc group, the resulting amines 15 were coupled with either (R) or (S) isomer of activated carboxylic acids to provide the designed inhibitors 16 (FIG. 7A).

### Example 11

### Synthesis of Protease Inhibitors Containing Cyclic Carbamates

### HE Series

The synthesis of protease inhibitors containing hydroxyethylene (HE) isostere starts with the synthesis of the core 17, which was obtained from L-phenylalanine in 5 steps. After coupling of R₄X₂CO₂H to 17, the dibenzyl protection was removed and the free amine 19 was coupled to the an activated acid to provide inhibitors 20 (FIG. 7B).

### Example 12

### Synthesis of Protease Inhibitors Containing Cyclic Carbamates

### Aza-HEA Series

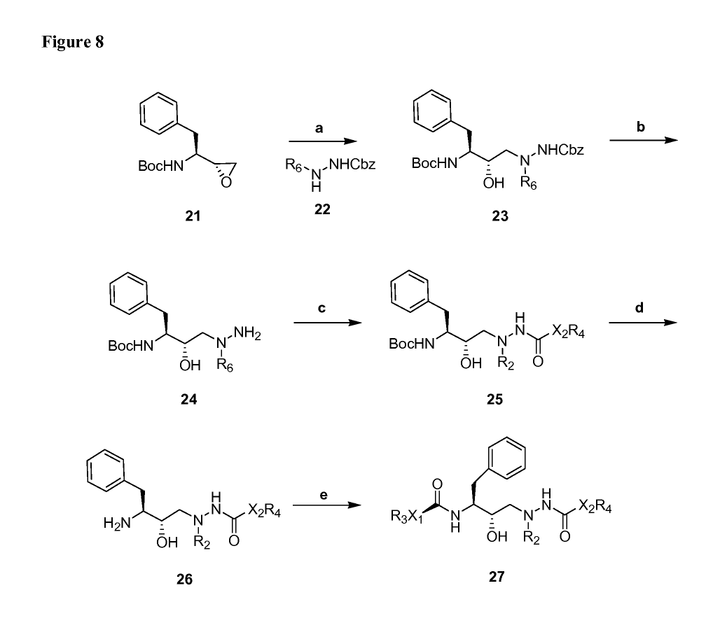

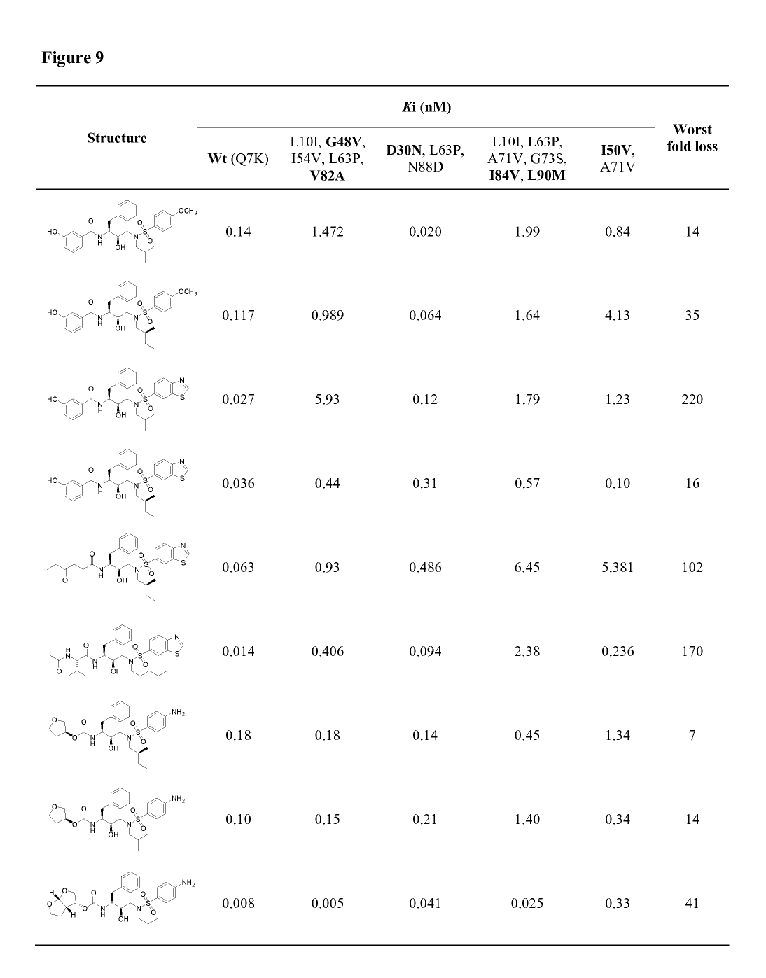

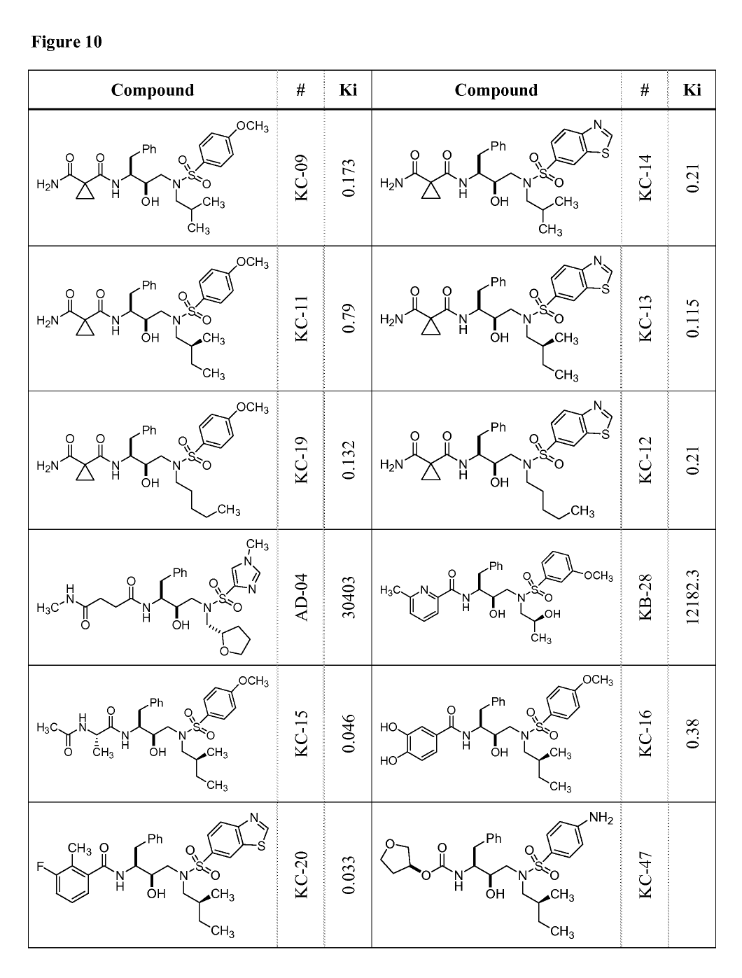

The synthesis of protease inhibitors containing aza-hydroxyethylamine (Aza-HEA) isostere is outlined in FIG. 8. The ring opening of chiral epoxide (1S,2R enantiomer) 21 with CBz protected hydrazine derivative 22 provided compound 23. Deprotection of CBz followed by the coupling with R₄X₂CO₂H gave compounds 24. Removal of the Boc protection and coupling with R₃X₁CO₂H provided the desired inhibitors 27.

### INCORPORATION BY REFERENCE

The contents of all cited references (including literature references, issued patents, published patent applications and GenBank Accession numbers as cited throughout this application) are hereby expressly incorporated by reference. When definitions of terms in documents that are incorporated by reference herein conflict with those used herein, the definitions used herein govern.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound, or a pharmaceutically acceptable salt or prodrug thereof, of formula I: 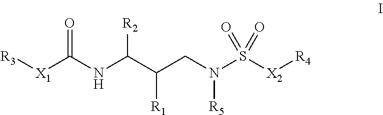
wherein, independently for each occurrence,
X₁ is absent, —O—, —S—, —NR— or 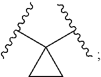
X₂ is absent, —O—, —S—, —NR— or 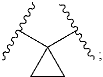
R₁ is —OH, —SH or —NHR;
R is hydrogen, alkyl, aralkyl, heteroaralkyl or acyl;
R₂ is hydrogen, alkyl, cycloalkyl, heterocyclyl, aryl, heteroaryl, (heterocyclyl)alkyl, aralkyl or heteroaralkyl;
R₃ is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl;
R₄ is alkyl, cycloalkyl, heterocyclyl, aryl, heteroaryl, (heterocyclyl)alkyl, aralkyl or heteroaralkyl;
R₅ is hydrogen, alkyl, (cycloalkyl)alkyl, amino, (amino)alkyl, amido, (amido)alkyl, (keto)alkyl, cycloalkyl, heterocyclyl, aryl, heteroaryl, (heterocyclyl)alkyl, aralkyl or heteroaralkyl; and
the stereochemical configuration at any stereocenter is R, S, or a mixture of these configurations;
provided that when X₁ is absent; R₃ is not 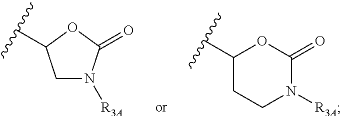
wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

2. The compound of claim 1, wherein X₁ is absent.

3-14. (canceled)

15. The compound of claim 1, wherein the compound is a compound of formula II: 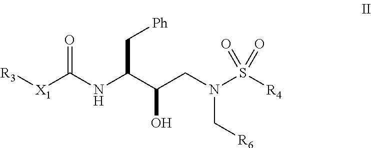
wherein, independently for each occurrence,
X₁ is absent, —O— or 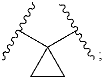
R₃ is alkyl, alkenyl, amino, (amino)alkyl, amido, (amido)alkyl, cycloalkyl, heterocyclyl, aryl, heteroaryl, (heterocyclyl)alkyl, aralkyl or heteroaralkyl;
R₄ is aryl, heteroaryl, aralkyl or heteroaralkyl; and
R₆ is alkyl, cycloalkyl, heterocyclyl, aryl, heteroaryl, aralkyl or heteroaralkyl;
provided that when X₁ is absent; R₃ is not 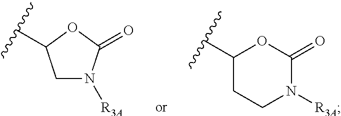
wherein R_{3A} is hydrogen, alkyl, alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

16. The compound of claim 15, wherein X₁ is absent.

17. (canceled)

18. The compound of claim 15, wherein R₃ is alkenyl, amino, (amino)alkyl, amido, (amido)alkyl, (keto)alkyl, cycloalkyl, cycloalkenyl, heterocyclyl, aryl, heteroaryl, (heterocyclyl)alkyl, aralkyl or heteroaralkyl.

19. (canceled)

20. The compound of claim 15, wherein R₄ is alkyl, aryl or heteroaryl.

21. The compound of claim 15, wherein R₆ is alkyl, (cycloalkyl)alkyl, amino, (amino)alkyl, amido, (amido)alkyl, (heterocyclyl)alkyl, aralkyl or heteroaralkyl.

22. (canceled)

23. (canceled)

24. The compound of claim 15, wherein R₄ is

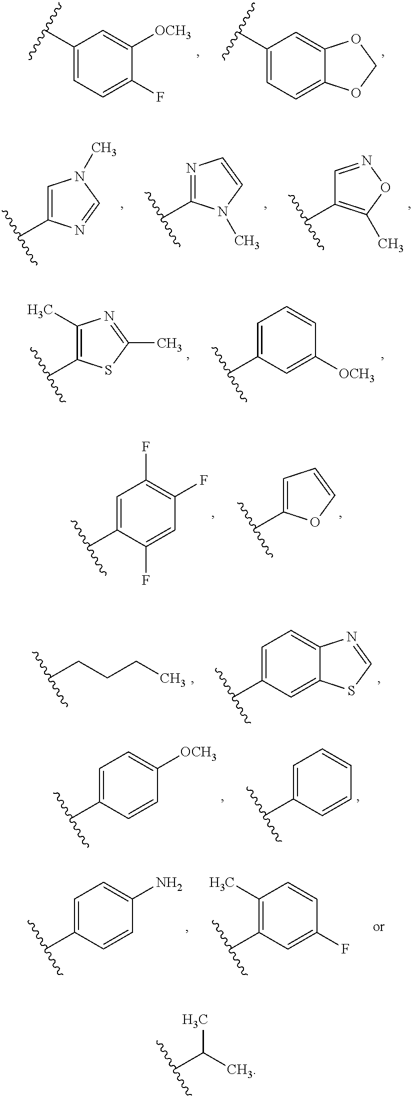

25. The compound of claim 15, wherein R₆ is

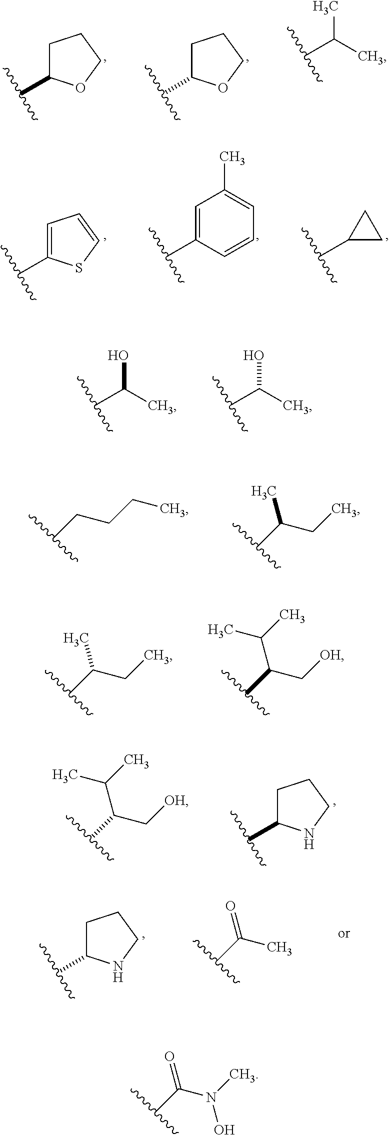

26. The compound of claim 15, wherein X₁ is absent; and R₃ is

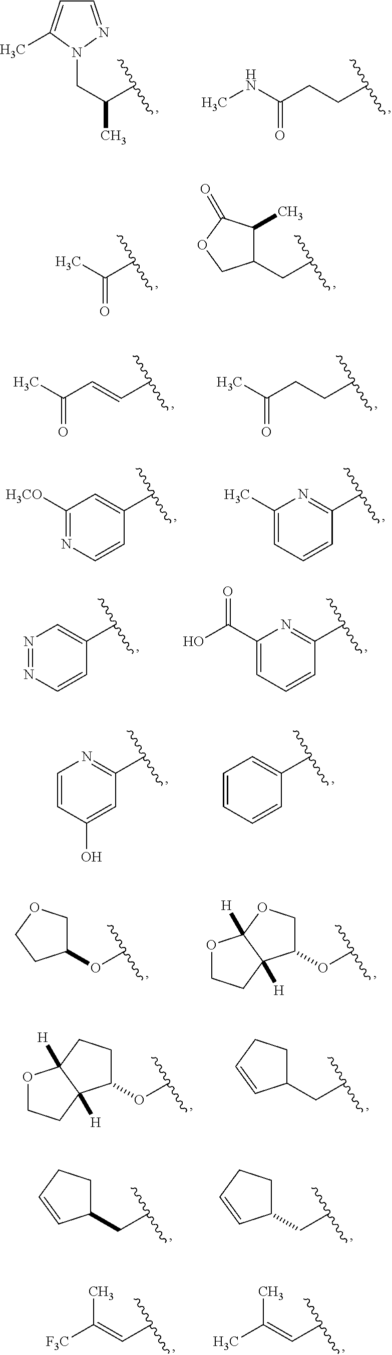

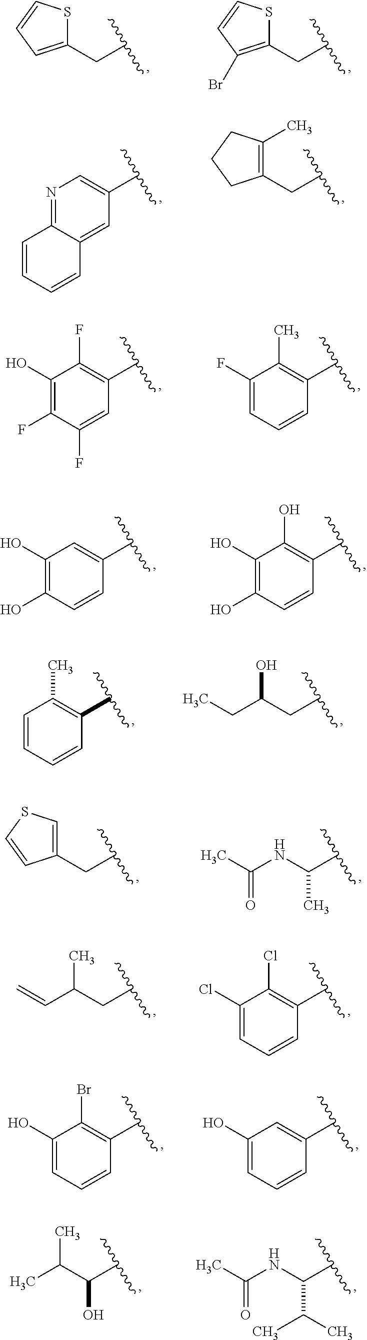

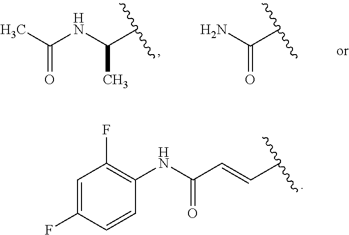

27-30. (canceled)

31. The compound of claim 1, wherein the compound is selected from the group consisting of

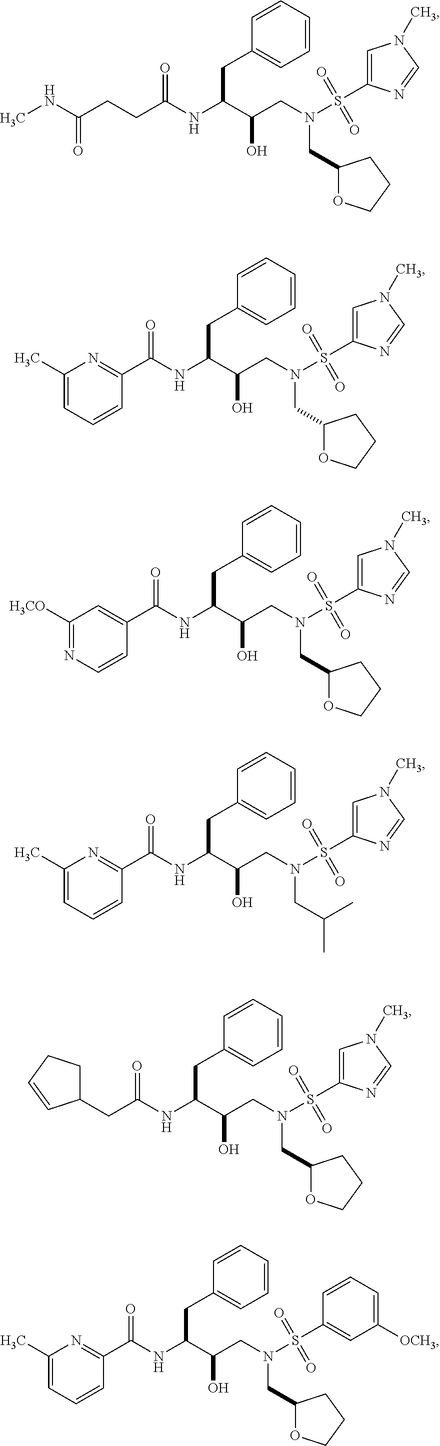

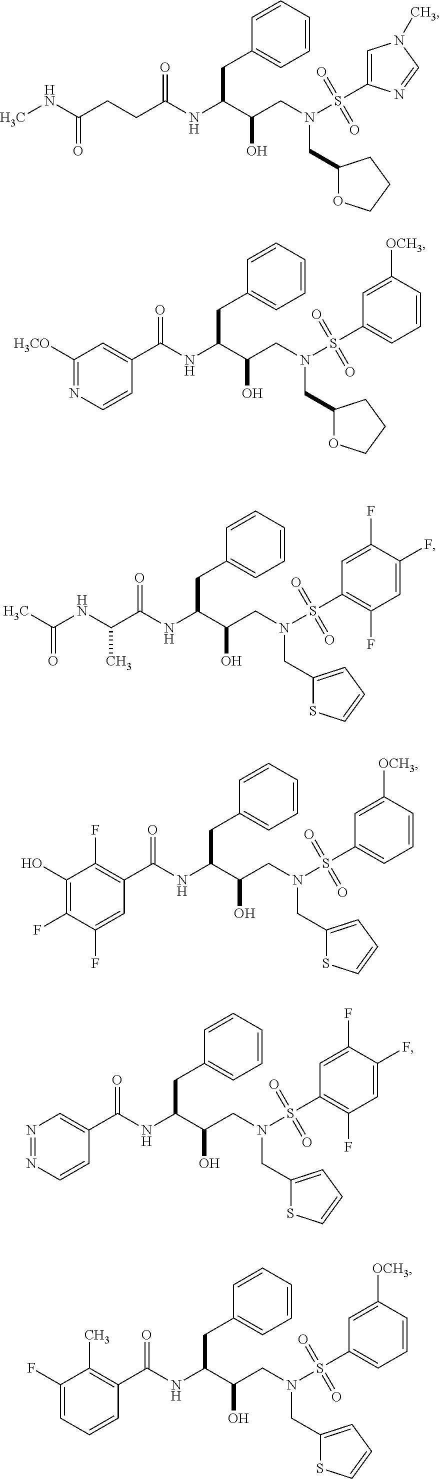

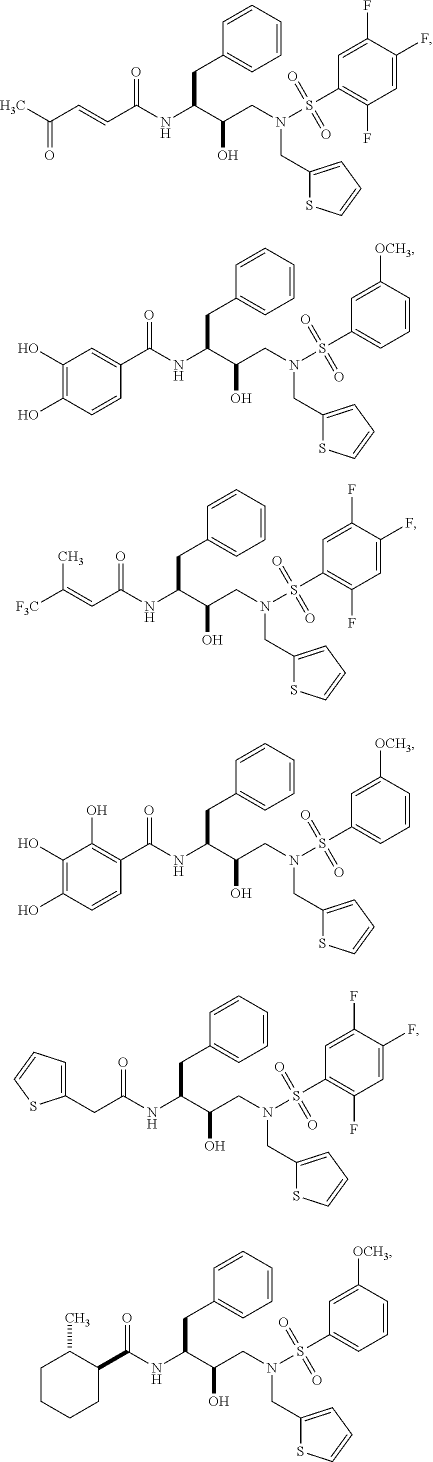

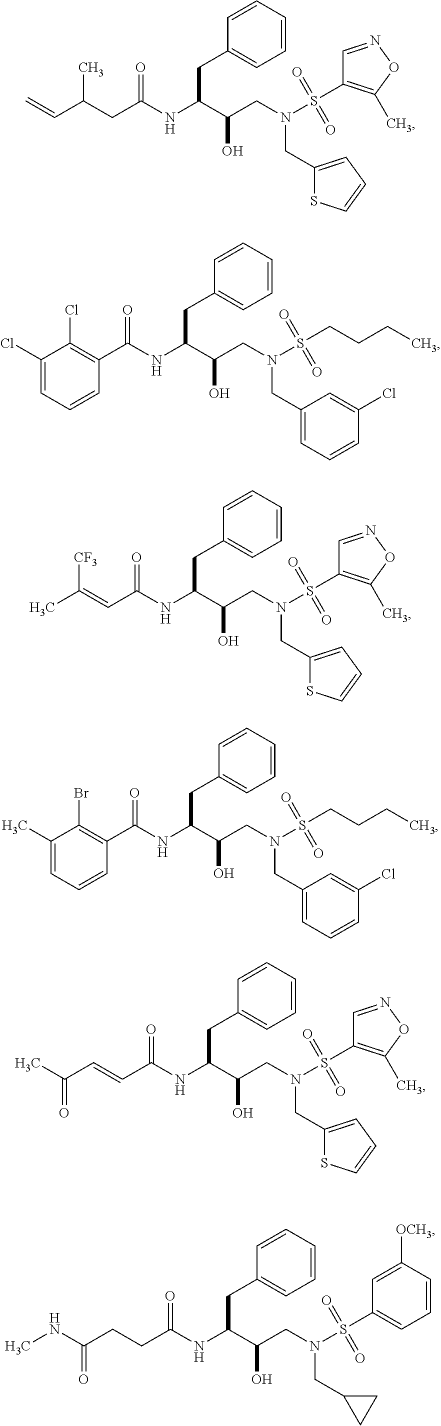

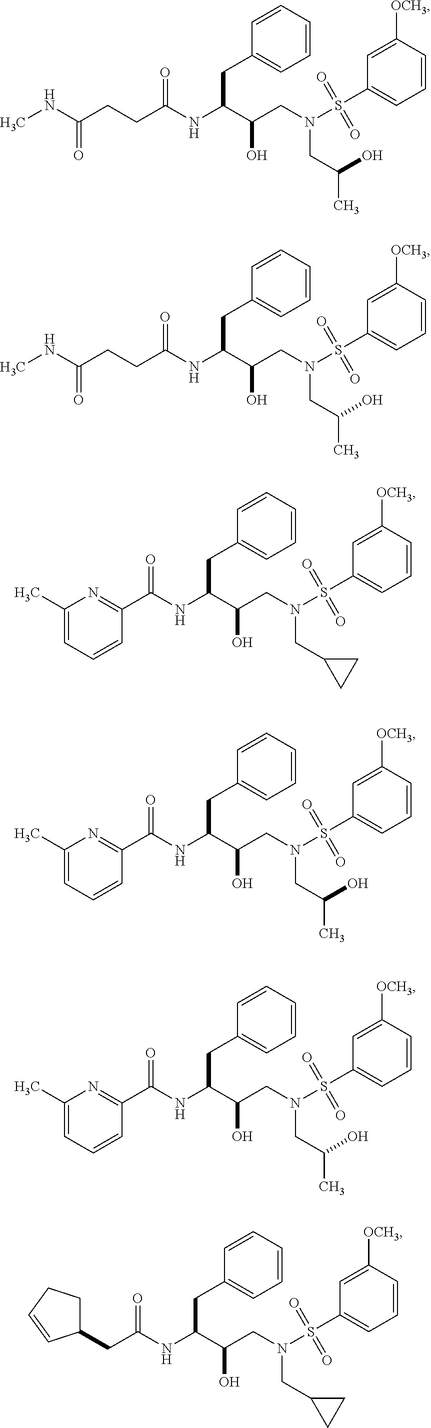

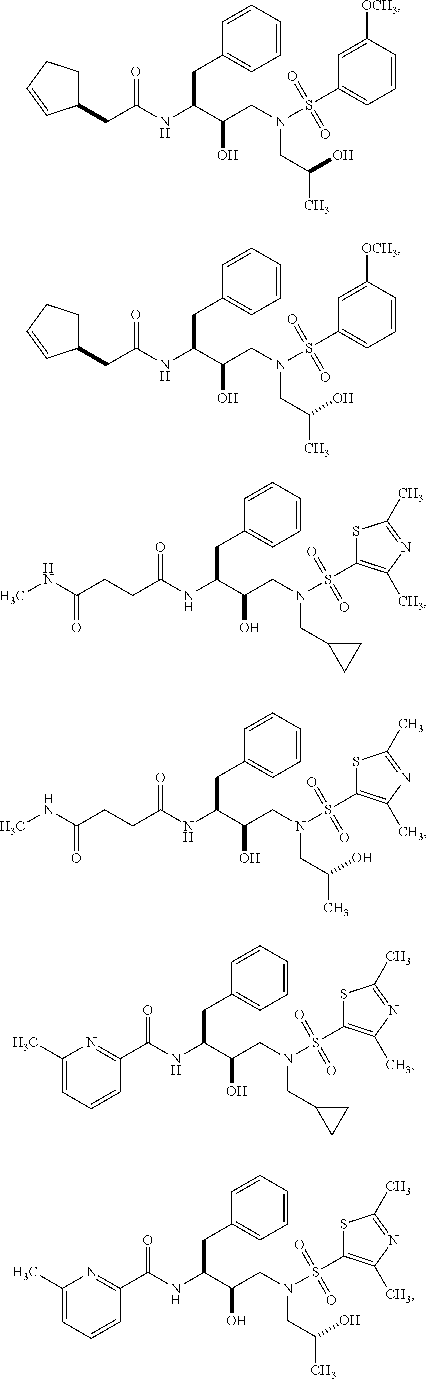

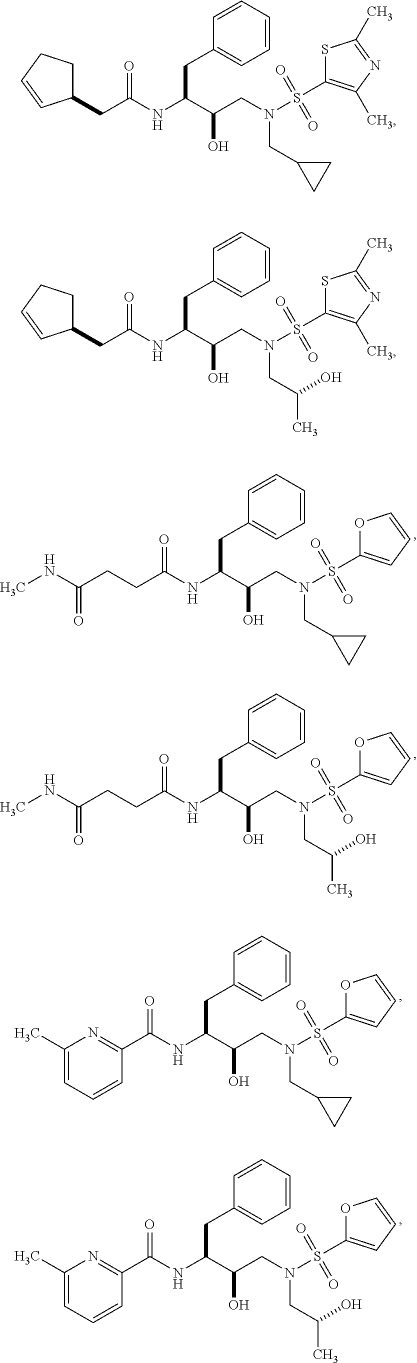

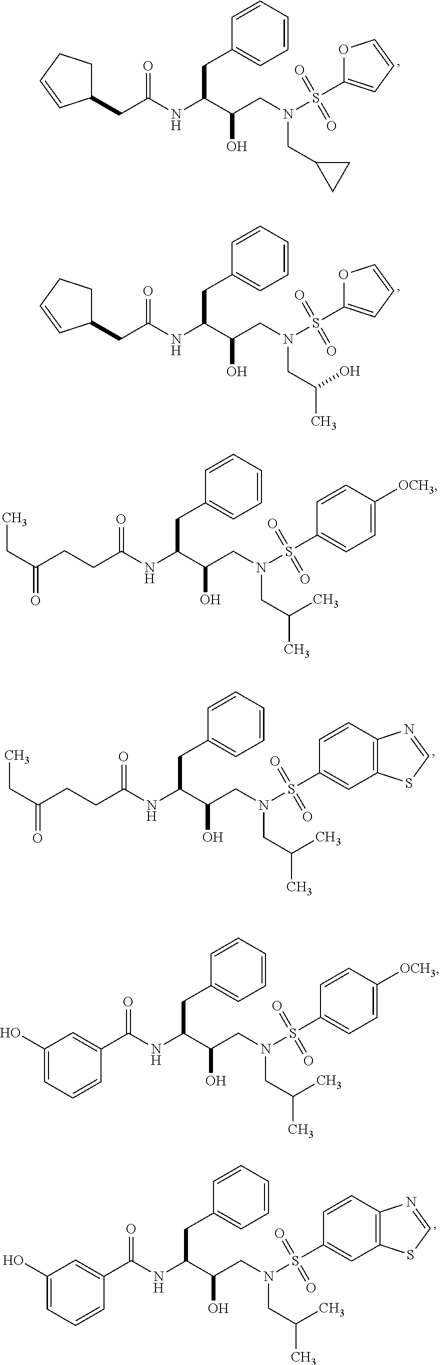

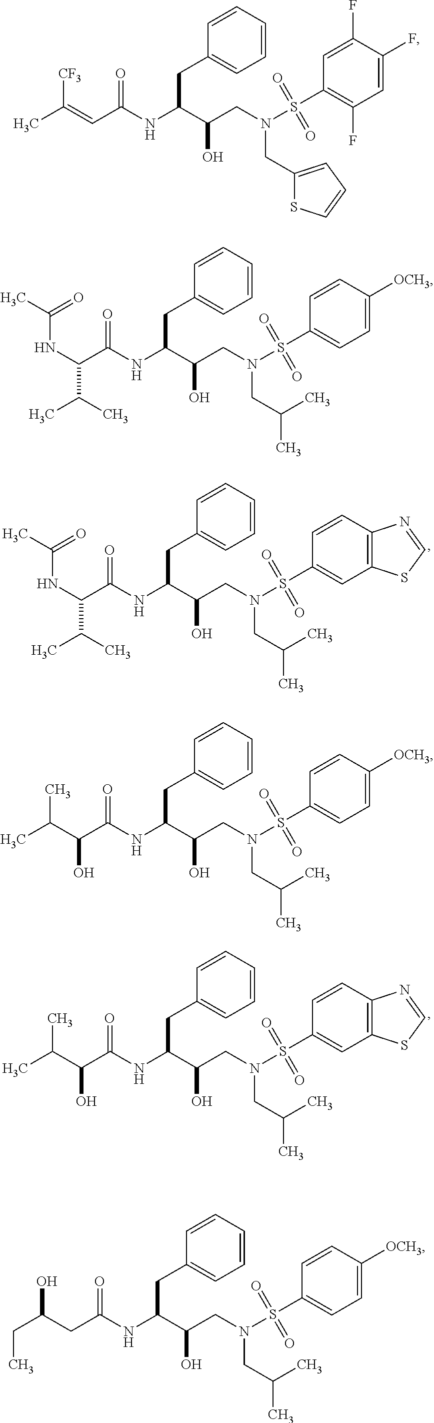

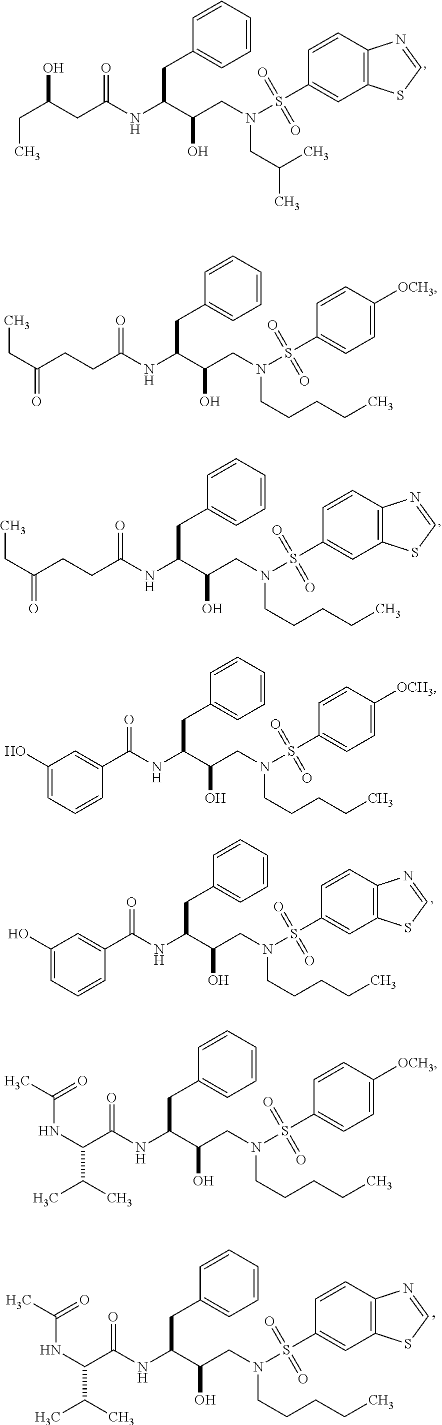

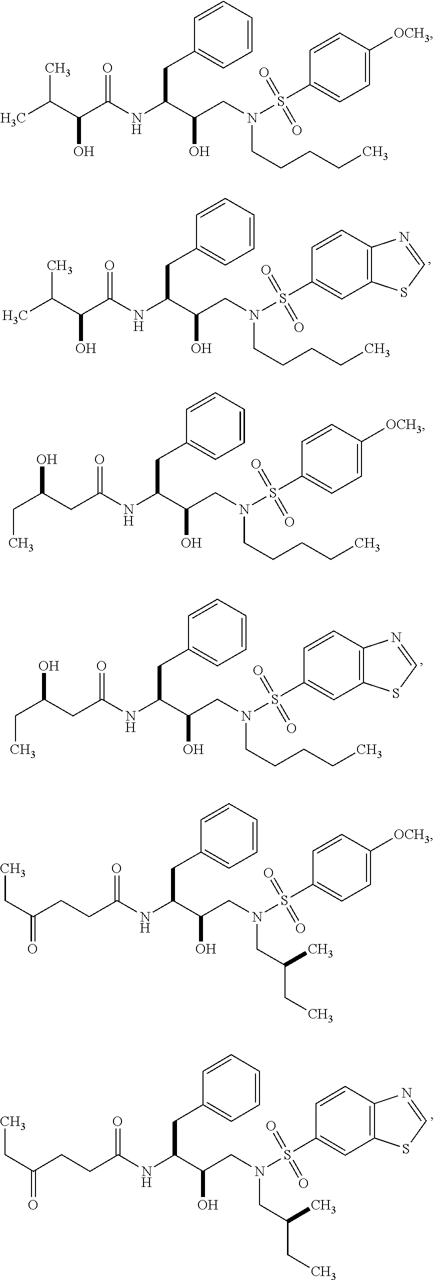

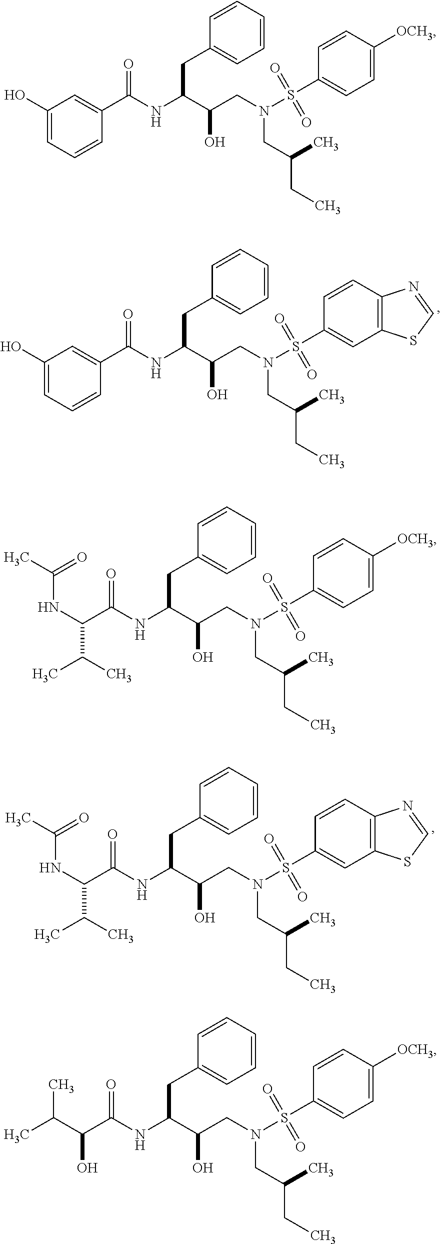

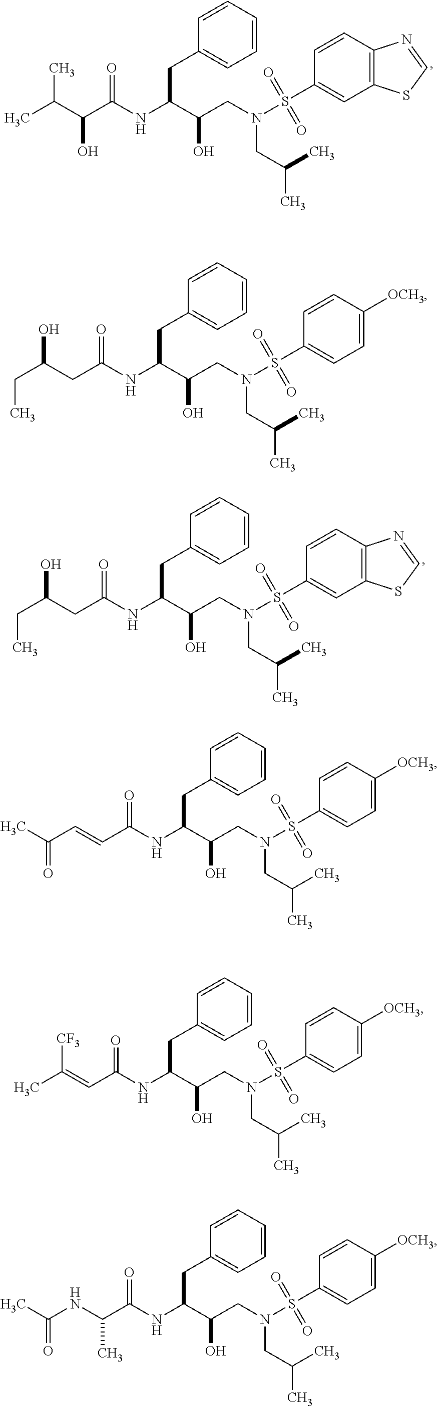

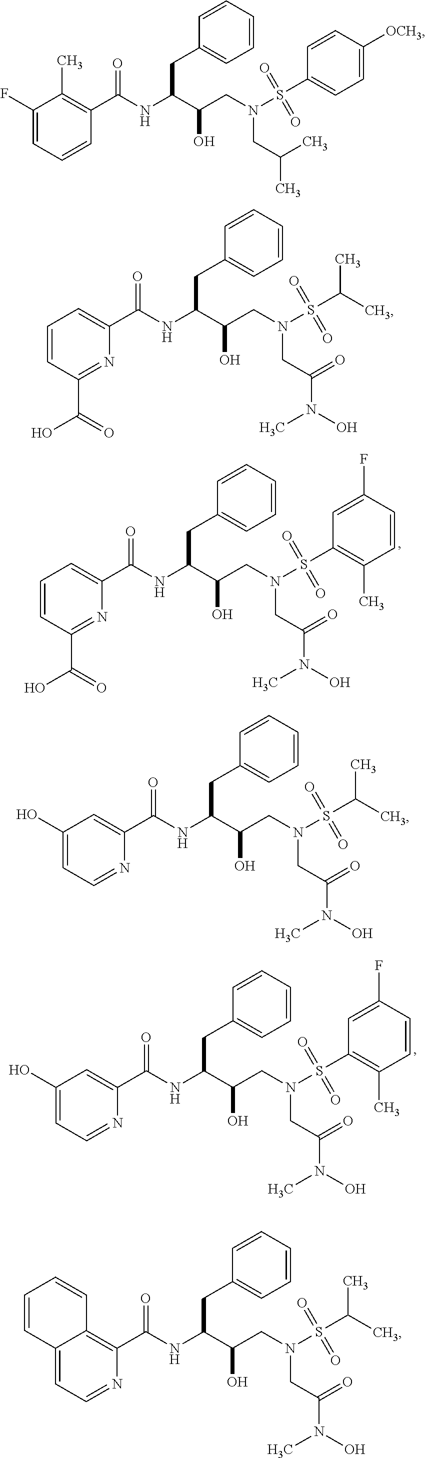

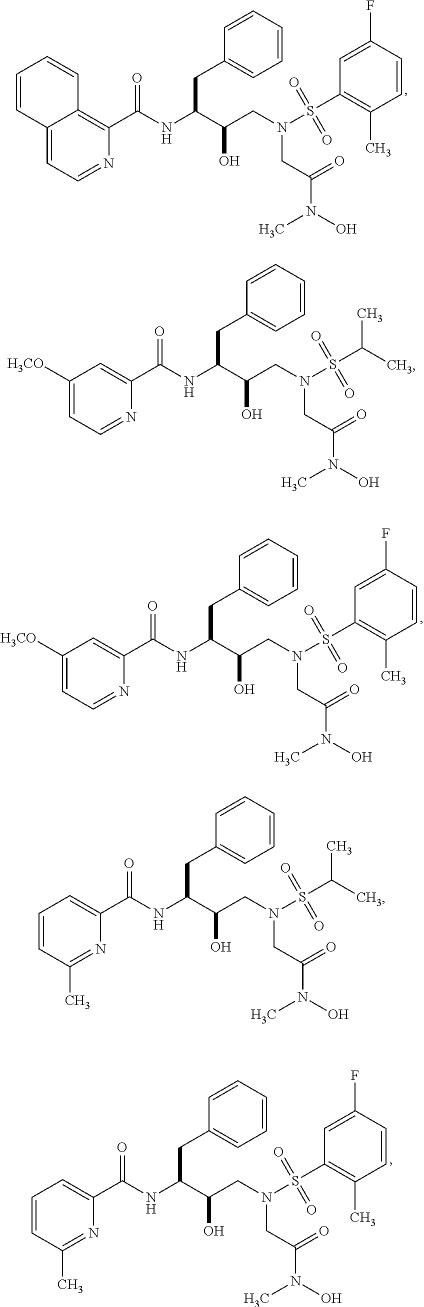

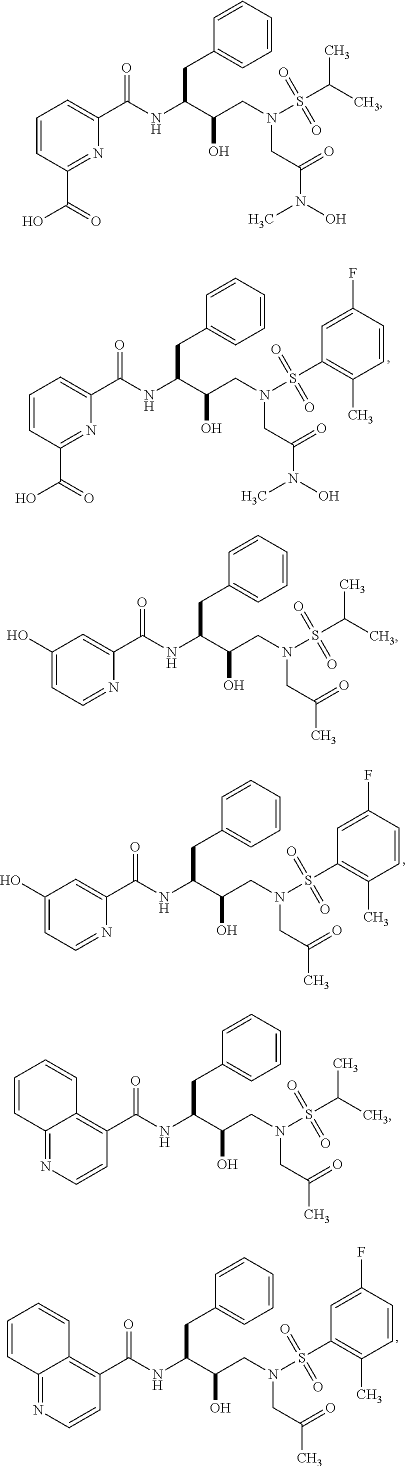

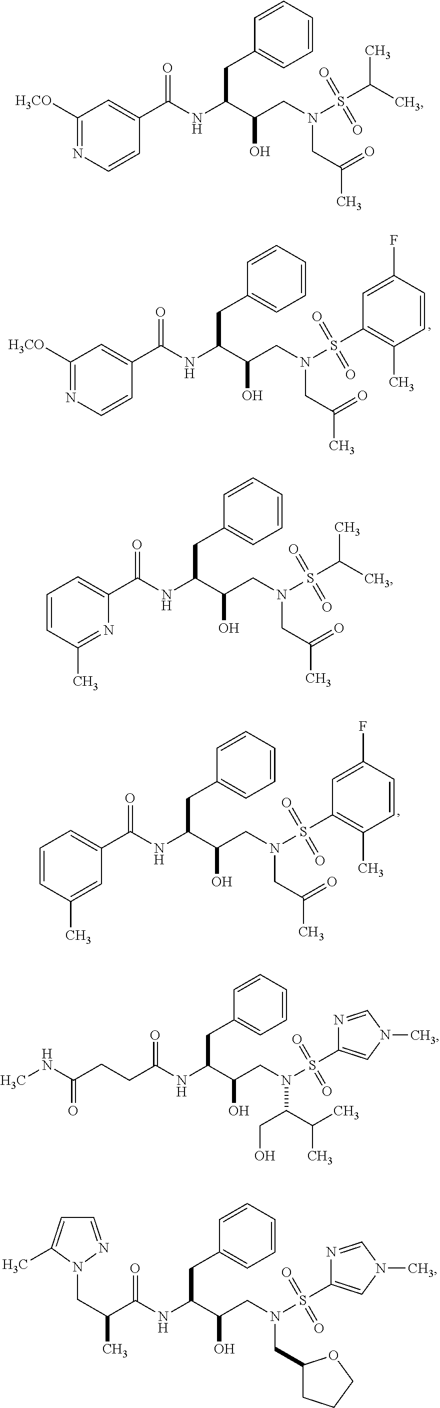

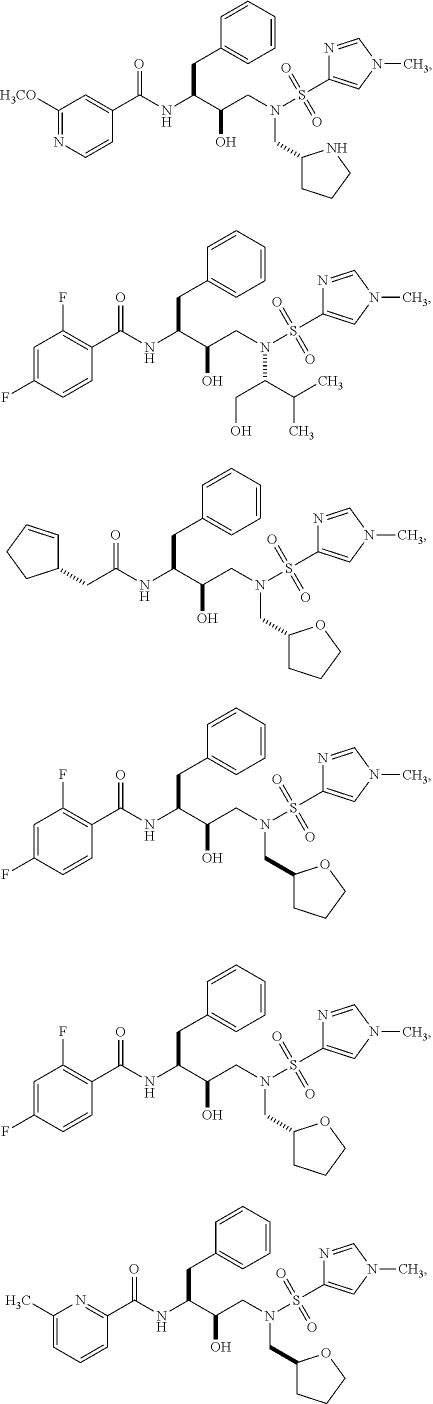

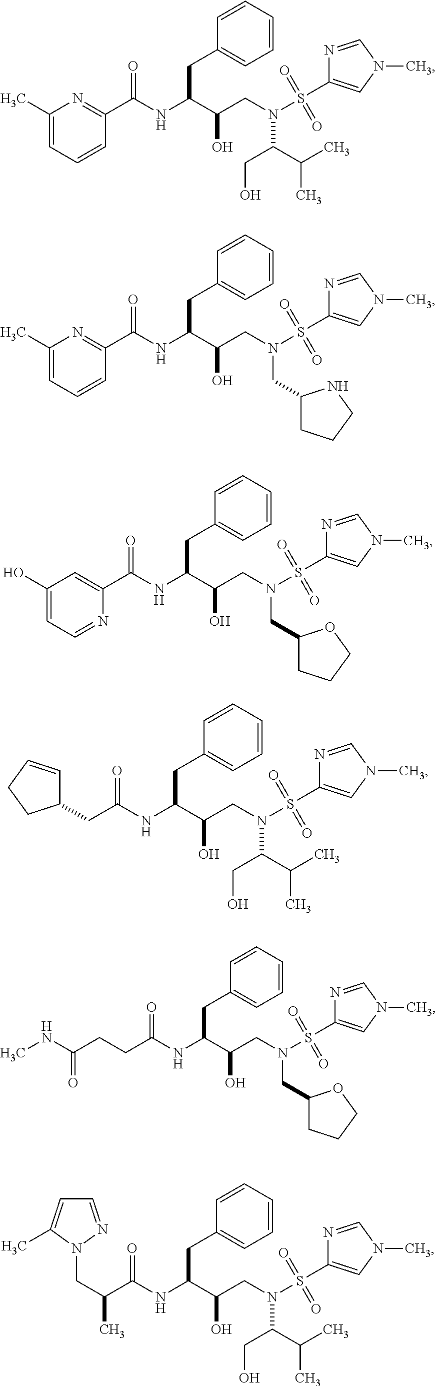

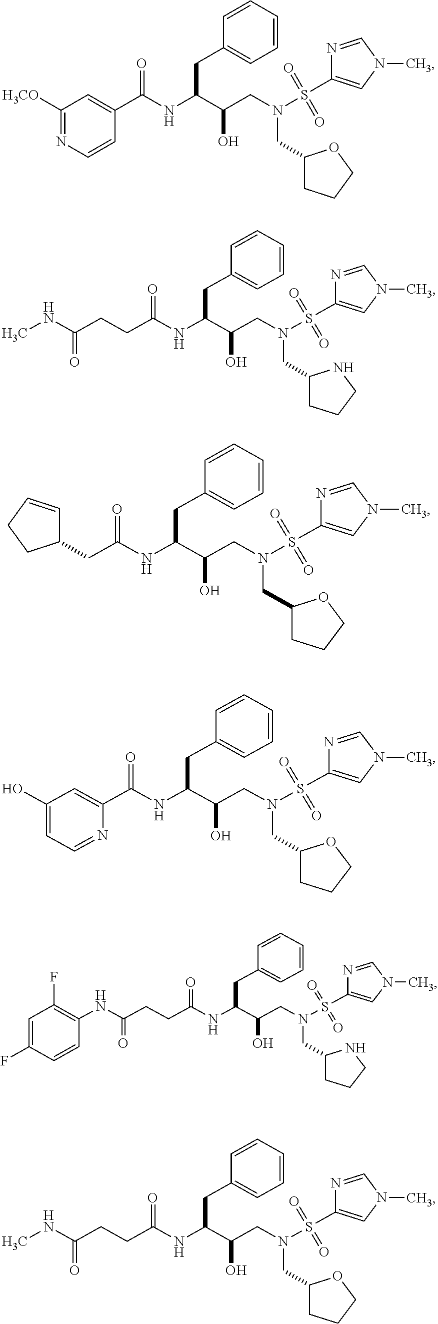

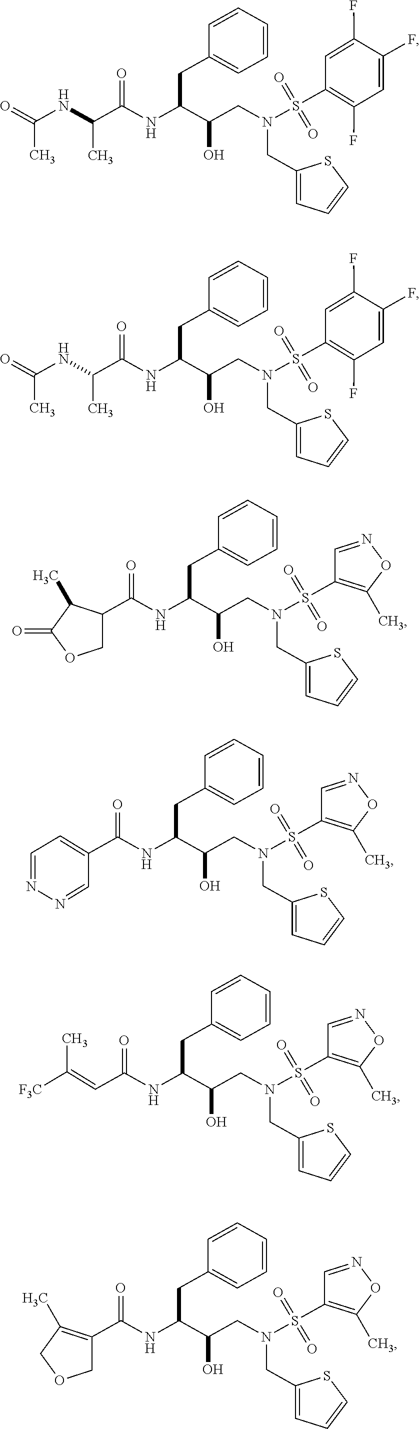

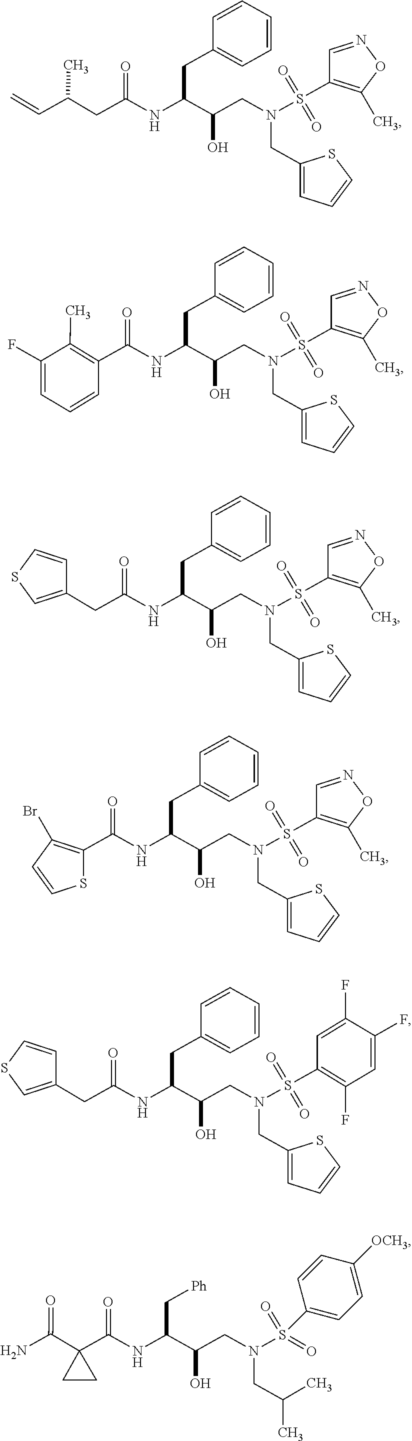

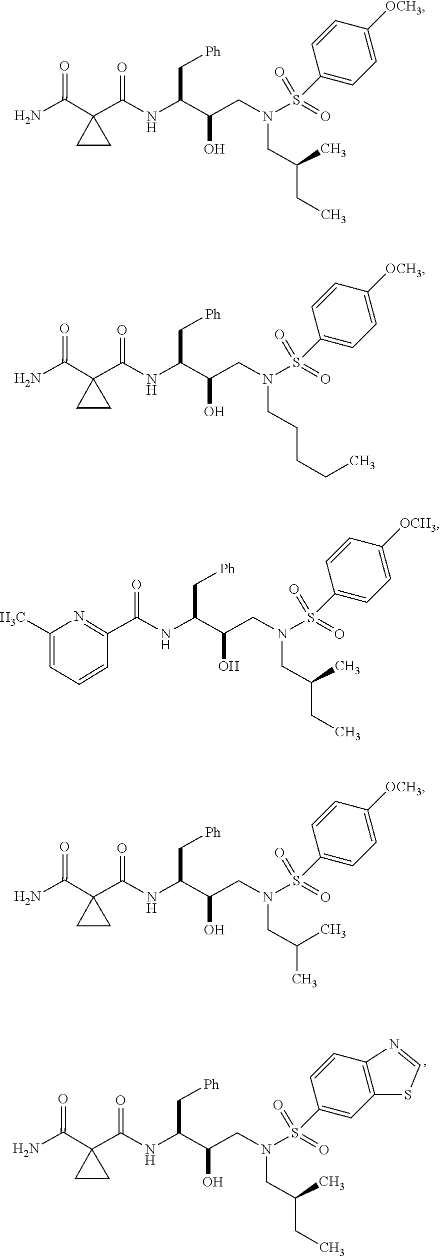

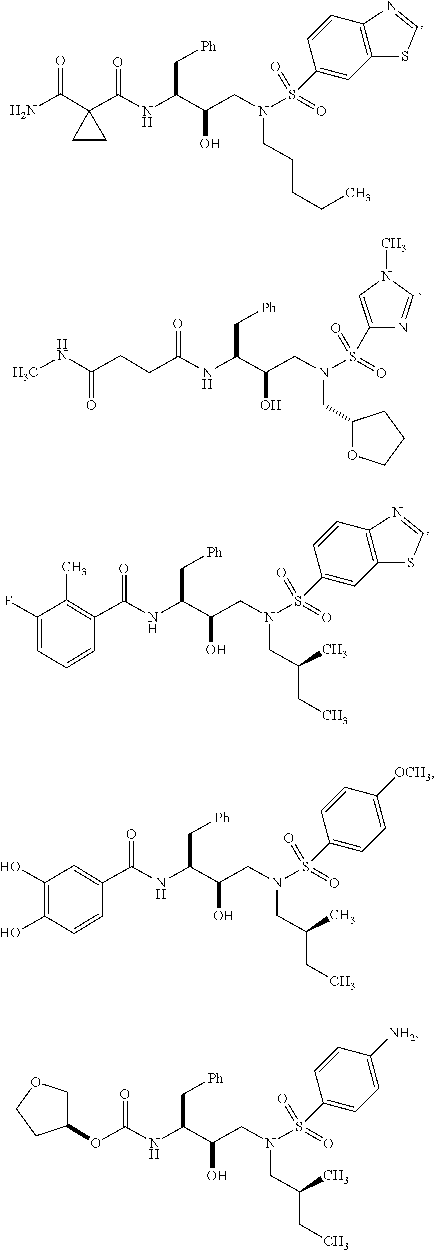

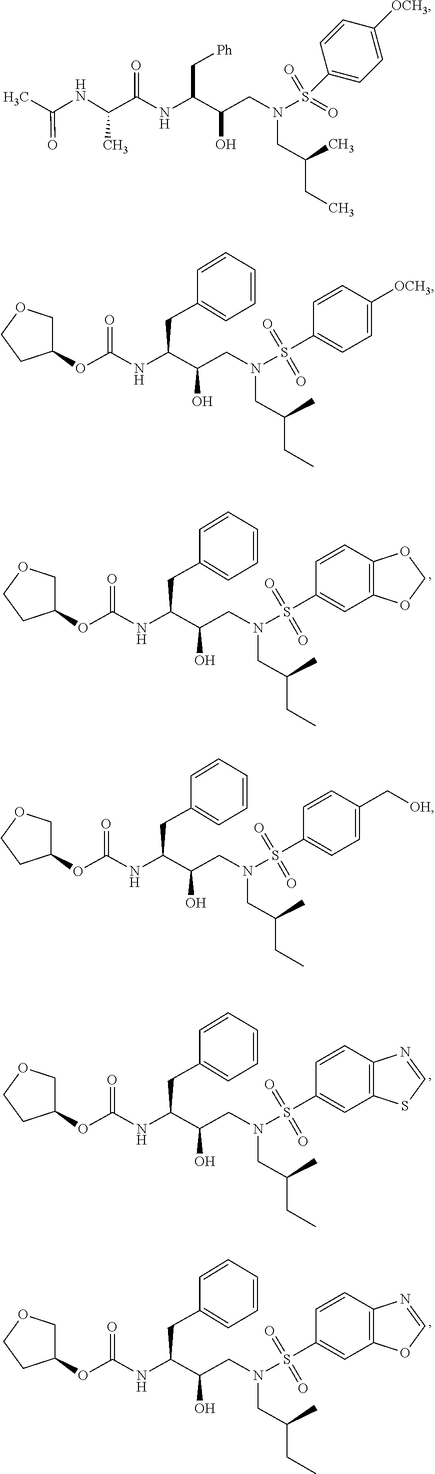

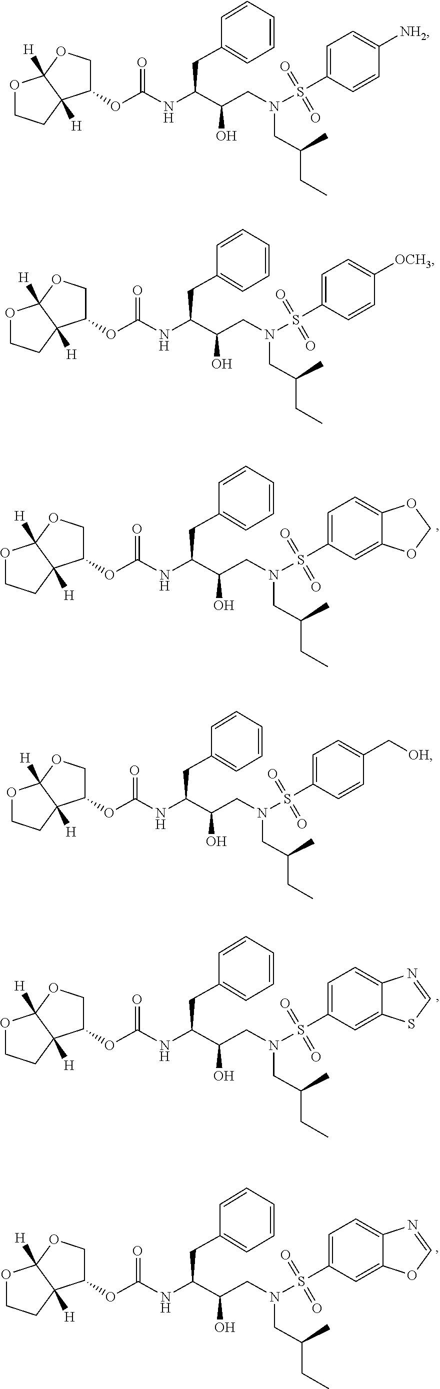

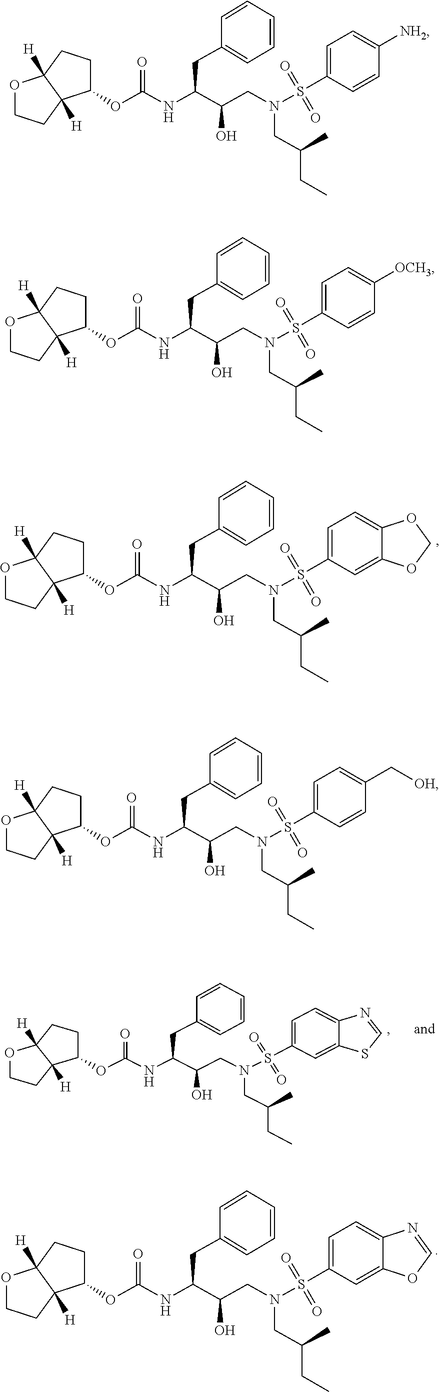

32-109. (canceled)

110. A compound, or a pharmaceutically acceptable salt or prodrug thereof, of formula XI: 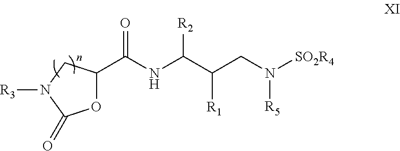
wherein, independently for each occurrence,
n is 1 or 2;
R₁ is —OH, —SH, or —NHR;
R is hydrogen, alkyl, aralkyl, heteroaralkyl or acyl;
R₂ is hydrogen, alkyl, cycloalkyl, heterocyclyl, aryl, heteroaryl, (heterocyclyl)alkyl, aralkyl or heteroaralkyl;
R₃ is hydrogen, alkyl, alkenyl, (amino)alkyl, (amido)alkyl, (keto)alkyl, cycloalkyl, cycloalkenyl, heterocyclyl, aryl, heteroaryl, (heterocyclyl)alkyl, aralkyl or heteroaralkyl;
R₄ is alkyl, cycloalkyl, heterocyclyl, aryl, heteroaryl, (heterocyclyl)alkyl, aralkyl or heteroaralkyl;
R₅ is hydrogen, alkyl, cycloalkyl, heterocyclyl, aryl, heteroaryl, (heterocyclyl)alkyl, aralkyl or heteroaralkyl; and
the stereochemical configuration at any undefined stereocenter is R, S, or a mixture of these configurations.

111-126. (canceled)

127. The compound of claim 110, wherein the compound is a compound of formula XII: 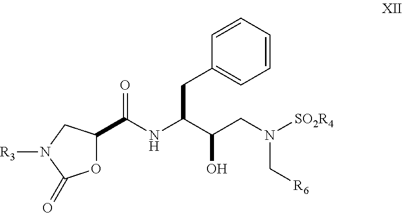
wherein, independently for each occurrence,
R₃ is alkyl, (amino)alkyl, (amido)alkyl, cycloalkyl, heterocyclyl, aryl, heteroaryl, (heterocyclyl)alkyl, aralkyl or heteroaralkyl;
R₄ is aryl, heteroaryl, aralkyl or heteroaralkyl; and
R₆ is alkyl, cycloalkyl, heterocyclyl, aryl, heteroaryl, aralkyl or heteroaralkyl;
or a pharmaceutically acceptable salt thereof.

128. The compound of claim 127, wherein R₃ is aryl or heteroaryl.

129. (canceled)

130. The compound of claim 127, wherein R₄ is aryl or heteroaryl.

131. (canceled)

132. The compound of claim 127, wherein R₆ is alkyl, heterocyclyl, aryl or heteroaryl.

133-141. (canceled)

142. The compound of claim 127, wherein R₃ is

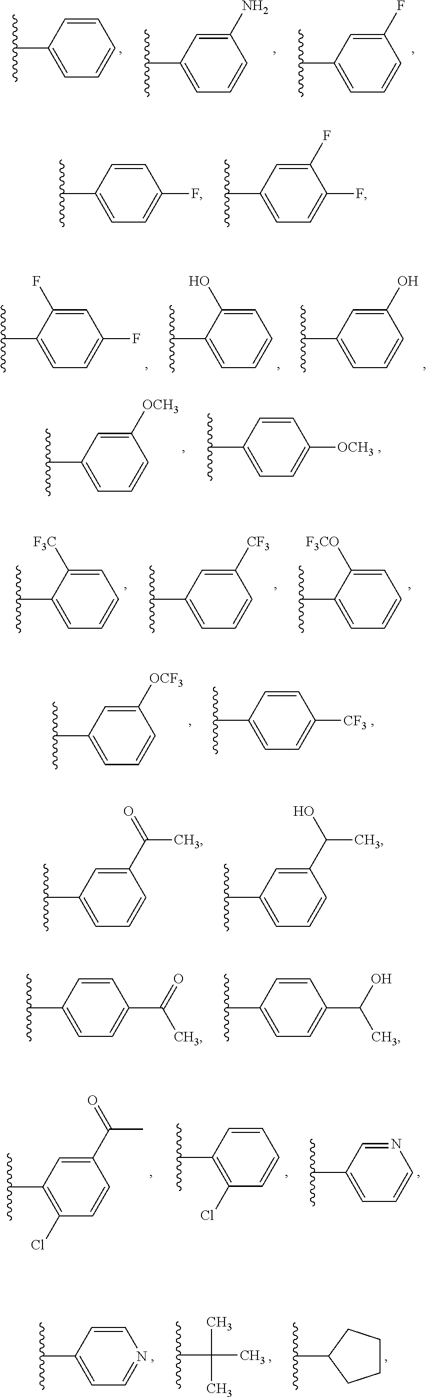

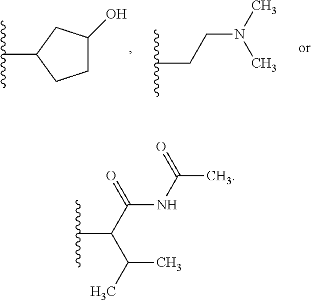

143. The compound of claim 127, wherein R₄ is 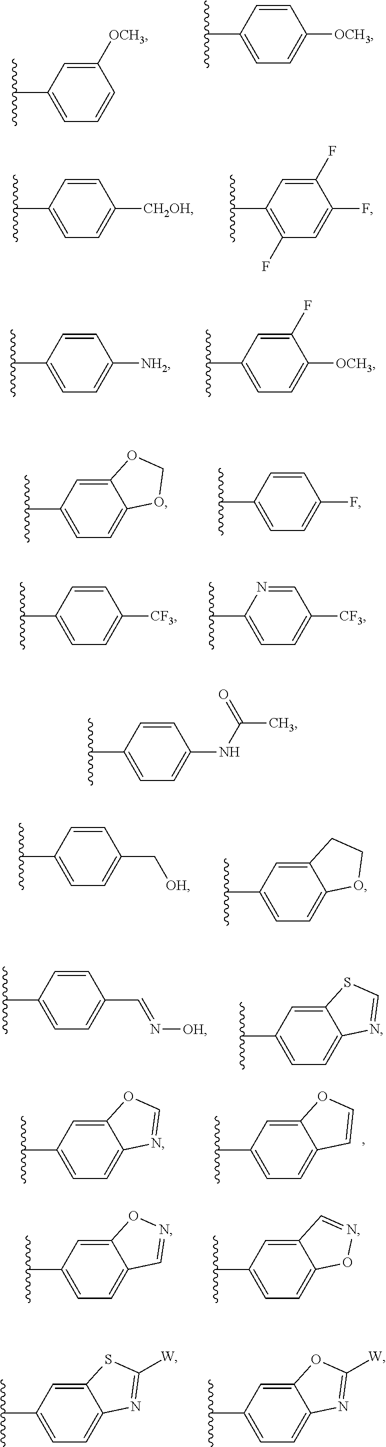 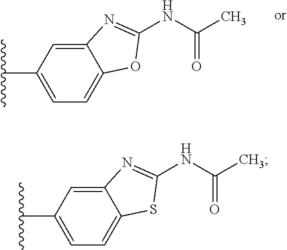
wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

144. The compound of claim 127, wherein R₆ is

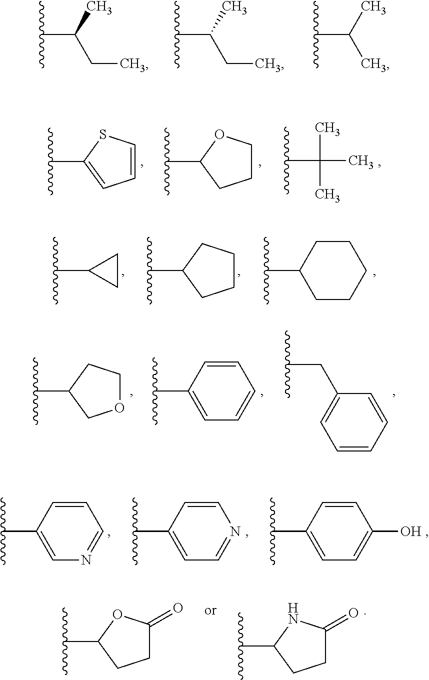

145-148. (canceled)

149. The compound of claim 110, wherein the compound is selected from the group consisting of: 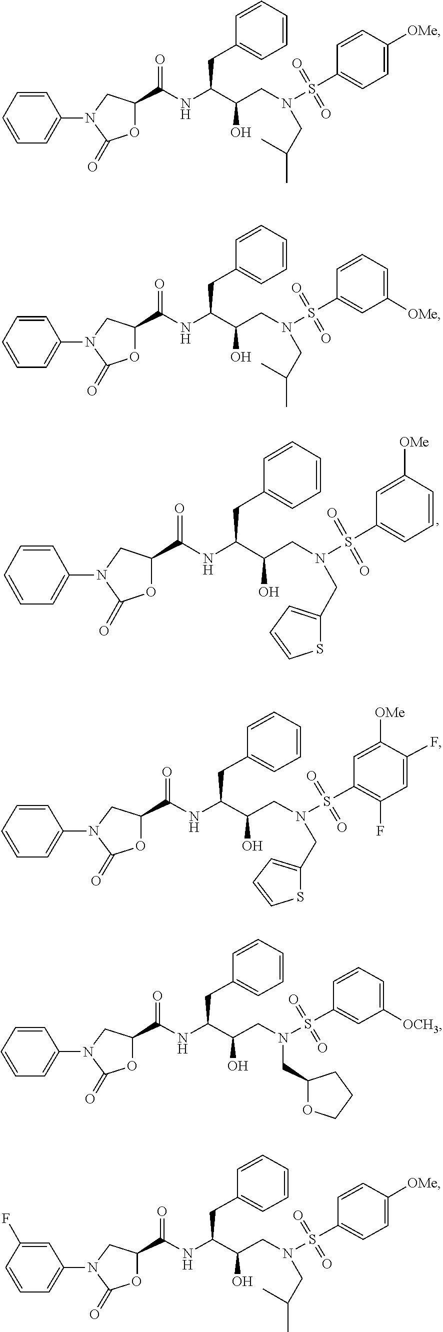 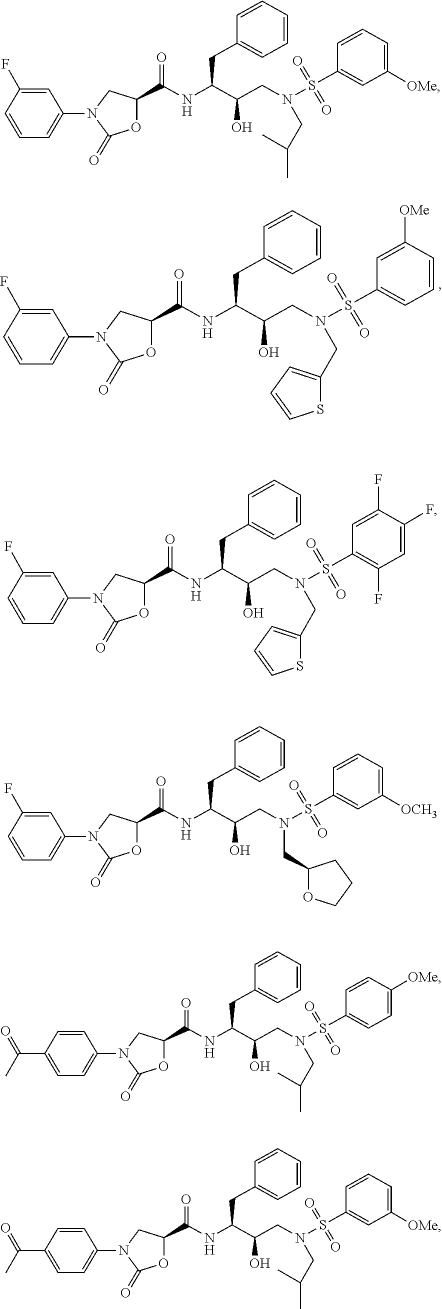 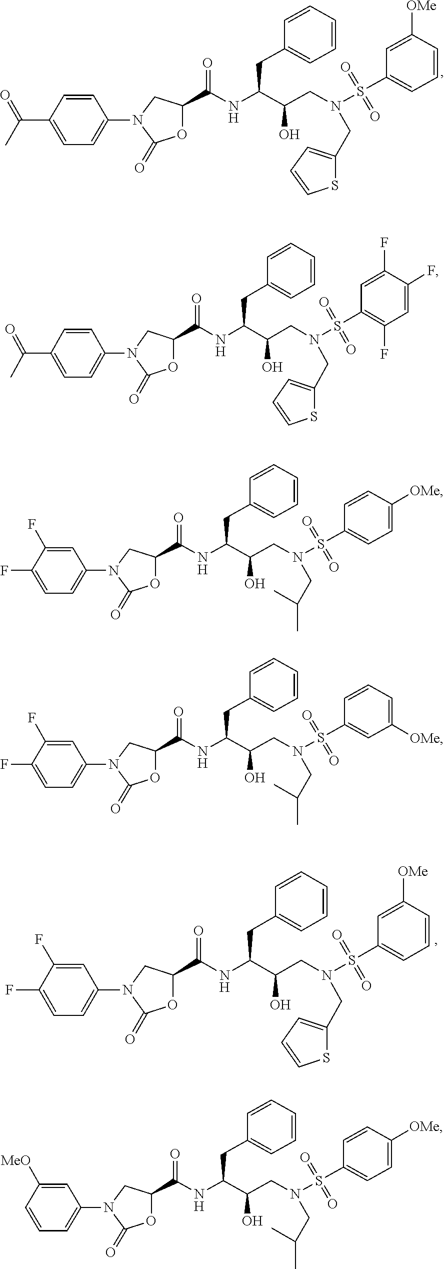 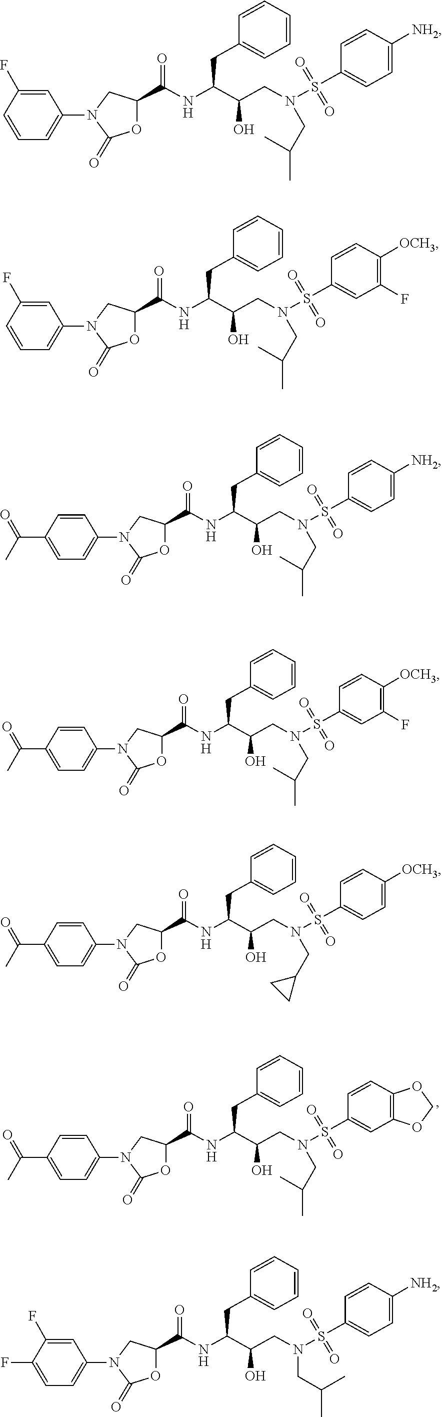 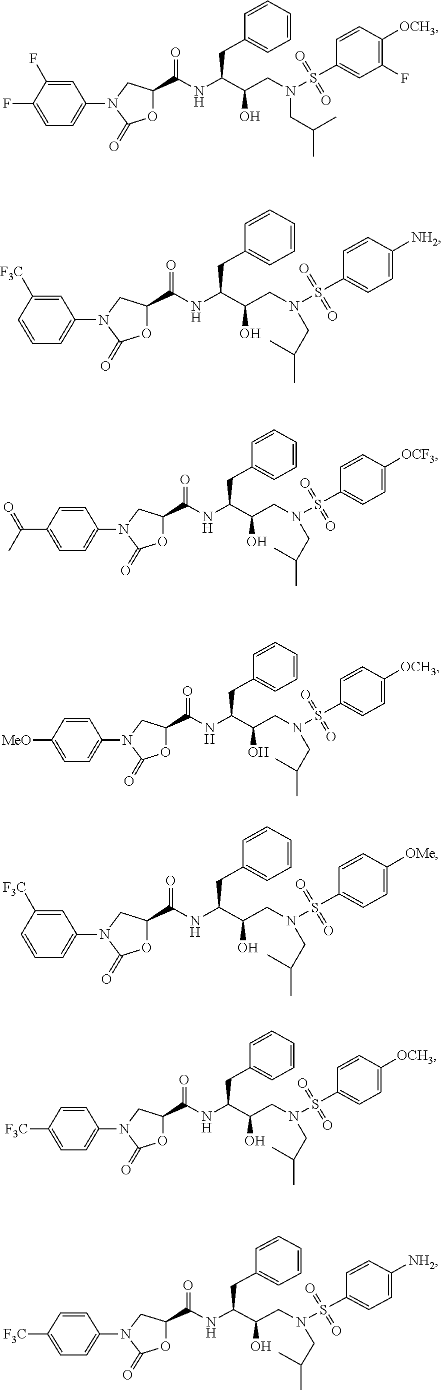 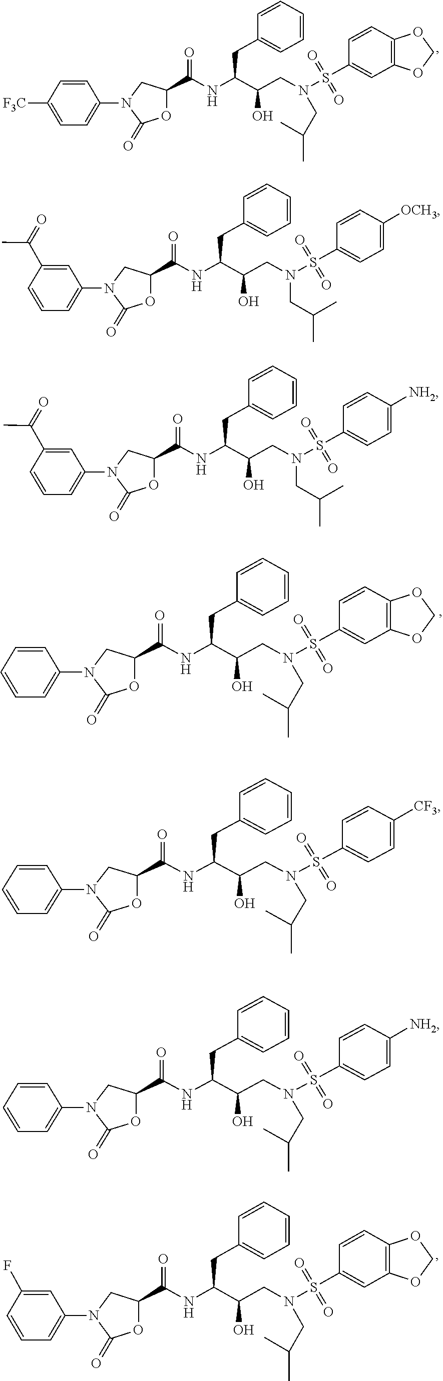 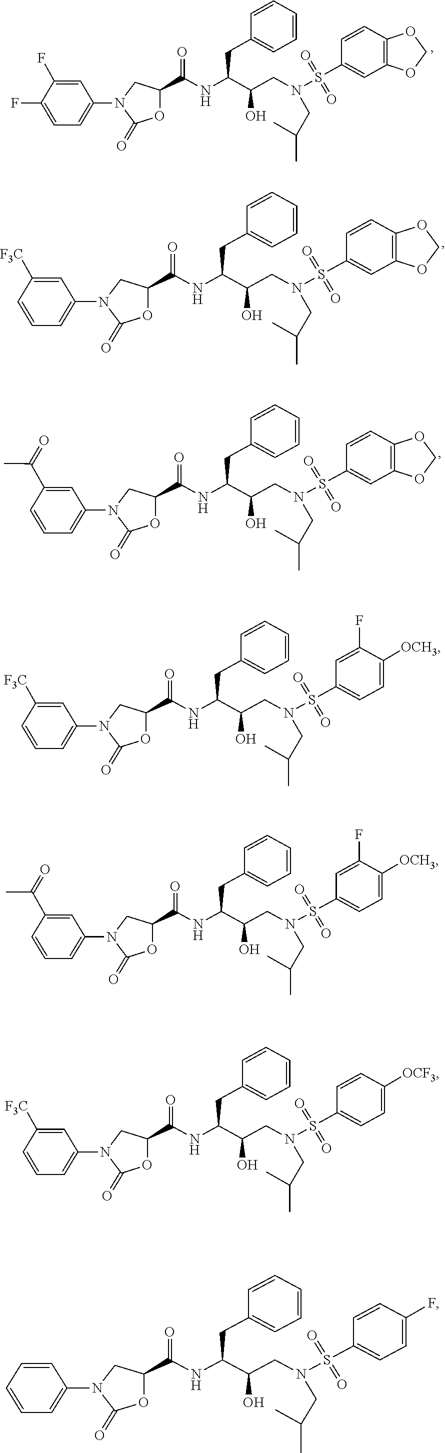 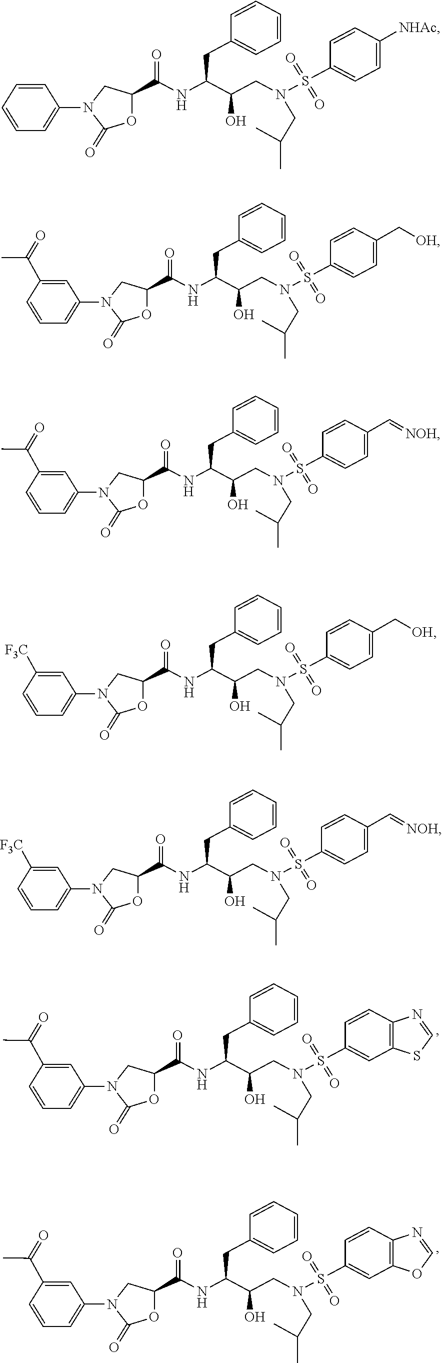 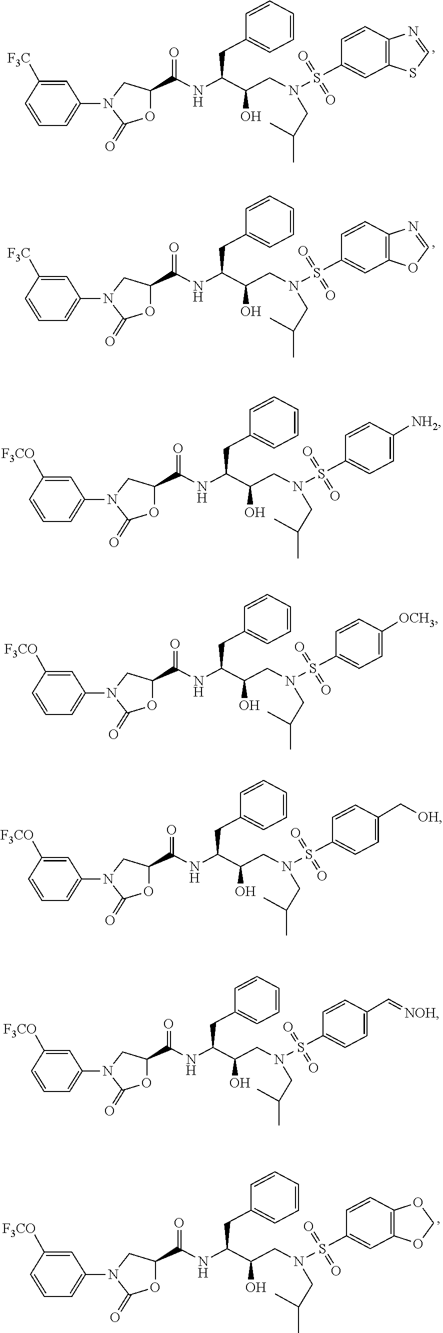 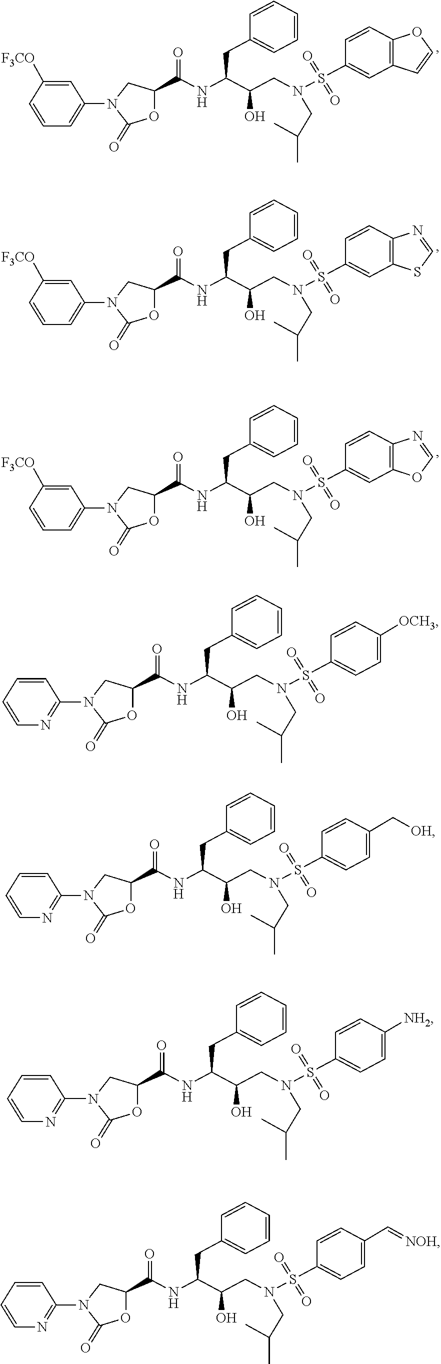 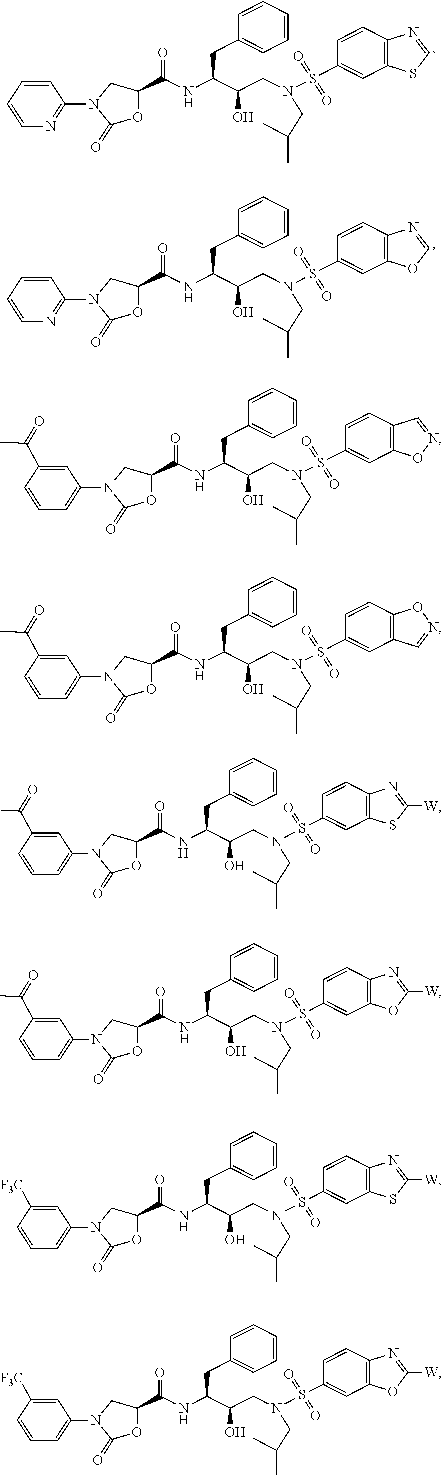 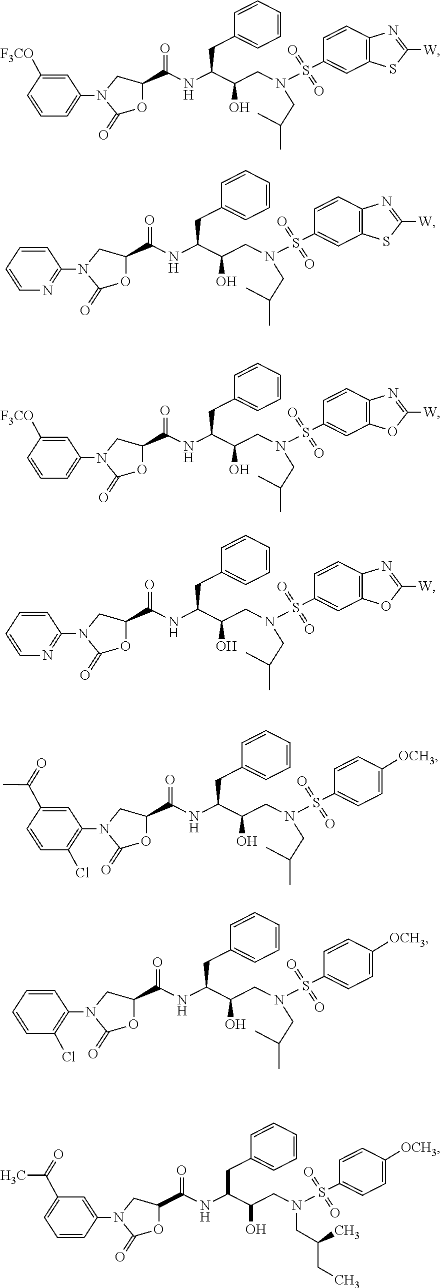 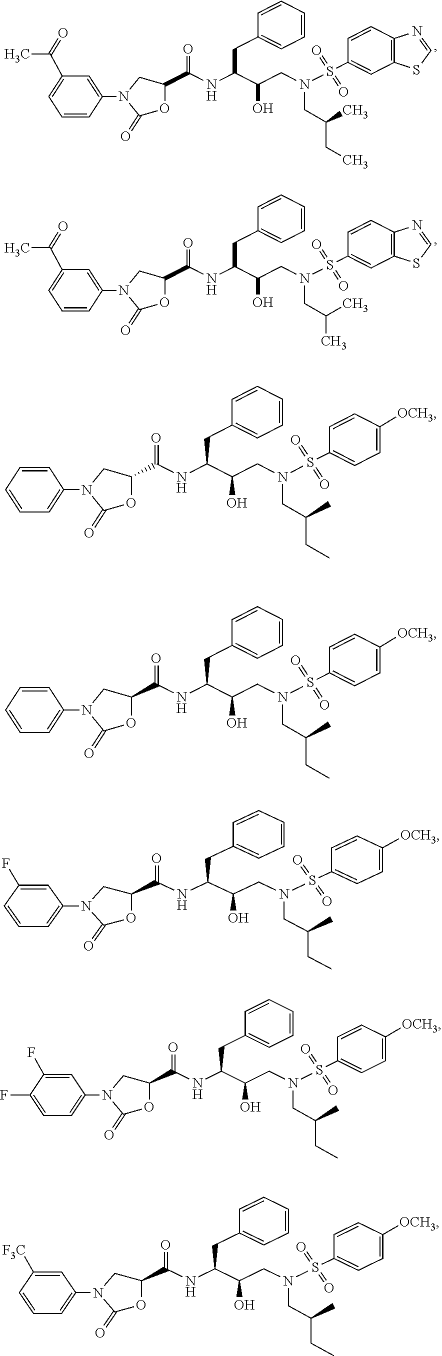 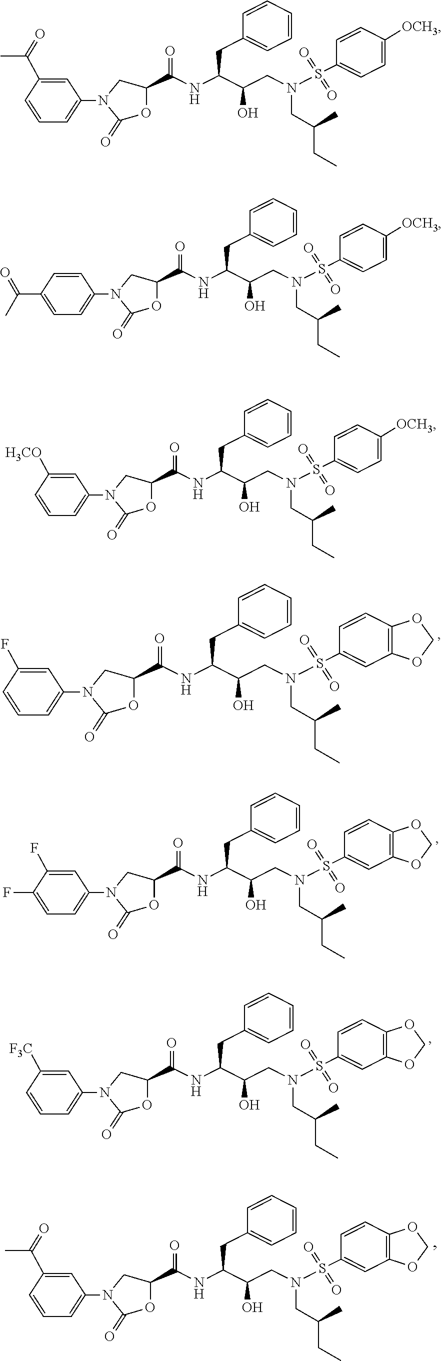 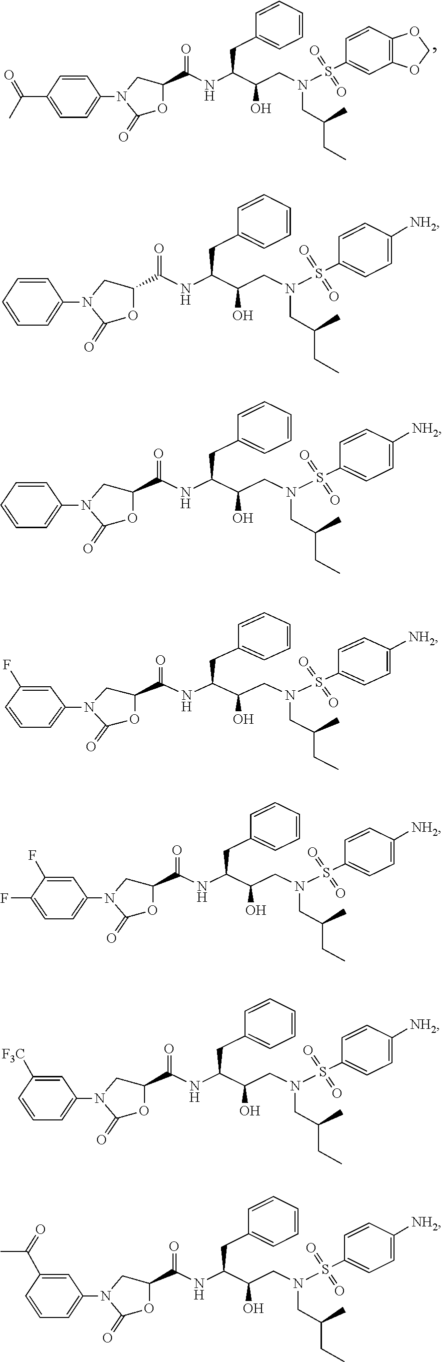 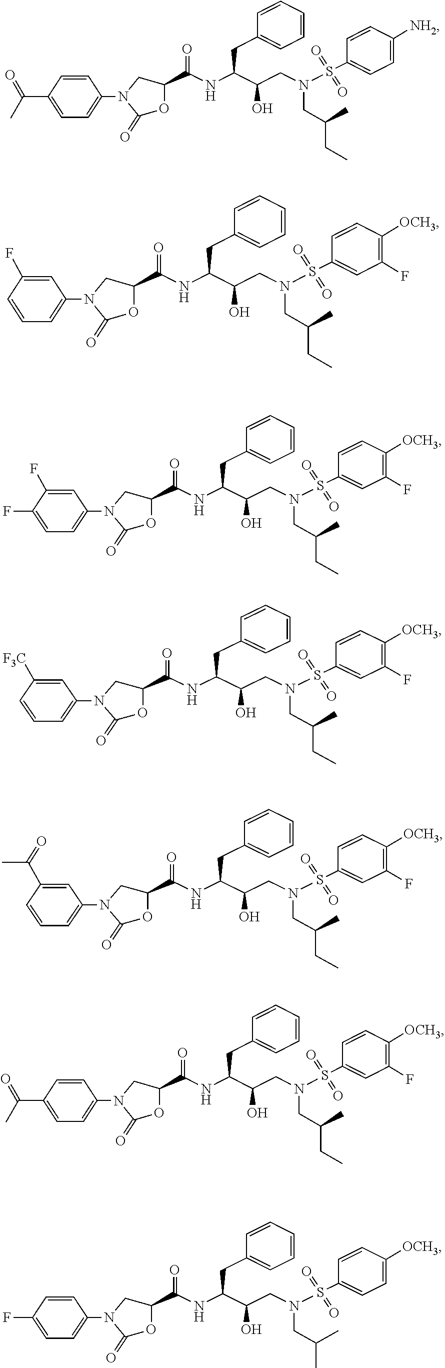 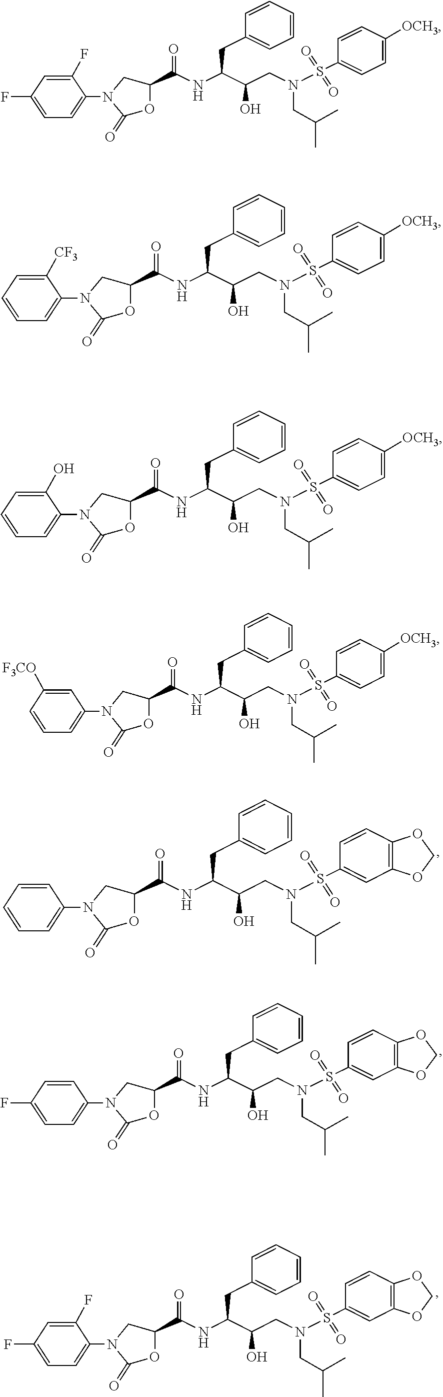 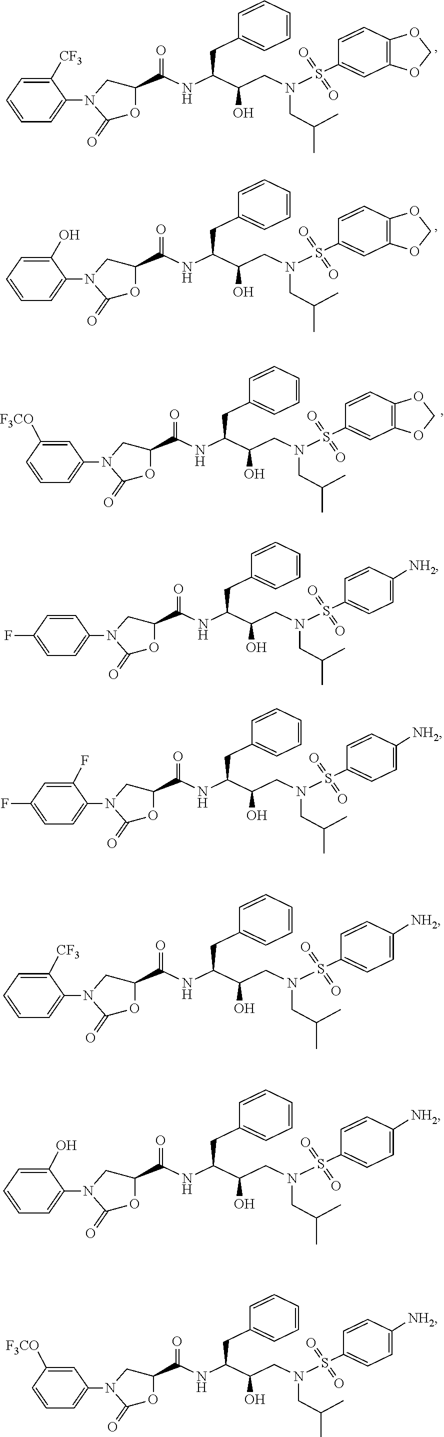 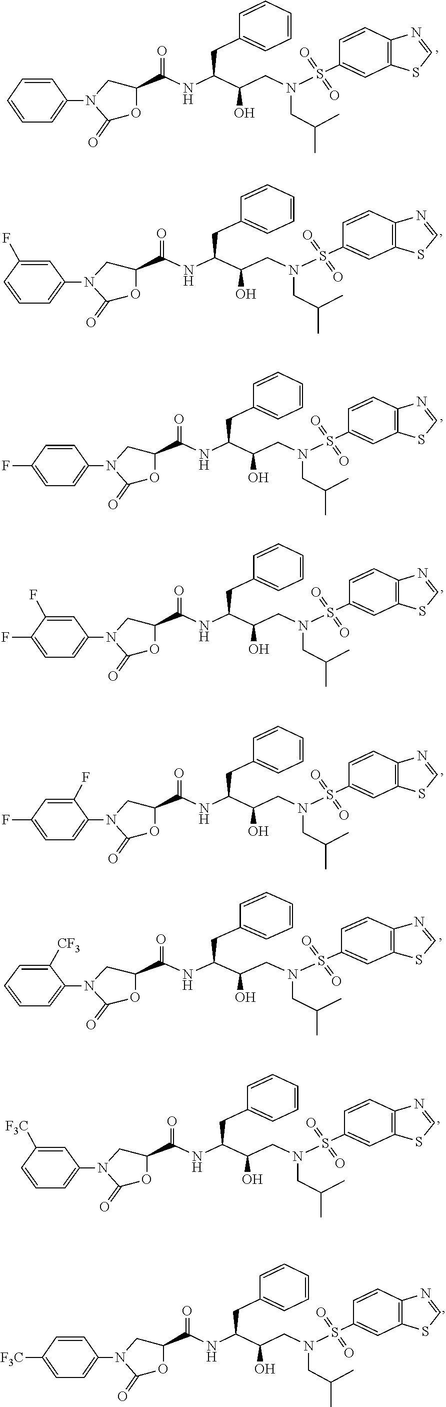 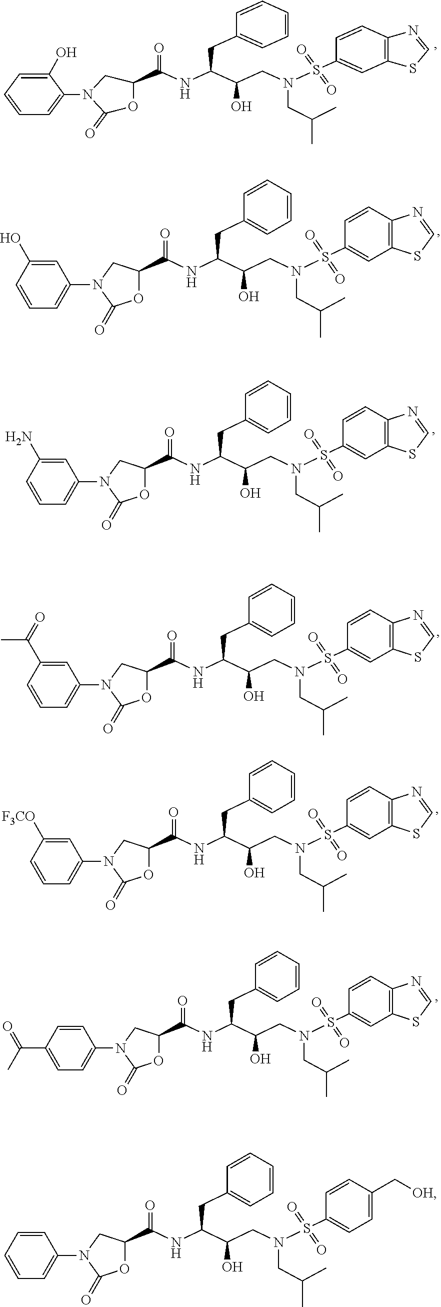 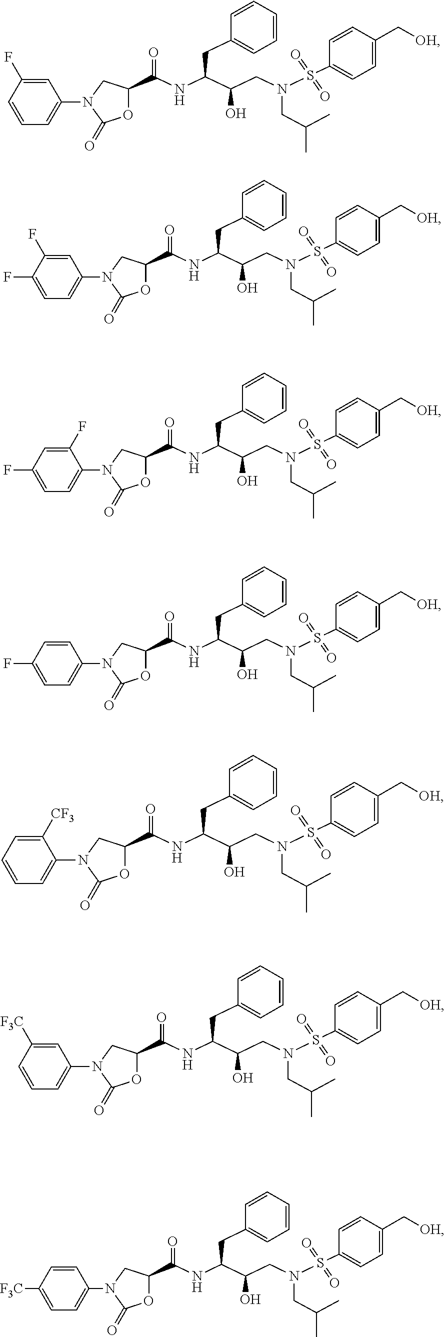 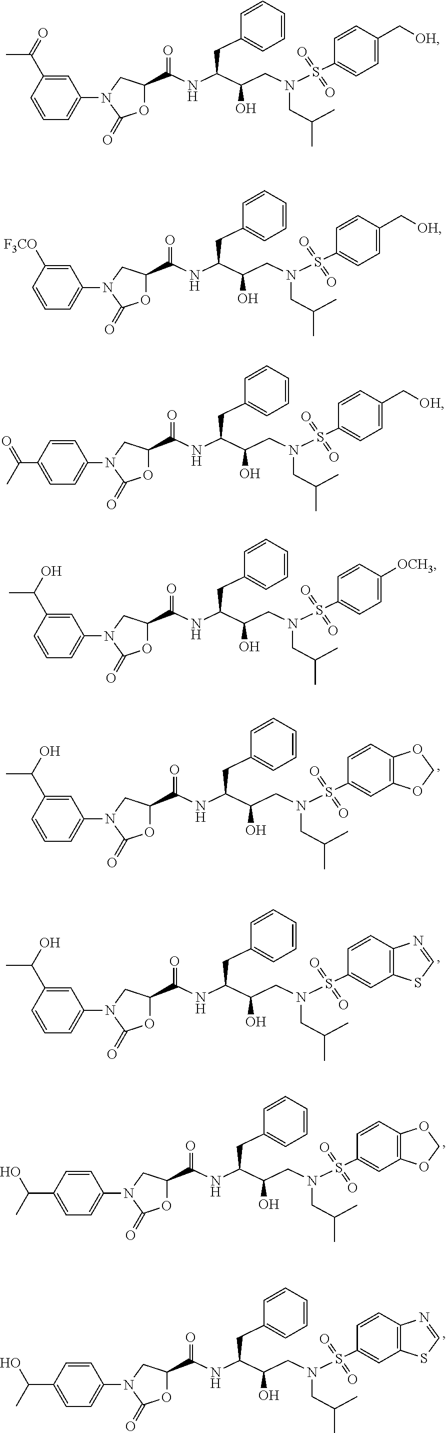 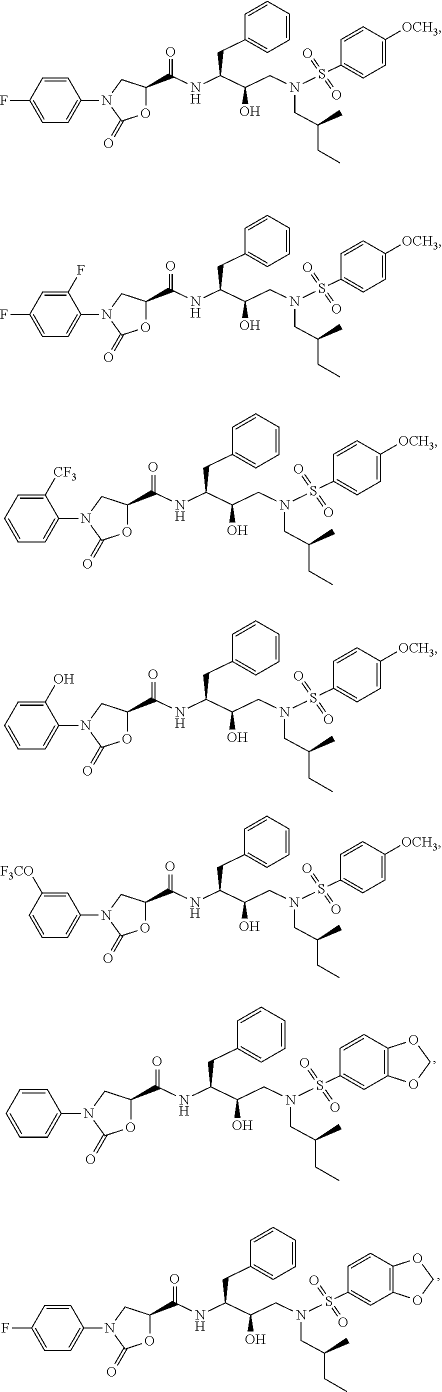 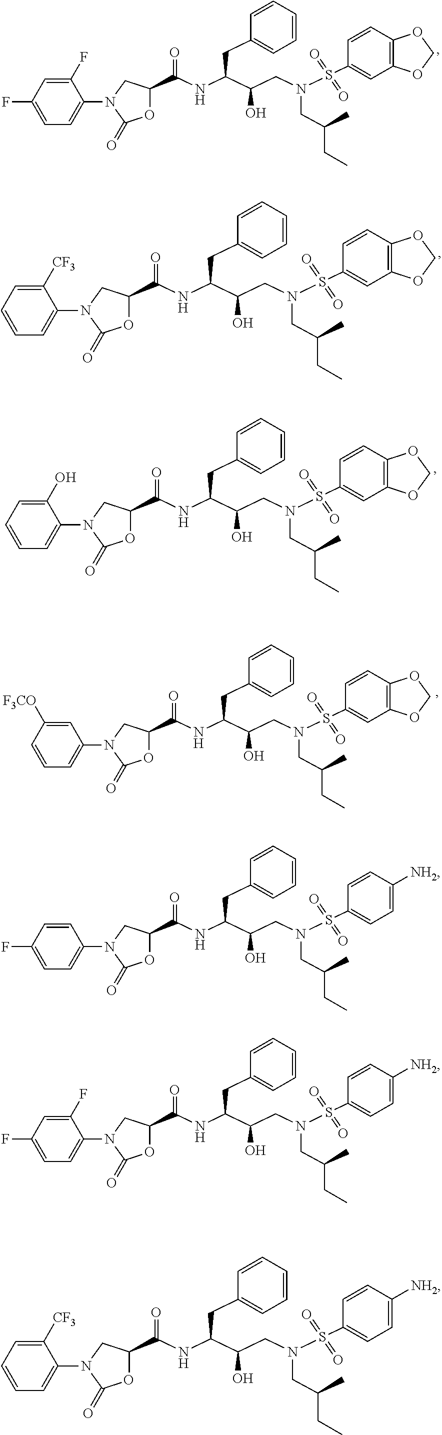 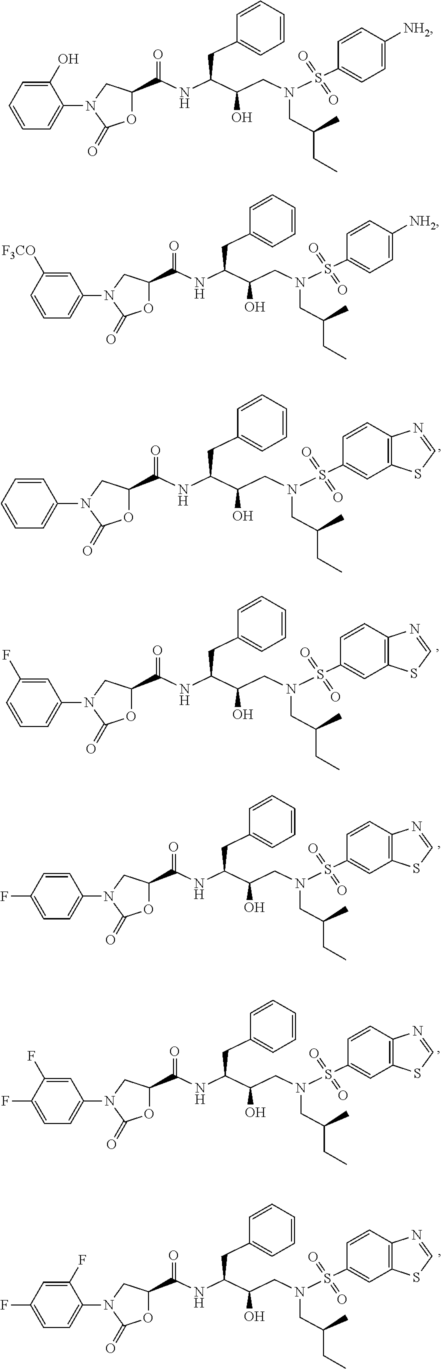 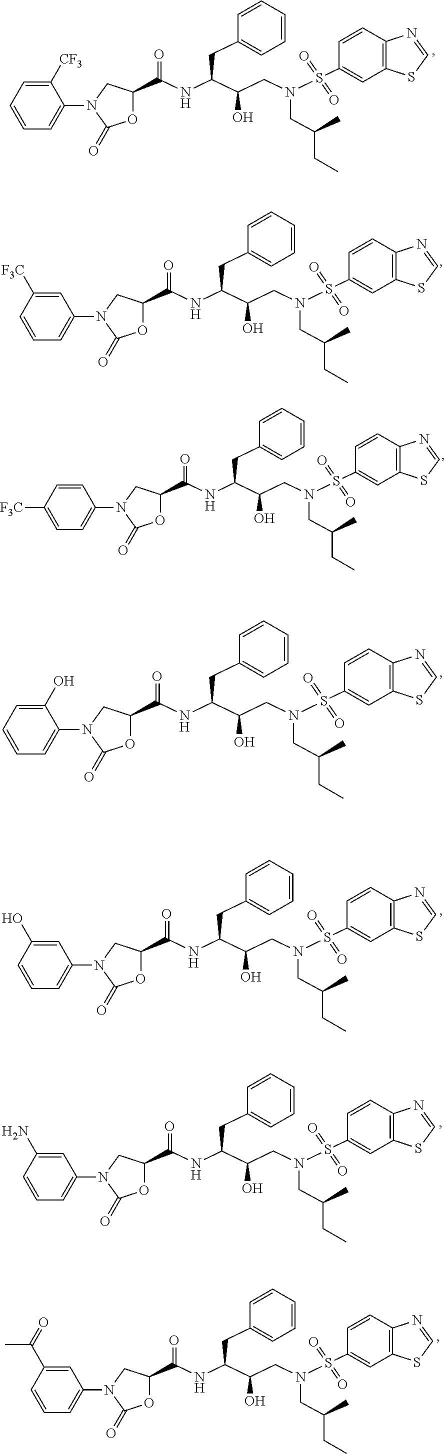 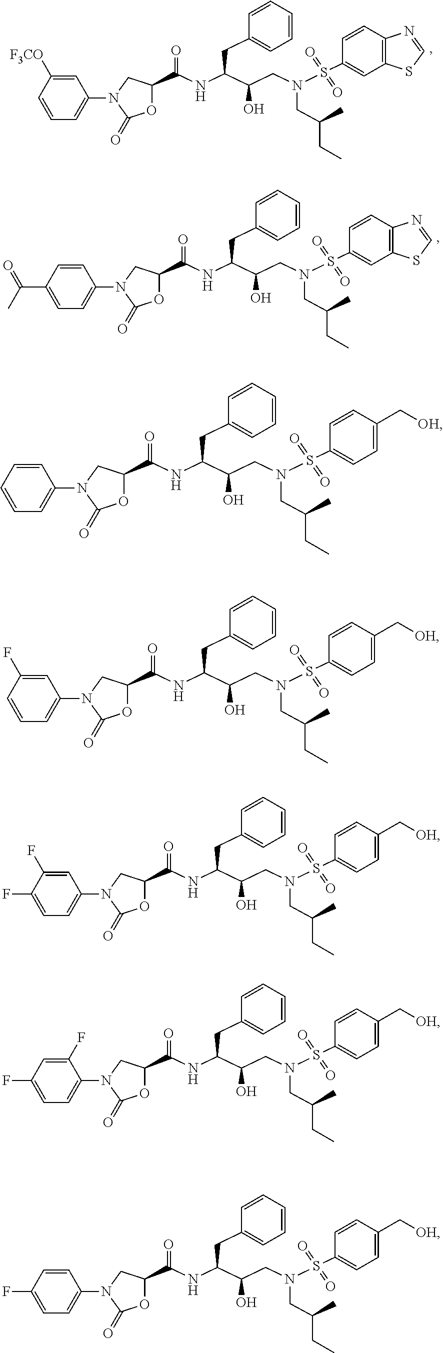 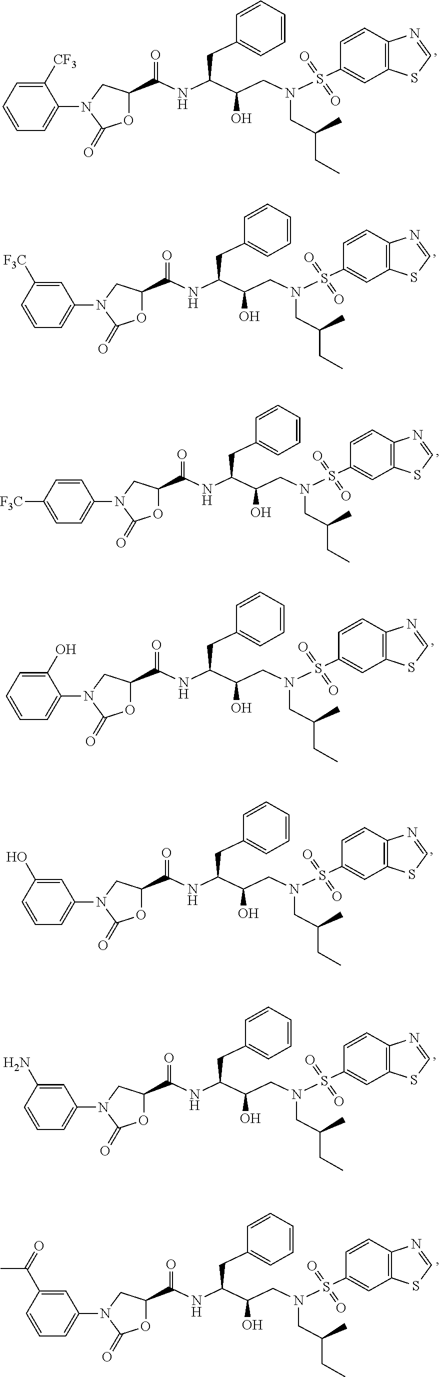 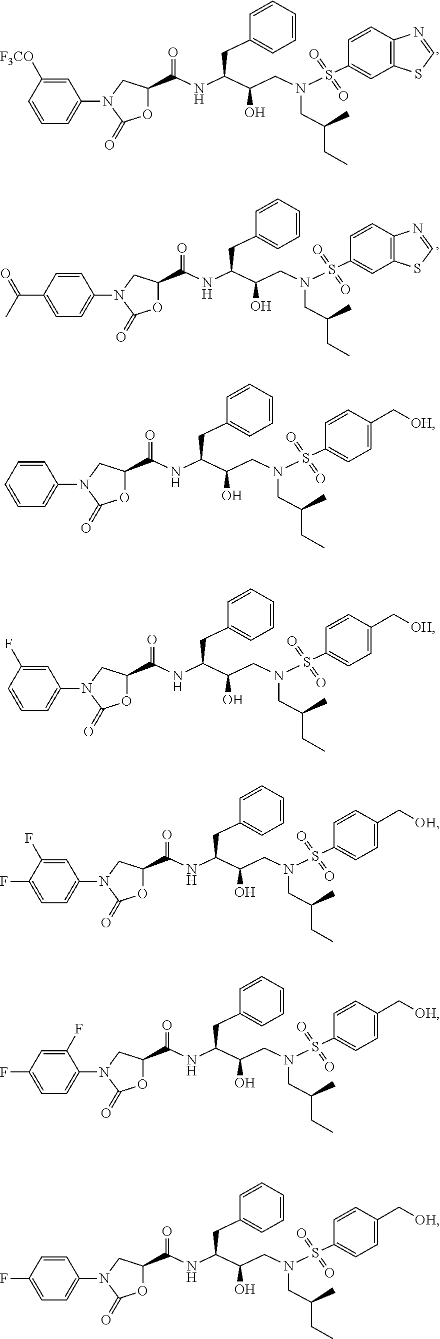 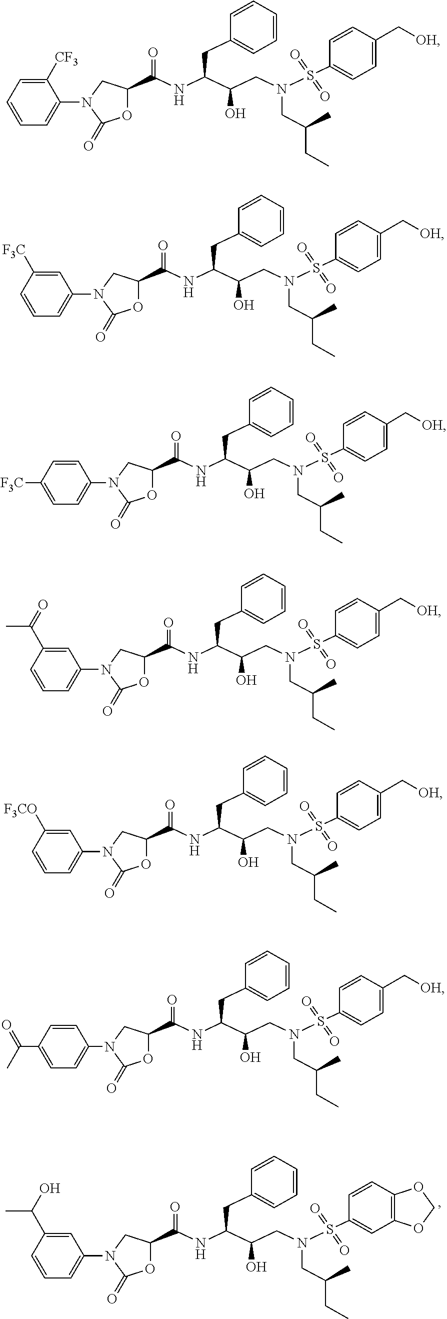 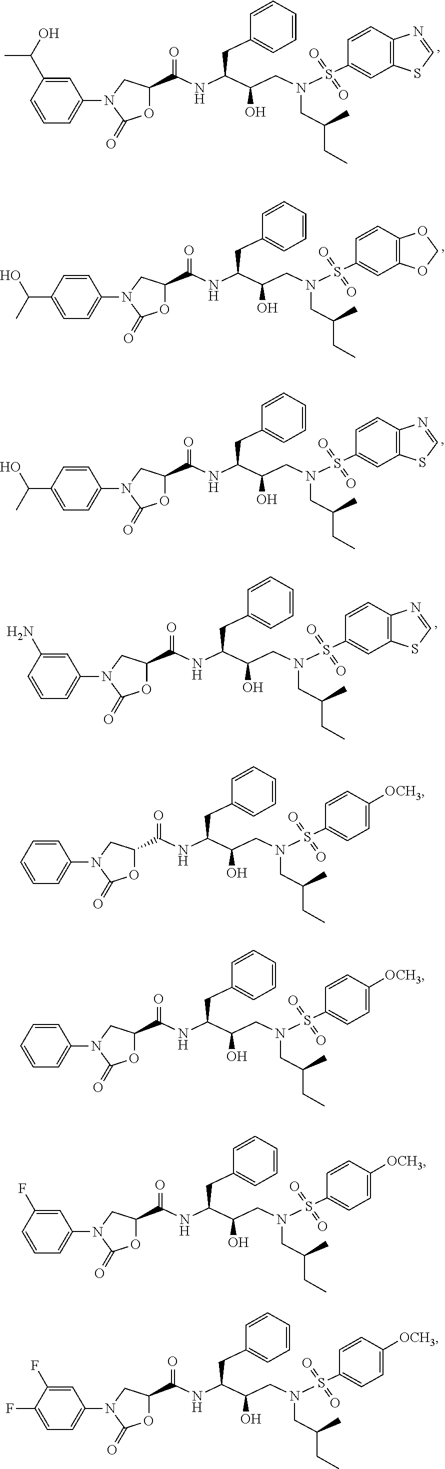 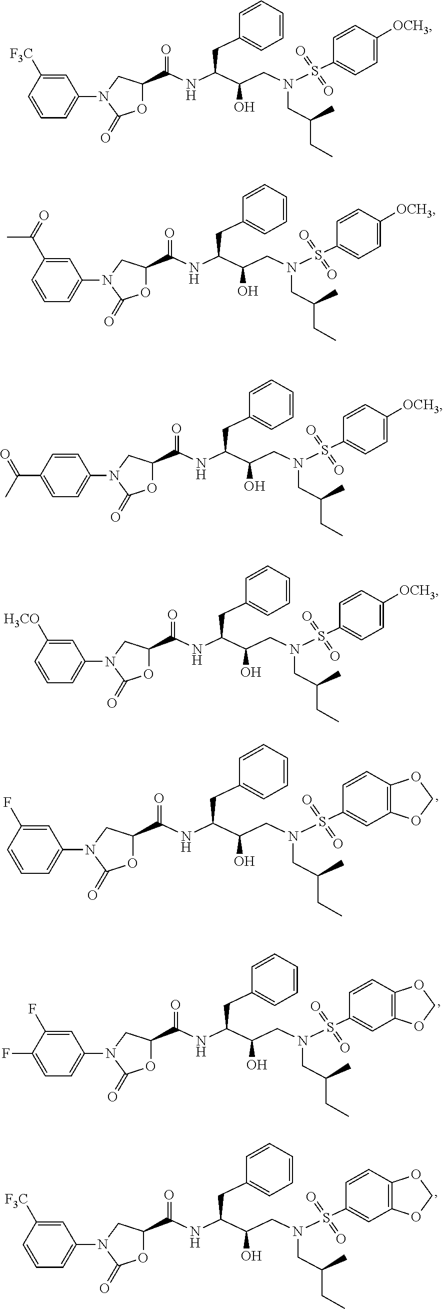 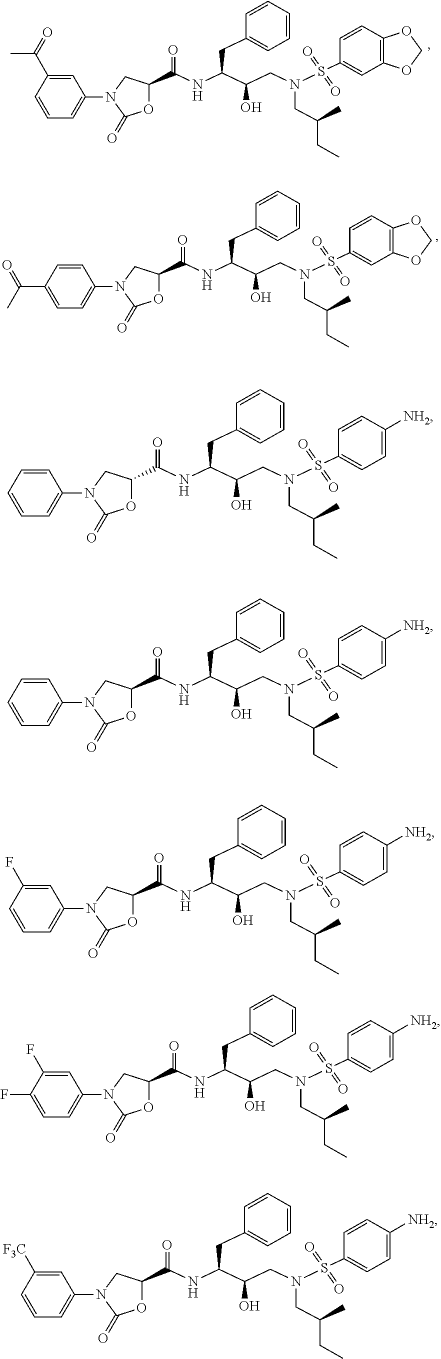 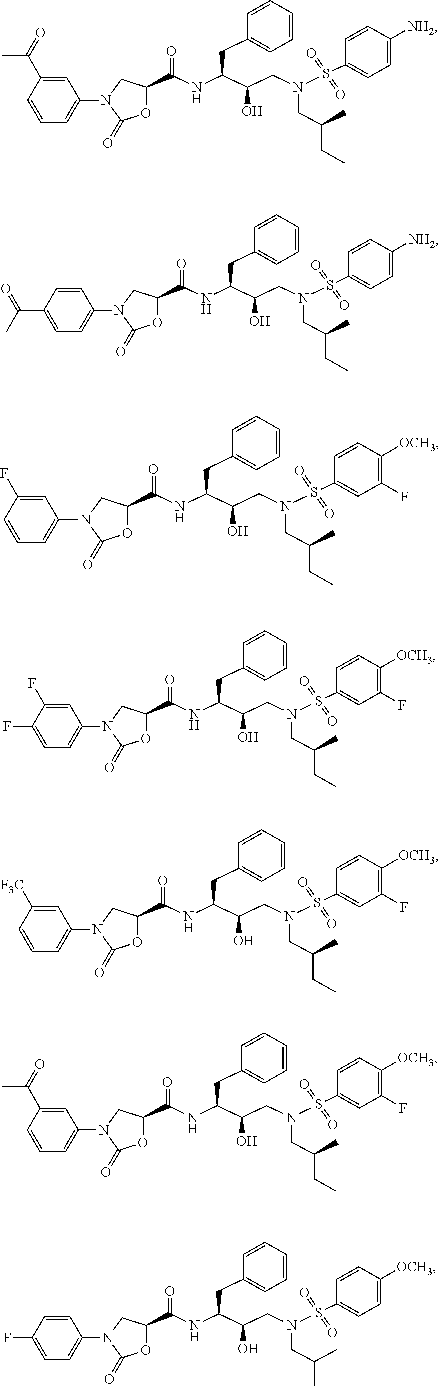 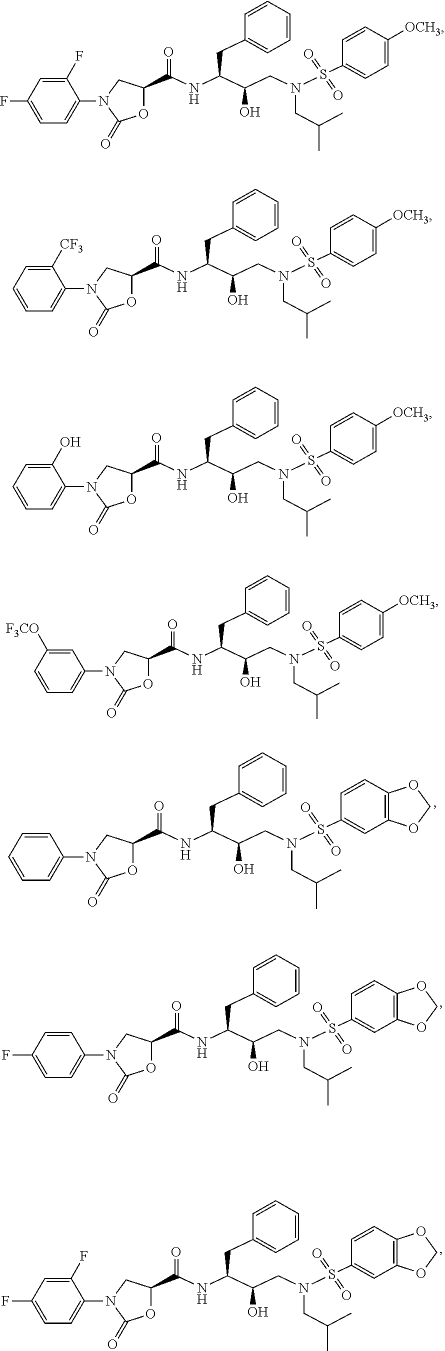 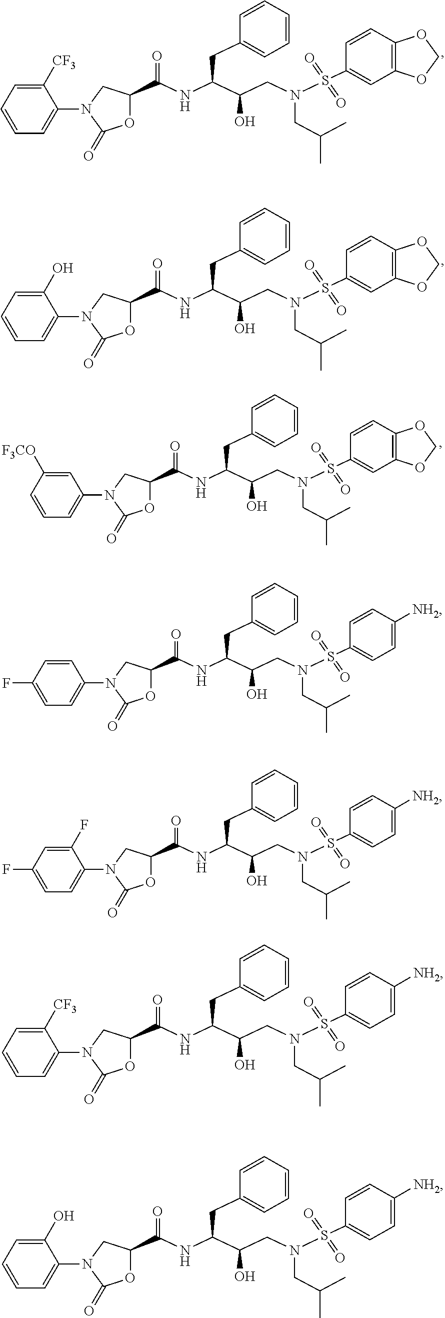 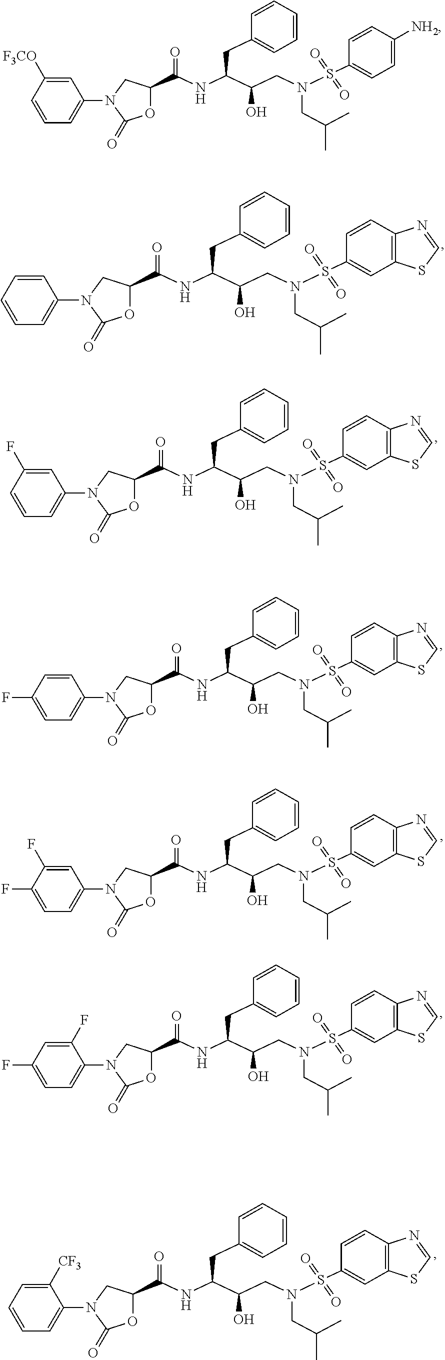 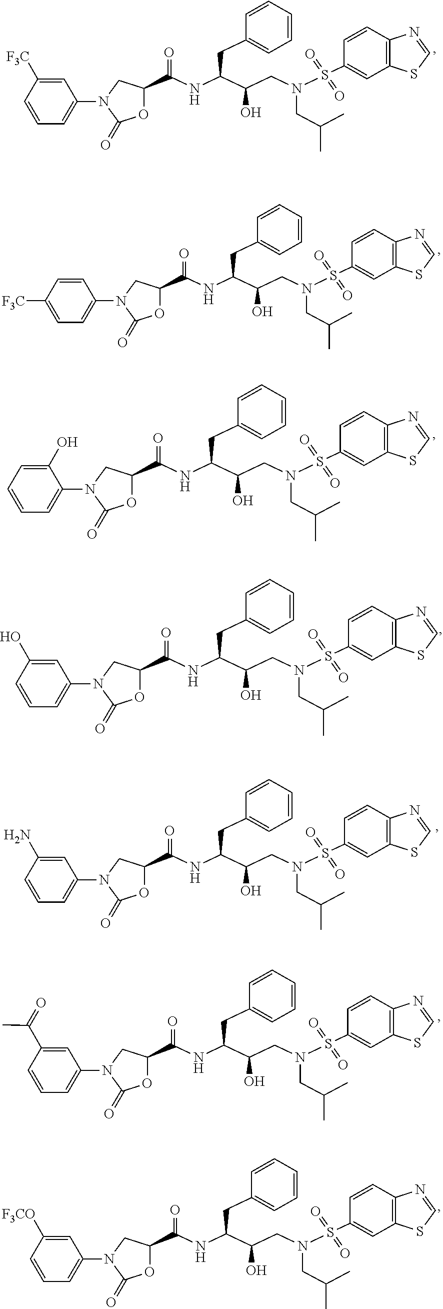 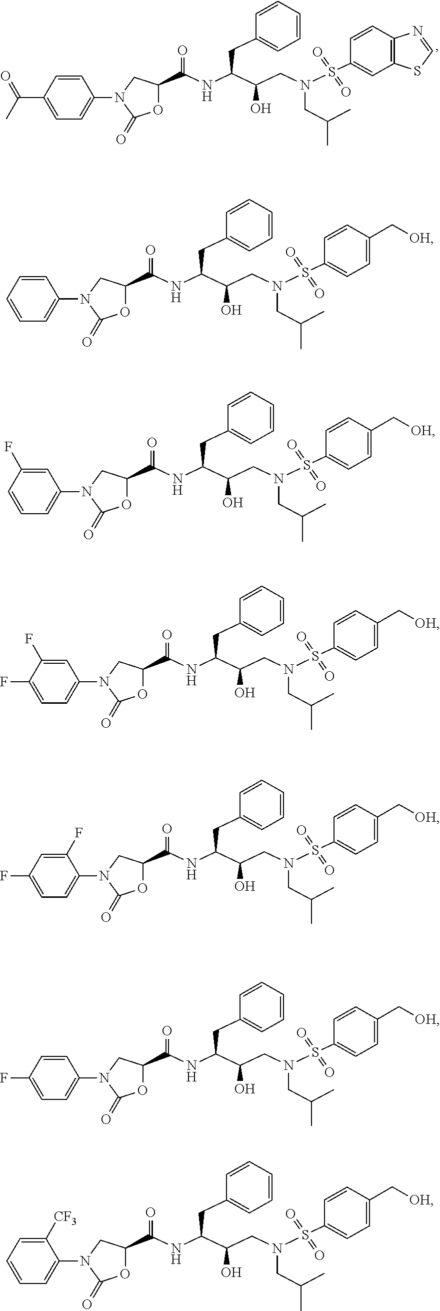 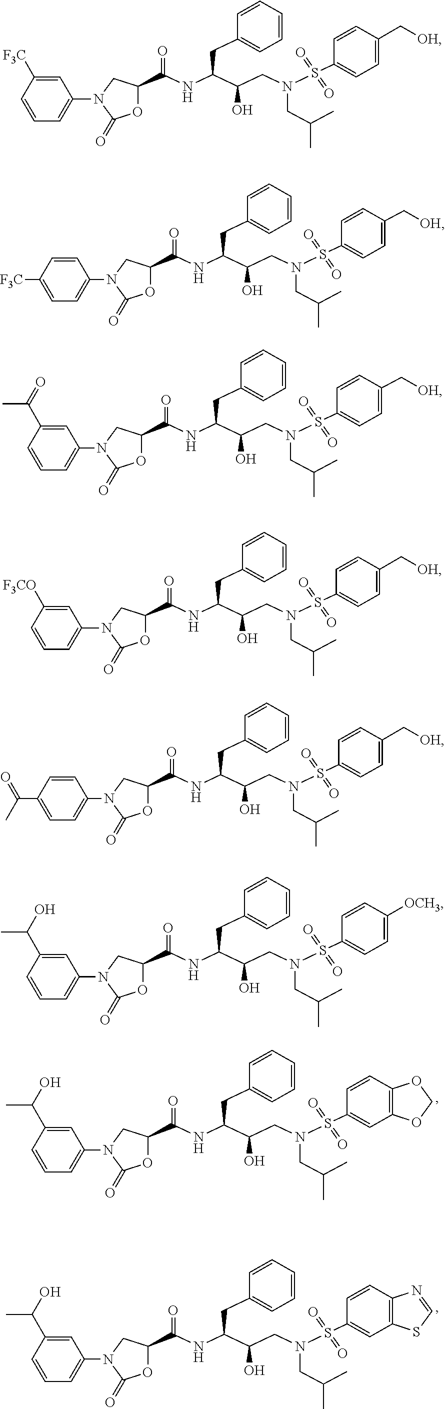 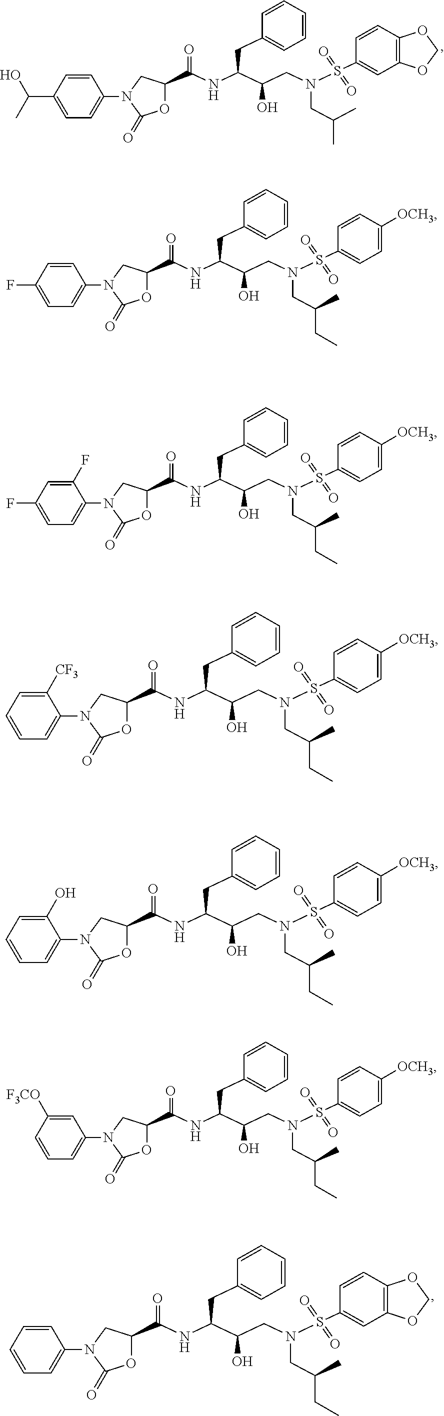 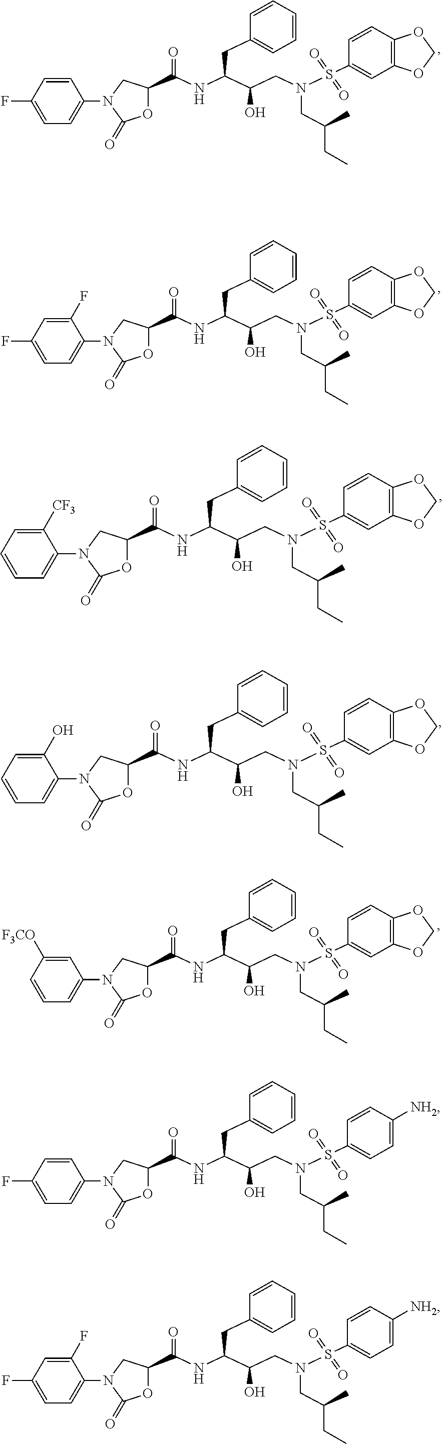 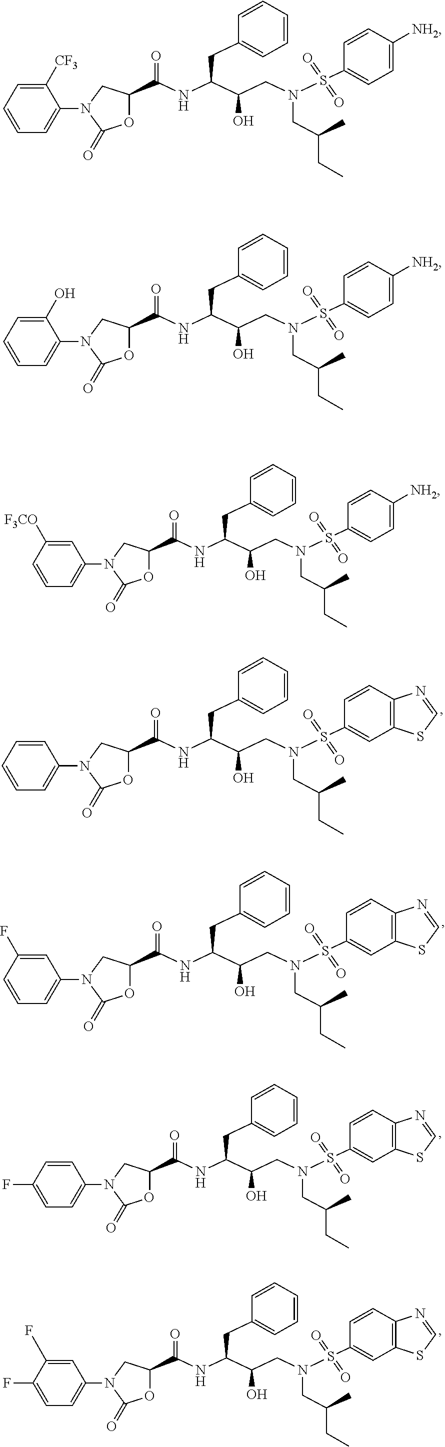 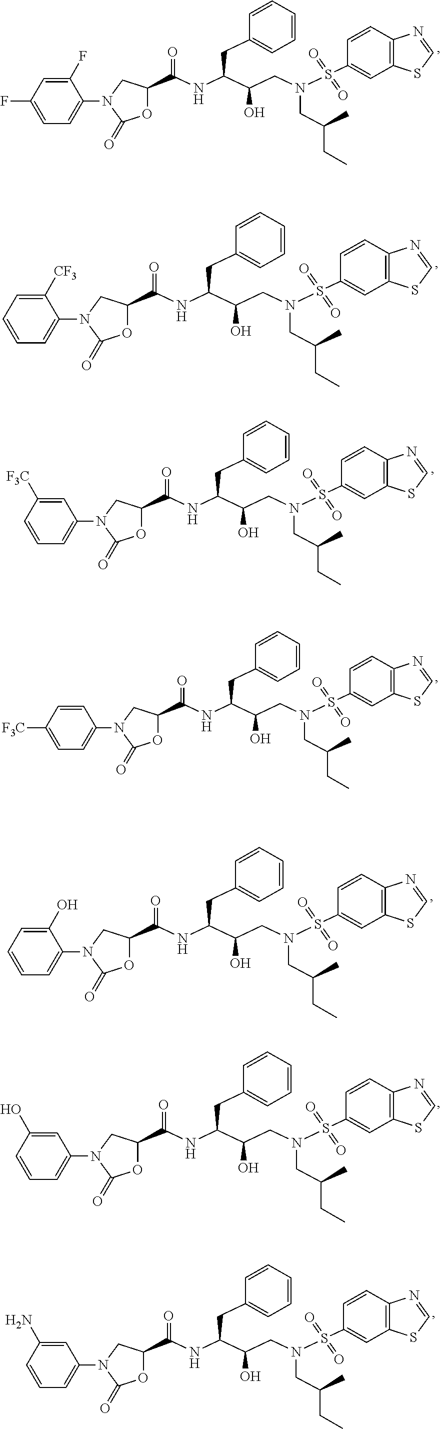 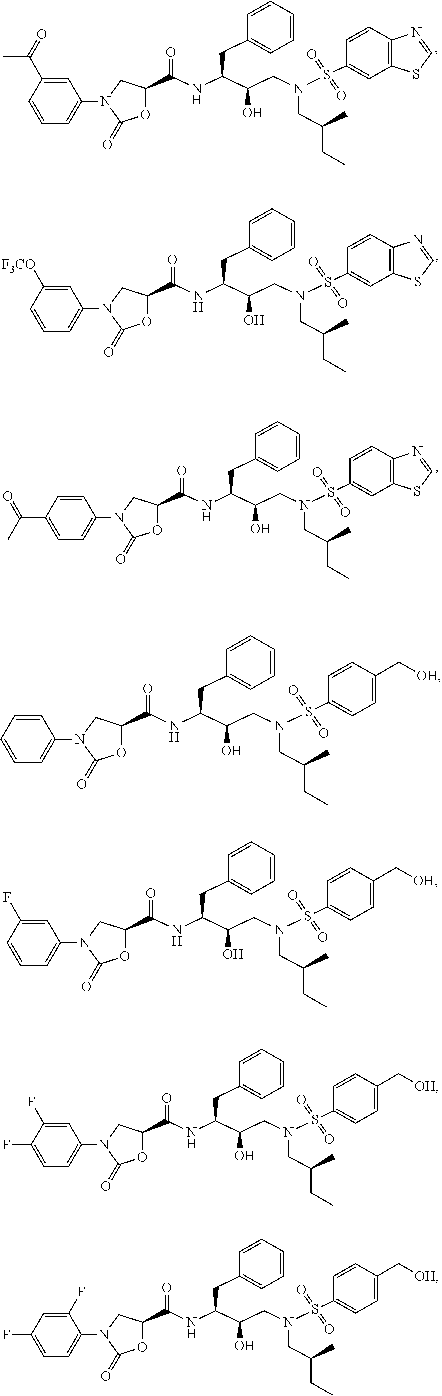 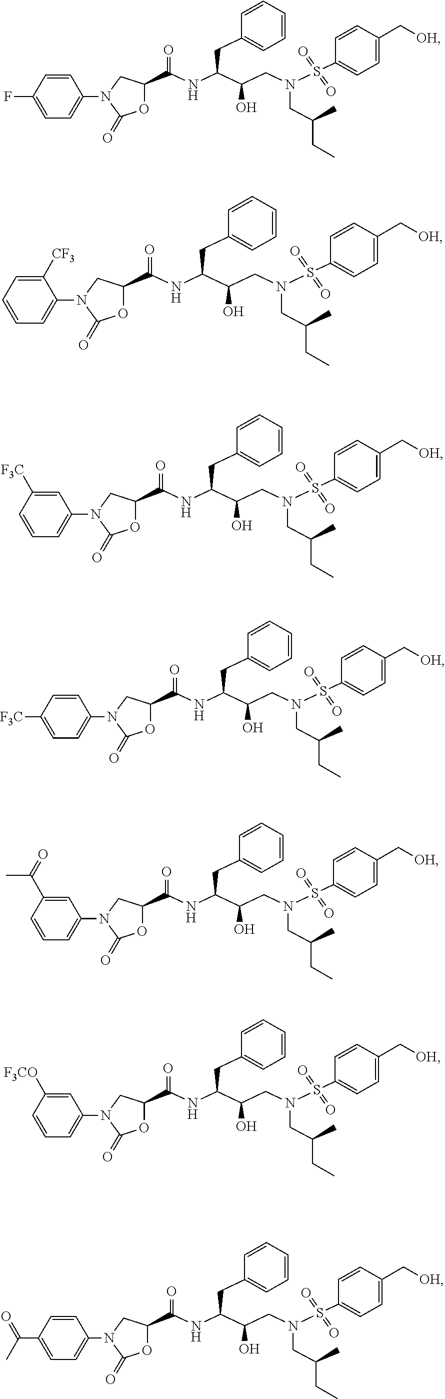 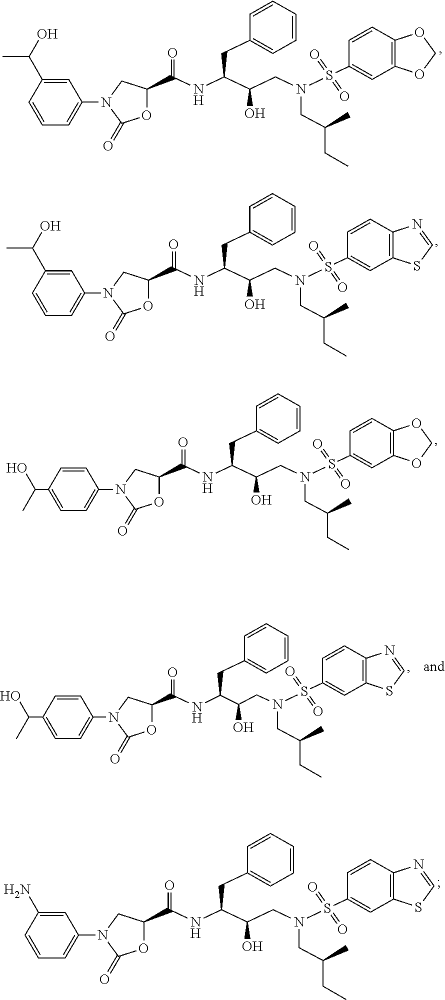
wherein, independently for each occurrence, W is selected, from the group consisting of —NHR⁷ or —NHR(CH₂)ₚN(R⁷)₂; R⁷ is selected from the group consisting of hydrogen, alkyl, aralkyl, heteroaralkyl and acyl; and p is 1-10 inclusive.

150-294. (canceled)

295. A pharmaceutical composition, comprising a pharmaceutical carrier and a therapeutically effective amount of a compound of claim 1.

296. A method for treating an HIV infection, comprising administering to a mammal in need thereof a therapeutically effective amount of a compound of claim 1.

297-311. (canceled)

312. A pharmaceutical composition, comprising a pharmaceutical carrier and a therapeutically effective amount of a compound of claim 110.

313. A method for treating an HIV infection, comprising administering to a mammal in need thereof a therapeutically effective amount of a compound of claims 110.
